(19) United States
(12) Patent Application Publication
(10) Pub. No.: US 2011/0201105 A1
(43) Pub. Date: Aug. 18, 2011

(54) LACTATION-ASSOCIATED POLYPEPTIDES

(75) Inventors: Christophe Marc Lefevre, Victoria (AU); Elie Khalil, Victoria (AU); Julie Sharp, Victoria (AU); Karensa Menzies, Victoria (AU); Kevin Roy Nicholas, Victoria (AU); Paul Sheehy, New South Wales (AU); Yvan Strahm, Bergen (NO); Lisa Riley, New South Wales (AU); Peter Williamson, New South Wales (AU); Kerry Daly, New south Wales (AU); Matthew Robert Digby, Victoria (AU)

(73) Assignees: Dairy Australia Limited, Southbank, Victoria (AU); Innovative Dairy Products PTY LTD, as trustee for the participants of The Cooperative Research Cente, Melbourne (AU)

(21) Appl. No.: 12/672,583

(22) PCT Filed: Aug. 8, 2008
(86) PCT No.: PCT/AU2008/001150

§ 371 Date: Feb. 22, 2011

(30) Foreign Application Priority Data
Aug. 8, 2007 (AU) 2007904259
Nov. 16, 2007 (AU) 2007906289

(51) Int. Cl. C12N 5/10; C12N 15/85; C07K 14/47; C07K 7/08; C07H 21/04

(52) U.S. Cl. 435/325; 435/320.1; 530/350; 530/324; 530/326; 536/23.5

(57) ABSTRACT

The invention relates to cDNA libraries obtained from Tammar wallaby (Macropus eugenii) mammary gland tissue at different times (including day 23 pregnancy, day 130 lactation, and day 260 lactation). Lactation-associated polypeptides were identified from these cDNA libraries, and bovine homologues of the wallaby proteins are also disclosed. The proteins have a range of activities including anti-apoptotic activity, pro- or anti-inflammatory activity, cathelicidin anti-microbial activity, induction of trefoil proteins (and protection of epithelial surfaces), increased cell proliferation, and induction of cell differentiation (and loss of pluripotency).

### TECHNICAL FIELD

The present invention relates generally to polypeptides the expression of which is altered during lactation in mammals. The invention also relates to polynucleotides encoding the same and to uses of these polypeptides and polynucleotides.

### BACKGROUND OF THE INVENTION

Mammalian milk is composed primarily of proteins, sugars, lipids and a variety of trace minerals and vitamins. Milk proteins not only provide nutrition for the developing offspring, but a complex range of biological activities tailored to age-specific needs of the offspring.

It is well recognized that milk composition changes during lactation, the most striking change being that from colostrum to milk shortly after parturition in most mammals. However a variety of other changes in milk composition occur throughout lactation. The extent and full biological significance of the changes is presently unknown although it is accepted that milk composition alterations at least in part reflect the changing needs of the offspring through stages of development and/or is regulate such developmental changes.

The major protein constituents of milk are the casein proteins, α-casein and β-casein, α-lactalbumin and β-lactoglobulin. Milk also contains significant antimicrobial and immune response mediators. Well known constituents include antibodies, lysozyme, lactoferrin complement proteins C3/C4, defensins, and interleukins including IL-1, IL-10 and IL-12. In addition to these a vast array of other proteins are also present in milk, many of which remain to be identified and characterized. A significant number of these uncharacterized proteins are likely to play a regulatory role and/or contribute to the development or protection of the offspring, for example by providing antimicrobial activities, anti-inflammatory activities or by boosting the immune system of the offspring. There is a clear need to elucidate the identities and activities of such proteins.

Marsupials have a number of unique features in their modes of reproduction and lactation which make them excellent model organisms for the study of changes in milk composition, and specifically milk proteins. Lactation in marsupials has been studied extensively; one of the most widely studied marsupials being the tammar wallaby (Macropus eugenii). The lactation cycle in the tammar wallaby can be divided into 4 phases, phase 1, phase 2A, phase 2B and phase 3 (see Nicholas et al., 1997, J Mammary Gland Blot Neoplasia 2: 299-310). The transition from one phase to the next correlates with significant alterations in milk composition, in particular in milk protein concentrations. Milk composition is specifically matched for the developmental stage of the offspring. Macropodids such as the tammar wallaby are capable of concurrent asynchronous lactation whereby individual teats produce milk with different compositions for pouch young of different ages. As such lactation can be independently regulated locally rather than systemically, determining the rate of growth and development of the young irrespective of the age of the young (Nicholas et al., 1997; Trott et al., 2003, Biol Reprod 68:929-936). Additionally, marsupial young are altricial and thus totally dependent on maternal milk in the early stages of life. For example, tammar wallaby pouch young have no immune system of their own for approximately the first 70 days and depend entirely on the protection offered by maternal milk. The above features, inter alia, make marsupials excellent experimental model organisms for the investigation of regulatory and bioactive proteins in milk. In addition, bovine polynucleotides and corresponding polypeptides will be tested to further elucidate regulatory and bioactive proteins in mammalian milk.

Further, with the rapid progress of comparative gene mapping techniques and genome sequencing technology, genetic studies in marsupials have already proven instrumental in the identification of novel genes in other species. For example, studies in the tammar wallaby led to the discovery of a candidate gene for mental retardation, RBMX, in humans (Delbridge of al., 1999, Nat Genet 22: 223-224).

The present invention is predicated on the inventors' use of the tammar wallaby and the cow as model systems for the identification of lactation-associated polypeptides secreted in mammalian milk and on the identification of bioactivities of secreted polypeptides including the identification of homologues.

### SUMMARY OF THE INVENTION

According to a first aspect of the present invention, there is provided a lactation-associated polypeptide, wherein said polypeptide is encoded by:

(a) a polynucleotide of a cDNA library as shown in Table 1; or

(b) a functionally equivalent variant, analogue, derivative or fragment of a polynucleotide of a cDNA library as shown in Table 1.

The polypeptide may be a secreted polypeptide.
The polypeptide may exhibit bioactivity. The bioactivity may include:

(a) regulation of mammary gland development including mammosphere formation;

(b) regulation of lactation;

(c) regulation of milk composition;

(d) growth-promoting activity, including cell proliferative activity, including cellular differentiation and/or morphology activity;

(e) pro- or anti-inflammatory activity;

(f) pro- or anti-apoptotic activity;

(g) anti-microbial activity;

(h) regulation of differentiation of embryonic stem cells;

(i) regulation of trefoil activity;

(j) cathelicidin activity;

(k) regulation of epithelial cells, including gut and/or skin epithelial cells; and/or

(l) regulation of growth of chicks.

According to a second aspect of the present invention, there is provided a lactation-associated polypeptide, wherein said polypeptide comprises an amino acid sequence selected from the group consisting of SEQ ID NOs: 31-59, 91-119, 152, 154, 156, 158, 160, 162, 164, 166, 171, 173, 175, 177, 179, 181, 184, 186, 188, 207, 215, 217 and 219 and or a functionally equivalent variant, analogue, derivative or fragment thereof.

According to a third aspect of the present invention, there is provided (a) a polynucleotide encoding a polypeptide of the first or second aspects, or a functionally equivalent variant, analogue, derivative or fragment thereof, or (b) any other polynucleotide that would hybridise to any one of the polynucleotides selected from (a) under conditions of high stringency.

According to a fourth aspect of the present invention, there is provided a lactation-associated polynucleotide, wherein said polynucleotide comprises (a) a nucleic acid sequence is selected from the group consisting of SEQ ID NOs: 1-29, 61-89, 151, 153, 155, 157, 159, 161, 163, 165, 167-170, 172, 174, 176, 178, 180, 183, 185, 190-192, 208, 216 and 218 or a functionally equivalent variant, analogue, derivative or fragment thereof, or (b) any other polynucleotide that would hybridise to any one of the polynucleotides selected from (a) under conditions of high stringency.

According to a fifth aspect of the present invention, there is provided a polypeptide encoded by a polynucleotide of the fourth aspect.

According to a sixth aspect of the present invention, there is provided an expression vector comprising a polynucleotide of the third or fourth aspects.

The polynucleotide may be operably linked to a promoter.

According to a seventh aspect of the present invention, there is provided a host cell transformed with an expression vector of the sixth aspect.

According to an eighth aspect of the present invention, there is provided a bioactive molecule, the molecule having cell growth-promoting activity, wherein the molecule comprises a polypeptide comprising an amino acid sequence selected from the group as set forth in SEQ ID NOs: 32, 34-35, 37, 42, 48, 51-53, 55, 92, 94-95, 97, 102, 108, 111-113, 115 and 152 or a functionally equivalent variant, analogue, derivative or fragment thereof.

According to a ninth aspect of the present invention, there is provided a bioactive molecule, the molecule having cell growth-promoting activity, wherein the molecule is encoded by (a) a polynucleotide comprising a nucleotide sequence selected from the group as set forth in SEQ ID NOs: 2, 4-5, 7, 12, 18, 21-23, 25, 62, 64-65, 67, 72, 78, 81-83, 85 and 151 or a functionally equivalent variant, analogue, derivative or fragment thereof, or (b) any other polynucleotide that would hybridise to any one of the polynucleotides selected from (a) under conditions of high stringency.

According to a tenth aspect of the present invention, there is provided a bioactive molecule, the molecule having pro-apoptotic activity, wherein the molecule comprises a polypeptide comprising an amino acid sequence selected from the group as set forth in SEQ ID NOs: 46-47, 106-107, and 156 or a functionally equivalent variant, analogue, derivative or fragment thereof.

According to an eleventh aspect of the present invention, there is provided a bioactive molecule, the molecule having pro-apoptotic activity, wherein the molecule is encoded by (a) a polynucleotide comprising a nucleotide sequence selected from the group as set forth in SEQ ID NOs: 16-17, 76-77, and 155 or a functionally equivalent variant, analogue, derivative or fragment thereof, or (b) any other polynucleotide that would hybridise to any one of the polynucleotides selected from (a) under conditions of high stringency.

According to a twelfth aspect of the present invention, there is provided a bioactive molecule, the molecule having anti-apoptotic activity, wherein the molecule comprises a polypeptide comprising an amino acid sequence selected from the group as set forth in SEQ ID NOs: 43 and 103 or a functionally equivalent variant, analogue, derivative or fragment thereof.

According to a thirteenth aspect of the present invention, there is provided a bioactive molecule, the molecule having anti-apoptotic activity, wherein the molecule is encoded by (a) a polynucleotide comprising a nucleotide sequence selected from the group as set forth in SEQ ID NOs: 13 and 73 or a functionally equivalent variant, analogue, derivative or fragment thereof, or (b) any other polynucleotide that would hybridise to any one of the polynucleotides selected from (a) under conditions of high stringency.

According to a fourteenth aspect of the present invention, there is provided a bioactive molecule, the molecule having pro-inflammatory activity, wherein the molecule comprises a polypeptide comprising an amino acid sequence selected from the group as set forth in SEQ ID NOs: 31, 35, 41, 45-46, 49, 91, 95, 101, 105-106, 109, 154 and 156 or a functionally equivalent variant, analogue, derivative or fragment thereof.

According to a fifteenth aspect of the present invention, there is provided a bioactive molecule, the molecule having pro-inflammatory activity, wherein the molecule is encoded by (a) a polynucleotide comprising a nucleotide sequence selected from the group as set forth in SEQ ID NOs: 1, 5, 11, 15-16, 19, 61, 65, 71, 75-76, 79, 153, and 155 or a functionally equivalent variant, analogue, derivative or fragment thereof, or (b) any other polynucleotide that would hybridise to any one of the polynucleotides selected from (a) under conditions of high stringency.

According to a sixteenth aspect of the present invention, there is provided a bioactive molecule, the molecule having anti-inflammatory activity, wherein the molecule comprises a polypeptide comprising an amino acid sequence selected from the group as set forth in SEQ ID NOs: 35-36, 41, 47, 95-96, 101, 107, or a functionally equivalent variant, analogue, derivative or fragment thereof.

According to a seventeenth aspect of the present invention, there is provided a bioactive molecule, the molecule having anti-inflammatory activity, wherein the molecule is encoded by (a) a polynucleotide comprising a nucleotide sequence selected from the group as set forth in SEQ ID NOs: 5-6, 11, 17, 65-66, 71, 77, or a functionally equivalent variant, analogue, derivative or fragment thereof, or (b) any other polynucleotide that would hybridise to any one of the polynucleotides selected from (a) under conditions of high stringency.

According to an eighteenth aspect of the present invention, there is provided a bioactive molecule, the molecule having the ability to regulate cellular morphology, wherein the molecule comprises a polypeptide comprising an amino acid sequence selected from the group as set forth in SEQ ID NO: 33, 37-40, 54, 93, 97-100, 114, 158 and 188 or a functionally equivalent variant, analogue, derivative or fragment thereof.

According to a nineteenth aspect of the present invention, there is provided a bioactive molecule, the molecule having the ability to regulate cellular morphology, wherein the molecule is encoded by (a) a polynucleotide comprising a nucleotide sequence selected from the group as set forth in SEQ ID NOs: 3, 7-10, 24, 63, 67-70, 84, 157, 167, 191 and 208 or a functionally equivalent variant, analogue, derivative or fragment thereof, or (b) any other polynucleotide that would hybridise to any one of the polynucleotides selected from (a) under conditions of high stringency.

According to a twentieth aspect of the present invention, there is provided a bioactive molecule, the molecule having the ability to influence cellular differentiation, wherein the molecule comprises a polypeptide comprising an amino acid sequence selected from the group as set forth in SEQ ID NOs: 37, 44, 49-50, 54-59, 97, 104, 109-110, 114-119, 158, 160, 162, 164, and 166 or a functionally equivalent variant, analogue, derivative or fragment thereof.

According to a twenty-first aspect of the present invention, there is provided a bioactive molecule, the molecule having the ability to influence cellular differentiation, wherein the molecule is encoded by (a) a polynucleotide comprising a nucleotide sequence selected from the group as set forth in SEQ ID NO: 7, 14, 19-20; 24-29, 67, 74, 79-80, 84-89, 157, 159, 161, 163, and 165 or a functionally equivalent variant, analogue, derivative or fragment thereof, or (b) any other polynucleotide that would hybridise to any one of the polynucleotides selected from (a) under conditions of high stringency.

According to a twenty-second aspect of the present invention, there is provided a bioactive molecule, the molecule having the ability to modulate the expression of trefoil proteins, wherein the molecule comprises a polypeptide comprising an amino acid sequence selected from the group as set forth in SEQ ID NOs: 31, 45-46, 49, 91, 105-106, 109, 179, 181, 184,186, 217 and 219 or a functionally equivalent variant, analogue, derivative or fragment thereof.

According to a twenty-third aspect of the present invention, there is provided a bioactive molecule, the molecule having the ability to modulate the expression of trefoil proteins, wherein the molecule is encoded by (a) a polynucleotide comprising a nucleotide sequence selected from the group as set forth in SEQ ID NO: 1, 15-16, 19, 61, 75-76, 79, 178, 180, 183, 185, 190, 192, 216, and 218 or a kinctionally equivalent variant, analogue, derivative or fragment thereof, or (b) any other polynucleotide that would hybridise to any one of the polynucleotides selected from (a) under conditions of high stringency.

According to a twenty-fourth aspect of the present invention, there is provided a bioactive molecule, the molecule having cathelicidin activity, wherein the molecule comprises a polypeptide comprising an amino acid sequence selected from the group as set forth in SEQ ID NO: 171, 173, 175, 177, 188, 207, and 215 or a functionally equivalent variant, analogue, derivative or fragment thereof.

According to a twenty-fifth aspect of the present invention, there is provided a bioactive molecule, the molecule having cathelicidin activity, wherein the molecule is encoded by (a) a polynucleotide comprising a nucleotide sequence selected from the group as set forth in SEQ ID NO: 167-169, 170, 172, 174, 176, 191, and 208 or a functionally equivalent variant, analogue, derivative or fragment thereof, or (b) any other polynucleotide that would hybridise to any one of the polynucleotides selected from (a) under conditions of high stringency.

According to a twenty-sixth aspect of the present invention, there is provided a bioactive molecule, the molecule having the ability to affect cell growth, wherein the molecule comprises a polypeptide compriting an amino acid sequence selected from the group as set forth in SEQ ID NO: 171, 173, 175, 177, 188, 207, and 215 or a functionally equivalent variant, analogue, derivative or fragment thereof.

According to a twenty-seventh aspect of the present invention, there is provided a bioactive molecule, the molecule having the ability to affect cell growth, wherein the molecule is encoded by (a) a polynucleotide comprising a nucleotide sequence selected from the group as set forth in SEQ ID NO: 167-169, 170, 172, 174, 176, 191, and 208 or a functionally equivalent variant, analogue, derivative or fragment thereof, or (b) any other polynucleotide that would hybridise to any one of the polynucleotides selected from (a) under conditions of high stringency.

According to a twenty-eighth aspect of the present invention, there is provided a bioactive molecule, the molecule having the ability to affect growth of chicks, wherein the molecule comprises a polypeptide comprising an amino acid sequence as set forth in SEQ ID NO: 34-35, 49, 94-95, 109, 171,173, and 177 or a functionally equivalent variant, analogue, derivative or fragment thereof.

According to a twenty-ninth aspect of the present invention, there is provided a bioactive molecule, the molecule having the ability to affect growth of chicks, wherein the molecule is encoded by (a) a polynucleotide comprising a nucleotide sequence as set forth in SEQ ID NO: 4-5, 19, 64-65, 79, 170, 172, and 176 or a functionally equivalent variant, analogue, derivative or fragment thereof, or (b) any other polynucleotide that would hybridise to any one of the polynucleotides selected from (a) under conditions of high stringency.

According to a thirtieth aspect of the present invention, there is provided a bioactive molecule, the molecule having anti-microbial activity, wherein the molecule comprises a polypeptide comprising an amino acid sequence as set forth in SEQ ID NO: 171, 173, 175, 177, 188, 207, and 215 or a functionally equivalent variant, analogue, derivative or fragment thereof.

According to a thirty-first aspect of the present invention, there is provided a bioactive molecule, the molecule having anti-microbial activity, wherein the molecule is encoded by (a) a polynucleotide comprising a nucleotide sequence as set forth in SEQ ID NO: 167-169, 170, 172, 174, 176, 191, and 208 or a functionally equivalent variant, analogue, derivative or fragment thereof, or (b) any other polynucleotide that would hybridise to any one of the polynucleotides selected from (a) under conditions of high stringency.

According to a thirty-second aspect of the present invention, there is provided a bioactive molecule, the molecule having the ability to modulate stem cell pluripotency, wherein the molecule comprises a polypeptide comprising an amino acid sequence as set forth in SEQ ID NO: 37, 44, 49-50, 54-59, 97, 104, 109-110, 114-119, 158, 160, 162, 164, and 166 or a functionally equivalent variant, analogue, derivative or fragment thereof.

According to a thirty-third aspect of the present invention, there is provided a bioactive molecule, the molecule having the ability to modulate stem cell pluripotency, wherein the molecule is encoded by (a) a polynucleotide comprising a nucleotide sequence as set forth in SEQ ID NO: 7, 14, 19-20, 24-29, 67, 74, 79-80, 84-89, 157, 159, 161, 163, and 165 or a functionally equivalent variant, analogue, derivative or fragment thereof, or (b) any other polynucleotide that would hybridise to any one of the polynucleotides selected from (a) under conditions of high stringency. According to a thirty-fourth aspect of the present invention, there is provided a method for screening a candidate polypeptide for bioactivity, wherein said method comprises:

(a) introducing into a suitable host cell a polynucleotide of the third, fourth, ninth, eleventh, thirteenth, fifteenth, seventeenth, nineteenth, twenty-first, twenty-third, twenty-fifth, twenty-seventh, twenty-ninth, thirty-first or thirty-third aspects, or an expression vector of the sixth aspect, encoding the candidate polypeptide;

(b) culturing the cell under conditions suitable for expression of the candidate polypeptide encoded by the polynucleotide;

(c) recovering the candidate polypeptide; and

(d) assaying the recovered candidate polypeptide for biological activity.

According to a thirty-fifth aspect of the present invention, there is provided a method for screening a candidate polypeptide for bioactivity, wherein said method comprises:

(a) introducing into a suitable host cell a polynucleotide of the third, fourth, ninth, eleventh, thirteenth, fifteenth, seventeenth, nineteenth, twenty-first, twenty-third, twenty-fifth, twenty-seventh, twenty-ninth, thirty-first or thirty-third aspects, or an expression vector of the sixth aspect, encoding the candidate polypeptide;

(b) culturing the cell under conditions suitable for expression of the candidate polypeptide encoded by the polynucleotide, and for secretion of the candidate polypeptide into the extracellular medium;

(c) recovering the candidate polypeptide; and

(d) assaying the recovered candidate polypeptide for biological activity.

In embodiments of the thirty-fourth and thirty-fifth aspects, the assaying in step (d) may comprise assaying for regulation of mammary gland development including mammosphere formation, regulation of lactation, regulation of milk composition, cell proliferative activity including cellular differentiation and/or morphology activity, pro- or anti-inflammatory activity, pro- or anti-apoptotic activity, anti-microbial activity, regulation of differentiation of embryonic stem cells, regulation of trefoil activity, cathelicidin activity and regulation of epithelial cells including gut and/or skin epithelial cells.

According to a thirty-sixth aspect of the present invention, there is provided a polypeptide screened according to the method of the thirty-fourth or thirty-fifth aspects.

According to a thirty-seventh aspect of the present invention, there is provided a method for screening a candidate mammal for lactation capability, wherein said method comprises:

(a) obtaining a biological sample from the candidate mammal; and

(b) determining the level of expression in the biological sample of one or more polynucleotides selected from the group consisting of SEQ ID NOs: 1-29, 61-89, 151, 153, 155, 157, 159, 161, 163, 165, 167-170, 172, 174, 176, 178, 180, 183, 185, 190-192, 208, 216 and 218 or a functionally equivalent variant, analogue, derivative or fragment thereof wherein the level of expression of the one or more polynucleotides is indicative of lactation capability.

According to a thirty-eighth aspect of the present invention, there is provided a mammal screened according to the method of the thirty-seventh aspect.

According to a thirty-ninth aspect of the present invention, there is provided a method for screening for a candidate molecule that modulates the expression of the polypeptide of the first, second, fifth, eighth, tenth, twelfth, fourteenth, sixteenth, eighteenth, twentieth, twenty-second, twenty-fourth, twenty-sixth, twenty-eighth, thirtieth, thirty-second or thirty-sixth aspects, wherein said method comprises:

(a) contacting said candidate molecule with said polypeptide or a polynucleotide encoding said polypeptide;

(b) assaying for the level of expression of said polypeptide or said polynucleotide wherein the level of expression of said polypeptide or said polynucleotide is indicative of the capacity of the candidate molecule to modulate expression of said polypeptide.

According to a fortieth aspect of the present invention, there is provided a molecule screened according to the method of the thirty-ninth aspect.

According to a forty-first aspect of the present invention, there is provided a method for isolating a lactation-associated polynucleotide or a functionally equivalent variant, analogue, derivative or fragment thereof in a mammal, wherein said method comprises:

(a) obtaining a biological sample from said mammal;

(b) contacting the biological sample with a first polynucleotide of the third, fourth, twelfth, fourteenth, sixteenth, eighteenth, twentieth, twenty-second, twenty-fourth, twenty-sixth, twenty-eighth, thirtieth or thirty-second aspects, or an expression vector of the sixth aspect;

(c) detecting hybridization between the first polynucleotide of the third, fourth, twelfth, fourteenth, sixteenth, eighteenth, twentieth, twenty-second, twenty-fourth, twenty-sixth, twenty-eighth, thirtieth or thirty-second aspects, or an expression vector of the sixth aspect and a second polynucleotide in the biological sample; and

(d) isolating the second polynucleotide in the biological sample.

According to a forty-second aspect of the present invention, there is provided a lactation-associated polynucleotide isolated according to the method of the forty-first aspect.

According to a forty-third aspect of the invention there is provided a polypeptide encoded by a polynucleotide of the forty-second aspect.

According to a forty-fourth aspect of the present invention, there is provided a pharmaceutical composition, wherein said composition comprises at least one of:

(a) a polypeptide of the first, second, fifth, eighth, tenth, twelfth, fourteenth, sixteenth, eighteenth, twentieth, twenty-second, twenty-fourth, twenty-sixth, twenty-eighth, thirtieth, thirty-second, thirty-sixth or forty-third aspects;

(b) a polynucleotide of the third, fourth, ninth, eleventh, thirteenth, fifteenth, seventeenth, nineteenth, twenty-first, twenty-third, twenty-fifth, twenty-seventh, twenty-ninth, thirty-first, thirty-third or forty-second aspects;

(c) an expression vector of the sixth aspect;

(d) a host cell of the seventh aspect; and/or

(e) a molecule of the fortieth aspect

together with a pharmaceutically acceptable carrier, diluent, excipient and/or adjuvant.

According to a forty-fifth aspect of the present invention, there is provided a method for treating a disease or disorder in a subject, wherein said method comprises administering to the subject at least one of:

(a) a polypeptide of the first, second, fifth, eighth, tenth, twelfth, fourteenth, sixteenth, eighteenth, twentieth, twenty-second, twenty-fourth, twenty-sixth, twenty-eighth, thirtieth, thirty-second, thirty-sixth or forty-third aspects;

(b) a polynucleotide or a functionally equivalent variant, analogue, derivative or fragment thereof of the third, fourth, ninth, eleventh, thirteenth, fifteenth, seventeenth, nineteenth, twenty-first, twenty-third, twenty-fifth, twenty-seventh, twenty-ninth, thirty-first, thirty-third or forty-second aspects;

(c) an expression vector of the sixth aspect;

(d) a host cell of the seventh aspect;

(e) a molecule of the fortieth aspect; or

(f) a composition of the forty-fourth aspect.

According to a forty-sixth aspect of the present invention, there is provided use of at least one of:

(a) a polypeptide of the first, second, fifth, eighth, tenth, twelfth, fourteenth, sixteenth, eighteenth, twentieth, twenty-second, twenty-fourth, twenty-sixth, twenty-eighth, thirtieth, thirty-second, thirty-sixth or forty-third aspects;

(b) a polynucleotide or a functionally equivalent variant, analogue, derivative or fragment thereof of the third, fourth, ninth, eleventh, thirteenth, fifteenth, seventeenth, nineteenth, twenty-first, twenty-third, twenty-fifth, twenty-seventh, twenty-ninth, thirty-first, thirty-third or forty-second aspects;

(c) an expression vector of the sixth aspect;

(d) a host cell of the seventh aspect; and/or

(e) a molecule of the fortieth aspect

in the manufacture of a medicament for treating a disease or disorder in a subject.

According to a forty-seventh aspect of the present invention, there is provided a method for preserving food, wherein said method comprises contacting the food with at least one of a polypeptide of the first, second, fifth, eighth, tenth, twelfth, fourteenth, sixteenth, eighteenth, twentieth, twenty-second, twenty-fourth, twenty-sixth, twenty-eighth, thirtieth, thirty-second, thirty-sixth or forty-third aspects or a molecule of the fortieth aspect.

According to a forty-eighth aspect of the present invention, there is provided a food supplement for animals, wherein said food supplement comprises at least one polypeptide of the first, second, fifth, eighth, tenth, twelfth, fourteenth, sixteenth, eighteenth, twentieth, twenty-second, twenty-fourth, twenty-sixth, twenty-eighth, thirtieth, thirty-second, thirty-sixth or forty-third aspects.

According to a forty-ninth aspect of the present invention, there is provided a method for enhancing the regeneration of epithelial cells in a subject, wherein said method comprises administering to the subject at least one of:

(a) a polypeptide of the first, second, fifth, eighth, tenth, twelfth, fourteenth, sixteenth, eighteenth, twentieth, twenty-second, twenty-fourth, twenty-sixth, twenty-eighth, thirtieth, thirty-second, thirty-sixth or forty-third aspects;

(b) a polynucleotide or a functionally equivalent variant, analogue, derivative or fragment thereof of the third, fourth, ninth, eleventh, thirteenth, fifteenth, seventeenth, nineteenth, twenty-first, twenty-third, twenty-fifth, twenty-seventh, twenty-ninth, thirty-first, thirty-third or forty-second aspects;

(c) an expression vector of the sixth aspect;

(d) a host cell of the seventh aspect;

(e) a molecule of the fortieth aspect; or

(f) a composition of the forty-fourth aspect.

The subject may have been, or may be being, subjected to chemotherapy.

### DEFINITIONS

The term “comprising” means “including principally, but not necessarily solely”. Furthermore, variations of the word “comprising”, such as “comprise” and “comprises”, have correspondingly varied meanings.

The term “high stringency” as used herein refers to the conditions under which two polynucleotides may be hybridized, and may include, for example, the concentration of salts and/or detergents in a solution, the temperature of a solution that is used during the hybridization of the two polynucleotides and time period of the hybridization. Accordingly, the term “high stringency” as used herein refers to conditions in a solution that are conducive to hybridization of two polynucleotides only where such polynucleotides share a high degree of homology. The degree of homology may include, but not be limited to, a range of from about 50% to 99%. Thus, “high stringency” conditions may involve, but are not limited to, the use of a wash buffer that comprises 0 to 10% sodium dodecyl sulfate and/or 0 to 1× sodium chloride-sodium citrate at a temperature in the range of from about 60° C. to 70° C., or any other combination of buffers, temperature or time period which would yield a “high stringency” solution for hybridization.

The terms “polypeptide”, “peptide” and “protein” are used interchangeably and refer to a polymer made up of amino acids linked together by peptide bonds.

The term “conservative amino acid substitution” refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution.

The terms “polynucleotide”, “nucleic acid” and “oligonucleotide” are used interchangeably and refer to a single- or double-stranded polymer of deoxyribonucleotide or ribonucleotide bases, or analogues, derivatives, or combinations thereof. The terms include reference to the specified sequence as well as to the sequence complementary thereto, unless otherwise indicated. It will be understood that “5′ end” as used herein in relation to a nucleic acid molecule corresponds to the N-terminus of the encoded polypeptide and “3′ end” corresponds to the C-terminus of the encoded polypeptide.

The term “analogue” when used in relation to a polynucleotide or residue thereof, means a compound having a physical structure that is related to a DNA or RNA molecule or residue, and preferably is capable of forming a hydrogen bond with a DNA or RNA residue or an analogue thereof (i.e., it is able to anneal with a DNA or RNA residue or an analogue thereof to form a base-pair). Such analogues may possess different chemical and biological properties to the ribonucleotide or deoxyribonucleotide residue to which they are structurally related. Methylated, iodinated, brominated or biotinylated residues are examples of analogues.

The term “analogue” as used herein with reference to a polypeptide means a polypeptide which is a derivative of the polypeptide of the invention, which derivative comprises addition, deletion or substitution of one or more amino acids, such that the polypeptide retains substantially the same function.

The term “derivative” when used in relation to a polynucleotide of the present invention include any functionally-equivalent nucleic acids, including any fusion molecules produced integrally (e.g., by recombinant means) or added post-synthesis (e.g., by chemical means). Such fusions may comprise one or both strands of the double-stranded oligonucleotide of the invention with RNA or DNA added thereto or conjugated to a polypeptide (e.g., puromycin or other polypeptide), a small molecule (e.g., psoralen) or an antibody.

The term “fragment” when used in relation to a polypeptide or polynucleotide molecule refers to a constituent of a polypeptide or polynucleotide. Typically the fragment possesses qualitative biological activity in common with the polypeptide or polynucleotide. The peptide fragment may be between about 5 to about 150 amino acids in length, between about 5 to about 100 amino acids in length, between about 5 to about 50 amino acids in length, or between about 5 to about 25 amino acids in length. Alternatively, the peptide fragment may be between about 5 to about 15 amino acids in length. However, fragments of a polynucleotide do not necessarily need to to encode polypeptides which retain biological activity. Rather, a fragment may, for example, be useful as a hybridization probe or PCR oligonucleotide. The fragment may be derived from a polynucleotide of the invention or alternatively may be synthesized by some other means, for example chemical synthesis.

The term “variant” as used herein refers to substantially similar sequences. Generally, polypeptide or polynucleotide sequence variants possess qualitative biological activity in common. Further, these polypeptide or polynucleotide sequence variants may share at least 50%, 55%, 60%, 65%, 70%, 75%, 80%, 85%, 90%, 95%, 96%, 97%, 98% or 99% sequence identity. Also included within the meaning of the term “variant” are homologues of polypeptides or polynucleotides of the invention. A homologue is typically a polypeptide or polynucleotide from a different species but sharing substantially the same biological function or activity as the corresponding polypeptide or polynucleotide disclosed herein. For example, homologues of polynucleotides disclosed herein may be from bovine species or humans. Typically homologues are identified and isolated by virtue of the sequence of a polynucleotide disclosed herein.

The term lactation-associated” as used herein in relation to a polypeptide, polynucleotide or molecule means that expression of the polypeptide, polynucleotide or molecule is altered during lactation as compared to basal levels of expression before or after lactation, or that expression of the polypeptide, polynucleotide or molecule is capable of altering lactation in any way, for example, including but not limited to regulation of mammary gland development including mammosphere formation, regulation of lactation timing, regulation of milk let down, regulation of milk volume, regulation of milk composition, cell proliferative activity including cellular differentiation and/or morphology activity. Expression of the polypeptide, polynucleotide or molecule may be increased or decreased during lactation, either at one point during the lactation cycle or over the course of lactation. For example, an increase or decrease in expression of the polypeptide, polynucleotide or molecule during lactation may be observed by comparing the level of expression prior to lactation initiation with the level of expression at involution, by comparing the level of expression across a lactation phase change, or by comparing the level of expression between any two time points in lactation.

The term “isolate” as used herein as it pertains to methods of isolating bioactive molecules means recovering a molecule from a cell culture medium substantially free of cellular material, although the molecule need not be free of all components of the media. For example a secreted polypeptide may be recovered in the extracellular media, such as the supematant, and still be “isolated”.

The term “modulate” as used herein refers to any increase or decrease in expression of a polypeptide, polynucleotide or molecule disclosed herein.

The terms “bioactive” and “biological activity” are used interchangeably and refer to a polypeptide, polynucleotide or molecule disclosed herein having a defined biological activity. Biological activities may include, but are not limited to, regulation of mammary gland development including mammosphere formation, regulation of lactation, regulation of milk composition, cell proliferative activity including cellular differentiation and/or morphology activity, pro- or anti-inflammatory activity, pro- or anti-apoptotic activity, anti-microbial activity, regulation of differentiation of embryonic stem cells, regulation of trefoil activity, cathelicidin activity and regulation of epithelial cells including gut and/or skin epithelial cells.

The term “secreted” as used herein means that the polypeptide is secreted from the cytoplasm of a cell, either as a cell membrane-associated polypeptide with an extracellular portion or is secreted entirely into the extracellular space.

### BRIEF DESCRIPTION OF THE DRAWINGS

A preferred form of the present invention will now be described by way of example with reference to the accompanying drawings:

FIG. 1. Microarray expression profiles. Each graph shows normalized expression intensities for a Lactation-Associated Molecule (LAM) (polynucleotides) which corresponds to a Tammar expressed sequence tag (EST). 33 LAMS (designated LAMS 1-33) are represented in FIG. 1. Three lines of varying darkness are depicted on each graph. The light grey lines represent single channel normalization of the average intensity from Cy3 fluorescence. The dark grey lines represent single channel normalization of the average intensity from Cy5 fluorescence. The black lines represent the average of these Cy3 and Cy5 channel intensities. The scale for each LAM intensity is relative, the highest individual spot intensity being 100 percent. All lines pass through the origin of the graph. Lactation phases are indicated as P (pregnancy), 2A, 2B and 3.

FIG. 2. Cellular growth assay with HC11 cells. Mouse mammary epithelial cells, HC11 cells, were grown in media conditioned with: mock transfection (control), LAM32, LAM32a or LAM32b for 72 hours. Samples were performed in triplicate and the cells from each triplicate counted in triplicate. LAM32, LAM32a or LAM32b had significantly more cells (P<0.05 using a t-test comparing control with sample). LAM 32 is represented as set forth in SEQ ID NO: 188, and two splice variants thereof, designated LAM32a and 32b as set forth in SEQ ID NO: 215 and 207 respectively.

FIG. 3. Cellular growth assay with mouse embryonic stem (ES) cells. LAM 32 as set forth in SEQ ID NO: 188, and two splice variants thereof, designated LAM32a and 32b as set forth in SEQ ID NO: 215 and 207 respectively were tested to determine the ability to affect growth of mouse ES cells.

FIG. 4. Cellular growth assay with AGS cells. Seven cathelicidin variants including cathelicidins 1, 4, and 6 which are known (the relevant sequences can be obtained using the following hyperlink: http://www.ncbi.nlm.nih.gov/entrez/query.fcgi?db=unigene&cmd=search&term=cathelicidin) and LAM 32.8-32.11 (i.e. cathelicidins 8, 9, 10 and 11) as disclosed in the present invention and represented herein as set forth in SEQ ID NOs: 170-177 were tested. AGS cells were used in this assay and the growth rate of these cells in the presence of the abovementioned cathelicidin variants were determined.

FIG. 5. Chick growth assay by measuring weight. 10 LAMs (being bovine homologues having homology to specific Tammar ESTs), 3 known bovine cathelicidins, and four bovine cathelicidin variants were tested. Conditioned media containing a LAM polypeptides or bovine cathelicidins or variants thereof was diluted in a total of 1 ml of normal saline. Eggs on day 16 of incubation were injected with 1 ml of the media containing the into the broad end of the egg. Chicks were weighed on day of hatching. The overall condition of the chick was assessed including the possible presence of stumpy legs and/or loss of down feathers.

FIG. 6. Cellular morphology assay. To determine the ability of LAMs to influence cellular morphology, bovine mammary epithelial cells were plated onto a extracellular matrix and the cells were subsequently allowed to migrate to form mammospheres in the presence or absence of each LAM. The figure contains Panel A (Negative control) and Panels B to G (Presence of a LAM for which one LAM polypeptide is represented in one panel).

FIG. 7. Cellular differentiation assay. To determine the ability of LAMs to influence cell differentiation, mouse embryonic stem (ES) cells were cultured in the presence of leukemia inhibitory factor (LIF) 1000 U/ml and the LAM in question a 1 in 5 dilution of the secreted polypeptides and cultured for 48 hours. The mouse ES cells contained an Oct4-βgal transgene, such that Oct4 expression (an indicator of pluripotency and hence an absence of diffferentiation) results in the cells staining blue. This figure contains Panel A (Negative control) and Panels B to L (Presence of a LAM for which one LAM polypeptide is represented in one panel).

FIG. 8. Microarray expression profiles for bovine RNA. Gene expression for each LAM represented on the Affymetrix bovine microarray. Samples show expression in five cows during pregnancy (green), lactation (Red) and involution (blue). The x axis shows the range of gene expression in the samples in log 2.

FIG. 9: Northern expression of clone LAM 32. Northern showing expression of cathelicidin in the tammar mammary gland at A: Day 13 pregnant, B: parturition, C: dayl, D: day2, E: day3, F: day10, G: day40, H: day87, I: day114, J: day150, K: day240, L: day 5 involution and M: day10 involution.

FIG. 10: SNPs derived from bovine poynucleotides as represented in LAMS 30, 31, and 32 which are represented as sequences of bovine cathelicidins and deduced encoded protein. Nucleotides in blue show variation between sequences in multiple databases. Nucleotide differences resulting in changed amino acids also show the resultant amino acid change.

FIGS. 11A and B: Peroxidase assay for trefoil activity. Supematants of HEK293 cells expressing tammar mammary clones were applied to the human gastric epithelial cell line, AGS, for 48 hours. The resultant AGS cell conditioned media containing stimulated AGS cell secreted proteins, and the AGS cells, were lysed. Samples of the AGS cell secreted proteins and cell lysates, and a recombinant positive control were transferred to a solid support and probed using a rabbit anti-trefoil 1 antibody and a secondary goat anti-rabbit antibody conjugated to horse radish peroxidase was used to detect the trefoil 1. A: Detection of secreted trefoil 1 in supematants from stimulated AGS cells. B: Detection of expression of trefoil 1 in stimulated AGS cell lysate samples.

FIG. 12: ERK phosphorylation assay. Aliquots of the samples were used to activate HSC-2 cells in a 96 well plate. Cells were also stimulated with an internal control for activation of cells (Stim=10% serum) or left unstimulated (unstim). Cells were lysed and assayed for ERK activation using TGR's proprietary assay technology (SureFire). The sample number on the x-axis corresponds to a LAM as set out in Table 24. The y-axis indicates relative fluorescence units. The results are presented as the mean+/−SEm of 3 separate samples from a single experiment.

FIG. 13: p38 MAPK stimulation in U937 cells. U937 Human monocytic cells were stimulated with TNFα (Stim) or the supplied test samples and assessed for activation of p38 MAPK. The sample number on the x-axis corresponds to a LAM as set out in Table 24. The results are the mean+/−SEm of 3 replicate cell stimulations from a single experiment.

FIG. 14: Matrigel outgrowth assay. Monolayers of cells (MDA-MD-MB231) were grown to 80% confluence and collected during log phase. 5×10³ cells were added per well (96 well format) on top of preset Matrigel (50 ul). 50 ul of bioactive supematant was added per well. Colony outgrowth was monitored over the course of the experiment and photographed after 2 (FIG. 14A) and 5 (FIG. 14B) days.

FIG. 15: Live cell count. Jurkat cells were cultured with supematant of cells expressing a LAM for 24 and 48 hours. Live cell counts are graphed (FIGS. 15A and 15B) for each supernatant. The cell count is relative to the control (Ptarget). Hence a value about 1 indicates an increase in live cell numbers relative to the control, and the inverse when less than 1. Y error bars indicate 1 SD.

FIG. 16: Cell viability. Jurkat cells were cultured with supematant of cells expressing a LAM for 24 and 48 hours. The percentage of live, viable cells are graphed (FIGS. 16A and 16B) for each supematant (including the Ptarget control). Y error bars indicate 1 SD.

FIG. 17: Live cell count. Kit 225 cells were cultured with 10 uL of supematant of cells expressing a LAM for 24 and 48 hours. The Kit 225 cells were cultured with 2 mediums—low IL-2 (indicated in the figure as “lo”) and high IL-2, (indicted as “hi”). Live cell counts are graphed (FIGS. 17A-17D) for each supematant. The cell count is relative to the control (Ptarget). Hence a value about 1 indicates an increase in live cell numbers relative to the control, and the inverse when less than 1. Y error bars indicate 1 SD.

FIG. 18: Cell viability. Kit 225 cells were cultured with 10 uL of supematant of cells expressing a LAM for 24 and 48 hours. The Kit 225 cells were cultured with 2 mediums—low IL-2 (indicated in the figure as “lo”) and high IL-2, (indicted as “hi”). The percentage of live, viable cells are graphed (FIGS. 18A-18D) for each supematant (including the Ptarget control). Y error bars indicate 1 SD.

FIG. 19: TCN1 (LAM02) induces OCT4 loss. Murine ES cells containing an Oct4-βgal transgene were cultured with TCN1 polypeptide. Loss of blue staining demonstrates a loss of OCT4 expression in mouse embryonic stems cells induced by bovine TCN1.

FIG. 20: TCN1 (LAM02) activates ERK1/2. Swiss 3T3 cells were treated with cell supematants containing bovine TCN 1 polypeptide. The cells were assayed for ERK1/2 activation. An increase in relative fluorescence units over the control indicates ERK activation.

FIG. 21: DGAT2 (LAM07) activates ERK1/2. Swiss 3T3 cells were treated with cell supematants containing bovine DGAT2 polypeptide. The cells were assayed for ERK1/2 activation. An increase in relative fluorescence units (y-axis) over the control indicates ERK activation.

FIG. 22: MGC14327 (LAM18) activates p38MAPK. RAW cells were treated with cell supematants containing bovine MGC14327 polypepetide. The cells were then assayed for p38 MAPK activation by the use of a p38MAPK specific fluorescent dye. An increase in re4lative fluorescent units (y-axis) over the control indicates p38 MAPK activation.

FIG. 23: MGC14327 (LAM18) activates ERK1/2. Swiss 3T3 cells were treated with cell supematants containing bovine MGC14327 polypepetide. The cells were assayed for ERK1/2 activation. An increase in relative fluorescence units (y-axis) over the control indicates ERK activation.

FIG. 24: IFITM3 (LAM20) induces OCT4 loss. Murine ES cells containing an Oct4-βgal transgene were cultured with cell supernatants containing IFITM3 polypeptide. Loss of blue staining demonstrates a loss of OCT4 expression in mouse embryonic stems cells induced by IFITM3.

FIG. 25: C1orf160 (LAM24) induces OCT4 loss. Murine ES cells containing an Oct4-βgal transgene were cultured with cell supernatants containing C1orf160 (chromosome 1 open reading frame 160) polypeptide. Loss of blue staining demonstrates a loss of OCT4 expression in mouse embryonic stems cells induced by C1orf160.

FIG. 26: CAMP or Cathelicidin antimicrobial peptide (LAM18) activates ERK1/2. Swiss 3T3 cells were treated with cell supematants containing CAMP polypepetide. The cells were assayed for ERK1/2 activation. An increase in relative fluorescence units (y-axis) over the control indicates ERK activation.

FIG. 27: Western blot analysis of LAM32 in milk streams. Samples of raw milk, various milk products and milk and whey ultrafiltration retentates and permeates were electrophoresed and LAM32 was detected by a polyclonal rabbit anti-bovine cathelicidin antibody and detected using goat anti-rabbit antibody conjugated for luminescent detection.

FIG. 28: Demonstration of successful expression and production of bovine cathelicidin in HEK293 conditioned media. HEK293 cells were transfected vectors expressing cathelicidin. Conditioned media from those cells was electrophoresed and cathelicidin orthologues detected by Western blotting using a rabbit anti-bovine cathelicidin antibody.

FIG. 29: Demonstration of cathelicidin in cows milk. Standard casein-whey fractionation was performed the fractions electrophoresed and cathelicidin orthologues detected by Western blotting using a rabbit anti-bovine cathelicidin antibody.

FIG. 30: Live cell count and cell viability. HuVEC cells were cultured with supematant of cells expressing a LAM for 24 and 48 hours. Live cell counts are graphed (FIG. 30A) for each supematant. The cell count is relative to the control (Ptarget). Hence a value about 1 indicates an increase in live cell numbers relative to the control, and the inverse when less than 1. Y error bars indicate 1 SD. HuVEC cells were cultured with supematant of cells expressing a LAM for 24 and 48 hours. The percentage of live, viable cells are graphed (FIG. 30B) for each supernatant (including the Ptarget control). Y error bars indicate 1 SD.

FIG. 31: Live cell count and cell viability. Jurkat cells were cultured with supernatant of cells expressing a LAM for 24 and 48 hours. Live cell counts are graphed (FIG. 31A) for each supernatant. The cell count is relative to the control (Ptarget). Hence a value about 1 indicates an increase in live cell numbers relative to the control, and the inverse when less than 1. Y error bars indicate 1 SD. Jurkat cells were cultured with supernatant of cells expressing a LAM for 24 and 48 hours. The percentage of live, viable cells are graphed (FIG. 31B) for each supernatant (including the Ptarget control). Y error bars indicate 1 SD.

FIG. 32: Live cell count and cell viability. THP-1 cells were cultured with supernatant of cells expressing a LAM for 24 and 48 hours. Live cell counts are graphed (FIGS. 32A-32C) for each supernatant. The cell count is relative to the control (Ptarget). Hence a value about 1 indicates an increase in live cell numbers relative to the control, and the inverse when less than 1. Y error bars indicate 1 SD. Jurkat cells were cultured with supernatant of cells expressing a LAM for 24 and 48 hours. The percentage of live, viable cells are graphed (FIGS. 32D-32F) for each supernatant (including the Ptarget control). Y error bars indicate 1 SD.

FIG. 33: Proliferation assay. MDA-MB-231 cells were cultured with bioactive supernatants for 3 and 6 days before being fixed and stained with Sulforhadamine B for 10 min, washed five times with 1% acetic acid and allowed to dry overnight. Absorbance at 540 nm was measured. Each time point was performed in triplicate. The proliferation curves show differences in the rate of proliferation between the controls and the presence of each bioactive (P values shown). The rate of proliferation is represented by the gradient.

### BEST MODE OF PERFORMING THE INVENTION

A variety of approaches have been adopted in an attempt to elucidate the identity of bioactive proteins in milk. However these approaches have met with limited success and it is accepted that the extent of bioactive proteins in milk has not been fully realized. Our understanding of human nutrition and development, and also our ability to manipulate milk production in domestic animals, will depend largely on increasing our understanding of milk composition and identifying molecules with specific biological activities in milk.

With the tammar wallaby and bovine experimental model organisms, the inventors have used a combination of microarray expression profiling and bioinformatics to identify lactation-associated polypeptides. The inventors have also used various screening assays to identify activities of such lactation-associated polypeptides, such as growth-promoting activity, pro-apoptotic activity, anti-apoptotic activity, pro-inflammatory activity, anti-inflammatory activity, anti-microbial activity, chick growth regulation, the ability to influence mammary epithelial cell growth and mouse embryonic stem cell growth.

A polypeptide identified according to the present invention as being lactation-associated may comprise an amino acid sequence encoded by a polynucleotide of a cDNA library as shown in Table 1, or a variant, analogue, derivative or fragment of a polynucleotide of a cDNA library as shown in Table 1.

A polypeptide identified according to the present invention as being lactation-associated may further comprise an amino acid sequence as set forth in any one of SEQ ID NOs: 31-59, 91-119, 152, 154, 156, 158, 160, 162, 164, 166, 171, 173, 175, 177, 179, 181, 184, 186, 188, 207, 215, 217 and 219. Where an amino acid sequence disclosed herein is the partial sequence of a lactation-associated polypeptide, the corresponding complete sequence may be readily obtained using molecular biology techniques well known to those skilled in the art. Accordingly, the scope of the present invention extends to the complete lactation-associated polypeptides comprising the partial sequences identified herein.

The present invention also provides polynucleotides, identified herein as being lactation-associated. A polynucleotide identified according to the present invention as being lactation-associated may comprise a polynucleotide of a cDNA library as shown in Table 1, or a functionally equivalent variant, analogue, derivative or fragment of a polynucleotide of a cDNA library as shown in Table 1.

A polynucleotide of the invention may comprise a nucleotide sequence as set forth in any one of SEQ ID NOs: 1-29, 61-89, 151, 153, 155, 157, 159, 161, 163, 165, 167-170, 172, 174, 176, 178, 180, 183, 185, 190-192, 208, 216 and 218. Where a nucleotide sequence disclosed herein is the partial sequence of a lactation-associated polynucleotide, the corresponding complete sequence may be readily obtained using molecular biology techniques well known to those skilled in the art. Accordingly, the scope of the present invention extends to the complete lactation-associated polynucleotides comprising the partial sequences identified herein.

The present invention also provides for methods for screening candidate polypeptides for bioactivity, for screening candidate mammals for lactation capability, for screening for candidate molecules that modulate the expression of the polypeptides of the present invention, and for isolating lactation-associated polynucleotides in a mammal.

Also contemplated are methods and compositions for treating mammals in need of treatment with effective amounts of polypeptides, polynucleotides, expression vectors, host cells, molecules or compositions of the invention. Such treatment may be for the therapy or prevention of a medical condition in which case an “effective amount” refers to a non-toxic but sufficient amount to provide the desired therapeutic effect. Such medical conditions may include microbial infections and wounds to the gut or skin. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

The present invention also provides methods for preserving food, wherein said methods comprise contacting the food with the polypeptides and/or molecules as disclosed herein.

The present invention further provides food supplement for mammals, wherein said food supplement comprises the polypeptides as disclosed herein.

### Polypeptides

Lactation-associated polypeptides of the invention may be regulatory proteins, involved in, for example, regulation of lactogenesis, regulation of lactation phase changes including those relating to changes in milk composition, or regulation of the timing of initiation of milk secretion or involution. Polypeptides of the invention may be bioactive molecules with biological activities of significance to the offspring, including providing nutrition, developmental cues or protection. For example, the bioactive molecules may have anti-microbial activity, anti-inflammatory activity, pro-inflammatory activity or immune response mediator activity. Accordingly, the invention provides methods of identifying such activities in polypeptides of the invention and compositions comprising polypeptides of the invention.

As exemplified herein, a number of the lactation-associated polypeptides of the invention have been identified as having one or more bioactivities, selected from the group consisting of: growth-promoting activity; pro-apoptotic activity; anti-apoptotic activity; pro-inflammatory activity; anti-inflammatory activity; chick growth regulation, the ability to influence mammary epithelial cell growth and mouse embryonic stem cell growth.

Polypeptides of the invention may have signal or leader sequences to direct their transport across a membrane of a cell, for example to secrete the polypeptide into the extracellular space. The leader sequence may be naturally present on the polypeptide amino acid sequence or may be added to the polypeptide amino acid sequence by recombinant techniques known to those skilled in the art.

In addition to the lactation-associated polypeptides comprising amino acid sequences set forth herein, also included within the scope of the present invention are functionally equivalent variants and fragments thereof.

### Polynucleotides

Embodiments of the present invention provide isolated polynucleotides, the expression of which is altered during lactation.

In addition to the lactation-associated polynucleotides comprising nucleotide sequences set forth herein, also included within the scope of the present invention are functionally equivalent variants and fragments thereof.

The present invention contemplates the use of polynucleotides disclosed herein and fragments thereof to identify and obtain corresponding partial and complete sequences from other species, such as bovine species and humans using methods of recombinant DNA well known to those of skill in the art, including, but not limited to southern hybridization, northern hybridization, polymerase chain reaction (PCR), ligase chain reaction (LCR) and gene mapping techniques. Polynucleotides of the invention and fragments thereof may also be used in the production of antisense molecules using techniques known to those skilled in the art.

As exemplified herein, homologues of lactation-associated polynucleotides of the present invention have been detected as expressed sequences in the bovine genome, using a bovine cDNA microarray. Further, several of these sequences have been shown to be highly expressed in either pregnant or lactating cows, thereby demonstrating the presence of lactation-associated to polynucleotides homologous to those of the present invention.

Accordingly, the present invention contemplates oligonucleotides and fragments based on the sequences of the polynucleotides disclosed herein for use as primers and probes for the identification of homologous sequences. Oligonucleotides are short stretches of nucleotide residues suitable for use in nucleic acid amplification reactions such as PCR, typically being at least about 10 nucleotides to about 50 nucleotides in length, more typically about 15 to about 30 nucleotides in length. Probes are nucleotide sequences of variable length, for example between about 10 nucleotides and several thousand nucleotides, for use in detection of homologous sequences, typically by hybridization. The level of homology (sequence identity) between sequences will largely be determined by the stringency of hybridization conditions. In particular the nucleotide sequence used as a probe may hybridize to a homologue or other functionally equivalent variant of a polynucleotide disclosed herein under conditions of low stringency, medium stringency or high stringency. Low stringency hybridization conditions may correspond to hybridization performed at 50° C. in 2×SSC. There are numerous conditions and factors, well known to those skilled in the art, which may be employed to alter the stringency of hybridization. For instance, the length and nature (DNA, RNA, base composition) of the nucleic acid to be hybridized to a specified nucleic acid; concentration of salts and other components, such as the presence or absence of formamide, dextran sulfate, polyethylene glycol etc; and altering the temperature of the hybridization and/or washing steps. For example, a hybridization filter may be washed twice for 30 minutes in 2×SSC, 0.5% SDS and at least 55° C. (low stringency), at least 60° C. (medium stringency), at least 65° C. (medium/high stringency), at least 70° C. (high stringency) or at least 75° C. (very high stringency).

In particular embodiments, the polynucleotides of the invention may be cloned into a vector. The vector may be a plasmid vector, a viral vector, or any other suitable vehicle adapted for the insertion of foreign sequences, their introduction into eukaryotic cells and the expression of the introduced sequences. Typically the vector is a eukaryotic expression vector and may include expression control and processing sequences such as a promoter, an enhancer, ribosome binding sites, polyadenylation signals and transcription termination sequences.

### Modulators

The polypeptides and polynucleotides of the present invention, and fragments and analogues thereof are useful for the screening and identification of compounds and agents that interact with these molecules. In particular, desirable compounds are those that modulate the activity of these polypeptides and polynucleotides. Such compounds may exert a modulatory effect by activating, stimulating, increasing, inhibiting or preventing expression or activity of the polypeptides and/or polynucleotides. Suitable compounds may exert their effect by virtue of either a direct (for example binding) or indirect interaction.

Compounds which bind, or otherwise interact with the polypeptides and polynucleotides of the invention, and specifically compounds which modulate their activity, may be identified by a variety of suitable methods. Interaction and/or binding may be determined using standard competitive binding assays or two-hybrid assay systems.

For example, the two-hybrid assay is a yeast-based genetic assay system typically used for detecting protein-protein interactions. Briefly, this assay takes advantage of the multi-domain nature of transcriptional activators. For example, the DNA-binding domain of a known transcriptional activator may be fused to a polypeptide, or fragment or analogue thereof, and the activation domain of the transcriptional activator fused to a candidate protein. Interaction between the candidate protein and the polypeptide, or fragment or analogue thereof, will bring the DNA-binding and activation domains of the transcriptional activator into close proximity. Interaction can thus be detected by virtue of transcription of a specific reporter gene activated by the transcriptional activator.

Alternatively, affinity chromatography may be used to identify polypeptide binding partners. For example, a polypeptide, or fragment or analogue thereof, may be immobilised on a support (such as sepharose) and cell lysates passed over the column. Proteins binding to the immobilised polypeptide, fragment or analogue can then be eluted from the column and identified. Initially such proteins may be identified by N-terminal amino acid sequencing for example.

Alternatively, in a modification of the above technique, a fusion protein may be generated by fusing a polypeptide, fragment or analogue to a detectable tag, such as alkaline phosphatase, and using a modified form of immunoprecipitation as described by Flanagan and Leder (1990). Methods for detecting compounds that modulate activity of a polypeptide of the invention may involve combining the polypeptide with a candidate compound and a suitable labelled substrate and monitoring the effect of the compound on the polypeptide by changes in the substrate (may be determined as a function of time). Suitable labelled substrates include those labelled for colourimetric, radiometric, fluorimetric or fluorescent resonance energy transfer (FRET) based methods, for example. Alternatively, compounds that modulate the activity of the polypeptide may be identified by comparing the catalytic activity of the polypeptide in the presence of a candidate compound with the catalytic activity of the polypeptide in the absence of the candidate compound.

The present invention also contemplates compounds which may exert their modulatory effect on polypeptides of the invention by altering expression of the polypeptide. In this case, such compounds may be identified by comparing the level of expression of the polypeptide in the presence of a candidate compound with the level of expression in the absence of the candidate compound.

Polypeptides of the invention and appropriate fragments and analogues can be used in high-throughput screens to assay candidate compounds for the ability to bind to, or otherwise interact therewith. These candidate compounds can be further screened against functional polypeptides to determine the effect of the compound on polypeptide activity.

It will be appreciated that the above described methods are merely examples of the types of methods which may be employed to identify compounds that are capable of interacting with, or modulating the activity of, polypeptides of the invention, and fragments and analogues thereof, of the present invention. Other suitable methods will be known to persons skilled in the art and are within the scope of the present invention.

Potential modulators, for screening by the above methods, may be generated by a number of techniques known to those skilled in the art. For example, various forms of combinatorial chemistry may be used to generate putative non-peptide modulators. Additionally, techniques such as nuclear magnetic resonance (NMR) and X ray crystallography, may be used to model the structure of polypeptides of the invention and computer predictions used to generate possible modulators (in particular inhibitors) that will fit the shape of the substrate binding cleft of the polypeptide.

By the above methods, compounds can be identified which either activate (agonists) or inhibit (antagonists) the expression or activity of polypeptides of the invention. Such compounds may be, for example, antibodies, low molecular weight peptides, nucleic acids or non-proteinaceous organic molecules.

Antagonists or agonists of polypeptides of the invention may include antibodies. Suitable antibodies include, but are not limited to polyclonal antibodies, monoclonal antibodies, chimeric antibodies, humanised antibodies, single chain antibodies and Fab fragments.

Antibodies may be prepared from discrete regions or fragments of the polypeptide of interest. An antigenic polypeptide contains at least about 5, and preferably at least about 10, amino acids. Methods for the generation of suitable antibodies will be readily appreciated by those skilled in the art. For example, a suitable monoclonal antibody, typically containing Fab portions, may be prepared using the hybridoma technology described in Antibodies'A Laboratory Manual, (Harlow and Lane, eds.) Cold Spring Harbor Laboratory, N.Y. (1988), the disclosure of which is incorporated herein by reference.

Similarly, there are various procedures known in the art which may be used for the production of polyclonal antibodies to polypeptides of interest as disclosed herein. For the production of polyclonal antibodies, various host animals, including but not limited to rabbits, mice, rats, sheep, goats, etc, can be immunized by injection with a polypeptide, or fragment or analogue thereof. Further, the polypeptide or fragment or analogue thereof can be conjugated to an immunogenic carrier, e.g., bovine serum albumin (BSA) or keyhole limpet hemocyanin (KLH). Also, various adjuvants may be used to increase the immunological response, including but not limited to Freund's (complete and incomplete), mineral gels such as aluminium hydroxide, surface active substances such as lysolecithin, pluronic polyols, polyanions, peptides, oil emulsions, keyhole limpet hemocyanins, dinitrophenol, and potentially useful human adjuvants such as BCG (bacille is Calmette-Guerin) and Corynebacterium parvum.

Screening for the desired antibody can also be accomplished by a variety of techniques known in the art. Assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, and immunoelectrophoresis assays, and the like (see, for example, Ausubel et al., eds, 1994, Current Protocols in Molecular Biology, Vol. 1, John Wiley & Sons, Inc., New York). Antibody binding may be detected by virtue of a detectable label on the primary antibody. Alternatively, the primary antibody may be detected by virtue of its binding with a secondary antibody or reagent which is appropriately labelled. A variety of methods are known in the art for detecting binding in an immunoassay and are within the scope of the present invention.

Embodiments of the invention may utilise antisense technology to inhibit the expression of a polynucleotide by blocking translation of the encoded polypeptide. Antisense technology takes advantage of the fact that nucleic acids pair with complementary sequences. Suitable antisense molecules can be manufactured by chemical synthesis or, in the case of antisense RNA, by transcription in vitro or in vivo when linked to a promoter, by methods known to those skilled in the art.

For example, antisense oligonucleotides, typically of 18-30 nucleotides in length, may be generated which are at least substantially complementary across their length to a region of the nucleotide sequence of the polynucleotide of interest. Binding of the antisense oligonucleotide to their complementary cellular nucleotide sequences may interfere with transcription, RNA processing, transport, translation and/or mRNA stability. Suitable antisense oligonucleotides may be prepared by methods well known to those of skill in the art and may be designed to target and bind to regulatory regions of the nucleotide sequence or to coding (exon) or non-coding (intron) sequences. Typically antisense oligonucleotides will be synthesized on automated synthesizers. Suitable antisense oligonucleotides may include modifications designed to improve their delivery into cells, their stability once inside a cell, and/or their binding to the appropriate target. For example, the antisense oligonucleotide may be modified by the addition of one or more phosphorothioate linkages, or the inclusion of one or morpholine rings into the backbone (so-called ‘morpholino’ oligonucleotides).

An alternative antisense technology, known as RNA interference (RNAi), may be used, according to known methods in the art (for example WO 99/49029 and WO 01/70949, the disclosures of which are incorporated herein by reference), to inhibit the expression of a polynucleotide. RNAi refers to a means of selective post-transcriptional gene silencing by destruction of specific mRNA by small interfering RNA molecules (siRNA). The siRNA is generated by cleavage of double stranded RNA, where one strand is identical to the message to be inactivated. Double-stranded RNA molecules may be synthesised in which one strand is identical to a specific region of an mRNA transcript and introduced directly. Alternatively corresponding dsDNA can be employed, which, once presented intracellularly is converted into dsRNA. Methods for the synthesis of suitable molecule for use in RNAi and for achieving post-transcriptional gene silencing are known to those of skill in the art.

A further means of inhibiting expression may be achieved by introducing catalytic antisense nucleic acid constructs, such as ribozymes, which are capable of cleaving mRNA transcripts and thereby preventing the production of wildtype protein. Ribozymes are targeted to and anneal with a particular sequence by virtue of two regions of sequence complementarity to the target flanking the ribozyme catalytic site. After binding the ribozyme cleaves the target in a site-specific manner. The design and testing of ribozymes which specifically recognise and cleave sequences of interest can be achieved by techniques well known to those in the art (for example Lieber and Strauss, 1995, Molecular and Cellular Biology, 15:540-551, the disclosure of which is incorporated herein by reference).

### Compositions

Compositions according to embodiments of the invention may be prepared according to methods which are known to those of ordinary skill in the art containing the suitable agents. Such compositions may include a pharmaceutically acceptable carrier, diluent and/or adjuvant. The carders, diluents and adjuvants must be “acceptable” in terms of being compatible with the other ingredients of the composition, and not deleterious to the recipient thereof. These compositions can be administered by standard routes. In general, the compositions may be administered by the parenteral, topical or oral route.

It will be understood that the specific dose level for any particular individual will depend upon a variety of factors including, for example, the activity of the specific agents employed, the age, body weight, general health, diet, the time of administration, rate of excretion, and combination with any other treatment or therapy. Single or multiple administrations of the agents or compositions can be carried out with dose levels and pattern being selected by the treating physician.

Generally, an effective dosage may be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range may be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage may be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Examples of pharmaceutically acceptable carriers or diluents are demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The compositions of the invention may be in a form suitable for parenteral administration, or in the form of a formulation suitable for oral ingestion (such as capsules, tablets, caplets, elixirs, for example).

For administration as an injectable solution or suspension, non-toxic parenterally acceptable diluents or carriers can include, Ringers solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

Formulations suitable for topical administration comprise active ingredients together with one or more acceptable carriers, and optionally any other therapeutic ingredients. Formulations suitable for topical administration include liquid or semi-liquid preparations suitable for penetration through the skin to the site of where treatment is required, such as lotions, creams, ointments, pastes or gels.

Creams, ointments or pastes according to the present invention are semi-solid formulations of the active ingredient for external application or for intra-vaginal application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols. The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which are incorporated herein by reference.

The present invention will now be further described in greater detail by reference to the following specific examples, which should not be construed as in anyway limiting the scope of the invention.

### EXAMPLES

### Example 1

### Tammar Wallaby cDNA Libraries

20 cDNA libraries were prepared from tammar wallaby mammary gland tissue as described below in Table 1. These libraries were derived from tissue isolated at different stages during pregnancy or the lactation cycles of wallabies. In some instances (see Table 1) the cDNA was treated, for example for size selection purposes or to remove known milk proteins, prior to ligation into the vector.

Library T20 represents a normalized library prepared (by LifeTechnologies) from equal parts of RNA isolated from pregnant tammar mammary gland at day 23 of gestation, lactating tammar mammary gland at days 55, 87, 130, 180, 220, 260 and from mammary gland after 5 days of involution (preceded by 45 days of lactation). The library was constructed from the pooled RNA using SuperScript II Rnase H-RT, directionally ligated into pCMV Sport 6.0 vector and transformed into ElectroMax DH10B cells.

Approximately 15,000 ESTs were derived from the lactating wallaby gland library described above. From the 15,000, 10,000 were used as elements in the tammar wallaby microarray analysis as decribed in Example 3.

TABLE 1
Tammar cDNA libraries generated in the present study

| | | | | Ligation |
|---|---|---|---|---|
| | Mammary Gland Tissue | | | insert:vector |
| Library | source | RNA purity | Treatment | ratio |
| T01 | Day 130 lactation | total RNA | none ¹ | 1:1 |
| T02 | Day 130 lactation | total RNA | none ¹ | 3:1 |
| T03 | Day 130 lactation | polyA+ RNA | none ¹ | 1:1 |
| T04 | Day 130 lactation | polyA+ RNA | none ¹ | 3:1 |
| T05 | Day 130 lactation | polyA+ RNA | cDNA size selected | 1:1 |
| | | | 0.5-1.0 kbp ¹ | |
| T06 | Day 130 lactation | polyA+ RNA | cDNA size selected | 3:1 |
| | | | 0.5-1.0 kbp ¹ | |
| T07 | Day 130 lactation | polyA+ RNA | cDNA size selected | 1:1 |
| | | | 1.0-2.0 kbp ¹ | |
| T08 | Day 130 lactation | polyA+ RNA | cDNA size selected | 3:1 |
| | | | 1.0-2.0 kbp ¹ | |
| T09 | Day 130 lactation | polyA+ RNA | cDNA size selected | 1:1 |
| | | | 2.0-4.0 kbp ¹ | |
| T10 | Day 130 lactation | polyA+ RNA | cDNA size selected | 3:1 |
| | | | 2.0-4.0 kbp ¹ | |
| T11 | Day 130 lactation | polyA+ RNA | Subtracted for α-casein, β-casein, | 1:1 |
| | | | κ-casein, α-lactalbumin, β- | |
| | | | lactoglobulin ² | |
| T12 | Day 130 lactation | polyA+ RNA | Subtracted for α-casein, β-casein, | 3:1 |
| | | | κ-casein, α-lactalbumin, β- | |
| | | | lactoglobulin ² | |
| T13 | Day 23 pregnancy | polyA+ RNA | none ¹ | 1:1 and 3:1 |
| | | | | combined |
| T14 | Day 260 lactation | polyA+ RNA | none ¹ | 1:1 and 3:1 |
| | | | | combined |
| T15 | Day 23 pregnancy | polyA+ RNA | cDNA synthesized using | 1:1 and 3:1 |
| | | | Thermoscript RT ¹ | combined |
| T16 | Day 23 pregnancy | polyA+ RNA | cDNA fragments purified though | 1:1 |
| | | | column as per manufacturers | |
| | | | instructions ³ | |
| T17 | Day 23 pregnancy | polyA+ RNA | cDNA fragments purified though | 3:1 |
| | | | column as per manufacturers | |
| | | | instructions ³ | |
| T18 | Day 4 lactation, non- | polyA+ RNA | cDNA fragments purified though | 1:1 |
| | sucked gland | | column as per manufacturers | |
| | | | instructions ³ | |
| T19 | Day 4 lactation, non- | polyA+ RNA | cDNA fragments purified though | 3:1 |
| | sucked gland | | column as per manufacturers | |
| | | | instructions ³ | |

| | |
|---|---|
| T20 | normalized library (printed on microarray) |

¹ Prepared using Clontech Smart cDNA Synthesis kit, cDNA cloned in pGEM-T
² Prepared using Clontech DNA-Select Subtraction kit, cDNA cloned in pGEM-T
³ Prepared using Clontech Smart cDNA Library Construction kit

### Example 2

### Lactation-Associated Molecules

The cDNA libraries generated in Example 1 were transformed into either DH10B or JM109 E. coli cells and plated on LB agar containing ampicillin. Individual colonies were picked and grown in LB media containing ampicillin for plasmid preparation and sequencing. The cDNA inserts were sequenced using primers specific to either the T7 or SP6 RNA polymerase promoters in the vector. Alternatively, and where appropriate, the SMART (Switching Mechanism at 5′ end of RNA Template) oligonucleotide (used in the preparation of the cDNA) was used to sequence specifically from the 5′ end of the cDNA. Sequencing was performed on an Applied Biosystems ABI 3700 automated sequencer, using Big-Dye Terminator reactions. The DNA base calling algorithm PHRED and sequence assembly algorithm PHRAP were used to generate the final sequence files.

The polynucleotides and polypeptides identified herein as lactation-associated are designated as Lactation-Associated Molecules (LAMs). As set out in Table 2, each LAM designation may comprise a set of molecules, for example, a Tammar wallaby polypeptide, its corresponding polynucleotide, its bovine polynucleotide orthologue and corresponding bovine polypeptide. Furthermore, some LAMs may additionally comprise an alternative bovine polynucleotide and/or polypeptide sequence. Such alternative sequences may or may not include single nucleotide polymorphisms (SNPs).

TABLE 2
Sequence identities for members of each LAM

| | SEQ ID NOS for each LAM member | | | | | |
|---|---|---|---|---|---|---|
| | | | | | Alternative | Alternative |
| | Tammar Wallaby | Tammar Wallaby | Bovine | Bovine | Bovine | Bovine |
| LAM | polynucleotide | polypeptide | polynucleotide | polypeptide | polynucleotide | polypeptide |
| No. | SEQ ID NO | SEQ ID NO | SEQ ID NO | SEQ ID NO | SEQ ID NO | SEQ ID NO |
| 1 | 1 | 31 | 61 | 91 | | |
| 2 | 2 | 32 | 62 | 92 | | |
| 3 | 3 | 33 | 63 | 93 | | |
| 4 | 4 | 34 | 64 | 94 | | |
| 5 | 5 | 35 | 65 | 95 | | |
| 6 | 6 | 36 | 66 | 96 | | |
| 7 | 7 | 37 | 67 | 97 | | |
| 8 | 8 | 38 | 68 | 98 | | |
| 9 | 9 | 39 | 69 | 99 | | |
| 10 | 10 | 40 | 70 | 100 | | |
| 11 | 11 | 41 | 71 | 101 | | |
| 12 | 12 | 42 | 72 | 102 | 151 | 152 |
| 13 | 13 | 43 | 73 | 103 | | |
| 14 | 14 | 44 | 74 | 104 | | |
| 15 | 15 | 45 | 75 | 105 | 153 | 154 |
| 16 | 16 | 46 | 76 | 106 | 155 | 156 |
| 17 | 17 | 47 | 77 | 107 | | |
| 18 | 18 | 48 | 78 | 108 | | |
| 19 | 19 | 49 | 79 | 109 | | |
| 20 | 20 | 50 | 80 | 110 | | |
| 21 | 21 | 51 | 81 | 111 | | |
| 22 | 22 | 52 | 82 | 112 | | |
| 23 | 23 | 53 | 83 | 113 | | |
| 24 | 24 | 54 | 84 | 114 | 157 | 158 |
| 25 | 25 | 55 | 85 | 115 | | |
| 26 | 26 | 56 | 86 | 116 | 159 | 160 |
| 27 | 27 | 57 | 87 | 117 | 161 | 162 |
| 28 | 28 | 58 | 88 | 118 | 163 | 164 |
| 29 | 29 | 59 | 89 | 119 | 165 | 166 |
| 30 | 178 | 179 | 180 | 181 | 190 | |
| 31 | 183 | 184 | 185 | 186 | 192 | |
| 32 | 167 | 188 | 208 | 220 | 191 | |
| 32a | 168 | 215 | | | | |
| 32b | 169 | 207 | | | | |
| 32.8 | | | 170 | 171 | | |
| 32.9 | | | 172 | 173 | | |
| 32.10 | | | 174 | 175 | | |
| 32.11 | | | 176 | 177 | | |
| 33 | 216 | 217 | 218 | 219 | | |

The Tammar EST sequences of SEQ ID NOs: 1 to 29, 167, 178, 183, and 216 were used to interrogate various sequence databases including the non-redundant GenBank coding sequence translations (+PDB+SwissProt+PIR+PRF), human Unigene, GenBank and the Bovine EST database.

The longest tammar contig sequence was identified using the http://vbc.med.monash.edu.au/˜clefevre/Wallaby/ database. This sequence was used to tblastx the bovine and monodelphis genomes using http://www.ensembl.org/index.html, and the bovine ESTs http://www.livestockgenomics.csiro.au/IBISS4/ databases. As would be known a person of skill in the art, Tblastx converts a nucleotide query sequence into protein sequences in all 6 reading frames and then compares this to an NCBI nucleotide database which has been translated on all six reading frames. Databases were tblastx using algorithms for distantly related sequences. Results were scrutinized for consistency of targets. Human homologues were identified by tblastx of human ESTs at http://www.ncbi.nlm.nih.gov/BLAST/ and the unigene links to proteins identified as the human orthologous protein.

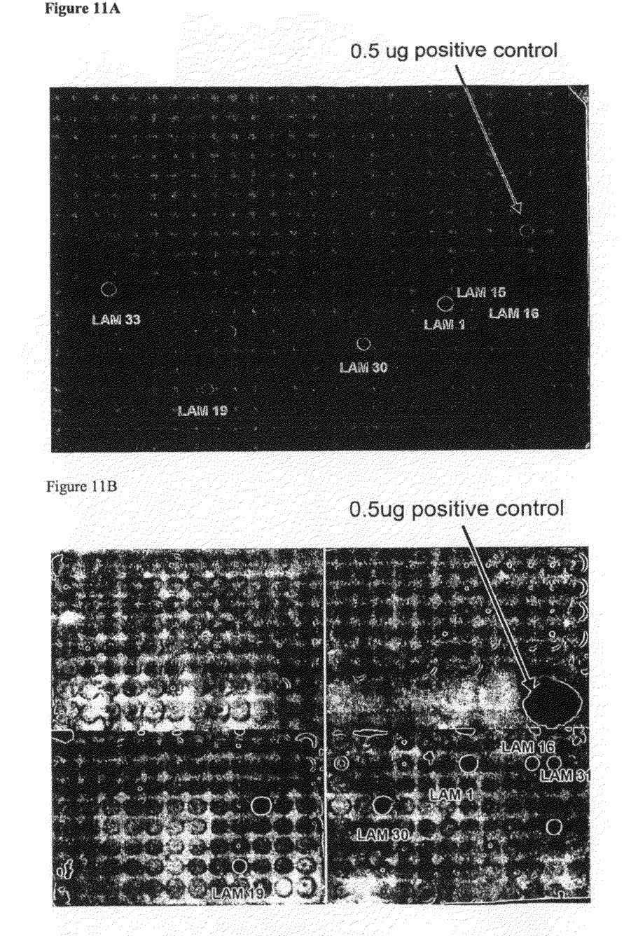

The nucleotide sequences comprising bovine homologues of the Tammar ESTs are shown and set forth as SEQ ID NOs: 61 to 89, 151, 153, 155, 157, 159, 161, 163, 165, 170, 172, 174, 176, 180, 185, 208, and 218. The associated predicted bovine amino acid sequences are also shown and set forth as SEQ ID NOs: 91 to 119, 152, 154, 156, 158, 160, 162, 164, 166, 171, 173, 175, 177, 181,186, and 219. In addition, bovine single nucleotide polymorphisms (SNPs) were elucidated for the majority of LAMs, and SNPs for LAMs 30 to 32 as set forth as SEQ ID NOs: 190, 191, and 192 are exemplified in FIG. 11.

Various identifying characteristics of the bovine polypeptides as set forth as SEQ ID NOs: 91 to 119, 181, 186, and 219 are listed in Table 3 below. A leader sequence prediction algorithm (Bannai et al., 2002, Extensive feature detection of N-terminal protein sorting signals, Bioinformatics, 18:298-305) was used to identify predicted leader sequences. The inclusion of a specific leader amino acid sequence in Table 3 indicates that a cleavage site is predicted, and thus suggests that the LAM polypeptide is secreted. The predicted molecular weights and isoelectric points were also determined. Where a leader sequence cleavage site is predicted, the estimated molecular weight shown is that of the mature polypeptide.

TABLE 3
Predicted characteristics of bovine polypeptides

| | Molecular weight | pl of mature | |
|---|---|---|---|
| LAM No | (Daltons) | polypeptide | Leader sequence prediction |
| LAM 1 | 46553 | 5.90 | Transmembrane |
| LAM 2 | ND | ND | MRPSGQLPLTGLLFFSLIPSQLCQI |
| LAM 3 | 49988 | 8.17 | none |
| LAM 4 | 18004 | 10.41 | MAELVKSKYGQVTEYTFTSANVSPSPSFLGEIHF |
| | | | QGVDCET |
| LAM 5 | 33254 | 8.20 | Transmembrane |
| LAM 6 | 76499 | 7.99 | MSGCGLFLCSVAARFCRAPA |
| LAM 7 | 34090 | 9.40 | MKTLIAAYSGVLRGTGSSILSALQDLFSVTWLNR |
| | | | SKVEKQLQVISVLQWVLSFLVLGVACSVI |
| LAM 8 | 30682 | 6.42 | MAAAARGSGRASAPGLFLVLLVPLLWAPAGVRA |
| | | | VP |
| LAM 9 | 14737.80 | 4.58 | None |
| LAM 10 | 53429 | 5.92 | None |
| LAM 11 | 22815 | 9.72 | None |
| LAM 12 | 54211.31 | 5.70 | MAGFPGLFPAGVLPALLLWVSMWGSP |
| LAM 13 | 14323.06 | 6.83 | MRLLVLAALLTVGAGQA |
| LAM 14 | 31333 | 7.74 | Transmembrane |
| LAM 15 | 130175.42 | 6.8415 | MDPPAGAAGRLLCPALLLLLLLPLPADARLAAAA |
| | | | ADPP |
| LAM 16 | 111221 | 5.91 | MNGAEAGEGDALASLAQSRHLACTSGLVVFRFP |
| | | | KNVQAAV |
| LAM 17 | 31136 | 5.33 | MLSETIVSEFPVYVLSSLISDTVXVLPMGKMAKM |
| | | | FSFILVTTALVMGRGSS |
| LAM 18 | 11343 | 9.30 | MLFSLRELVQWLGFATFEIFVHLLALLVFSVLLAL |
| | | | RVD |
| LAM 19 | 24076 | 7.14 | MTTNTSPMHPYWPRHLRLDNFVPNDYPTWHILA |
| | | | GLFSVSGVLVVATWLLSGRAAV |
| LAM 20 | 15710 | 6.39 | Transmembrane |
| LAM 21 | ND | ND | None |
| LAM 22 | 14779 | 6.73 | None |
| LAM 23 | ND | ND | MKLDIQCEQLSDARWTELLPLIQQYEVVRLDD |
| LAM 24 | 23070 | 6.04 | Transmembrane |
| LAM 25 | 55110.46 | 8.36 | Transmembrane |
| LAM 26 | 33800 | 9.84 | Transmembrane |
| LAM 27 | 98533 | 6.79 | MASSAQSGGSSGGPAVPTVQRGIVKMVLSGCAII |
| | | | VRGQP |
| LAM 28 | 33875 | 6.11 | MAPMGIRLSPLGVAVFCLLGLGVLYHLYSGFLAG |
| | | | RFSLFGLGGEPGGGAAGP |
| LAM 29 | 123798.74 | 6.35 | MAEAAPHHPALPSGLLELCALLGAPRDS |
| LAM 30 | 45993 | 8.77 | MLFRNRFVLLLALAALLAFVSLSLQ |
| LAM 31 | 80700 | 6.69 | MATYLEFIQQNEERDGVRFSWNVWPSSRLEATR |
| | | | MVVPLACLLTPLKERP |
| LAM 32 | 14141 | 6.73 | METPRASLSLGRWSLWLLLLGLALPSAQR |
| LAM 33 | 34217 | 8.98 | MLKSRLRMFLNELKLLVLTGGGRPRAEPQP |

### Example 3

### Tammar Wallaby Microarray Expression Profiling

cDNA inserts containing the Tammar wallaby ESTs of SEQ ID NOs: 1 to 29, 167, 178, 183, and 216 were amplified using 17 and SP6 primers and Perkin-Elmer Taq polymerase. The resulting amplified DNA samples and Amersham's Lucidia scorecard DNA were spotted onto glass slides by the Peter MacCallum Microarray Facility (under contract). Total RNA from pregnant and lactating tammar wallaby mammary gland was extracted from tissues using Tripure Isolation Reagent (Roche), and further purified using Qiagen RNeasy columns. RNA was labeled using amino allyl reverse transcription followed by Cy3 and Cy5 coupling. Samples of 50 ug total RNA and Amersham's Lucidia Scorecard Mix were reverse transcribed in 87 ng/ul oligo dT Promega MMLV reverse transcriptase, RNAseH and 1× buffer at 42° C. for 2.5 hours. The resultant products were hydrolyzed by incubation at 65° C. for 15 minutes in the presence of 33 mM NaOH, 33 mM EDTA and 40 mM acetic acid. The cDNA was then adsorbed to a Qiagen QIAquick PCR Purification column.

Coupling of either Cy3 or Cy5 dye was performed by incubation with adsorbed cDNA in 0.1M sodium bicarbonate for 1 hour at room temperature in darkness, followed by elution in 80 ul water. Labeled cDNA was further purified using a second Qiagen QIAquick PCR Purification column. Cy3 and Cy5 labeled probes in a final concentration of 400 ug/ml yeast tRNA, 1 mg/ml human Cot-1 DNA, 200 ug/ml polydT₅₀, 1.2× Denhart's, 1 mg/ml herring sperm DNA, 3.2×SSC, 50% formamide and 0.1% SDS were heated to 100° C. for 3 minutes and then hybridized with microarray spotted cDNAs at 42° C. for 16 hours.

Microarrays were washed in 0.5×SSC, 0.01% SDS for 1 minute, 0.5×SSC for 3 minutes then 0.006×SSC for 3 minutes at room temperature in the dark. Slides were scanned and the resulting images processed using Biorad Versarray software.

Data from spot intensities were either cross channel Loess normalized or single channel normalized. Cross channel normalization was performed using the Versarray software using the parameters shown in Table 4.

TABLE 4
Parameters used for cross channel normalization

| | |
|---|---|
| Background method | “Local ring, Offset: 1, Width: 2, Filter: 0 |
| | Erosion: 0” |
| Net intensity | Raw intensity - Median background (Ignore |
| measurement method | negatives) |
| Net intensity | “Cross-channel, Local regression (Loess), |
| Normalization | Median” |
| Cell shape | Ellipse |
| Cell size | 30 × 30 pixels |

Single channel normalization used the Bioconductor software (Smyth and Speed, 2003, Normalization of cDNA microarray data, Methods 2003 31:265-73, see LIMMA http://bioinf.wehi.edu.au/limma) on data generated from the Versarray image analysis.

Microarray analysis of gene expression was performed using the cross phase comparisons as shown in Table 5.

TABLE 5
Microarray analysis of gene expression
Mammary tissue samples:

| Phase 1 tissue | Phase 2A tissue | Phase 2B tissue | Phase 3 tissue |
|---|---|---|---|
| day 5 Pregnancy | day 1 Lactation | day 130 Lactation | day 213 Lactation |
| day 22 Pregnancy | day 5 Lactation | day 168 Lactation | day 220 Lactation |
| day 25 Pregnancy | day 80 Lactation | day 180 Lactation | day 260 Lactation |

| Phase 1-2A Comparisons | | | | | | | | |
|---|---|---|---|---|---|---|---|---|
| Comparisons | | | Phase 2A-2B Comparisons Phase | | | 2B-3 | | |
| Cy3 | | Cy5 | Cy3 | | Cy5 | Cy3 | | Cy5 |
| 5P | versus | 80L | 80L | versus | 168L | 130L | versus | 260L |
| 5P | versus | 1L | 130L | versus | 1L | 130L | versus | 213L |
| 22P | versus | 5L | 168L | versus | 80L | 168L | versus | 220L |
| 22P | versus | 80L | | | | 168L | versus | 260L |
| 25P | versus | 1L | | | | 180L | versus | 213L |
| 25P | versus | 51 | | | | 168L | versus | 213L |
| 5L | versus | 22P | | | | 260L | versus | 130L |
| 80L | versus | 22P | | | | 213L | versus | 130L |
| 1L | versus | 25P | | | | 220L | versus | 168L |
| 5L | versus | 25P | | | | 260L | versus | 168L |
| 213L | versus | 168L | | | | | | |

Number: number of days
P: Pregnant
L: Lactating

A total of 398 elements were identified as being differentially expressed and subsequently cloned into vectors for functional analysis. After initial testing, LAMs 1-33 as set out in Tables 2 and 3 were selected and further tested for specific activities as set out below.

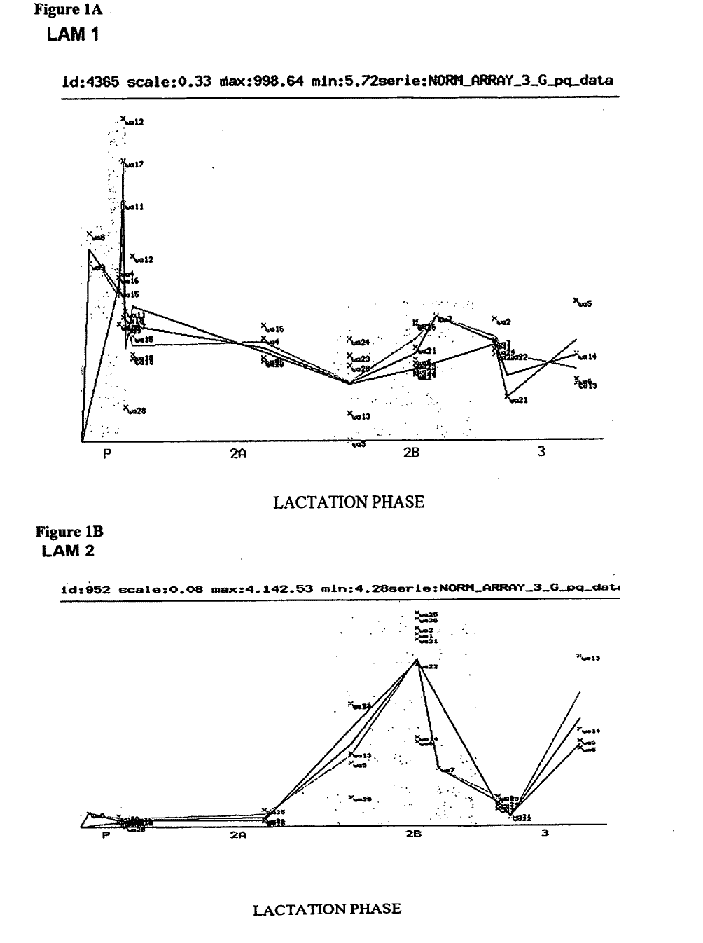

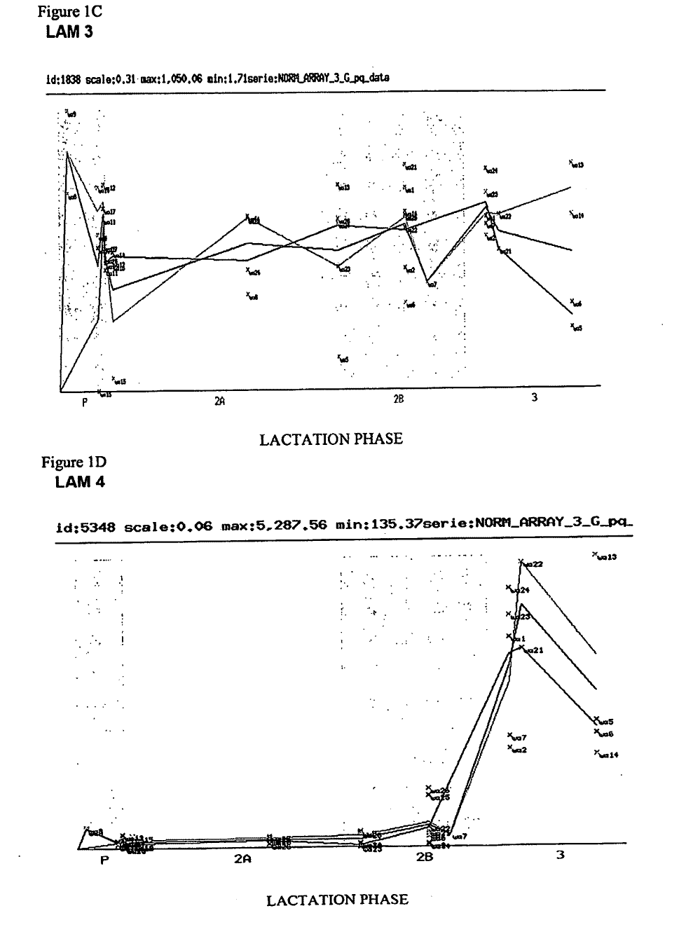

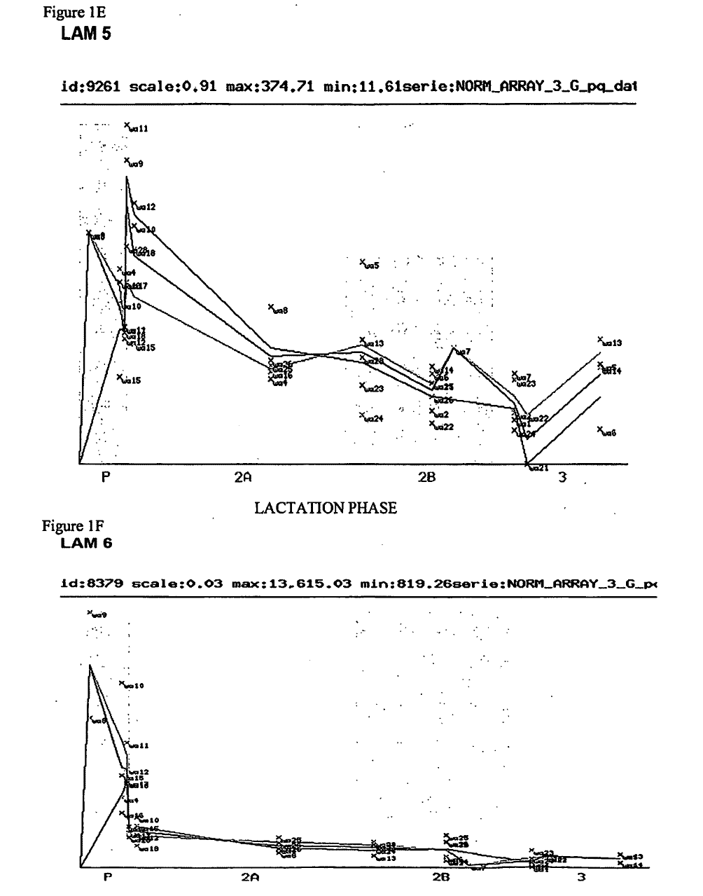

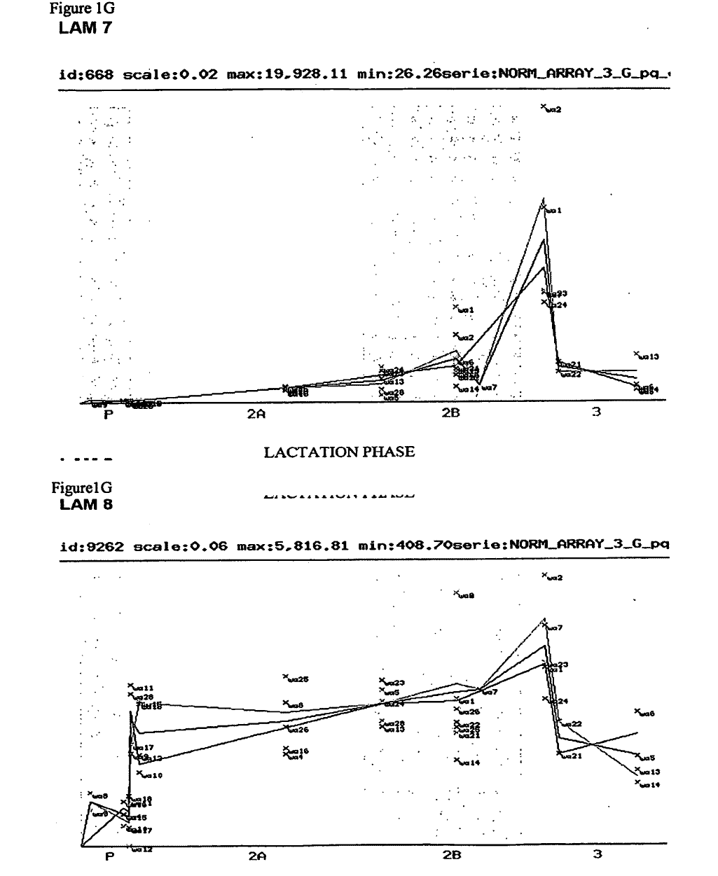

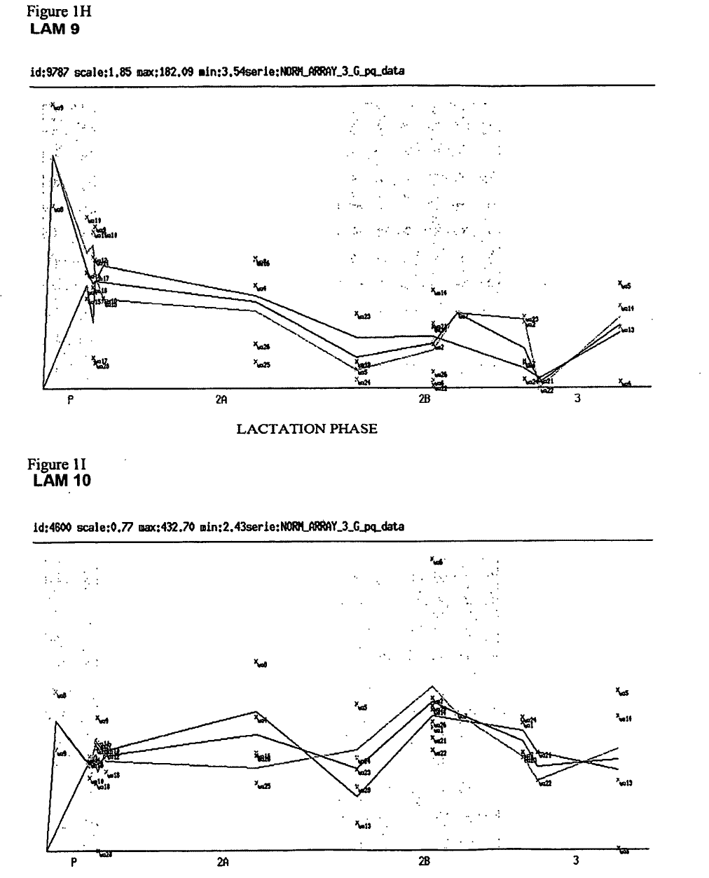

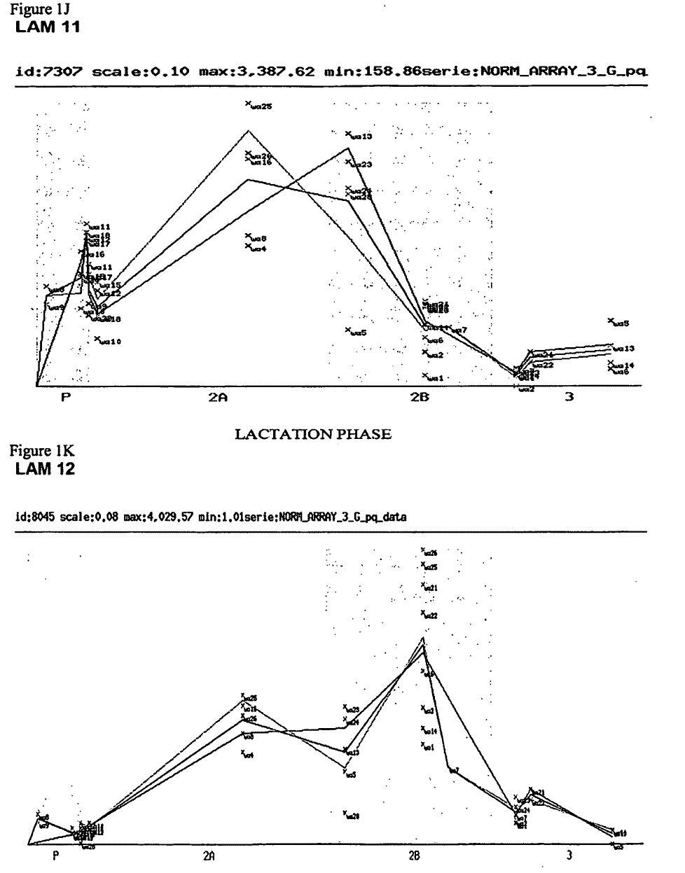

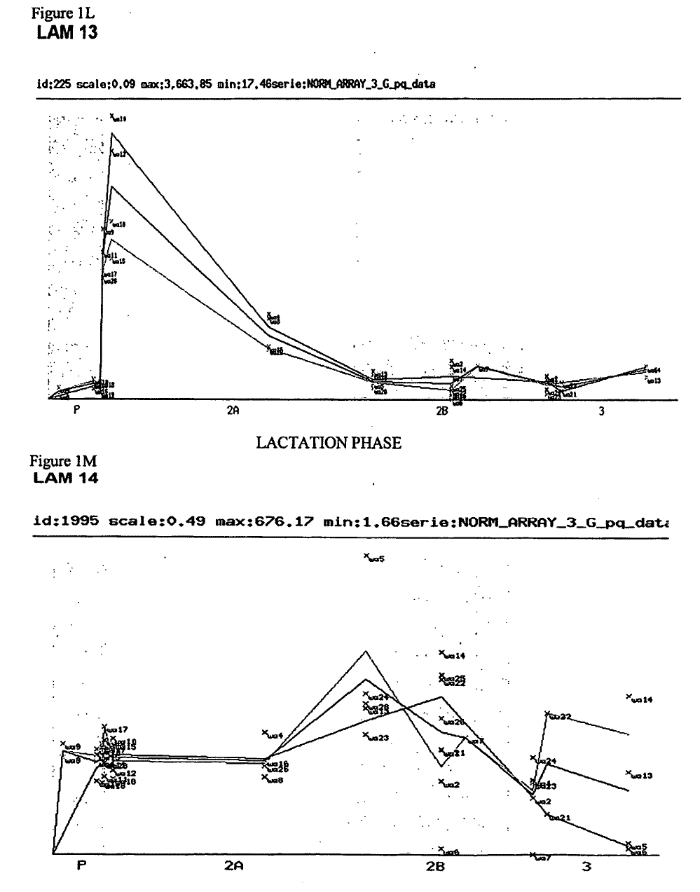

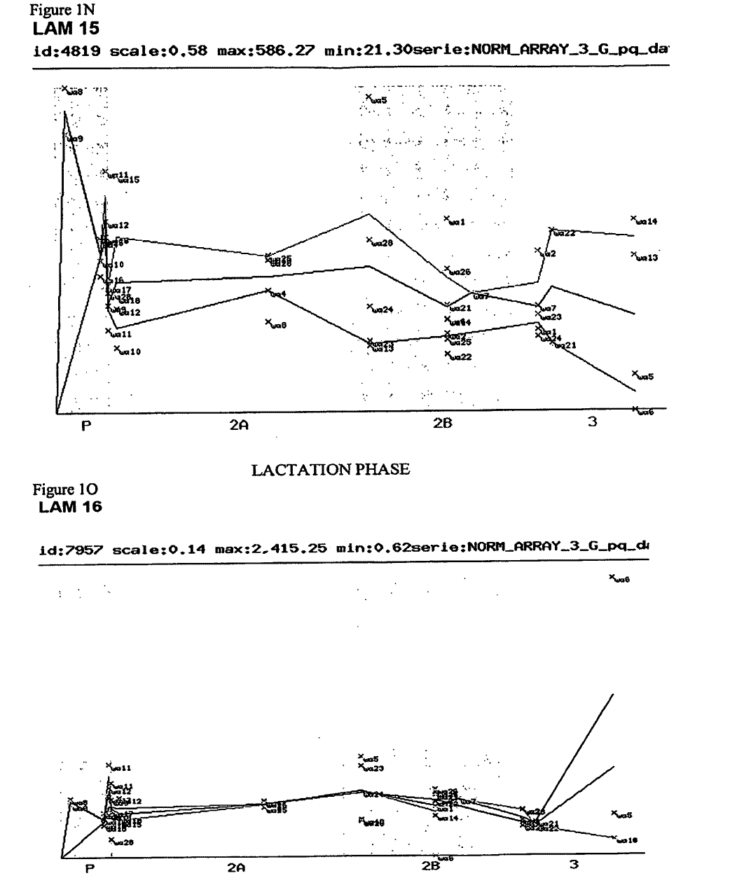

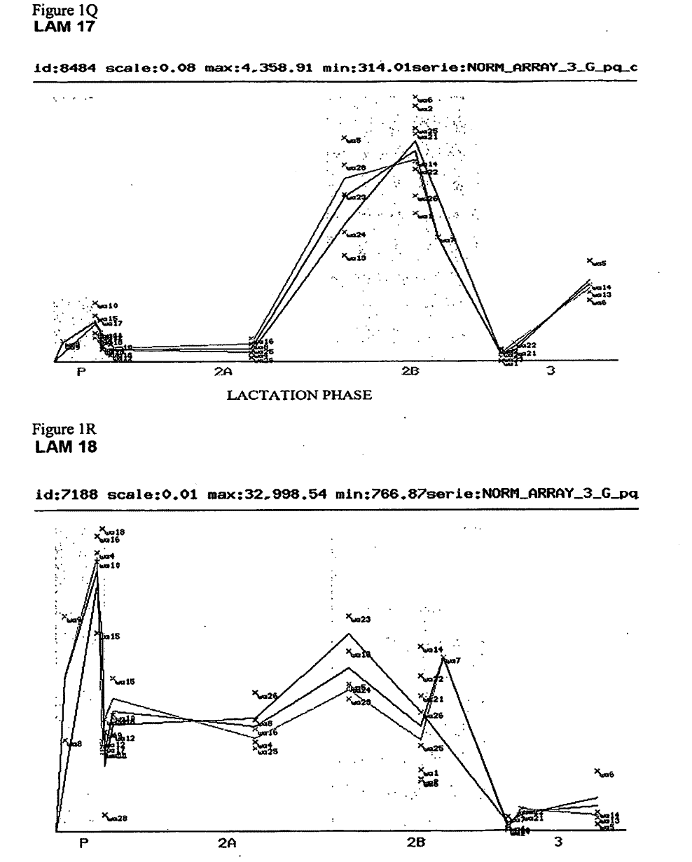

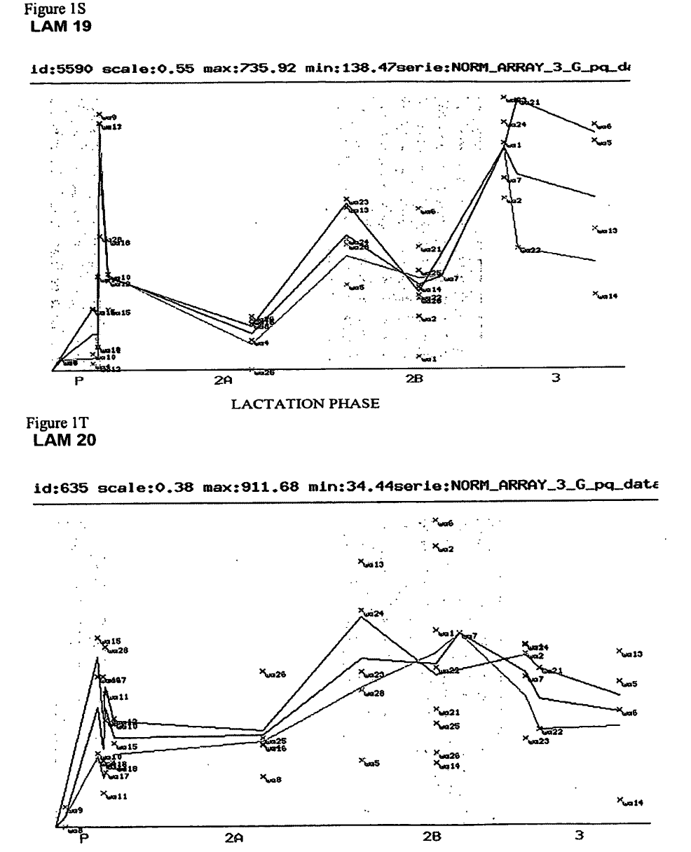

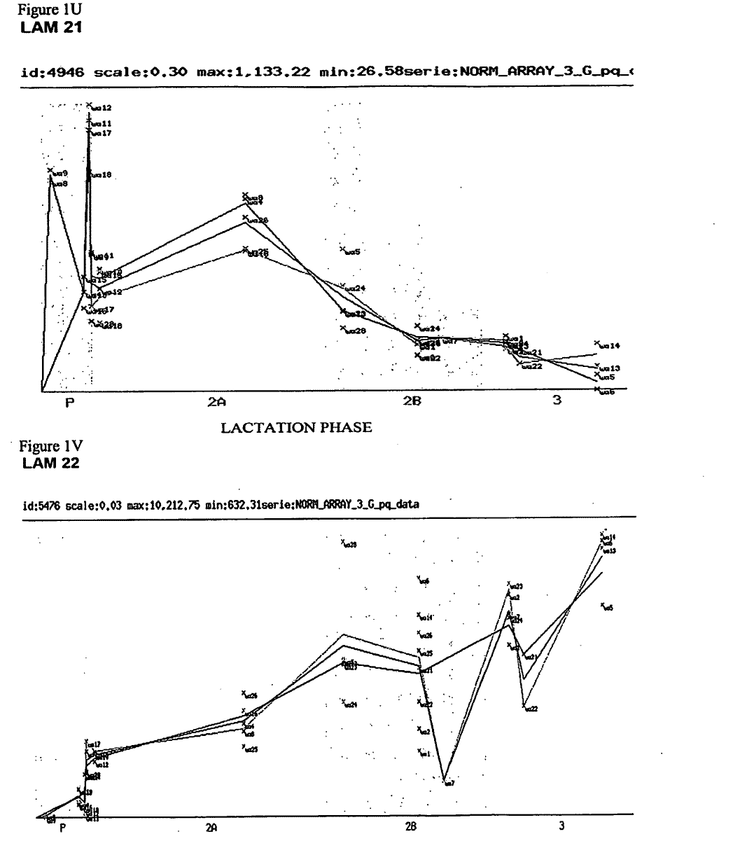

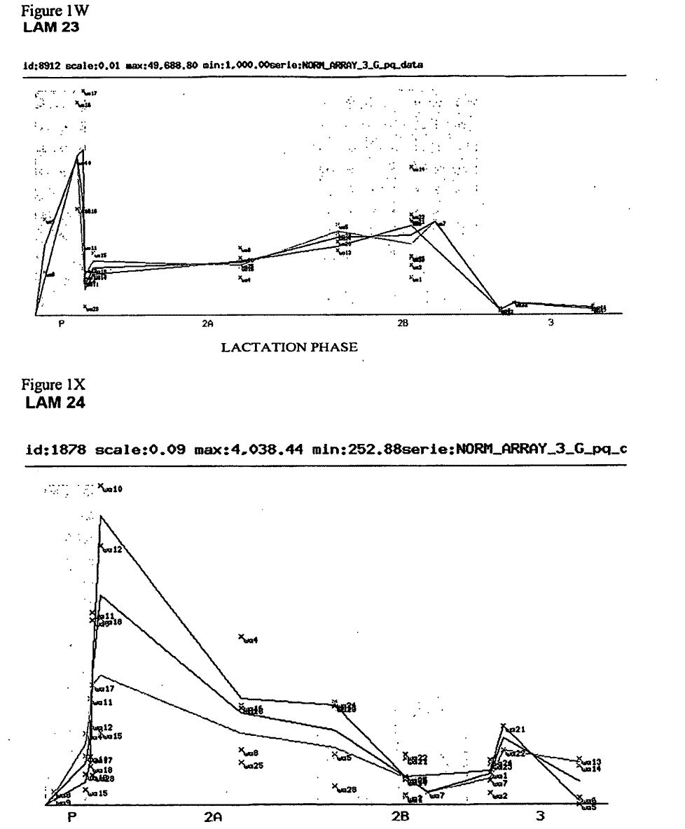

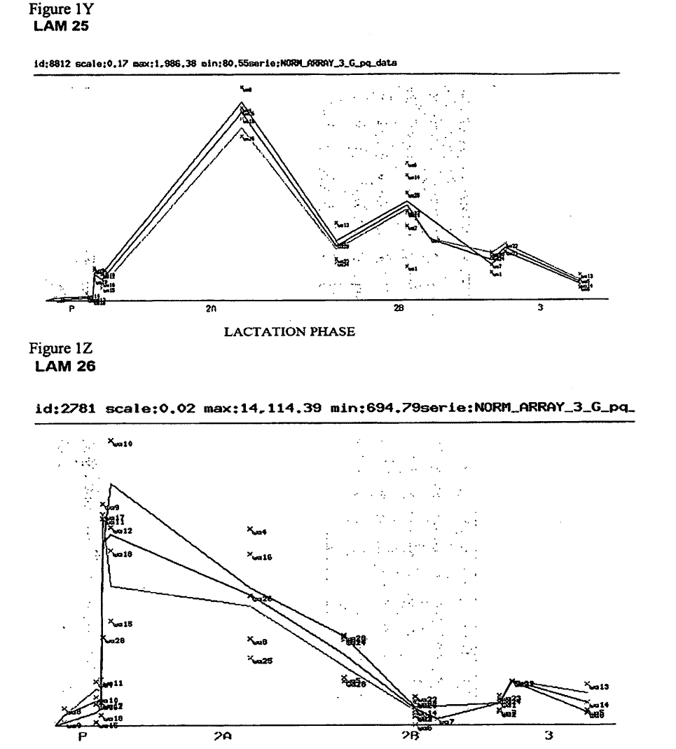

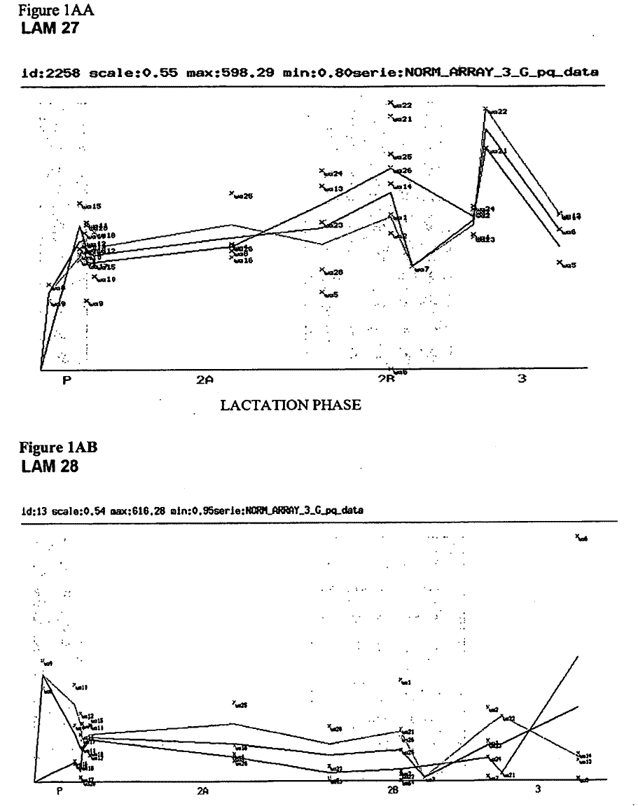

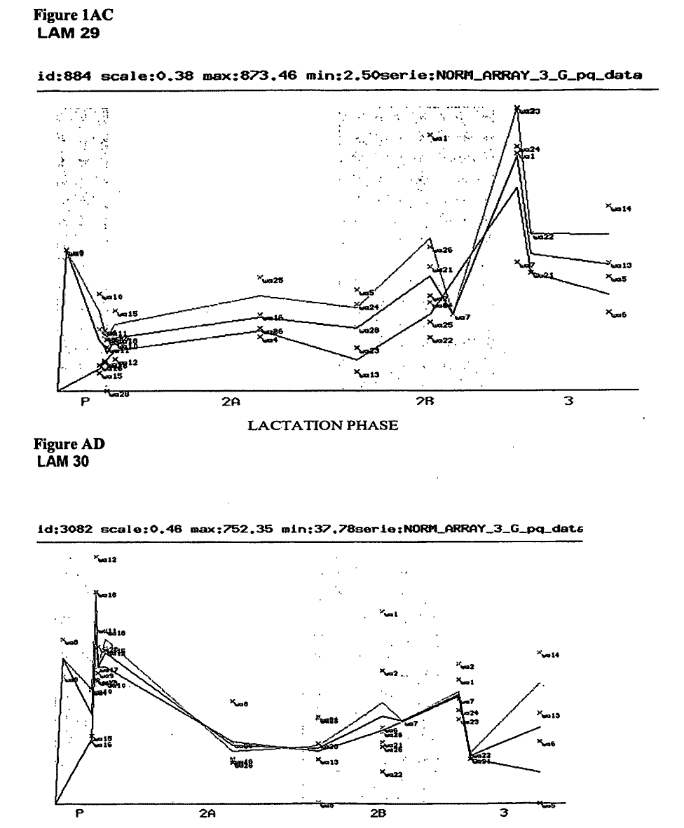

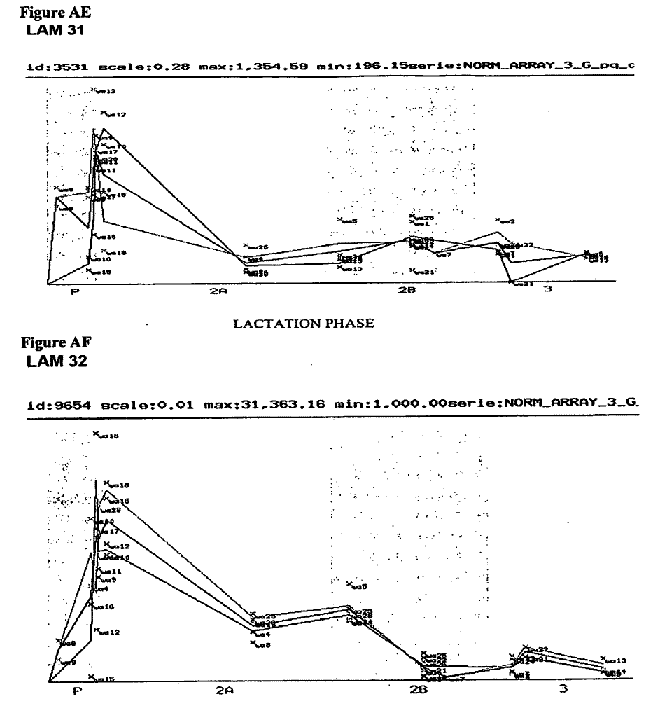

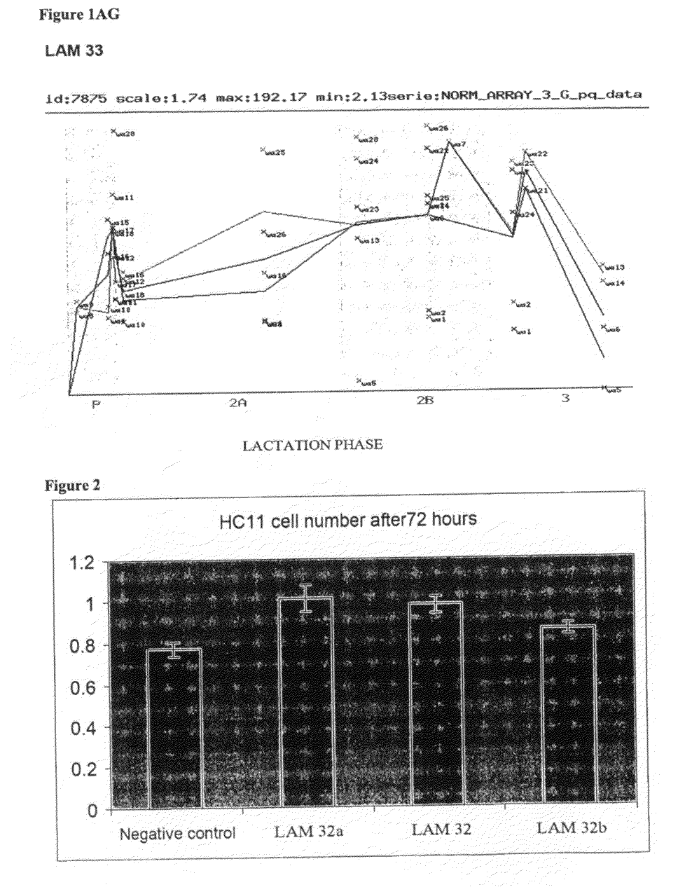

The results of the lactation-associated microarray expression profiling for Tammar wallaby LAMs 1 to 33 are exemplified and provided in FIG. 1. The graphs show the normalized spot intensities for each Tammar LAM across each of the phases of the Tammar lactation cycle: P, pregnancy; Phase 2A, first 100 days of lactation; Joey permanently attached); Phase 2B, days 100-200 of lactation (joeys begin to relinquish teat, growth rate slows but physiological development is advanced); and Phase 3, day 200+ of lactation (joey in and out of pouch). All LAMs were clearly expressed in mammary tissue of the Tammar wallaby and show changes in expression levels across lactation. For each LAM at least one 5-fold change (increase or decrease) in expression was observed.

### Example 4

### Biological Activities of Secreted Polypeptides

Plasmids containing Tammar wallaby LAMs were directionally cloned into the expression vector pCMV Sport 6.0, and then transfected into the human kidney cell line HK293. A total of 1 μg of LAM-containing plasmid DNA and 10 ng of pEGFP-C1 plasmid was introduced into 70% confluent HK293 cells in 2 cm² wells containing 500 ul of opti-MEM-1 media. Transfection success was assessed by observing green fluorescence of cells by fluorescent microscopy. After 48 hours conditioned media containing the secreted polypeptide was collected and frozen at −20° C. The media containing the secreted polypeptides was then used directly in a variety of bioactivity assays. For the assays described below, samples of secreted polypeptides were aliquoted into individual wells of 96 or 384 well plates and stored, prior to assaying, either frozen or lyophilized.

A negative control created by transfection with an insertless vector was used in all assays. For the assays described in Examples 4A, 4B, 4C and 4D results for samples were in the form of relative fluorescence units (RFU) as described herein. A result was considered positive for an assay (and hence the LAM was considered to display the relevant activity) where the sample produced RFU approximately two standard deviations from the mean. In some cases, RFU less than two standard deviations from the mean were considered significant if the LAM sample in question produced a positive result in another assay.

In accordance with the best mode of performing the invention provided herein, additional specific examples of biological activity assays are outlined below. The following are to be construed as merely illustrative examples of assays and not as a limitation of the scope of the present invention in any way.

### Example 4A

### ERK Assay for Cell Growth-Promoting Activity

Extracellular signal-regulated protein kinase (ERK) is a common and central signal transduction pathway component of tyrosine kinase receptor. Activation of ERK is indicative of an extracellular proliferation signal and provides an index of a growth promoting agent.

Swiss 3T3 fibroblast cells were plated into 384 well plates, grown to confluence and starved overnight with serum-free medium. Cells were then treated for 10 minutes with a LAM polypeptide prepared as described above. Cells were then lysed and assayed for activation of ERK. Samples were assessed for changes in the activity of ERK.

The results of ERK activation assays are shown in Table 6 as relative fluorescence units (RFU). Samples producing levels of ERK activation significantly above the mean are boxed in Table 6 and indicate a growth-promoting activity. These same samples are equated with LAM numbers as represented in Table 7. Activation of ERK by increasing concentrations of betacellulin was used as a positive control in each case.

TABLE 6
ERK activation data from TGR assay

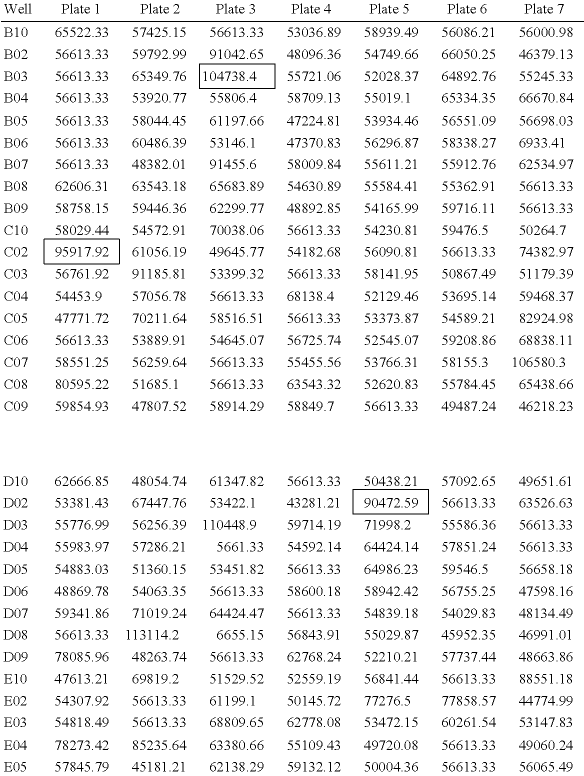

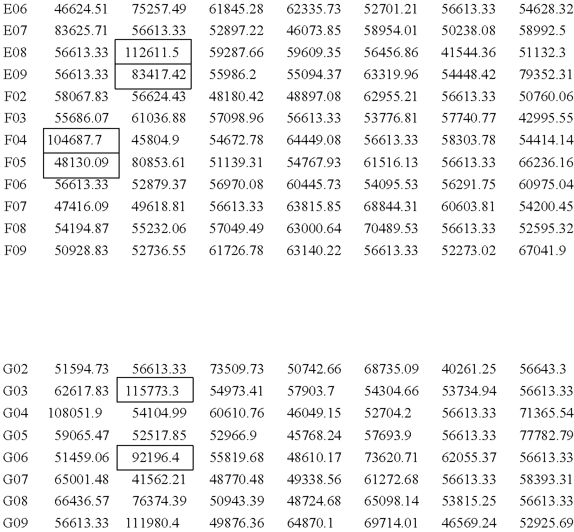

TABLE 7
LAMs showing ERK activation (indicative
of cell growth promoting activity)

| | Position in TGR | | | |
|---|---|---|---|---|
| LAM No | Assay | RFU | Mean RFU | SD RFU |
| LAM 2 | Plate 1 - F4 | 104688 | 56613 | 11843 |
| LAM 4 | Plate 1 - C2 | 95918 | 56613 | 11843 |
| LAM 5 | Plate 2 - G3 | 115773 | 56613 | 11843 |
| LAM 7 | Plate 2 - E8 | 112612 | 56613 | 11843 |
| LAM 12 | Plate 2 - G6 | 92196 | 56613 | 11843 |
| LAM 18 | Plate 2 - E4 | 85236 | 56613 | 11843 |
| LAM 21 | Plate 5 - D2 | 90473 | 56613 | 11843 |
| LAM 22 | Plate 2 - E9 | 83417 | 56613 | 11843 |
| LAM 23 | Plate 2 - F5 | 80854 | 56613 | 11843 |
| LAM 25 | Plate 3 - B3 | 104738 | 56613 | 11843 |

The negative control was 65439 RFU which was 0.5 standard deviations from the mean.

### Example 4B

### Cell Viability Assay to Assess Anti- and Pro-Apoptotic Effects

Vinblastine is a commonly used cytotoxic agent used in chemotherapy. It induces apoptosis in a wide variety of cell types. Caspase activation and DNA fragmentation are hallmarks of the apoptotic process.

Aliquots of the secreted polypeptide samples in 96 well plates were pipetted onto HSC-2 oral epithelial cells and the cells left for 24 hours. After this time, the cells were treated with vinblastine to induce apoptosis. After a further 48 hours, the cells were analyzed for survival using a vital dye, namely Alamar Blue (resazurin) fluorescent dye (TGR BioSciences Pty Ltd, Adelaide, Australia). This dye method is a TGR proprietary assay technology and as such would be known by a person skilled in the art. Internal controls for induction of cell death via apoptosis as well as assay performance were also included on each plate. Cell survival measurements with this technique reflected the degree of apoptosis. As shown in Tables 8 and 10, boxed results were significant.

TABLE 8
Anti-apoptotic data from TGR assay

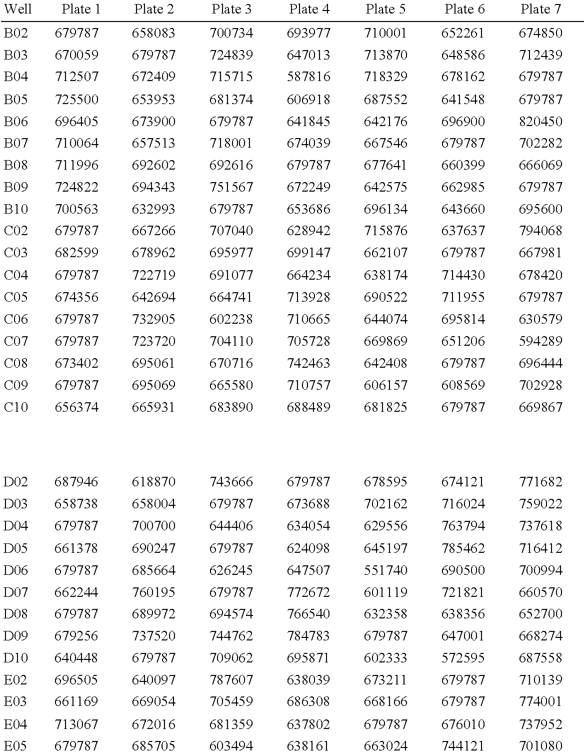

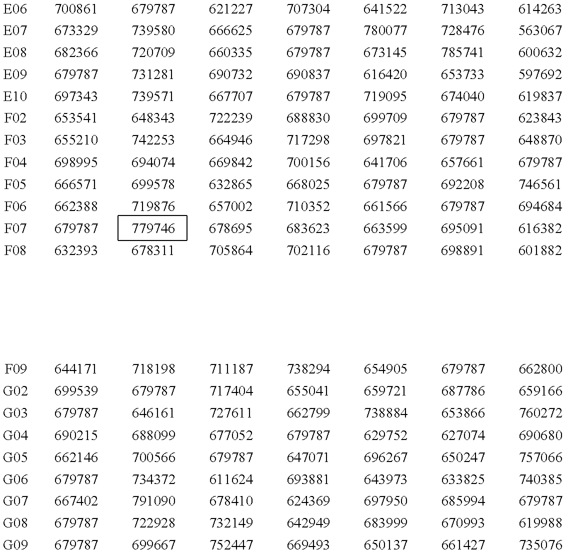

TABLE 9
LAMs showing anti-apoptotic activity

| | Position in TGR | | | |
|---|---|---|---|---|
| LAM No | Assay | RFU | Mean RFU | SD RFU |
| LAM 13 | Plate 2 - F7 | 779746 | 679787 | 39919 |

The negative control was 696444 RFU.

TABLE 10
Pro-apoptotic data from TGR assay

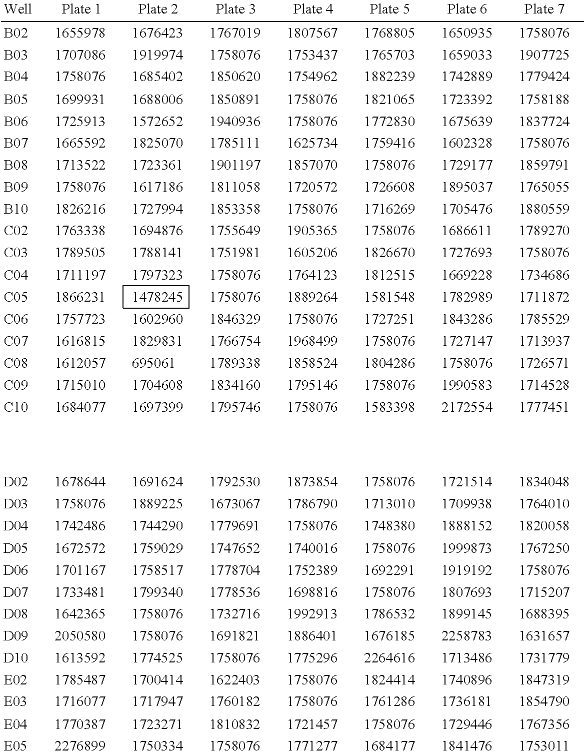

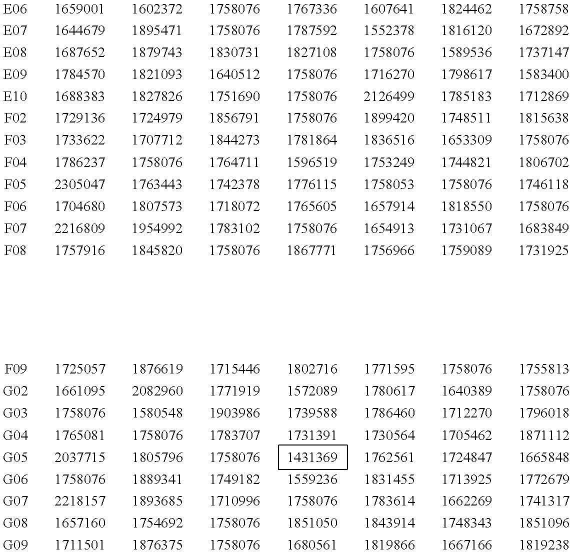

TABLE 11
LAMs showing pro-apoptotic activity

| | Position in TGR | | | |
|---|---|---|---|---|
| LAM No | Assay | RFU | Mean RFU | SD RFU |
| LAM 16 | Plate 4 - G5 | 1431369 | 1758076 | 108156 |
| LAM 17 | Plate 2 - C5 | 1478245 | 1758076 | 108156 |

As can be seen from Tables 8-11, LAM 13 displayed an RFU value significantly above the mean, reflecting anti-apoptotic activity, and LAMs 16 and 17 displayed an RFU value significantly below the mean, reflecting pro-apoptotic activity.

### Example 4C

### p38 MAPK Assay of Inflammatory Activity

p38 MAP kinase (MAPK) is also known as Mitogen-Activated Protein Kinase 14, MAP Kinase p38, p38 alpha, Stress Activated Protein Kinase 2A (SAPK2A), RK, MX12, CSBP1 and CSBP2. p38 is involved in a signaling system that controls cellular responses to cytokines and stress and p38 MAPK is activated by a range of cellular stimuli including osmotic shock, lipopolysaccharides (LPS), inflammatory cytokines, UV light and growth factors. Therefore, activation of p38 MAPK is an indicator of pro-inflammatory activity.

Activation of p38 MAPK was assessed in RAW macrophage cells. These cells were plated into 384 well plates, grown to confluence, starved for 3 hours with serum-reduced medium, and then treated for 30 minutes with the secreted polypeptide LAM samples. Cells were then lysed and assayed for p38 MAPK activation by the use of a p38 MAPK specific fluorescent dye (TGR BioSciences Pty Ltd, Adelaide, Australia). This dye method is a TGR proprietary assay technology and as such would be known by a person skilled in the art. Internal controls for cell activation of p38 MAPK and assay performance were also included in unused wells.

The results are shown below as RFU in Tables 12 and 13. An RFU significantly above the mean reflects pro-inflammatory activity of the sample. As shown in Table 12, significant samples are boxed.

TABLE 12
p38 MAPK activation reflecting pro-inflammatory activity

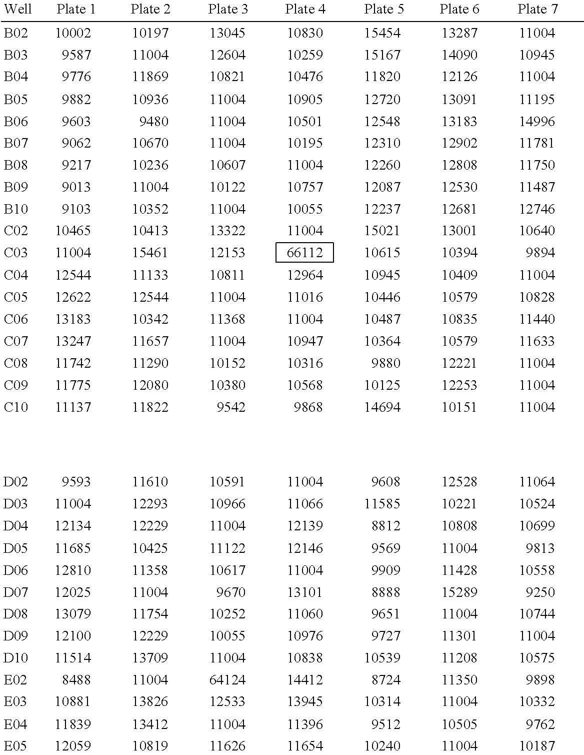

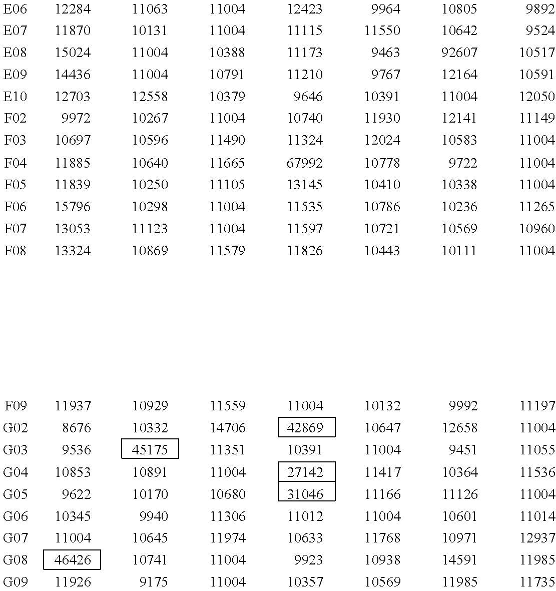

TABLE 13
p38 activation for LAMs- pro-inflammatory activity

| | Position in TGR | | | |
|---|---|---|---|---|
| LAM No | Assay | RFU | Mean RFU | SD RFU |
| LAM 1 | Plate 4 - G2 | 42869 | 12253 | 7395 |
| LAM 5 | Plate 2 - G3 | 45175 | 12253 | 7395 |
| LAM 11 | Plate 1 - G8 | 46426 | 12253 | 7395 |
| LAM 15 | Plate 4 - G4 | 27142 | 12253 | 7395 |
| LAM 16 | Plate 4 - G5 | 31046 | 12253 | 7395 |
| LAM 19 | Plate 4 - C3 | 66112 | 12253 | 7395 |

The negative control was 11004 RFU.

As can be seen from Tables 12 and 13, LAMs 1, 5, 11, 15, 16 and 19 displayed an RFU value significantly above the mean, reflecting pro-inflammatory activity.

### Example 4D

### p38 MAPK Assay of Inflammatory Activity in the Presence of LPS

Activation of p38 MAPK was assessed in RAW macrophage cells in the presence of lipopolysaccharide (LPS), which is a potent stimulator of the immune system and which mimics bacterial infection, thereby providing a model system for inflammation. Cells were plated into 384 well plates, grown to confluence, starved for 3 hours with serum-reduced medium, and then treated for 30 minutes with the secreted polypeptide LAM samples. Cells then received LPS for 30 minutes to stimulate p38 MAPK. After this time, cells were then lysed and assayed for p38 MAPK activation by the use of a p38 MAPK specific fluorescent dye (TGR BioSciences Pty Ltd, Adelaide, Australia). This dye method is a TGR proprietary assay technology and as such would be known by a person skilled in the art.

Internal controls for cell activation of p38 MAPK and assay performance were also included in unused wells. The results are shown as RFU in Tables 14 and 15. An RFU significantly below the mean reflects anti-inflammatory activity of the sample. As shown in Table 14, significant samples are boxed.

TABLE 14
p38 MAPK inhibition reflecting-anti-inflammatory activity

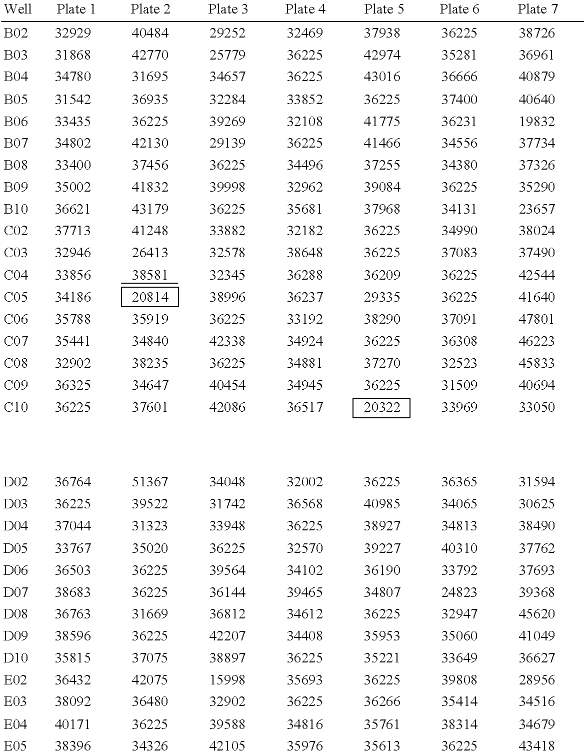

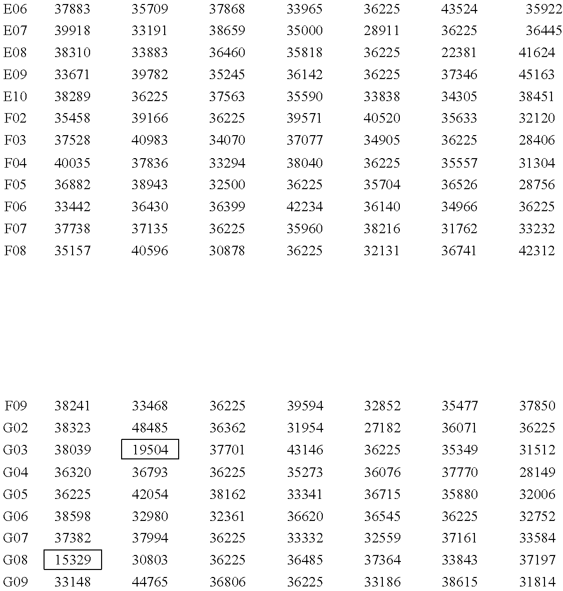

TABLE 15
p38 inhibition for LAMs- anti-inflammatory activity

| | Position in TGR | | | |
|---|---|---|---|---|
| LAM No | Assay | RFU | Mean RFU | SD RFU |
| LAM 5 | Plate 2 - G3 | 19504 | 35980 | 4227 |
| LAM 6 | Plate 5 - C10 | 20322 | 35980 | 4227 |
| LAM 11 | Plate 2 - G8 | 15329 | 35980 | 4227 |
| LAM 17 | Plate 2 - C5 | 20814 | 35980 | 4227 |

The negative control was 45833 RFU.

As can be seen from Tables 14 and 15, LAMs 5, 6, 11 and 17 displayed an RFU value significantly below the mean, reflecting anti-inflammatory activity.

### Example 4E

### Induction of Trefoil Proteins

Trefoils, and specifically Trefoil 1, are known to protect epithelial surfaces, and in addition, to accelerate repair of the epithelium of the gastrointestinal tract. Trefoils are in clinical trials for several applications, including the amelioration of the effects of cancer therapies on the gastrointestinal tract (http://www.thegicompany.com/pages/tech_itf.html).

LAM 30 (SEQ ID NOs: 178-181, and 190) and 31 (SEQ ID NOs: 183-186, and 192) were found to have projected bioactivities as determined by database analysis in the induction of expression of trefoil proteins.

In addition, supernatants of HEK293 cells expressing tammar mammary clones were applied to the human gastric epithelial cell line, AGS, for 48 hours. The resultant AGS cell conditioned media containing stimulated AGS cell secreted proteins (i.e. supernatant) were collected and the AGS cells were lysed. Samples of the supernatant, cell lysate, and a recombinant positive control were transferred to a solid support and probed using a rabbit anti-trefoil 1 antibody freely supplied by Dr Andy Giraud, University of Melbourne under a materials transfer agreement and a secondary goat anti-rabbit antibody conjugated to horseradish peroxides was used to detect LAMs.

Specifically, FIG. 11A is a photograph showing secreted trefoil 1 in supernatants collected from stimulated AGS cells. FIG. 11B shows intracellular trefoil 1 by testing cell lysate of stimulated AGS cells.

As represented in Table 16, LAMs 1, 15, 16, 19, 30, 31 and 33 were shown to enhance trefoil 1 expression and/or secretion by the visualization of the peroxidase reaction in specific wells as depicted in FIGS. 11A and 11B. As can be observed and summarized in Table 16, LAMs 1, 16, 19, and 30 were able to enhance trefoil 1 expression in AGS cells as well as enhance the secretion of trefoil 1 as detected in the supernatant. However some LAMs, namely LAMs 15, 31, and 33 were only involved in either enhanced secretion or enhanced expression.

TABLE 16
Trefoil-inducing activity for LAMs

| Lam No | Function |
|---|---|
| LAM 1 | enhanced expression and secretion |
| LAM 15 | enhanced secretion |
| LAM 16 | enhanced expression and secretion |
| LAM 19 | enhanced expression and secretion |
| LAM 30 | enhanced expression and secretion |
| LAM 31 | enhanced expression |
| LAM 33 | enhanced secretion |

### Example 4F

### Cathelicidins

Cathelicidins are antimicrobial peptides within neutrophils that assist in deterring bacterial infections. Cathelicidins are defined as small peptides less than 100 amino acid residues and are important effector molecules in innate immunity. They are mainly found in the peroxidase negative granules of neutrophils. Furthermore, these cathelicidins can be found in species as diverse as trout to humans demonstrating a wide range of antibacterial actions which resides in the non-conserved C-terminal region of these cathelicidin proteins. However, the human cathelicidin hCAP-18 has also been found in various epithelial sites, mast cells and subpopulations of monocytes and lymphocytes. Cathelicidins have not previously been found to be involved with lactation or any other aspects of parturition.

LAMs 32, 32a, 32b, 32.8, 32.9, 32.10 and 32.11 have been found by database analysis to putatively function as cathelicidins. The tammar wallaby molecules LAM 32a and 32b were found to be functionally equivalent variants of LAM 32. The bovine molecules LAM 32.8, 32.9, 32.10 and 32.11 are named herein as Cathelicidin 8, 9, 10 and 11, respectively. These bovine cathelicidin molecules were isolated using PCR primers based on known cathelicidins, namely bovine cathelicidins 1 to 7 found in Unigene database software.

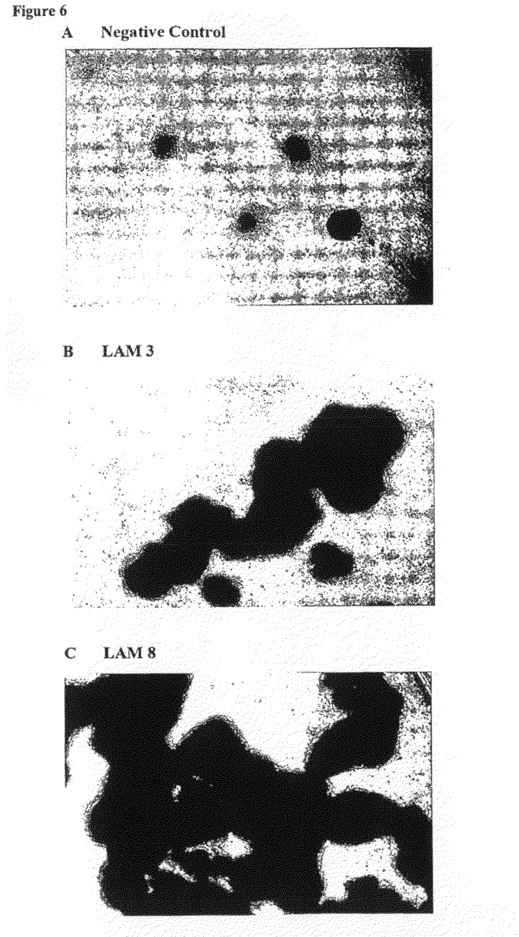

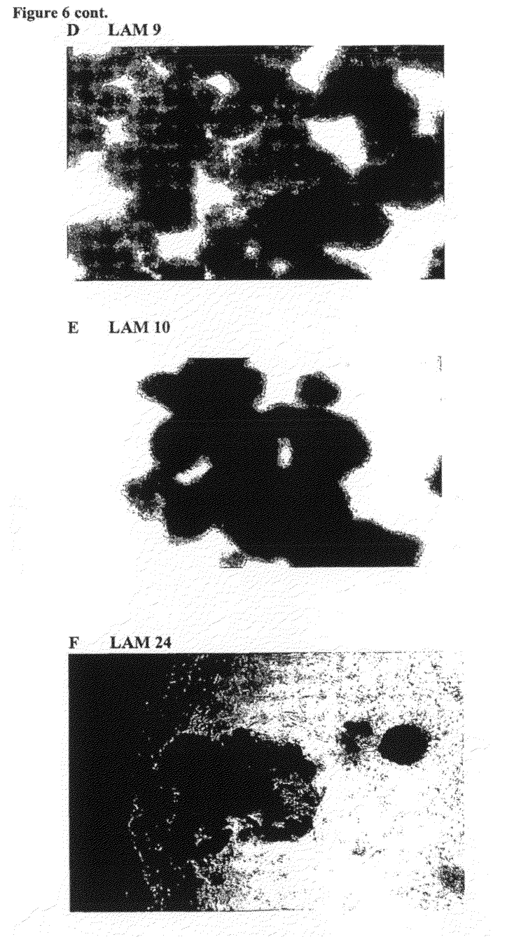

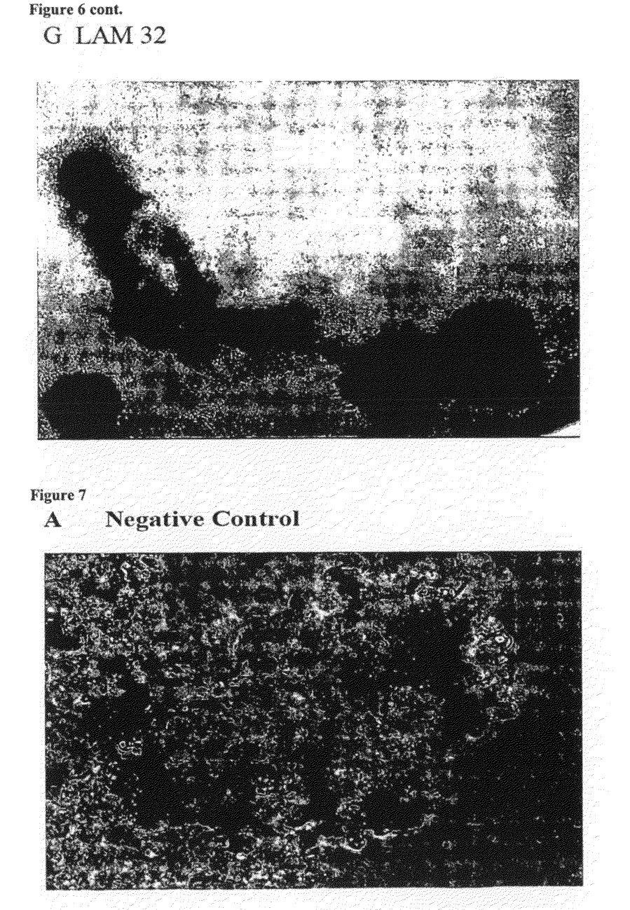

LAM 32 was shown to be involved in cellular morphology as represented in FIG. 6. Cell growth analysis involving the splice functionally equivalent variants of LAM 32, namely LAM 32a and 32b, is presented below.

### Example 4F1

### Determination of Cell Growth with Cathelicidins in HC11 Cells

As shown in FIG. 2 equal numbers of mouse mammary HC11 cells were grown in the presence of either a negative control or tammar wallaby cathelicidin-associated polypeptide, namely LAM 32 (SEQ ID NO: 188), LAM 32a (SEQ ID NO: 215) or LAM 32b (SEQ ID NO: 207) in order to determine the effect of these LAMs on cellular growth.

HC11 cells were initially plated at 50 percent confluency and cultured for 72 hours in a 1:10 dilution of serum and antibiotic free Optimem and media conditioned with cathelicidin associated polypeptides. The medium was removed and then washed gently for 2 minutes with Mg²⁺- and Ca²⁺-free phosphate buffered saline (PBS), 0.05% Trypsin and 1:5000 Versene to disrupt cell aggregates. Cells were collected by scraping and washed twice by resuspension in complete medium followed by centrifugation. The cells were again resuspended and counted in triplicate using a Luber haemocytometer.

As shown in FIG. 2 and Table 17, all cathelicidins produced significantly different growth from the control.

TABLE 17
Determination of cell growth with tammar wallaby cathelicidin-associated polypeptides in
HC11 cells.

| Cathelicidin | | | | | | | |
|---|---|---|---|---|---|---|---|
| associated | Rep. | Rep. | Rep. | Rep. | | | |
| polypeptides | 1 | 2 | 3 | 4 | Mean | STDEV | P value |
| −ve control | 0.727 | 0.76 | 0.786 | 0.808 | 0.77025 | 0.034874776 | — |
| LAM 32 | 0.985 | 0.927 | 0.968 | 1.026 | 0.9765 | 0.041008129 | 0.001015693 |
| LAM 32a | 0.958 | 0.977 | 1.008 | 1.102 | 1.01125 | 0.063913353 | 0.000443171 |
| LAM 32b | 0.89 | 0.841 | 0.862 | 0.832 | 0.85625 | 0.025773048 | 0.028962566 |

Rep: replicate
STDEV: standard deviation
−ve control: negative control

The cells were counted in triplicate using a Luber haemocytometer. The negative control was media alone (i.e. conditioned media without insertless vector). The samples were deemed statistically significant using a t-test comparing the control with the sample if the values of cell growth were 2 standard deviations higher than the mean of the negative control.

### Example 4F2

### Determination of Cell Growth with Cathelicidins in Mouse ES Cells

As shown in FIG. 3, equal numbers of mouse ES cells were grown in the presence of either a negative control or tammar wallaby cathelicidin associated polypeptide, namely LAM 32 (SEQ ID NO: 188), LAM 32a (SEQ ID NO: 215) or LAM 32b (SEQ ID NO: 207) in order to determine the effect of these LAMs on cellular growth.

Mouse ES cells were plated at 50 percent confluency and cultured for 24 hours in a 1:10 dilution of serum and antibiotic free Optimem and media conditioned with cathelicidin associated polypeptides. The medium was removed and then washed gently for 2 minutes with Mg²⁺- and Ca²⁺-free phosphate buffered saline 0.05% Trypsin and 1:5000 Versene to disrupt cell aggregates. Cells were collected by scraping and washed twice by resuspension in complete medium followed by centrifugation. The cells were again resuspended and counted in triplicate using a Luber haemocytometer. As shown in FIG. 3 and Table 18, LAMs 32a and 32b produced significantly different growth from the control.

TABLE 18
Determination of cell growth using the tammar wallaby cathelicidin associated
polypeptides LAMs 32, 32a and 32b in mouse ES cells

| Cathelicidin | | | | | | |
|---|---|---|---|---|---|---|
| associated | | | | | | |
| polypeptides | Rep. 1 | Rep. 2 | Rep. 3 | Mean | STDEV | P value |
| −ve control | 0.34 | 0.474 | 0.352 | 0.388666667 | 0.074144004 | — |
| LAM 32 | 0.721 | 0.548 | 0.784 | 0.684333333 | 0.122197927 | 0.059090014 |
| LAM 32a | 0.575 | 0.605 | 0.517 | 0.565666667 | 0.044736264 | 0.014319968 |
| LAM 32b | 0.676 | 0.677 | 0.693 | 0.682 | 0.009539392 | 0.011460202 |

Rep: replicate
STDEV: standard deviation
−ve control: negative control

The cells were counted in triplicate using a Luber haemocytometer. The negative control was media alone (i.e. conditioned media without insertless vector). The samples were deemed statistically significant using a t-test comparing the control with the sample if the values of cell growth were 2 standard deviations higher than the mean of the negative control.

### Example 4F3

### Wallaby Alignment of Cathelicidin Proteins

cDNA sequence alignment of predicted tammar wallaby cathelicidin isoforms expressed in the mammary gland was undertaken. 165 novel expression sequence tags were identified and subjected to sequence alignment analysis. Based on these cDNA sequence alignments, two variant tammar sequences were identified as predicted cathelicidins, designated LAM 32a and 32b. These data are shown in Table 19, with sequences appearing (from top to bottom) as follows: (1) Contiguous sequence containing LAM 32b (variant of LAM 32), (2) Contiguous sequence containing LAM 32, (3) contiguous sequence No. 1 identified but not subjected to further analysis, (4) Contig containing LAM 32a (variant of LAM 32)and (5) contiguous sequence No. 2 identified but not subjected to further analysis.

TABLE 19
Wallaby alignment of cathelicidin proteins

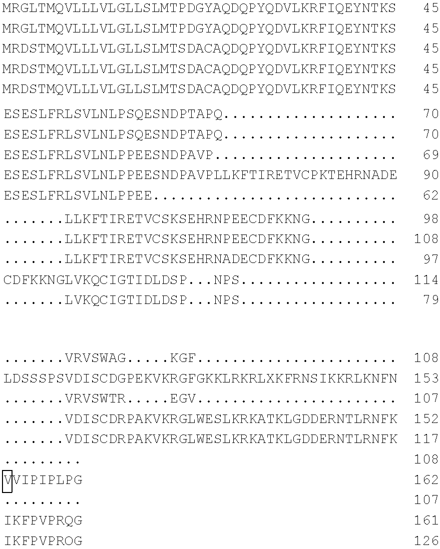

### Example 4F4

### Bovine and Wallaby Alignment of Cathelicidin Proteins

The wallaby cathelicidin proteins LAM 32 and 32b were aligned with the known bovine Cathelicidins 1 to 7. Signal sequences and conserved cathelicidin domains are shown in Table 20.

The alignment below comprises bovine cathelicidins isolated from the bovine mammary based on similarity with tammar mammary cathelicidin LAM 32 and 32b. The signal sequences are underlined and bolded. The cathelicidin motif is shown in italics and bolded. We note that the C-terminal region after the cathelicidin motif shows no conservation between variants and encodes the known antimicrobial peptide regions.

TABLE 20
Bovine and wallaby alignment of cathelicidin proteins

| | |
|---|---|
| Cath12 | METQRASLSLGRCSLWLLLLGLVLPSASAQALS [symbol] |
| Cath14 | MQTQRASLSLGRWSLWLLLLGLVVPSASAQALS [symbol] |
| Cath13 | METQRASLSLGRWSLWLLLLGLVLPSASAQALS [symbol] |
| Cath17 | METQRASFSLGRSSLWLLLLGLVVPSASAQDLS [symbol] |
| Cath15 | METQRASLSLGRWSLWLLLLGLALPSASAQALS [symbol] |
| Cath16 | METQRASLSLGRWSLWLLLLGLALPSASAQALS [symbol] |
| Cath11 | METPRASLSLGRWSLWLLLLGLALPSASAQALS [symbol] |
| LAM 32b | M---RG-LTMQVLLLVLGLLSLMTPLGYAQDQP [symbol] |
| LAM32 | M---RG-LTMQVLLLVLGLLSLMTPLGYAQDQP [symbol] |
| | 61 71 81 91 101 111 |
| Cath12 | [symbol] - [symbol] [symbol] [symbol] VKQCVGTVTLDPSNDQ |
| Cath14 | [symbol] - [symbol] [symbol] [symbol] RVKQCVGTVTLDPSNDQ |
| Cath13 | [symbol] - [symbol] [symbol] [symbol] LVKQCVGTITLDQSDDL |
| Cath17 | [symbol] [symbol] [symbol] VKQCVGTVTRYWIRGD |
| Cath15 | [symbol] [symbol] [symbol] LLKECVGTVTLDQVGSN |
| Cath16 | [symbol] [symbol] [symbol] VKQCVGTVTLDAVKGK |
| Cath11 | [symbol] - [symbol] [symbol] [symbol] LLKRCEGTVTLDQVRGN |
| LAM 32b | [symbol] - [symbol] [symbol] [symbol] ----------------- |
| LAM 32 | [symbol] - [symbol] [symbol] [symbol] LVEECIGTVDLDSSSPS |
| | 121 131 141 151 161 171 |
| Cath12 | FDINCNELQSVRFRPPIRRPPIRPPFYPPFRPPIRPPIFPPIRPPFRPPLGPFPGRR--- |
| Cath14 | FDLNCNELQSVIL------P-WKWPWWPWRRG---------------------------- |
| Cath13 | FDLNCNELQSVRR----IRP--RPPRLPRPRPRPLPFPRPGPRPIPRPLPFPRPGPRPIP |
| Cath17 | FDITCNNIQSAGL--------FRRLRDSIRRGQQK--ILEKARRIGERIKDIFRG----- |
| Cath15 | FDITCAVPQSVGG------------LRSLGRKILR--AWKRYGPIIVPIIRIG------- |
| Cath16 | INVTCEELQSVGR------------FKRFRKKFKK--LFKKLSP-VIPLLHLG------- |
| Cath11 | FDITCNNHQSIRIT-----------KQPWAPPQAA----RLCRIVVIRVCR--------- |
| LAM 32b | VRVSWAG-----KGF--------------------------------------------- |
| LAM 32 | VDISCDGPEKVKRGFG------KKLRKRLKKFRNS--IKKRLKNFNVVIPIPLPG----- |
| | 181 191 |
| Cath12 | ----------------- |
| Cath14 | ----------------- |
| Cath13 | RPLPFPRPGPRPIPRPL |
| Cath17 | ----------------- |
| Cath15 | ----------------- |
| Cath16 | ----------------- |
| Cath11 | ----------------- |
| LAM 32b | ----------------- |
| LAM 32 | ----------------- |

Bold Underlined = Signal Sequence
Bold Italics = Conserved cathelicidin domain

### Example 4F5

### Bovine and Wallaby Cathelicin Domains

Cathelicidins are recognized by a conserved “cystatin-like” domain motif. Tammar wallaby cathelicidin LAM 32 (SEQ ID NO: 188) was aligned with bovine Cathelicidin 1, yielding a similarity of 52.6% and an identity of 39.7%. The similarity between the bovine and tammar wallaby is highlighted in a conserved motif as shown below in Table 21.

TABLE 21
Bovine and wallaby cathelicin domains

Tammar wallaby (LAM 32):
YQDVLNRFIQEYNTKSESESLFRLSVLNLPSQESNDPTAPQLLKFTIRETVCSKSEHRNPEECDFKKNGLVEECIGTV
Bovine (CATHL1):
YREAVLRAVDQLNEQSSEPNIYRLLELDQPPQDDEDPDSPKRVSFRVKETVCSRTTQQPPEQCDFKENGLLKRCEGTV
Alignment: Percent Similarity: 52.564 Percent Identity: 39.744

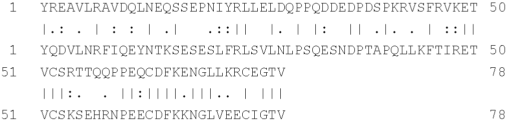

### Example 4F6

### Determination of Cell Growth Using Bovine Cathelicidin Variants in AGS Cells

A cellular growth assay was performed using seven bovine cathelicidin (CATHL) variants, namely CATHL1, 4, and 6 which are known in the art (the relevant sequences can be obtained using the following hyperlink: http://www.ncbi.nlm.nih.gov/entrez/query.fcqi?db=unigene&cmd=search&term=cathelicidin) and bovine Cathelicidins 8, 9, 10 and 11 (being LAMs 32.8, 32.9, 32.10 and 32.11, respectively) as identified herein and which are derived from LAM 32. The bovine cathelicidin associated LAMs were derived by sequence comparison to the tammar polynucleotide sequence of LAM 32.

The growth rate of AGS cells was assessed in the presence of the cathelicidins. Equal numbers of human gastric epithelial AGS cells were cultured in either control media or media conditioned with bovine cathelicidins for 72 hours in a 1:10 dilution of serum and antibiotic-free Optimem. Cells were then fixed and a Sulforhodamine B (SRB) colorimetric assay was undertaken as described in Journal of Immunological Methods 208: 151-158, K. T. Papazisis, G. D. Geromichalos, K. A. Dimitriadis, A. H. Kortsaris (1997), ‘Optimization of the sulforhodamine B colorimetric assay’.

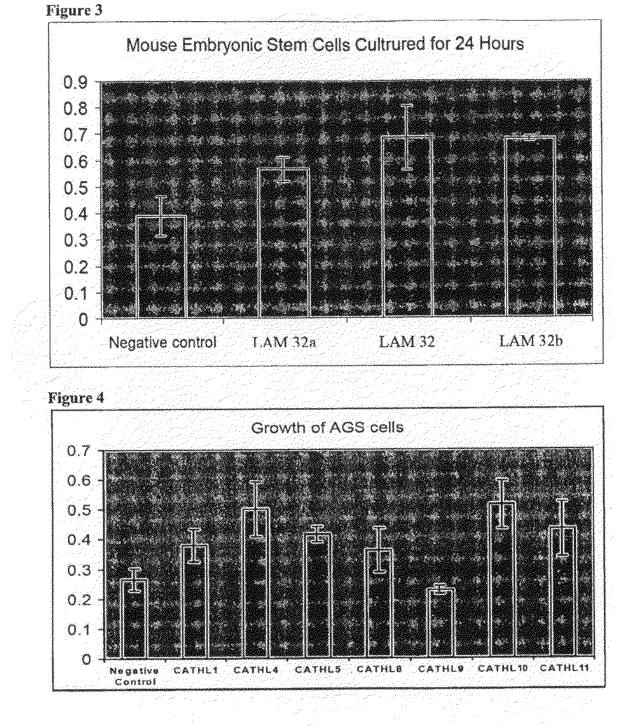

Briefly, cells were fixed directly by incubation with 10% trichloroacetic acid for 1 hour at 4° C., washed with water, air-dried overnight and then stained with 0.4% w/v sulforhodamine B, 1% acetic acid solution for 10 minutes at room temperature. Cell were again washed with 1% acetic acid and air-dried, then counted in triplicate. All samples except CATHL9 showed a significantly higher number of cells, and therefore higher rates of cell growth, than the negative control (i.e. P<0.05 using a t-test comparing control with sample) as shown in FIG. 4 and Table 22.

TABLE 22
Determination of cell growth using bovine cathelicidin variants in AGS cells

| Bovine | | | | | | | |
|---|---|---|---|---|---|---|---|
| cathelicidins | Replicate | Replicate | Replicate | Replicate | | | |
| variants | 1 | 2 | 3 | 4 | Mean | STDEV | P value |
| −ve control | 0.247 | 0.316 | 0.233 | 0.265 | 0.265 | 0.036 | — |
| CATHL1 | 0.412 | 0.419 | 0.303 | 0.378 | 0.378 | 0.053 | 0.005 |
| CATHL4 | 0.626 | 0.465 | 0.409 | 0.500 | 0.500 | 0.092 | 0.010 |
| CATHL5 | 0.423 | 0.441 | 0.382 | 0.415 | 0.415 | 0.025 | 0.000 |
| CATHL8 | 0.441 | 0.382 | 0.261 | 0.361 | 0.361 | 0.075 | 0.037 |
| CATHL9 | 0.246 | 0.220 | 0.220 | 0.229 | 0.229 | 0.012 | 0.090 |
| CATHL10 | 0.602 | 0.405 | 0.534 | 0.514 | 0.514 | 0.082 | 0.011 |
| CATHL11 | 0.365 | 0.366 | 0.563 | 0.431 | 0.431 | 0.093 | 0.034 |

Rep: replicate
STDEV: standard deviation
−ve control: negative control

### Example 4F7

### Chick Growth in the Presence of Cathelicidins

Conditioned media containing LAM polypeptides or bovine cathelicidins or variants thereof were diluted in a total of 1 ml of normal saline. Eggs on day 16 of incubation were injected in ovo with 1 ml of LAM polypeptides or bovine cathelicidins or variants thereof into the broad end of the egg. Chicks were weighed on day of hatching. Controls included saline only, or saline and 10 mg/ml of a peptide as a random peptide control.

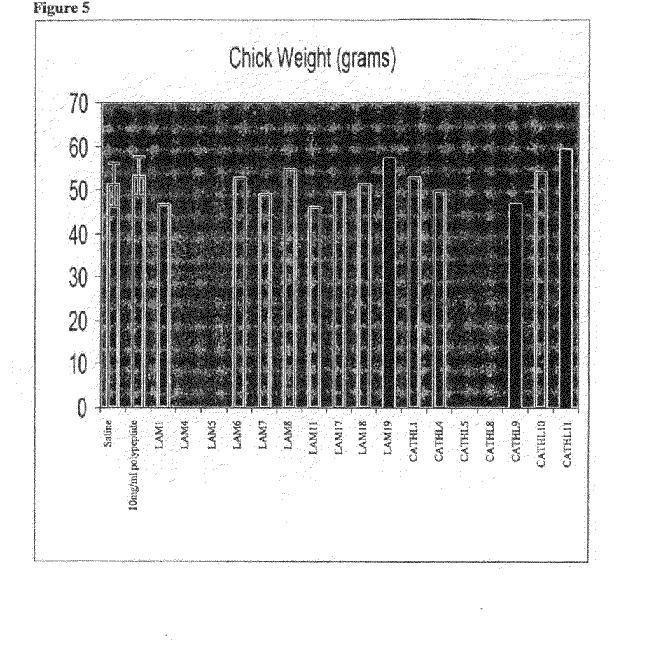

As shown in FIG. 5 and Table 23, bovine polypeptides corresponding to LAM 4 (SEQ ID NO: 94), LAM 5 (SEQ ID NO: 95), CATHL5 (known sequence) and LAM 32.8 (i.e. CATHL8; SEQ ID NO: 171) caused death of chicks. Furthermore, LAM 32.9 (i.e. CATHL9; SEQ ID NO: 173) caused stumpy legs and loss of down. Also, LAM 32.11 (i.e. CATHL11; SEQ ID NO: 177) produced a chick larger than the controls which was greater than 2 standard deviations from the mean in relation to the body weights in grams of all the chicks tested in this experiment.

As such, CATHL11 can trigger accelerated growth in chicks and possibly other animals. The bovine polypeptide corresponding to LAM 19 (SEQ ID NO: 109) was also included as it falls just outside 2 standard deviations from the median of chick weights.

TABLE 23
Chick growth in the presence of cathelicidin variants

| LAM Polypeptides or Bovine | Chick weight |
|---|---|
| Cathelicidins | (grams) |
| LAM 1 | 46.7 |
| LAM 4 | Killed |
| LAM 5 | Killed |
| LAM 6 | 52.6 |
| LAM 7 | 49.1 |
| LAM 8 | 54.4 |
| LAM 11 | 46.1 |
| LAM 17 | 49.2 |
| LAM 18 | 51.1 |
| LAM 19 | 57.1 |
| CATHL1 | 52.8 |
| CATHL4 | 49.7 |
| CATHL5 | Killed |
| CATHL8 | Killed |
| CATHL9 | 46.5 |
| CATHL10 | 53.8 |
| CATHL11 | 59 |
| Saline | 51.0 |
| Saline + 10 mg/ml | 53.1 |
| polypeptide | |

As set out in Table 23, the left column indicates the polypeptide used in the assay while the right column contains chick weight in grams as a measurement of chick growth. Bolded numbers represent a chick(s) larger than the controls and more than 2 standard deviations from the mean. In addition, bolded numbers represent chicks that have been killed. The mean represents the weight of all the chicks tested within this experiment.

### Example 4G

### Mammary Epithelial Cell Functioning

The morphology of mammary epithelium changes significantly as it moves from a non-milk secreting epithelium to a highly secretory epithelium. Two aspects of mammary epithelial cell functioning were investigated, namely cellular morphology and cellular differentiation.

### Example 4G1

### Cellular Morphology

Candidate LAM polypeptides that are able to regulate the function and differentiation of the mammary gland were screened by culturing bovine mammary epithelium in the presence of a 1:5 dilution of the secreted polypeptide LAMs for 48 hours as described for the TGR assays in Example 4. Cells were examined microscopically for gross morphological changes and for their ability to induce cell differentiation.

The results can be seen in FIG. 6, wherein 7 micrographs (designated A to G) depicting bovine mammary epithelial cells demonstrated that cellular morphology was influenced by exposure to LAMs 3, 7, 8, 9, 10, 24, and 32. Each of these LAMs induced a significant change in quaternary structure and/or cellular morphology.

### Example 4G2

### Cellular Differentiation

To determine the ability of LAMs to influence cellular differentiation, mouse embryonic stem (ES) cells were cultured in the presence of a leukemia inhibitory factor (LIF) 1000 U/ml and a 1:5 dilution of the secreted polypeptide LAMs for 48 hours. The mouse ES cells contained an Oct4-βgal transgene, such that Oct4 expression (an indicator of pluripotency and hence an absence of differentiation) resulted in the cells staining blue.

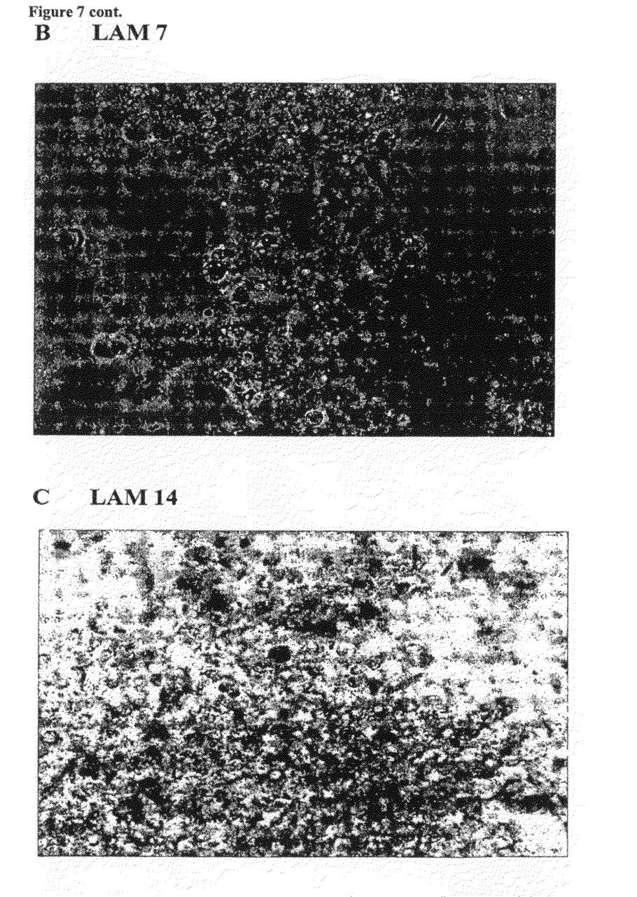

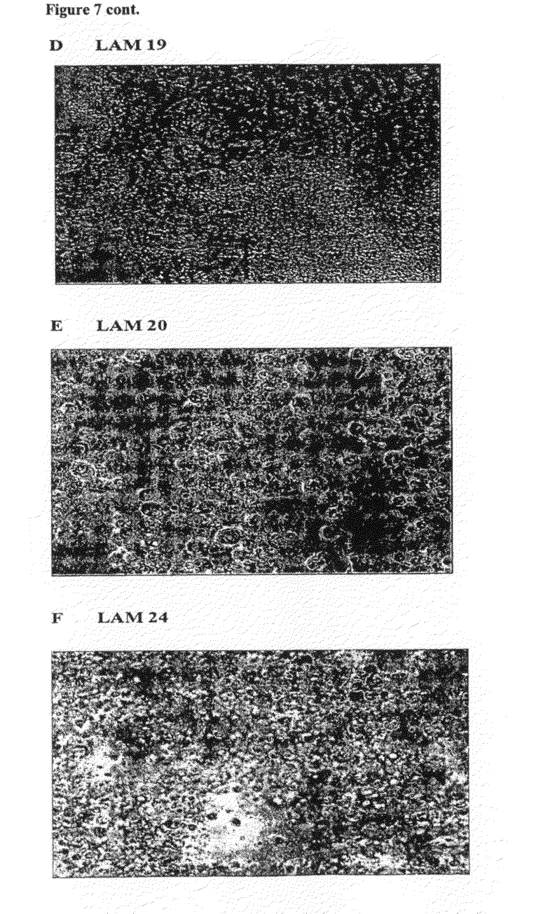

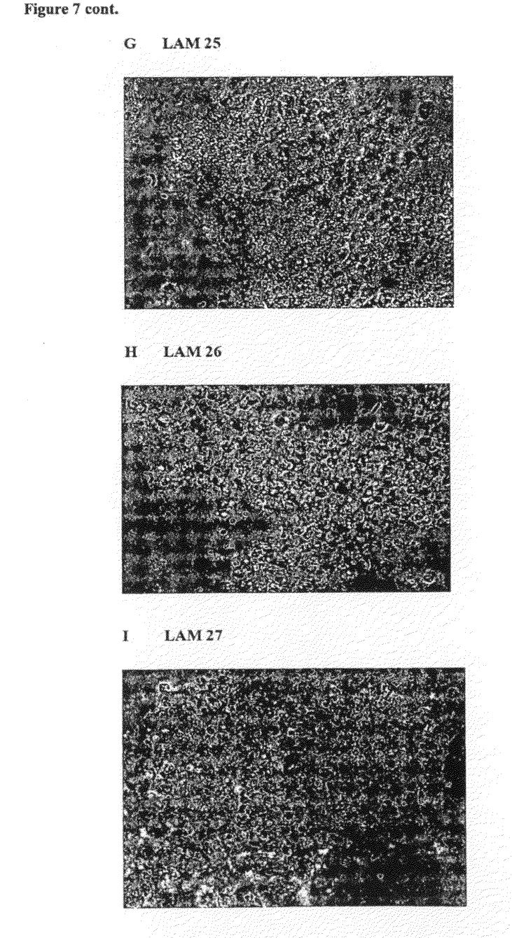

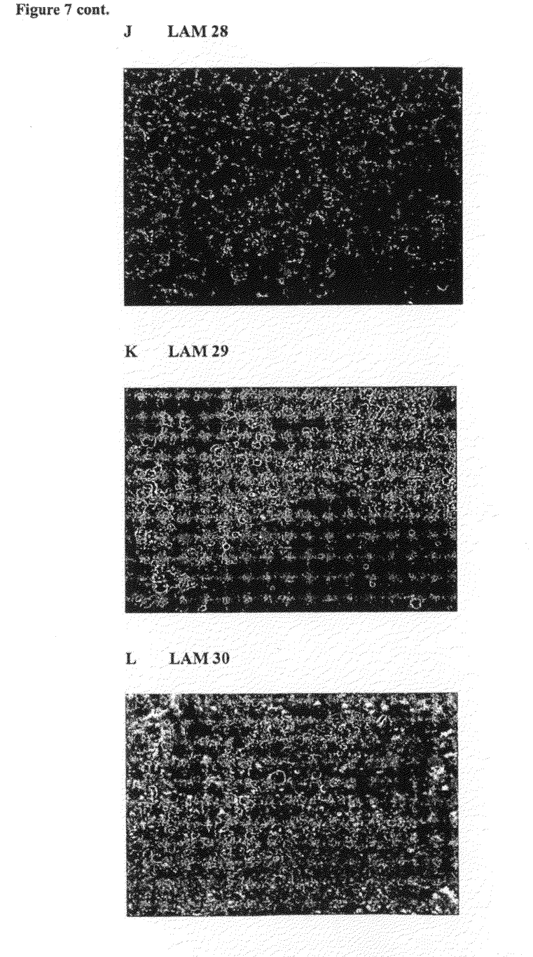

Tammar wallaby polypeptide LAMs 7, 14, 19, 20, 24, 25, 26, 27, 28 and 29 induced cellular differentiation, as exemplified by the micrographs (designated A to L) shown in FIG. 7.

### Example 5

### Bovine Microarray Studies

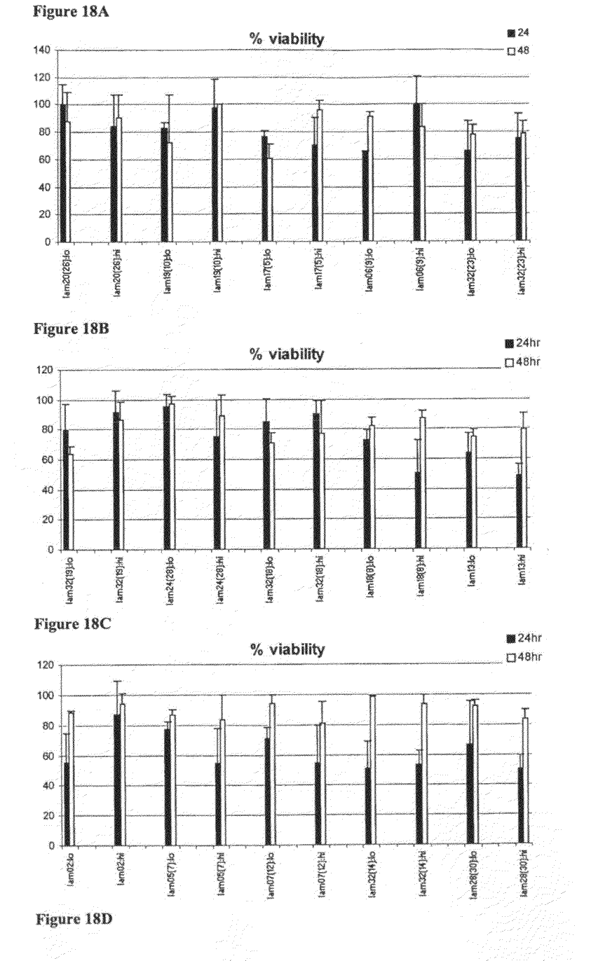

Total mammary RNA was prepared from 3 pregnant 9-22 days prior to parturition, 3 cows at approximately 30 days of lactation and 3 cows in early involution. Microarray analysis as shown in FIG. 18 was performed on bovine Affymetrix microarrays under contract by the Australian Genome Research Facility.

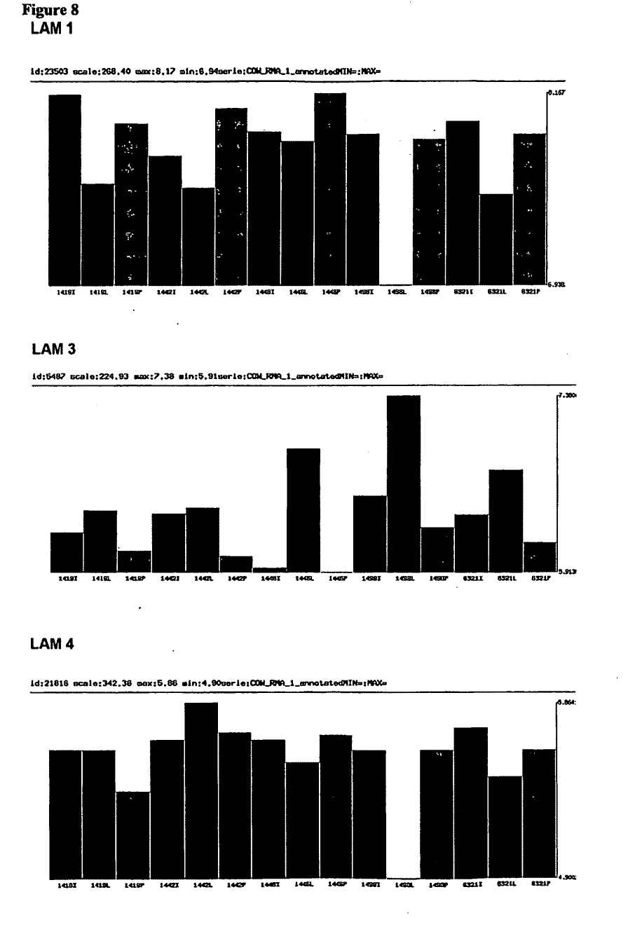

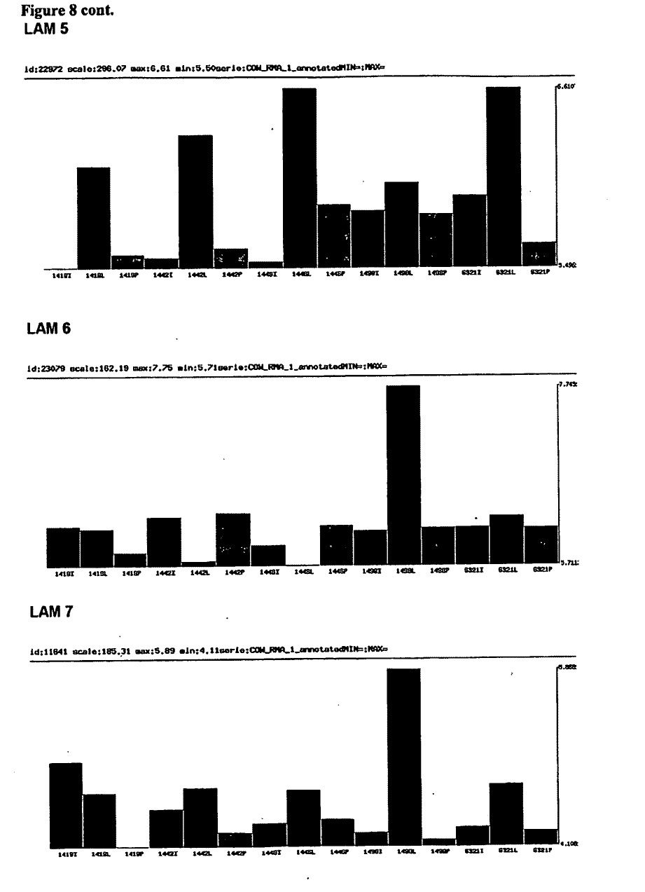

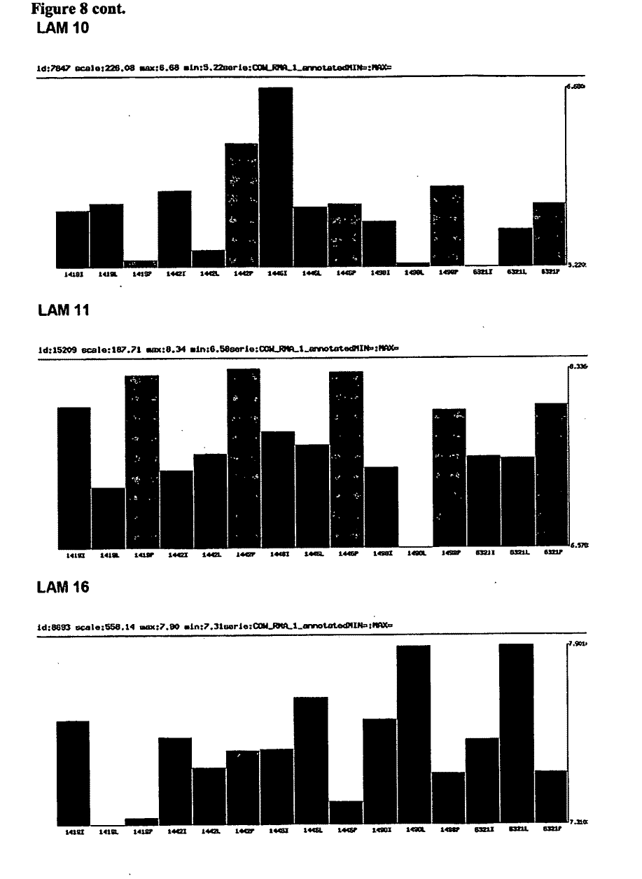

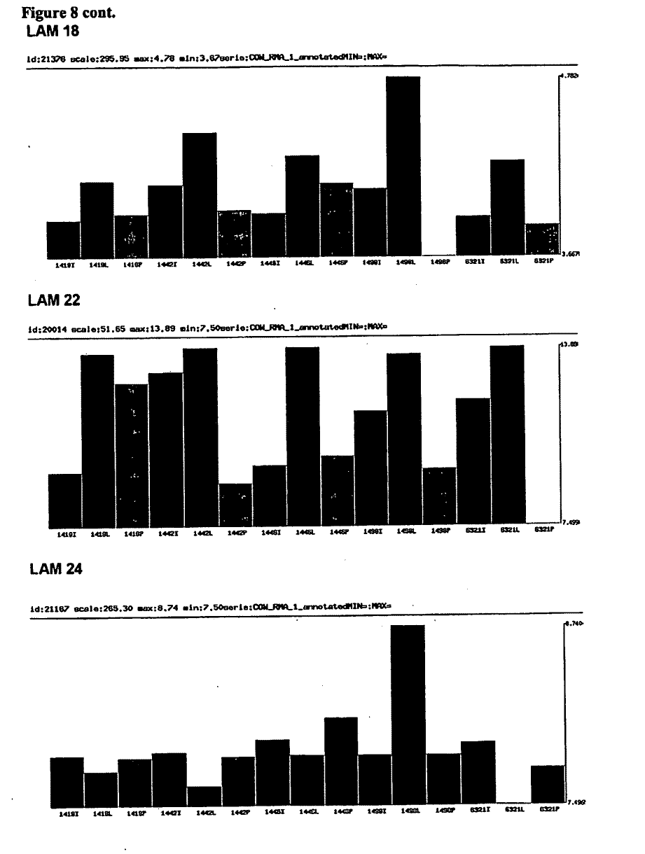

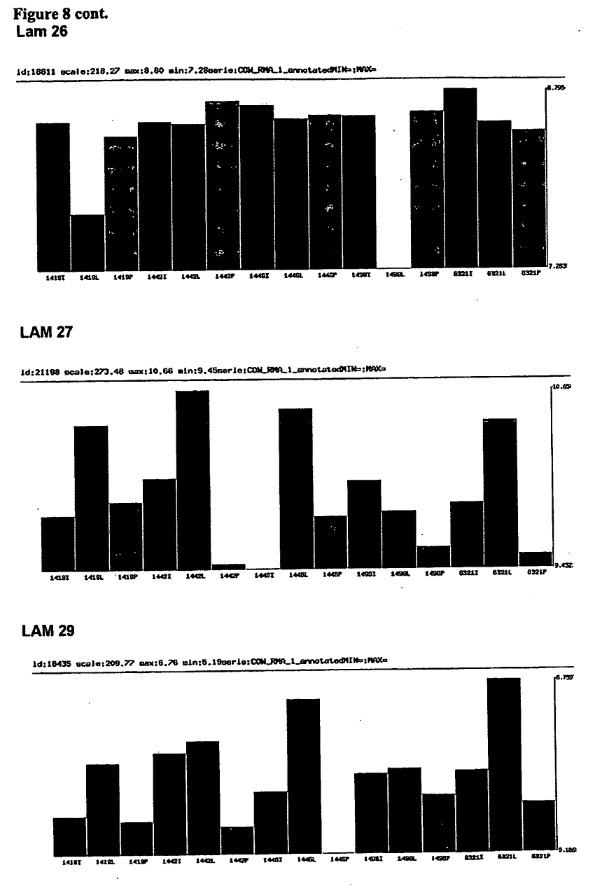

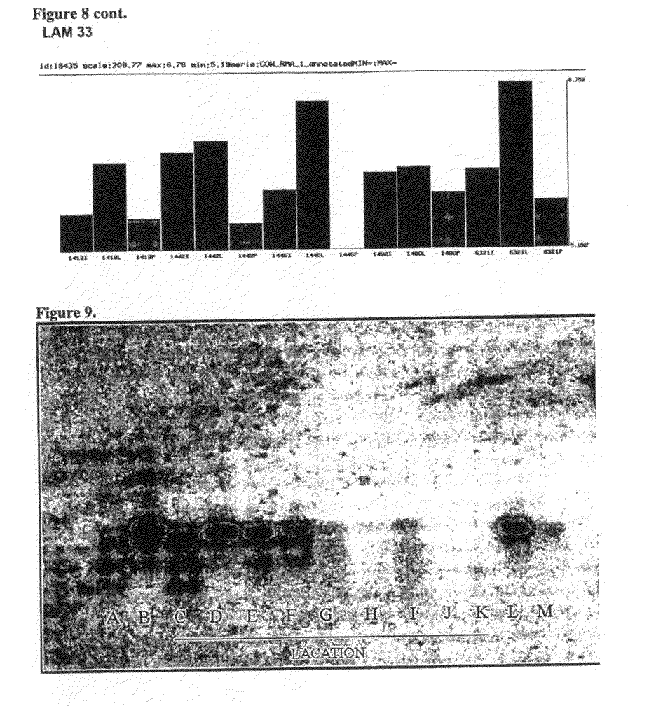

Gene expression for each LAM as represented in FIG. 8 was performed on the Affymetrix bovine microarray and subsequent expression was displayed in five cows during pregnancy (green), lactation (Red) and involution (blue). The x axis shows the range of gene expression in the samples in log2.

### Example 5A

### Assay for Increased Protein Secretion

In a ³⁵S-methonine protein synthesis assay, bovine mammary epithelial cells can be plated onto an extracellular matrix in 96 well plates. After 5 days in culture, cells can be incubated in methionine free medium for 1 hour and then labeled with ³⁵S-methionine for a 4 hour period. Cells can then be exposed to the expressed peptides during this time. Cell media can be collected and protein precipitated from the media, as well as cells being harvested. Cell extracts and protein precipitated from the media can then be counted using a liquid scintillation counter. This enables both cellular and secreted protein synthesis to be determined relative to an appropriate control.

### Example 5B

### Antibacterial Assays

Bacteria can be cultured in the presence of conditioned media, and the effects on growth and viability of the organisms assessed. Target organisms may include human pathogens including Helicobacter pylori, which is the major cause of gastric ulcers and gastric cancer.

### Example 6

### Northern Expression

Using standard techniques well known to a person of skill in the art, tammar polynucleotide LAM 32 was used as a probe in a Northern blot analysis to demonstrate the expression of cathelicidins in the tammar mammary gland. The results are shown in FIG. 9 for A: Day 13 pregnant, B: parturition, C: day1, D: day2, E: day3, F: day10, G: day40, H: day87, I: day114, J: day150, K: day240, L: day 5 involution and M: day10 involution. These results demonstrate that sample B showed a strong indication of cathelicidin at parturition, while samples D, E and L also showed distinct cathelicidin presence.

### Example 7

### Bovine SNPs for LAM 30 to 32

Single nucleotide polymorphisms (SNPs) are DNA sequence variations that occur when a single nucleotide (A, T, C, or G) in the genome sequence is altered. SNPs were obtained for the majority of LAMS as disclosed herein using the Commonwealth Scientific and Industrial Research Organisation (CSIRO) publicly available database IBISS at http://www.livestockgenomics.csiro.au/ibiss.

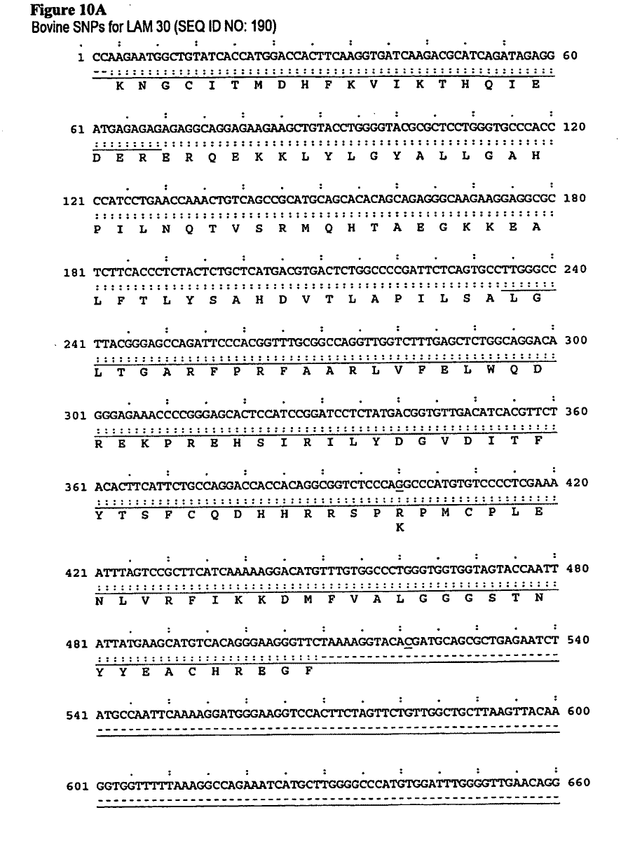

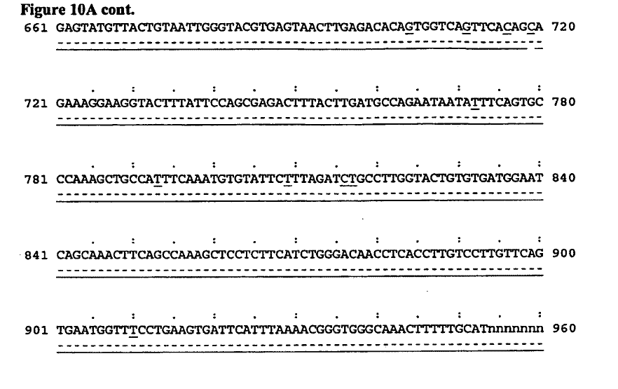

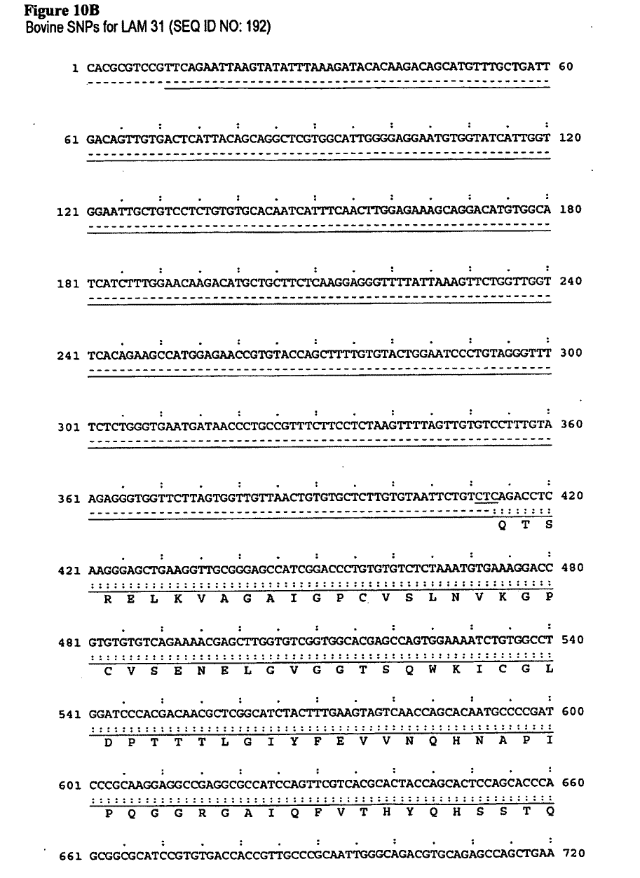

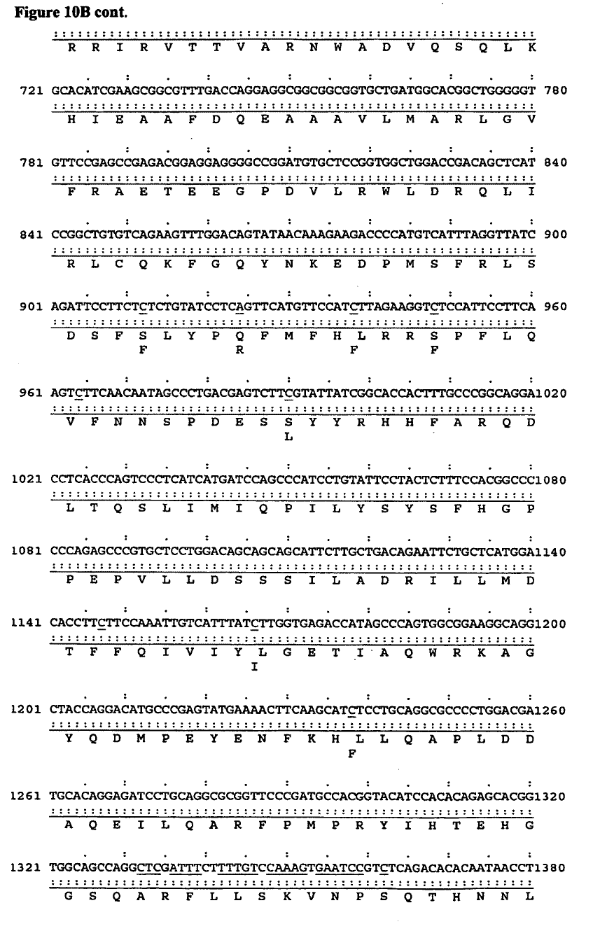

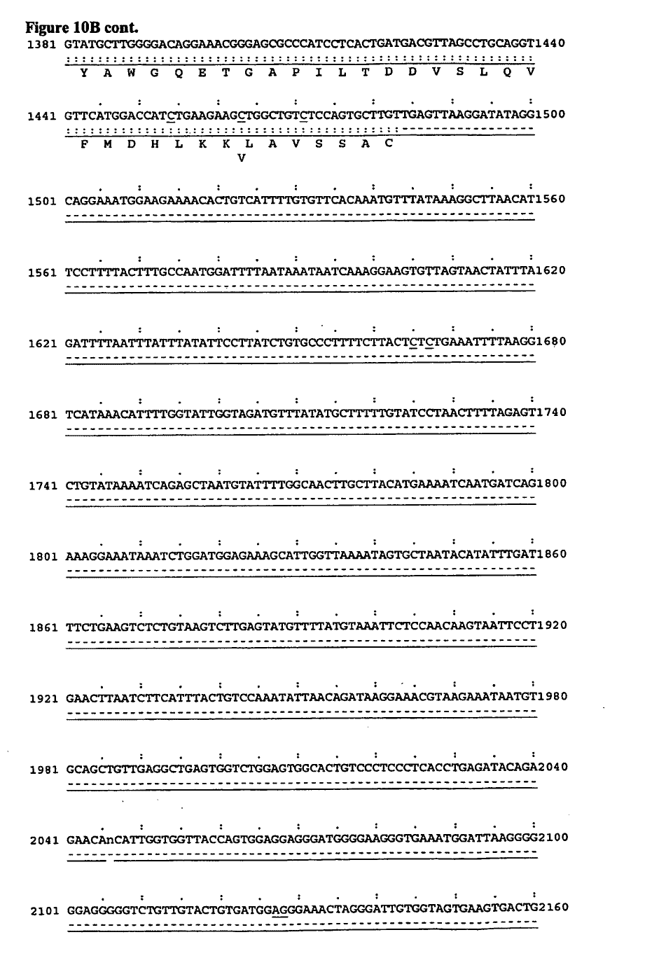

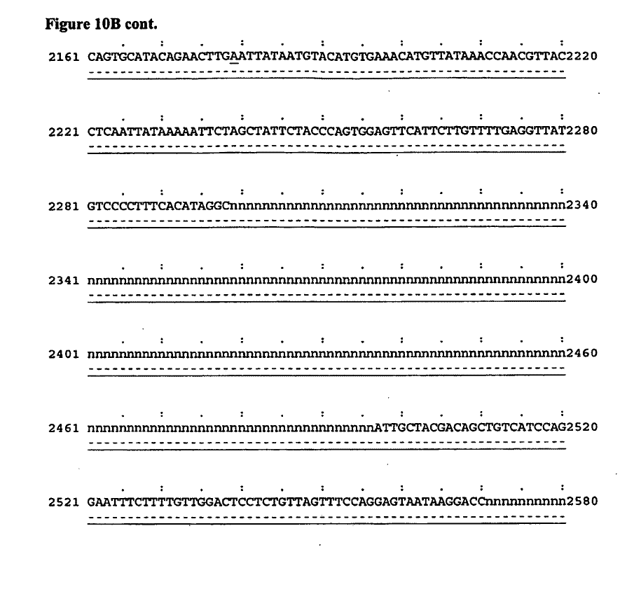

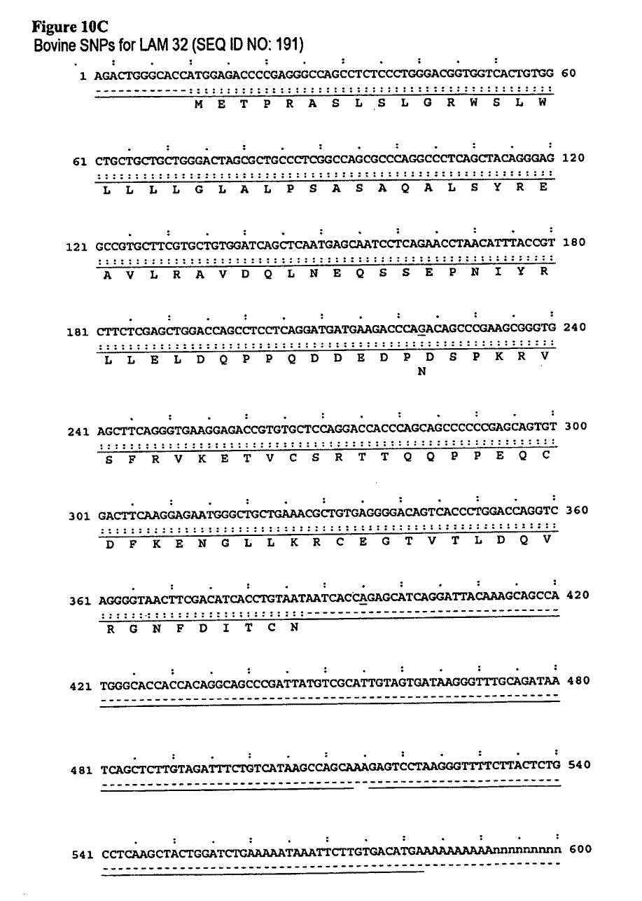

To exemplify the SNPs data, FIGS. 10A, 10B, and 10C recite SNP sequences for bovine polynucleotides as represented in LAM 30 (SEQ ID No 190), LAM 31 (SEQ ID No 192) and LAM 32 (SEQ ID No 191).

### Example 8

### Assays of Biological Activities of LAMs

Selected bovine LAMs were assayed for biological activities as described in earlier examples. The results of these assays are provided as follows.

### Example 8A

### Results of TGR BioSciences Assays of CRC-IDP Samples ERK Assay for Assessment of Cell Activation Toward Growth

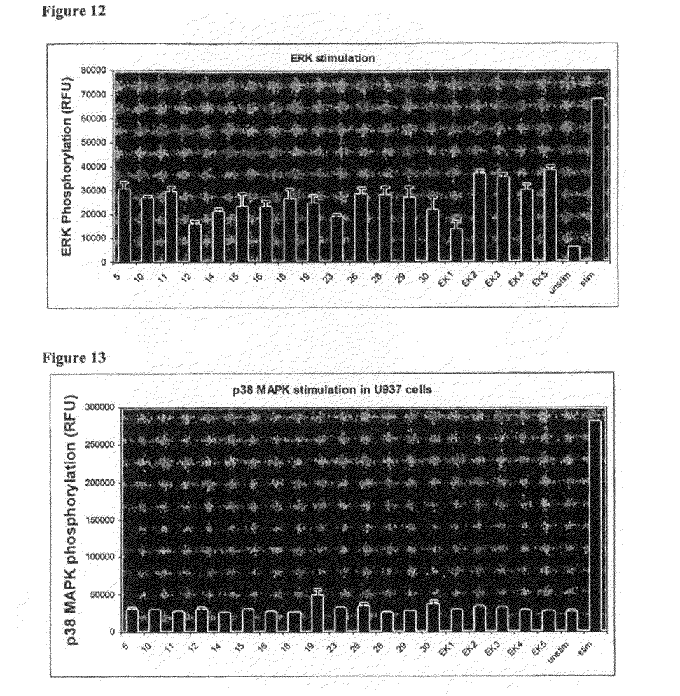

Aliquots of the CRC-IDP samples were used to activate HSC-2 cells in a 96 well plate. Confluent and serum-starved cells were presented with the samples at a final dilution of 1:2. Cells were also stimulated with an internal control for activation of cells (Stim=10% serum) or left unstimulated (unstim). After 10 minutes with the CRC-IDP samples or control stimulus, cells were lysed and assayed for ERK activation using TGR's proprietary assay technology (SureFire). FIG. 12 presents the results as the mean+/−SEm of 3 separate samples. The data is from a single experiment.

The cells were responsive to receptor stimulation of ERK, as indicated by the control stimulus (Serum) as opposed to unstimulated cells (Unstim). All of the test samples contained activity that activated ERK phosphorylation, which is almost certainly due to receptor stimulation at the cell surface. Samples varied in activity toward ERK activation. The weakest sample was EK1. It is anticipated that further characterization would involve full dose-response analysis of selected samples.

Descriptions of the LAM used in the experiments and noted in FIGS. 12 and 13 are shown in Table 24

TABLE 24
Descriptions of LAMs noted in FIGS. 12 and 13

| | | | tammar |
|---|---|---|---|
| Sample | LAM | | ERK |
| number | Number | Description | activity |
| 5 | LAM07 | DGAT2: Diacylglycerol O-acyltransferase | yes |
| | | homolog 2 (mouse) | |
| 10 | LAM19 | EBP: Emopamil binding protein (sterol | |
| | | isomerase) | |
| 11 | LAM08 | TMEM165: TPA regulated locus | |
| 12 | LAM17 | FGL2: Fibrinogen like-1 | |
| 14 | LAM32 | CATHL1 | yes |
| 15 | LAM32 | CATHL2 | yes |
| 16 | LAM32 | LOC786887 Bovine similar to cathelicidin | yes |
| 18 | LAM32 | CAMP (variant 1) | yes |
| 19 | LAM32 | CATHL5 | yes |
| 23 | LAM32 | CATHL (variant 2) | yes |
| 26 | LAM20 | IFITM1: Interferon induced transmembrane | |
| | | protein 1 | |
| 28 | LAM24 | C1orf160: Chromosome 1 open reading | |
| | | frame 160 | |
| 29 | LAM28 | C20orf195 | |
| 30 | LAM23 | RNH1 | |
| EK1 | | Negative control - empty pTarget vector | |
| EK2 | | APRIL | |
| EK3 | LAM13 | PLA2G1B | |
| EK4 | LAM02 | TCN clone 1 | yes |
| EK5 | LAM02 | TCN clone 2 | yes |

p38 MAP Kinase Activation as an index of Inflammatory Activity Using U937 Monocytes

U937 Human monocytic cells were stimulated with TNFα (Stim) or the supplied test samples at a 1:2 final dilution, and incubated for 20 minutes. After this period, cells were lysed and assessed for activation of p38 MAPK as an index of a response to a potentially inflammatory molecule. FIG. 13 indicates that whereas TNFα induced potent activation of p38 MAPK, there was little evidence that any of the samples supplied had a similar activity for this cell line. Results are the mean+/−SEm of 3 replicate cell stimulations. The data were obtained from a single experiment.

### Summary

All of the CRC-IPD samples contained molecules that, to varying degree, activated ERK phosphorylation in HSC-2 cells. Therefore, it is likely that many of the components of these samples possess growth promoting activity for cells.

In contrast, there was no evidence for pro-inflammatory activity of the samples on U937 cells as judged by ability to activate p38 MAPK phosphorylation. However, it may be of interest to examine effects of samples on another cell line, such as the RAW macrophage.

The ability of samples to inhibit TNFα-induced p38 MAPK activation in U937 cells was unable to be determined as there was too much experimental variation to reach a convincing conclusion anything from the data. However, it is possible that the RAW cells stimulated with LPS would provide a better assay system for this to be carried out.

### Example 8B

### MDA-MD-231 Matrigel Outgrowth Assay

### Method

Monolayers of cells (MDA-MD-MB231) were grown in DMEM+10% FCS to 80% confluence and collected during log phase. Cells were removed from plastic using versene/trypsin (2 min/370C) and resuspended in 10 ml media. Cell were washed thrice with PBS and centrifugation. 5×103 cells were added per well (96 well format) on top of preset Matrigel (50 ul). 50 ul of bioactive supernatant was added per well. Colony outgrowth was monitored over the course of the experiment and photographed after 2 and 5 days.

### Results

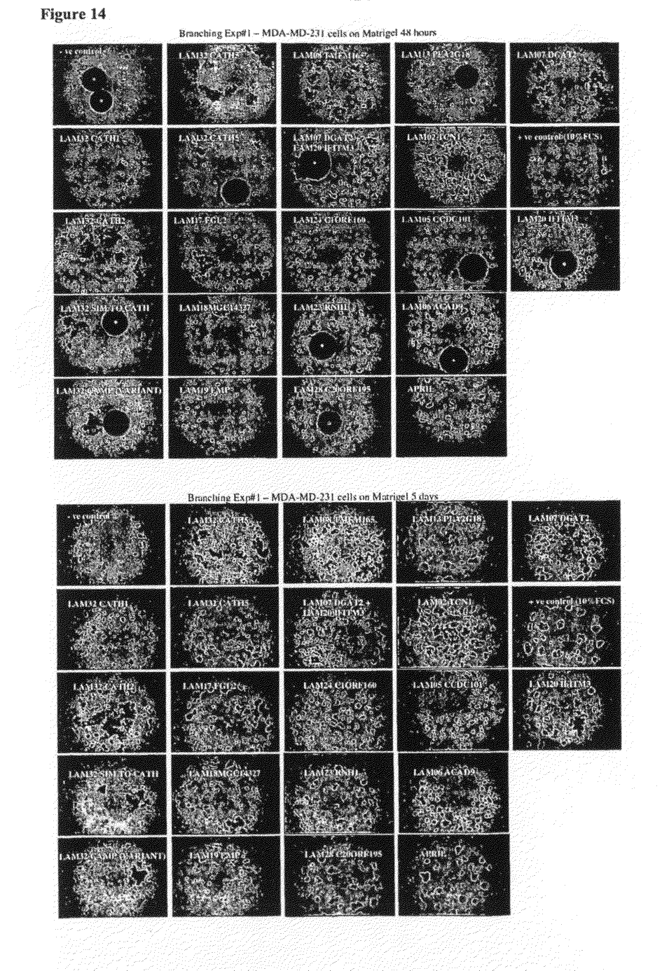

Invasive behaviour of cancer cells is reflected in cell culture by their ability to grow into a gel which is rich in extracellular matrix proteins (Matrigel). This invasive behaviour is a complex mechanism utilizing the ability of the cells to degrade the surrounding Matrix, form branching outgrowths and move within the matrix by enhancing cell motility. MDA-MB-231 cells usually form stellate colonies under these condition exhibiting filopodia) structures invading the surrounding matrix. The results observed here indicate that addition of LAM02 significantly increased branching morphology after 48 hours (FIG. 14A) compared to the control (OPTIMEM), while LAM05 inhibited all growth and branching of cells for up to 5 days. After 5 days (FIG. 14B) LAM23, LAM28 LAM13, LAM02 and APRIL showed increased proliferation of cells compared to the control (OPTIMEM) suggesting these bioactives would be candidates for further testing in proliferation based assays. Positive control cells (10% FCS in OPTIMEM) did not show extensive branching morphology as expected and this assay may be best repeated by embedding the cells within Matrigel.

### Interpretation of Results

The increased ability of LAM02 treated cells to from enhanced branching may indicate the cells have enhanced cell motility and invasive potential indicating that LAM02 may act to signal to the cell to stimulate these mechanisms. Similar mechanisms are used by normal cells in processes such as angiogenesis. The increased proliferation after 5 days for LAM23, LAM28 LAM13, LAM02 and APRIL treated cells implies that these molecules may act to stimulate cell growth.

The decrease in branching and proliferation exhibited by LAM05 treated cells indicates that this molecule may inhibit the process of cancer growth and invasion and therefore represents a potentially interesting molecule in the search for new cancer therapies and treatments.

### Example 8C

### Jurkat Cells

Jurkat cells are an immortalized human T cell line (Djordejevic et at AIDS Res. Hum. Retroviruses; 2004; 20(5); 547-555).

Jurkat cells were cultured with 10 uL of supernatant of cells expressing a LAM for 24 and 48 hours. Supernatant treated live cell counts are graphed relative to the control Ptarget count. Cell cultures were seeded at 4×10⁵ cells/mL and kept at 37° C. and 5% CO₂.

Cell counts of both live and dead jurkat cells were performed to assess the effect of the supernatants on proliferation and viability.

### Cell Counts—Live Cells

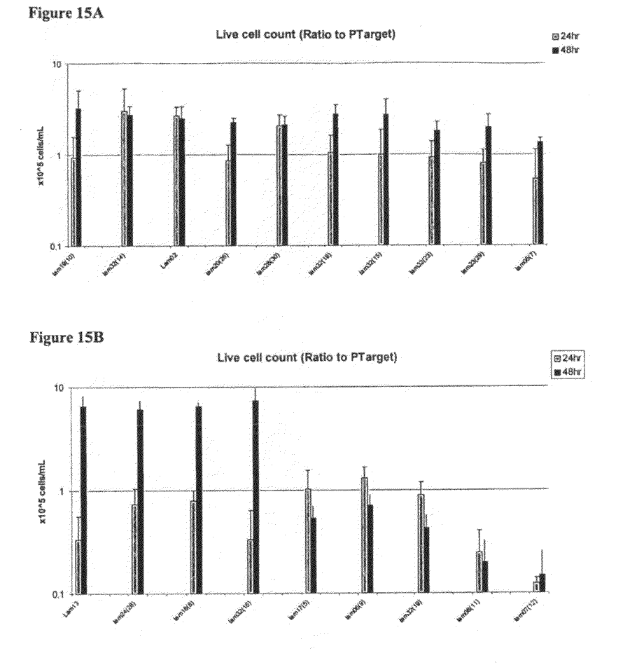

Live cell counts are graphed (FIGS. 15A and 15B) for each supernatant. The cell count is relative to the control (Ptarget). Hence a value about 1 indicates an increase in live cell numbers relative to the control, and the inverse when less than 1. Y error bars indicate 1 SD.

### Viability

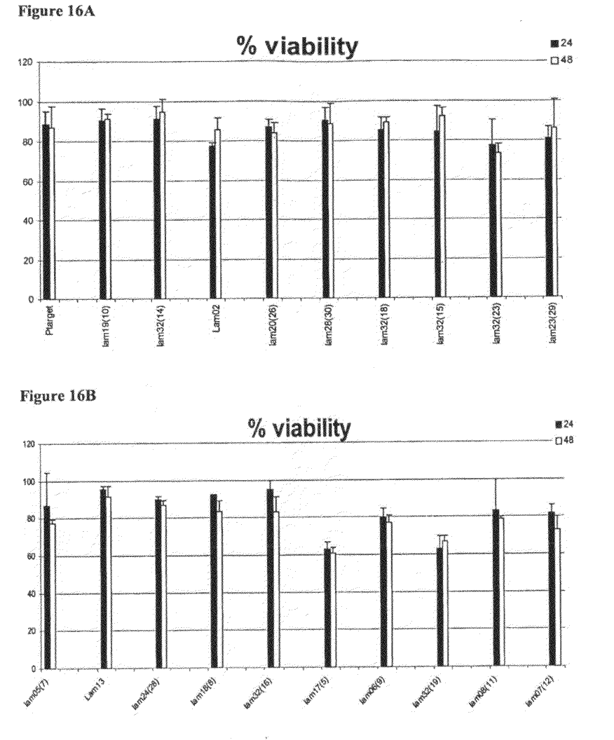

The percentage of live, viable cells are graphed (FIGS. 16A and 16B) for each supernatant (including the Ptarget control). Y error bars indicate 1 SD.

### Example 8D

### Kit 225 Cells

Kit 225 cells are an immortalized human, II-2 dependent T cell line (Sawami et at J. Cell Physiol.; 1992; 151(2); 367-377.

The cells were cultured with 10 uL of supernatant of cells expressing a LAM for 24 and 48 hours. Supernatant treated live cell counts graphed relative to the control Ptarget. Cell cultures were seeded at 4×10⁵ cells/mL and kept at 37° C. and 5% CO₂. Kit cells were cultured with 2 mediums—low IL-2 and high IL-2.

Cell counts of both live and dead kit 225 cells were performed to assess the effect of the supernatants on proliferation and viability.

### Cell Counts—Live

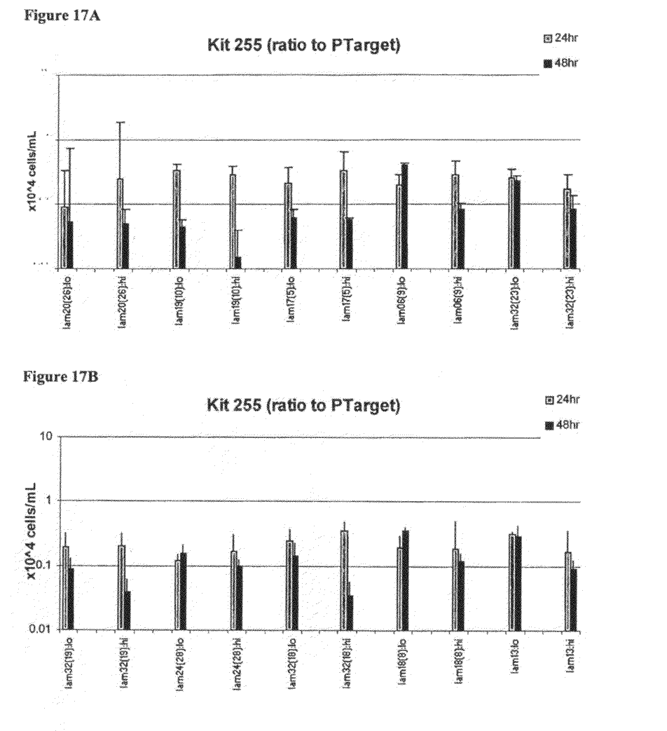

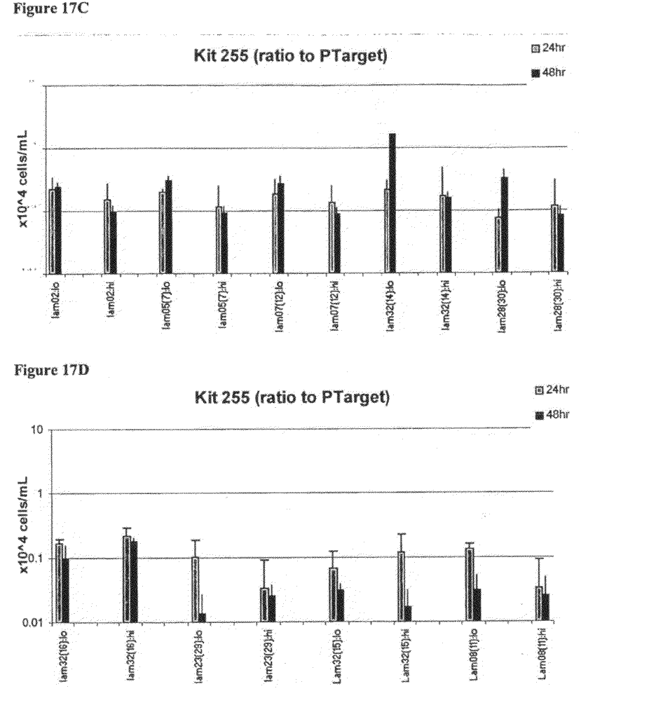

Live cell counts are graphed (FIGS. 17A-17D) for each supernatant. The cell count is to relative to the control (Ptarget). Hence a value about 1 indicates an increase in live cell numbers relative to the control, and the inverse when less than 1. Y error bars indicate 1 SD.

### Viability

The percentage of live, viable cells are graphed (FIGS. 18A-18D) for each supernatant (including the Ptarget control). Y error bars indicate 1 SD.

### Example 8E

### LAM 02

LAM02 is also designated as TCN1 or Transcobalamin I which is a vitamin B12 binding protein of the R binder family.

Following the method described in Example 4G2 in relation to FIG. 7 the inventors demonstrated a loss of OCT4 expression in mouse embryonic stems cells induced by bovine TCN1 (FIG. 19).

Following the method described in Example 8A in relation to FIG. 12 the inventors demonstrated ERK1/2 activation in Swiss 3T3 cells in response to bovine TCN1 (FIG. 20).

### Example 8F

### LAM 07

LAM07 is also designated as DGAT2 or Diacylglycerol O-acyltransferase homolog 2 (mouse).

Following the method described in Example 8A in relation to FIG. 12 the inventors demonstrated ERK1/2 activation in Swiss 3T3 cells in response to bovine DGAT2 (FIG. 21). Each experiment was conducted in quadruplicate.

### Example 8G

### LAM18

LAM18 is also designated as a hypothetical protein MGC14327.

Following the method described in Example 8A in relation to FIG. 13 the inventors demonstrated p38 MAPK stimulation in U937 cells in response to wallaby MGC14327 (FIG. 22).

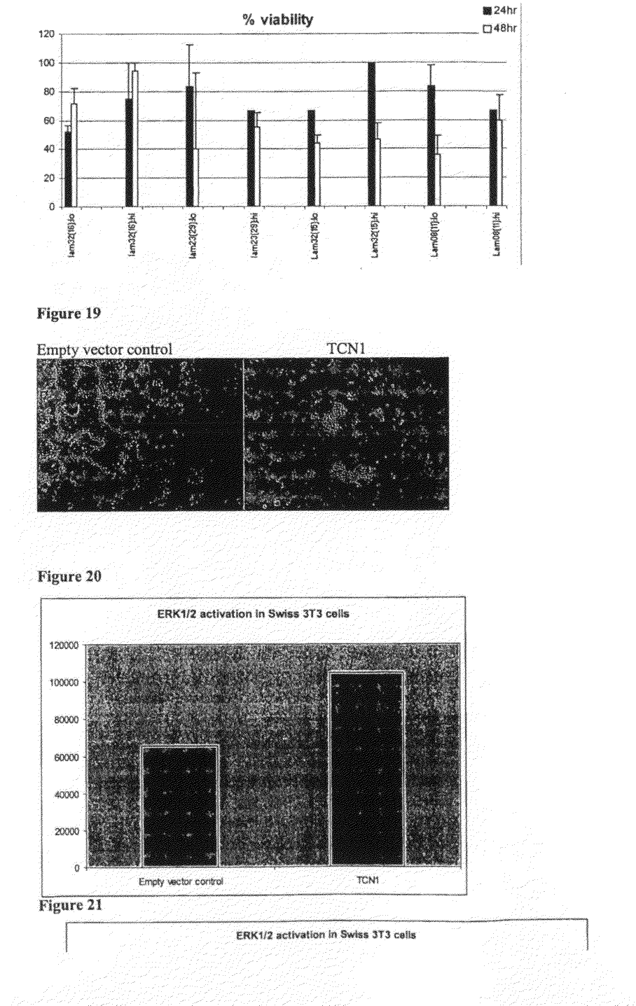

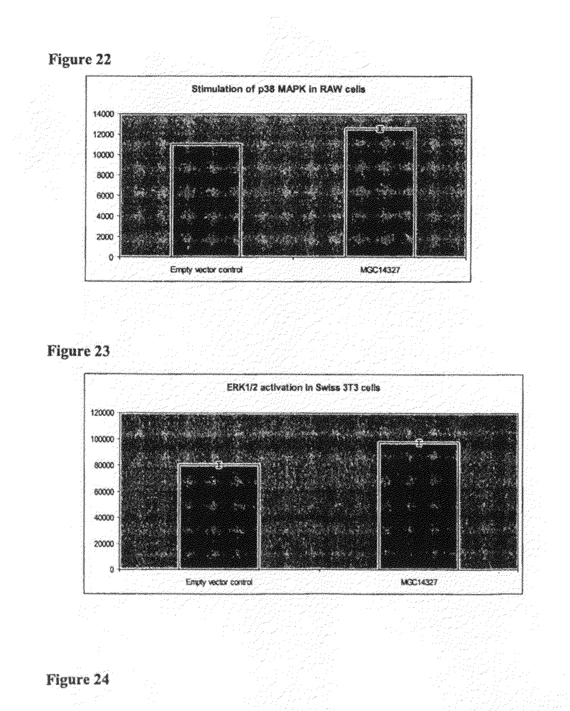

Following the method described in Example 8A in relation to FIG. 12 the inventors demonstrated ERK1/2 activation in Swiss 3T3 cells in response to bovine MGC14327 (FIG. 23). Each experiment was conducted in quadruplicate.

### Example 8H

### LAM20

LAM20 is also designated as IFITM3 or Interferon induced transmembrane protein 3 (1-8U)

Following the method described in Example 4G2 in relation to FIG. 7 This is correct the inventors demonstrated a loss of OCT4 expression in mouse embryonic stems cells induced by bovine IFITM3 (FIG. 24).

### Example 8I

### LAM24

LAM24 is also designated as C1orf160 or Chromosome 1 open reading frame 160.

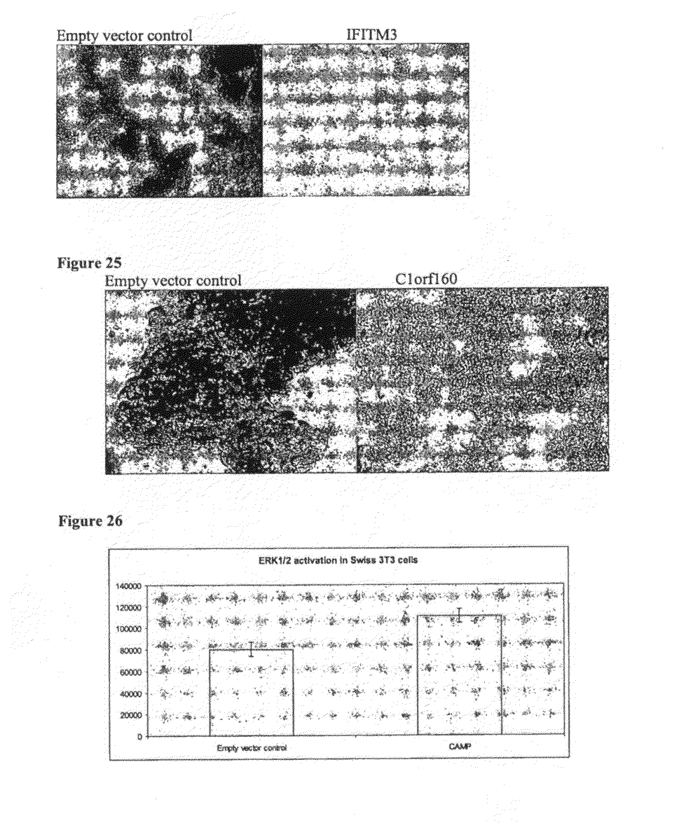

Following the method described in Example 4G2 in relation to FIG. 7 the inventors demonstrated a loss of OCT4 expression in mouse embryonic stems cells induced by bovine C1orf160 (FIG. 25).

### Example 8J

### LAM32

LAM32 is also designated as CAMP or Cathelicidin antimicrobial peptide

Following the method described in Example 8A in relation to FIG. 12 the inventors demonstrated ERK1/2 activation in Swiss 3T3 cells in response to bovine CAMP (FIG. 26). Each experiment was conducted in quadruplicate.

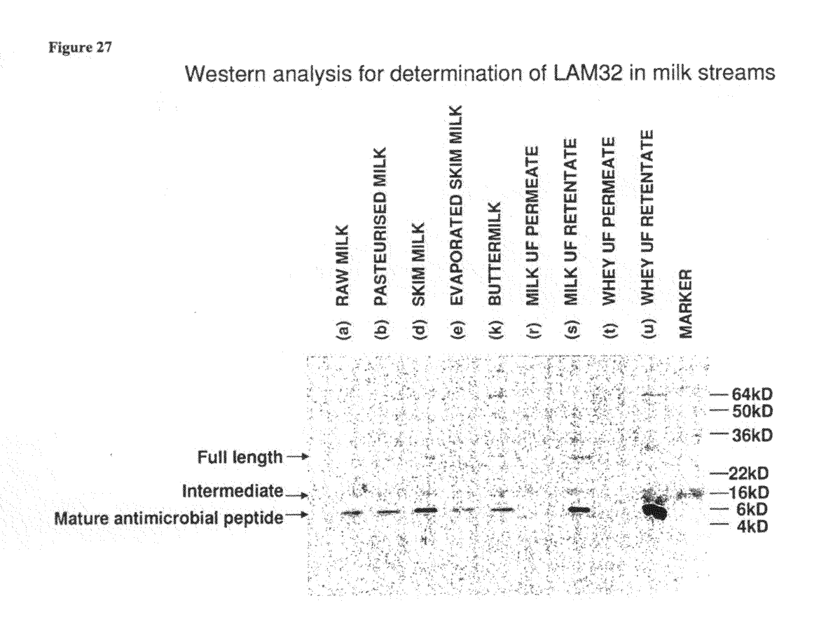

The expression of LAM32 in milk streams was also investigated by Western blotting. Milk stream samples from a commercial dairy processing factory were loaded into polyacrylamide gels and electrophoresed for about 1 hour. The polypeptides were transferred to nitrocellulaose membranes by Western blot then the immobilized polypeptides were probed with polyclonal rabbit anti-bovine cathelicidin antibody followed by goat anti-rabbit antibody conjugated for luminescence detection. FIG. 27 illustrates that LAM32 polypeptide can be found in raw milk, pasteurized milk, skim milk, evaporated skim milk and buttermilk. In addition LAM32 polypeptide can be found in milk and whey ultrafiltration retentates but not permeates (ultrafiltration permeate<10,000 Daltons).

### Example 8K

### Expression and Production of Bovine Cathelicidin Orthologues

Proteins in transfected HEK293 cell conditioned media were separated by 20% SDS-PAGE and transferred to a PDF membrane support. The membrane was blocked with 1% fish gelatine for 4 hours then incubated overnight in a 1:500 dilution of anti-cow cathelicidin. Reactive antibodies were detected using an anti-rabbit secondary antibody.

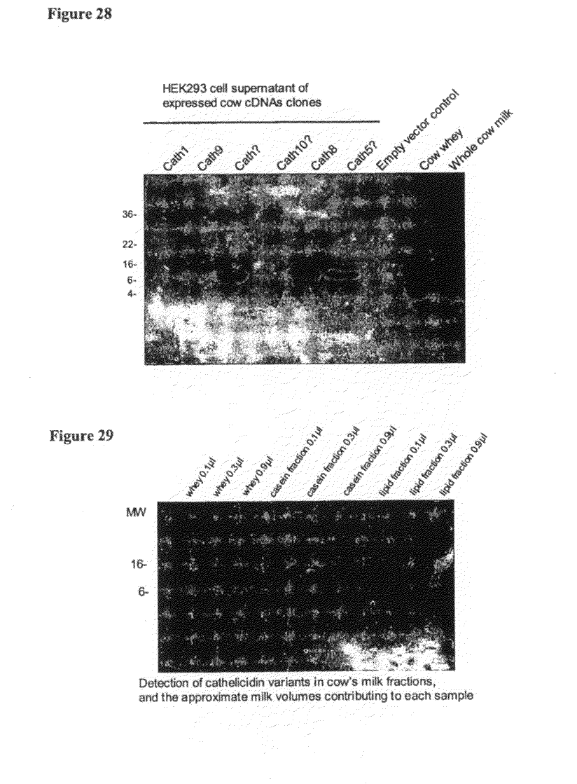

FIG. 28 demonstrates successful expression and production of bovine cathelicidin orthologues in transfected HEK 293 cell conditioned media.

FIG. 29 demonstrates multiple cathelicidin species in cow's milk by western blotting. A major band is present in the whey fraction and two major bands present in the casein fraction. Standard casein-whey fractionation was performed by high speed centrifugation.

The anti-bovine cathelicidin antibody was produced in rabbits to a synthetic peptide CEANLYRLLELDPPPK, where EANLYRLLELDPPPK represents a residues 51 to 65 of all bovine cathelicidin variants.

The inoculation regimen used to produce the anti-bovine cathelicidin antibody is shown in Table 25.

TABLE 25
Inoculation regimen

| Day | Action |
|---|---|
| 0 | Inoculate with 200 μg peptide in Complete Freund's Adjuvant |
| 14 | Inoculate with 100 μg peptide in Complete Freund's Adjuvant |
| 28 | Inoculate with 100 μg peptide in Complete Freund's Adjuvant |
| 42 | Inoculate with 100 μg peptide in Complete Freund's Adjuvant |
| 56 | Inoculate with 100 μg peptide in Complete Freund's Adjuvant |
| 63 | Bleed |

### Example 8L

### Summary of Biological Activities of Selected LAMs

Table 26 summarises the findings of the preceding examples as they relate to the biological activities of selected LAMs

TABLE 26
Summary of Biological activities of selected LAMs

| | | | | Prolifer- | PRO- | ANTI- | PRO- | | | |
|---|---|---|---|---|---|---|---|---|---|---|
| GENE ID | ANNOTATION | EST | ERK 1/2 | ation | P38 | P38 | APOP | MORPH | D* | T* |
| ANGPTL5 | Angiopoietin-like 5 | SGT20g4_B08 | | | | + | + | | | |
| C1orf160 | Chromosome 1 open reading frame 160 | SGT20k3_B07 | | | | | | + | + | |
| CAMP | Cathelicidin antimicrobial peptide | SGT20p4_G03 | + | + | | | | + | | |
| DGAT2 | Diacylglycerol O-acyltransferase homolog 2 | SGT20m5_H01 | + | | | | | + | + | |
| | (mouse) | | | | | | | | | |
| EBP | Emopamil binding protein (sterol isomerase) | SGT2011_C03 | | | +† | | | | + | + |
| IFITM3 | Interferon induced transmembrane protein 3 (1- | SGT2014_H04 | | | | | | | + | |
| | 8U) | | | | | | | | | |
| IMPAD1 | Inositol monophosphatase domain containing 1 | SGT20c1_F10 | | | | | | | + | |
| MGC14327 | Hypothetical protein MGC14327 | SGT20k4_C03 | + | | + | | | | | |
| RNH1 | Ribonuclease/angiogenin inhibitor 1 | SGT20o1_C06 | | | | | | | + | |
| TCN1 | Transcobalamin I (vitamin B12 binding protein, | SGT20g3_A01 | + | | | | | | + | |
| | R binder family) | | | | | | | | | |
| TMEM165 | Transmembrane protein 165 | SGT20n2_H05 | | | | | | + | | |

*D = Differentiation, T = Trefoil

### Example 8M

### HuVec Cells

HuVec are a human umbilical vein endothelial cell line (Galdal K S et al., Br. J. Haematol.; 1984; 58(4); 617-625).

HuVec—cultured with 10 uL of supernatant for 24 and 48 hours. Supernatant treated live cell counts are graphed relative to the control Ptarget count. Cell cultures were seeded at 4×10⁵ cells/mL and kept at 37° C. and 5% CO₂.

Cell counts of both live and dead HuVec cells were performed to assess the effect of the supernatants on proliferation and viability.

### Cell Counts—Live Cells

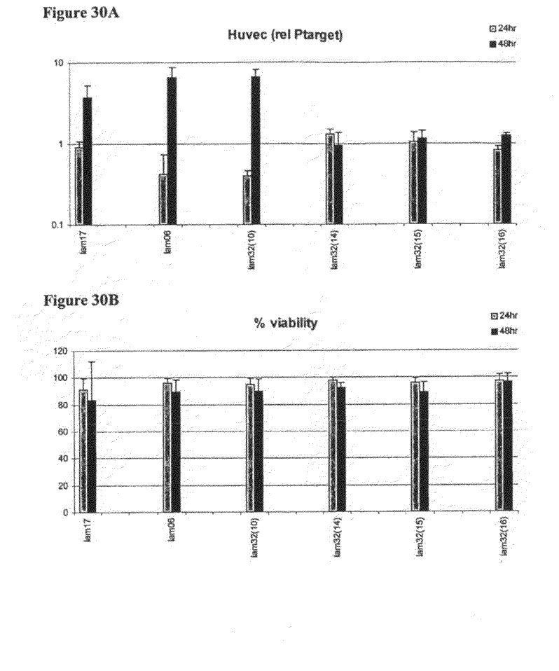

Live cell counts are graphed (FIG. 30A) for each supernatant. The cell count is relative to the control (Ptarget). Hence a value about 1 indicates an increase in live cell numbers relative to the control, and the inverse when less than 1. Y error bars indicate 1 SD.

### Viability

The percentage of live, viable cells are graphed (FIG. 30B) for each supernatant (including the Ptarget control). Y error bars indicate 1 SD.

### Example 8N

### Jurkat Cells

Jurkat cells are an immortalized human T cell line (Djordejevic of at AIDS Res. Hum. Retroviruses; 2004; 20(5); 547-555.

Jurkat cells—cultured with 10 uL of supernatant for 24 and 48 hours. Supernatant treated live cell counts are graphed relative to the control Ptarget count. Cell cultures were seeded at 4×10⁵ cells/mL and kept at 37° C. and 5% CO₂.

Cell counts of both live and dead jurkat cells were performed to assess the effect of the supernatants on proliferation and viability.

Cell Counts—Live cells

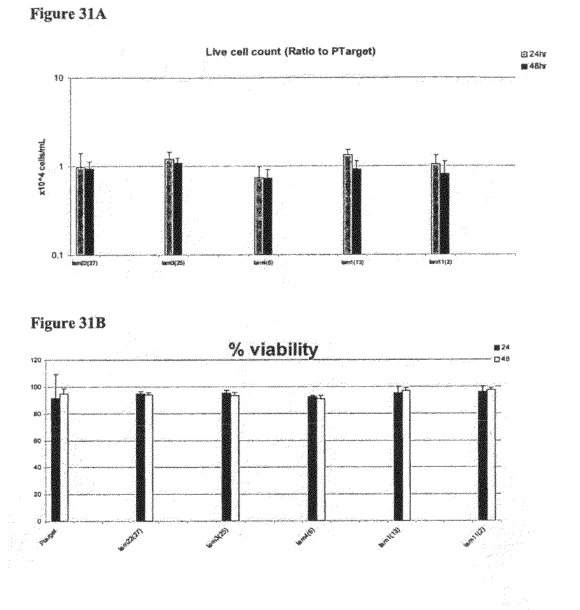

Live cell counts are graphed (FIG. 31A) below for each supernatant. The cell count is relative to the control (Ptarget). Hence a value about 1 indicates an increase in live cell numbers relative to the control, and the inverse when less than 1. Y error bars indicate 1 SD.

### Viability

The percentage of live, viable cells are graphed (FIG. 31B) for each supernatant (including the Ptarget control). Y error bars indicate 1 SD.

### Example 8O

### THP-1 Cells

THP-1 cells area human acute monocytic leukemia cell line (Tsuchiya S. et al., Int. J. Cancer, 1980, 26(2); 171-176).

THP-1 cells—cultured with 10 uL of supernatant for 24 and 48 hours. Supernatant treated live cell counts are graphed relative to the control Ptarget count. Cell cultures were seeded at 4×10⁵ cells/mL and kept at 37° C. and 5% CO₂.

Cell counts of both live and dead THP-1 cells were performed to assess the effect of the supernatants on proliferation and viability.

### Cell Counts—Live Cells

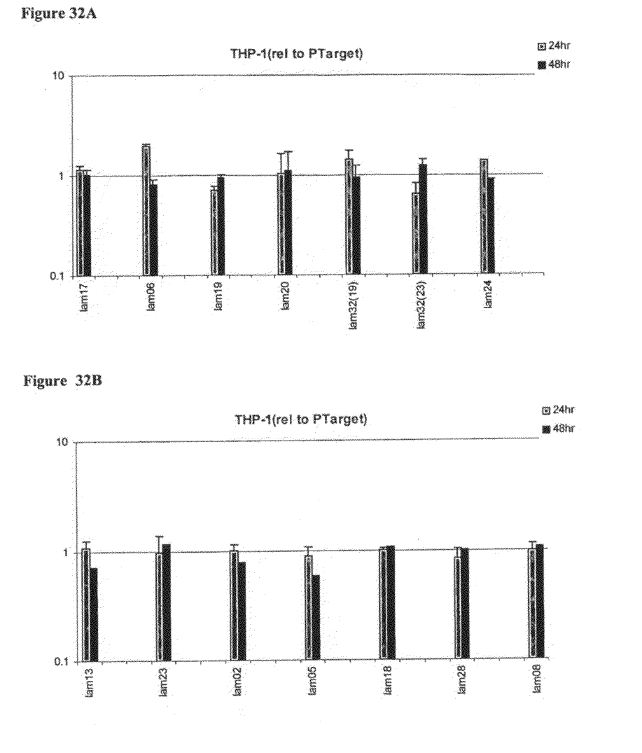

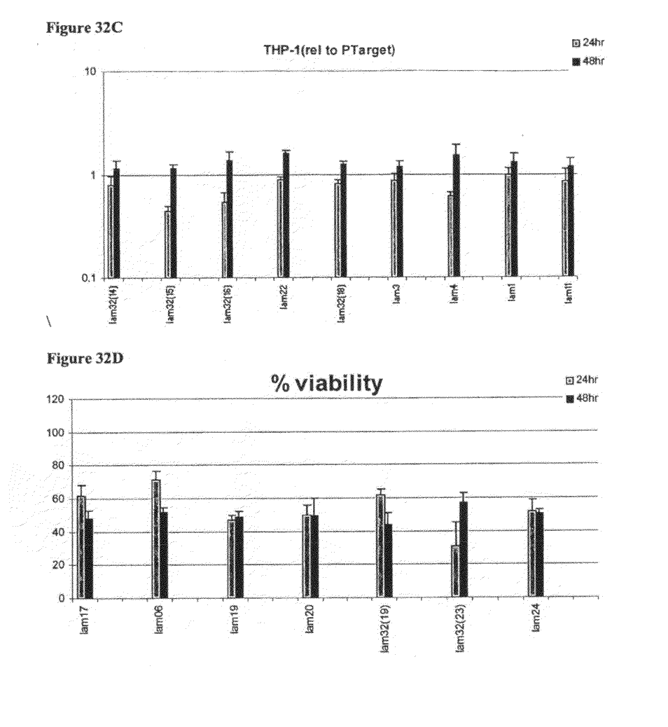

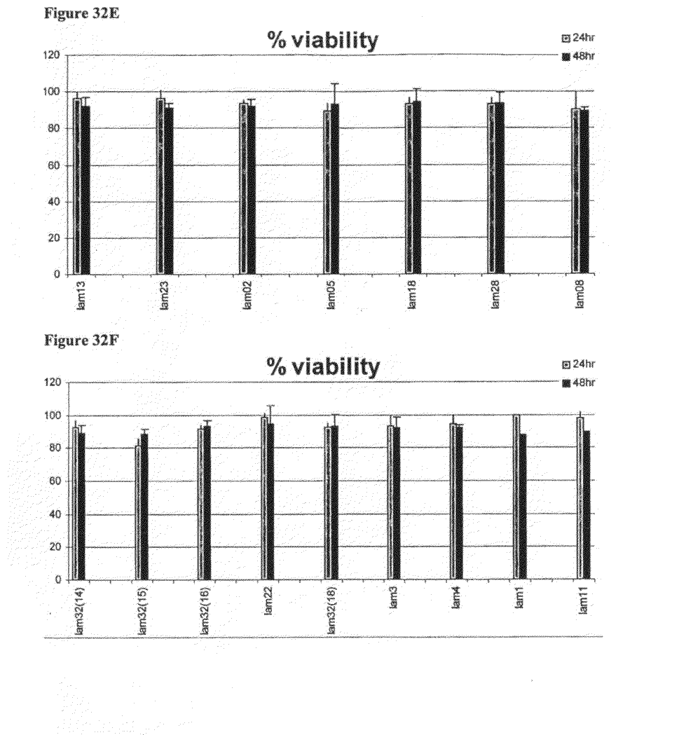

Live cell counts are graphed (FIGS. 32A-32C) below for each supernatant. The cell count is relative to the control (Ptarget). Hence a value about 1 indicates an increase in live cell numbers relative to the control, and the inverse when less than 1. Y error bars indicate 1 SD.

### Viability

The percentage of live, viable cells are graphed (FIGS. 32D-32F) for each supernatant (including the Ptarget control). Y error bars indicate 1 SD.

### Example 8P

### Proliferation Assay

MDA-MB-231 cells were plated (1000 cells/well) in 96 well plate formats with 100 μl growth media (DMEM/10% foetal calf serum). After one day media was removed, bioactive supematants (50 μl) were added and cells were grown for a further 3, and 6 days before being fixed with 10% TCA (1 hour/4° C.), washing five times with H₂O and allowed to dry overnight. After all plates were collected, cells were stained with Sulforhodamine B for 10 min, washed five times with 1% acetic acid and allowed to dry overnight. The following day 100 μl of 10 mM TRS (unbuffered) was added and plates were read on a plate reader at 540 nm. Each time point was performed in triplicate. Error bars are shown.

Statistical analyses were performed by t-test against the standard (10% FCS).

### Results

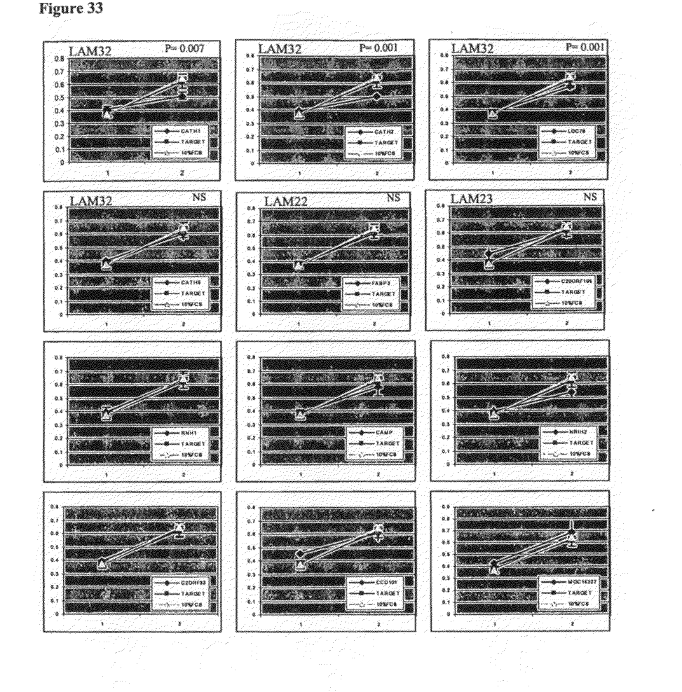

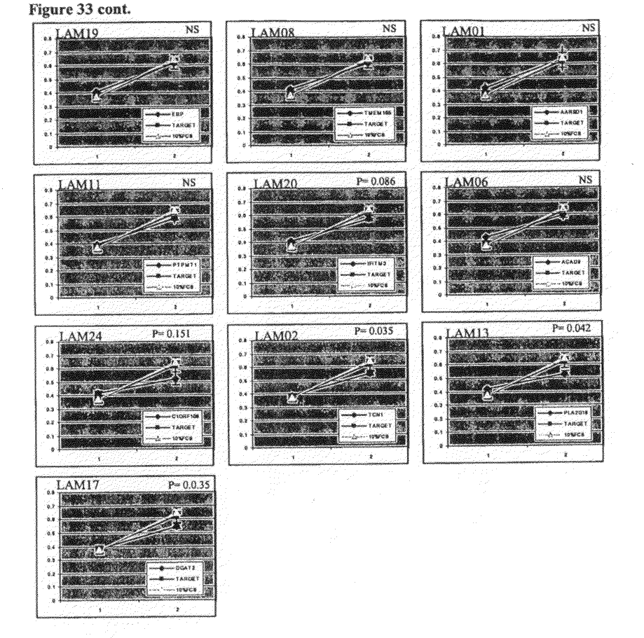

Proliferation curves (FIG. 33) show differences in the rate of proliferation between the controls and the presence of each bioactive (P values are shown). Note that rate of proliferation is represented by the gradient.

1. A lactation-associated polypeptide, wherein said polypeptide is encoded by:
(a) a polynucleotide of a cDNA library as shown in Table 1; or
(b) a functionally equivalent variant, analogue, derivative or fragment of a polynucleotide of a cDNA library as shown in Table 1.

2. The polypeptide according to claim 1, wherein said polypeptide is a secreted polypeptide.

3. The polypeptide according to claim 1 or claim 2, wherein the polypeptide exhibits a bioactivity selected from the group comprising:
(a) regulation of mammary gland development including mammosphere formation;
(b) regulation of lactation;
(c) regulation of milk composition;
(d) growth-promoting activity, including cell proliferative activity, including cellular differentiation and/or morphology activity;
(e) pro- or anti-inflammatory activity;
(f) pro- or anti-apoptotic activity;
(g) anti-microbial activity;
(h) regulation of differentiation of embryonic stem cells;
(i) regulation of trefoil activity;
(j) cathelicidin activity;
(k) regulation of epithelial cells, including gut and/or skin epithelial cells; and/or
(l) regulation of growth of chicks.

4. A lactation-associated polypeptide, wherein said polypeptide comprises an amino acid sequence selected from the group consisting of SEQ ID NOs: 31-59, 91-119, 152, 154, 156, 158, 160, 162, 164, 166, 171, 173, 175, 177, 179, 181, 184, 186, 188, 207, 215, 217 and 219 and or a functionally equivalent variant, analogue, derivative or fragment thereof.

5. A polynucleotide encoding the polypeptide according to any one of claims 1 to 4, or a functionally equivalent variant, analogue, derivative or fragment thereof.

6. A polynucleotide that hybridizes to the polynucleotide according to claim 5 under conditions of high stringency.

7. A lactation-associated polynucleotide, wherein said polynucleotide comprises:
(a) a nucleic acid sequence selected from the group consisting of SEQ ID NOs: 1-29, 61-89, 151, 153, 155, 157, 159, 161, 163, 165, 167-170, 172, 174, 176, 178, 180, 183, 185, 190-192, 208, 216 and 218 or a functionally equivalent variant, analogue, derivative or fragment thereof; or
(b) a polynucleotide that hybridizes to the polynucleotide selected from (a) under conditions of high stringency.

8. A polypeptide encoded by the polynucleotide according to claim 7.

9. An expression vector comprising the polynucleotide according to any one of claims 5 to 7, wherein the polynucleotide is operably linked to a promoter.

10. A host cell transformed with the expression vector according to claim 9.

11. A bioactive molecule, wherein said molecule has cell growth-promoting activity, and wherein the molecule comprises a polypeptide comprising an amino acid sequence selected from the group as set forth in SEQ ID NOs: 32, 34-35, 37, 42, 48, 51-53, 55, 92, 94-95, 97, 102, 108, 111-113, 115 and 152 or a functionally equivalent variant, analogue, derivative or fragment thereof.

12. A bioactive molecule, wherein said molecule has cell growth-promoting activity, and wherein the molecule is encoded by:
(a) a polynucleotide comprising a nucleotide sequence selected from the group as set forth in SEQ ID NOs: 2, 4-5, 7, 12, 18, 21-23, 25, 62, 64-65, 67, 72, 78, 81-83, 85 and 151 or a functionally equivalent variant, analogue, derivative or fragment thereof; or
(b) a polynucleotide that hybridizes to the polynucleotide selected from (a) under conditions of high stringency.

13. A bioactive molecule, wherein said molecule has pro-apoptotic activity, and wherein the molecule comprises a polypeptide comprising an amino acid sequence selected from the group as set forth in SEQ ID NOs: 46-47, 106-107, and 156 or a functionally equivalent variant, analogue, derivative or fragment thereof.

14. A bioactive molecule, wherein said molecule has pro-apoptotic activity, and wherein the molecule is encoded by:
(a) a polynucleotide comprising a nucleotide sequence selected from the group as set forth in SEQ ID NOs: 16-17, 76-77, and 155 or a functionally equivalent variant, analogue, derivative or fragment thereof; or
(b) a polynucleotide that hybridizes to the polynucleotide selected from (a) under conditions of high stringency.

15. A bioactive molecule, wherein said molecule has anti-apoptotic activity, and wherein the molecule comprises a polypeptide comprising an amino acid sequence selected from the group as set forth in SEQ ID NOs: 43 and 103 or a functionally equivalent variant, analogue, derivative or fragment thereof.

16. A bioactive molecule, wherein said molecule has anti-apoptotic activity, and wherein the molecule is encoded by:
(a) a polynucleotide comprising a nucleotide sequence selected from the group as set forth in SEQ ID NOs: 13 and 73 or a functionally equivalent variant, analogue, derivative or fragment thereof; or
(b) a polynucleotide that hybridizes to the polynucleotide selected from (a) under conditions of high stringency.

17. A bioactive molecule, wherein said molecule has pro-inflammatory activity, and wherein the molecule comprises a polypeptide comprising an amino acid sequence selected from the group as set forth in SEQ ID NOs: 31, 35, 41, 45-46, 49, 91, 95, 101, 105-106, 109, 154 and 156or a functionally equivalent variant, analogue, derivative or fragment thereof.

18. A bioactive molecule, wherein said molecule has pro-inflammatory activity, and wherein the molecule is encoded by:
(a) a polynucleotide comprising a nucleotide sequence selected from the group as set forth in SEQ ID NOs: 1, 5, 11, 15-16, 19, 61, 65, 71, 75-76, 79, 153, and 155 or a functionally equivalent variant, analogue, derivative or fragment thereof; or
(b) a polynucleotide that hybridizes to the polynucleotide selected from (a) under conditions of high stringency.

19. A bioactive molecule, wherein said molecule has anti-inflammatory activity, and wherein the molecule comprises a polypeptide comprising an amino acid sequence selected from the group as set forth in SEQ ID NOs: 35-36, 41, 47, 95-96, 101, 107, or a functionally equivalent variant, analogue, derivative or fragment thereof.

20. A bioactive molecule, wherein said molecule has anti-inflammatory activity, and wherein the molecule is encoded by:
(a) a polynucleotide comprising a nucleotide sequence selected from the group as set forth in SEQ ID NOs: 5-6, 11, 17, 65-66, 71, 77, or a functionally equivalent variant, analogue, derivative or fragment thereof; or
(b) a polynucleotide that hybridizes to the polynucleotide selected from (a) under conditions of high stringency.

21. A bioactive molecule, wherein said molecule has the ability to regulate cellular morphology, and wherein the molecule comprises a polypeptide comprising an amino acid sequence selected from the group as set forth in SEQ ID NO: 33, 37-40, 54, 93, 97-100, 114, 158 and 188 or a functionally equivalent variant, analogue, derivative or fragment thereof.

22. A bioactive molecule, wherein said molecule has the ability to regulate cellular morphology, and wherein the molecule is encoded by:
(a) a polynucleotide comprising a nucleotide sequence selected from the group as set forth in SEQ ID NOs: 3, 7-10, 24, 63, 67-70, 84, 157, 167, 191 and 208 or a functionally equivalent variant, analogue, derivative or fragment thereof; or
(b) a polynucleotide that hybridizes to the polynucleotide selected from (a) under conditions of high stringency.

23. A bioactive molecule, wherein said molecule has the ability to influence cellular differentiation, and wherein the molecule comprises a polypeptide comprising an amino acid sequence selected from the group as set forth in SEQ ID NOs: 37, 44, 49-50, 54-59, 97, 104, 109-110, 114-119, 158, 160, 162, 164, and 166 or a functionally equivalent variant, analogue, derivative or fragment thereof.

24. A bioactive molecule, wherein said molecule has the ability to influence cellular differentiation, and wherein the molecule is encoded by:
(a) a polynucleotide comprising a nucleotide sequence selected from the group as set forth in SEQ ID NO: 7, 14, 19-20, 24-29, 67, 74, 79-80, 84-89, 157, 159, 161, 163, and 165 or a functionally equivalent variant, analogue, derivative or fragment thereof; or
(b) a polynucleotide that hybridizes to the polynucleotide selected from (a) under conditions of high stringency.

25. A bioactive molecule, wherein said molecule has the ability to modulate the expression of trefoil proteins, and wherein the molecule comprises a polypeptide comprising an amino acid sequence selected from the group as set forth in SEQ ID NOs: 31, 45-46, 49, 91, 105-106, 109, 179, 181, 184,186, 217 and 219 or a functionally equivalent variant, analogue, derivative or fragment thereof.

26. A bioactive molecule, wherein said molecule has the ability to modulate the expression of trefoil proteins, and wherein the molecule is encoded by:
(a) a polynucleotide comprising a nucleotide sequence selected from the group as set forth in SEQ ID NO: 1, 15-16, 19, 61, 75-76, 79, 178, 180, 183, 185, 190, 192, 216, and 218 or a functionally equivalent variant, analogue, derivative or fragment thereof; or
(b) a polynucleotide that hybridizes to the polynucleotide selected from (a) under conditions of high stringency.

27. A bioactive molecule, wherein said molecule has cathelicidin activity, and wherein the molecule comprises a polypeptide comprising an amino acid sequence selected from the group as set forth in SEQ ID NO: 171, 173, 175, 177, 188, 207, and 215 or a functionally equivalent variant, analogue, derivative or fragment thereof.

28. A bioactive molecule, wherein said molecule has cathelicidin activity, and wherein the molecule is encoded by:
(a) a polynucleotide comprising a nucleotide sequence selected from the group as set forth in SEQ ID NO: 167-169, 170, 172, 174, 176, 191, and 208 or a functionally equivalent variant, analogue, derivative or fragment thereof; or
(b) a polynucleotide that hybridizes to the polynucleotide selected from (a) under conditions of high stringency.

29. A bioactive molecule, wherein said molecule has the ability to affect cell growth, and wherein the molecule comprises a polypeptide comprising an amino acid sequence selected from the group as set forth in SEQ ID NO: 171, 173, 175, 177, 188, 207, and 215 or a functionally equivalent variant, analogue, derivative or fragment thereof.

30. A bioactive molecule, wherein said molecule has the ability to affect cell growth, and wherein the molecule is encoded by:
(a) a polynucleotide comprising a nucleotide sequence selected from the group as set forth in SEQ ID NO: 167-169, 170, 172, 174, 176, 191, and 208 or a functionally equivalent variant, analogue, derivative or fragment thereof; or
(b) a polynucleotide that hybridizes to the polynucleotide selected from (a) under conditions of high stringency.

31. A bioactive molecule, wherein said molecule has the ability to affect growth of chicks, and wherein the molecule comprises a polypeptide comprising an amino acid sequence as set forth in SEQ ID NO: 34-35, 49, 94-95, 109, 171,173, and 177 or a functionally equivalent variant, analogue, derivative or fragment thereof.

32. A bioactive molecule, wherein said molecule has the ability to affect growth of chicks, and wherein the molecule is encoded by:
(a) a polynucleotide comprising a nucleotide sequence as set forth in SEQ ID NO: 4-5, 19, 64-65, 79, 170, 172, and 176 or a functionally equivalent variant, analogue, derivative or fragment thereof; or
(b) a polynucleotide that hybridizes to the polynucleotide selected from (a) under conditions of high stringency.

33. A bioactive molecule, wherein said molecule has anti-microbial activity, and wherein the molecule comprises a polypeptide comprising an amino acid sequence as set forth in SEQ ID NO: 171, 173, 175, 177, 188, 207, and 215 or a functionally equivalent variant, analogue, derivative or fragment thereof.

34. A bioactive molecule, wherein said molecule has anti-microbial activity, and wherein the molecule is encoded by:
(a) a polynucleotide comprising a nucleotide sequence as set forth in SEQ ID NO: 167-169, 170, 172, 174, 176, 191, and 208 or a functionally equivalent variant, analogue, derivative or fragment thereof; or
(b) a polynucleotide that hybridizes to the polynucleotide selected from (a) under conditions of high stringency.

35. A bioactive molecule, wherein said molecule has the ability to modulate stem cell pluripotency, and wherein the molecule comprises a polypeptide comprising an amino acid sequence as set forth in SEQ ID NO: 37, 44, 49-50, 54-59, 97, 104, 109-110, 114-119, 158, 160, 162, 164, and 166 or a functionally equivalent variant, analogue, derivative or fragment thereof.

36. A bioactive molecule, wherein said molecule has the ability to modulate stem cell pluripotency, and wherein the molecule is encoded by:
(a) a polynucleotide comprising a nucleotide sequence as set forth in SEQ ID NO: 7, 14, 19-20, 24-29, 67, 74, 79-80, 84-89, 157, 159, 161, 163, and 165 or a functionally equivalent variant, analogue, derivative or fragment thereof; or
(b) a polynucleotide that hybridizes to the polynucleotide selected from (a) under conditions of high stringency.

37. A method for screening a candidate polypeptide for bioactivity, wherein said method comprises:
(a) introducing into a suitable host cell the polynucleotide according to any one of claims 5 to 7, or the expression vector according to claim 9, encoding the candidate polypeptide;
(b) culturing the cell under conditions suitable for expression of the candidate polypeptide encoded by the polynucleotide;
(c) recovering the candidate polypeptide; and
(d) assaying the recovered candidate polypeptide for biological activity.

38. A method for screening a candidate polypeptide for bioactivity, wherein said method comprises:
(a) introducing into a suitable host cell the polynucleotide according to any one of claims 5 to 7, or the expression vector according to claim 9, encoding the candidate polypeptide;
(b) culturing the cell under conditions suitable for expression of the candidate polypeptide encoded by the polynucleotide, and for secretion of the candidate polypeptide into the extracellular medium;
(c) recovering the candidate polypeptide; and
(d) assaying the recovered candidate polypeptide for biological activity.

39. The method according to claim 38, wherein the assaying in step (d) comprises assaying for any one or more or the following: regulation of mammary gland development including mammosphere formation, regulation of lactation, regulation of milk composition, cell proliferative activity including cellular differentiation and/or morphology activity, pro- or anti-inflammatory activity, pro- or anti-apoptotic activity, anti-microbial activity, regulation of differentiation of embryonic stem cells, regulation of trefoil activity, cathelicidin activity and regulation of epithelial cells including gut and/or skin epithelial cells.

40. A polypeptide screened according to the method of any one of claims 37 to 39.

41. A method for screening a candidate mammal for lactation capability, wherein said method comprises:
(a) obtaining a biological sample from the candidate mammal; and
(b) determining the level of expression in the biological sample of one or more polynucleotides selected from the group consisting of SEQ ID NOs: 1-29, 61-89, 151, 153, 155, 157, 159, 161, 163, 165, 167-170, 172, 174, 176, 178, 180, 183, 185, 190-192, 208, 216 and 218 or a functionally equivalent variant, analogue, derivative or fragment thereof wherein the level of expression of the one or more polynucleotides is indicative of lactation capability.

42. A mammal screened according to the method of claim 41.

43. A method for screening for a candidate molecule that modulates the expression of the polypeptide according to any one of claim 1 to 3 or 8, or the bioactive molecule according to any one of claims 11 to 36, wherein said method comprises:
(a) contacting said candidate molecule with said polypeptide or said bioactive molecule, or a polynucleotide encoding said polypeptide or said bioactive molecule; and
(b) assaying for the level of expression of said polypeptide, said bioactive molecule or said polynucleotide
wherein the level of expression of said polypeptide or said bioactive molecule or said polynucleotide is indicative of the capacity of the candidate molecule to modulate expression of said polypeptide or said bioactive molecule.

44. A molecule screened according to the method of claim 43.

45. A method for isolating a lactation-associated polynucleotide or a functionally equivalent variant, analogue, derivative or fragment thereof in a mammal, wherein said method comprises:
(a) obtaining a biological sample from said mammal;
(b) contacting the biological sample with a first polynucleotide comprising the polynucleotide according to any one of claims 5 to 7;
(c) detecting hybridization between the first polynucleotide and a second polynucleotide in the biological sample; and
(d) isolating the second polynucleotide in the biological sample.

46. A lactation-associated polynucleotide isolated according to the method of claim 45.

47. A polypeptide encoded by the polynucleotide according to claim 46.

48. A pharmaceutical composition, wherein said composition comprises at least one of:
(a) the polypeptide according to any one of claim 1 to 3, 8, 40 or 47;
(b) the bioactive molecule according to anyone of claims 11 to 36;
(c) the polynucleotide according to any one of claim 5 to 7 or 46;
(d) the expression vector according to claim 9;
(e) the host cell according to claim 10; and
(f) the molecule according to claim 44.
together with a pharmaceutically acceptable carrier, diluent, excipient and/or adjuvant.

49. A method for treating a disease or disorder in a subject, wherein said method comprises administering to the subject at least one of:
(a) the polypeptide according to any one of claim 1 to 3, 8, 40 or 47;
(b) the bioactive molecule according to anyone of claims 11 to 36;
(c) the polynucleotide according to any one of claim 5 to 7 or 46;
(d) the expression vector according to claim 9;
(e) the host cell according to claim 10;
(f) the molecule according to claim 44; and
(g) the composition according to claim 48.

50. A method for enhancing the regeneration of epithelial cells in a subject, wherein said method comprises administering to the subject at least one of:
(a) the polypeptide according to any one of claim 1 to 3, 8, 40 or 47;
(b) the bioactive molecule according to anyone of claims 11 to 36;
(c) the polynucleotide according to any one of claim 5 to 7 or 46;
(d) the expression vector according to claim 9;
(e) the host cell according to claim 10;
(f) the molecule according to claim 44; and
(g) the composition according to claim 48.

51. The method according to claim 50, wherein the subject has been, or is being, subjected to chemotherapy.

52. A use of at least one of:
(a) the polypeptide according to any one of claim 1 to 3, 8, 40 or 47;
(b) the bioactive molecule according to anyone of claims 11 to 36;
(c) the polynucleotide according to any one of claim 5 to 7 or 46;
(d) the expression vector according to claim 9;
(e) the host cell according to claim 10;
(f) the molecule according to claim 44; and
(g) the composition according to claim 48 in the manufacture of a medicament for treating a disease or disorder in a subject.

53. A method for preserving food, wherein said method comprises contacting the food with at least one of:
(a) the polypeptide according to any one of claim 1 to 3, 8, 40 or 47;
(b) the bioactive molecule according to anyone of claims 11 to 36;
(c) the molecule according to claim 44; and
(d) the composition according to claim 48.

54. A food supplement for animals, wherein said food supplement comprises at least one of:
(a) the polypeptide according to any one of claim 1 to 3, 8, 40 or 47;
(b) the bioactive molecule according to anyone of claims 11 to 36;
(c) the molecule according to claim 44; and
(d) the composition according to claim 48.


SEQUENCE LISTING (220 sequences)

SEQ ID NO: 1 - DNA, 984 bp, Macropus eugenii
aaatggaataagcaggctggtaccggtccggaattcccgggatgaacaaatagttgctatcgagcagagtgtcaatgagaagatccgaaatcggctacctgtgactgtgatggaactgagcctggatgatcctgaggtggagaaggtgaagggcttgggtttaccagatgaccatgcagggcctattcgtgtcatcaccattgagggtgtcgactccaatatgtgctgtggaactcacgtgagaaatctcagcgacctgcaagtcattaagatcctaggtacagagaaagggaaaaagaacaagaccaacctagtgtttctagcagggaaccgggtactgaagtggatggagaaaagtcatggagttgaaaaggccttgacggcattactcaagtgtggagtagaagaccatgcagaggctgttcagaaactgcagaactcagccaagctgctccagaagaataatgtgaatctgctcagagacttggccattctcactgcccacagtcttcgagacagtccagacaggggaggagtgattgtcttacacaggaaagatggtgactctgagtttatgaacatcgttgctaatgagtttgggatagaggacacccttctcttcttaaccgtaggggacgagaaaggtgcggggctcttcttacttgcagggccatttgaggcagtggatatgttgggacccagggtagctgaattgcttcaaggcaaaggagctgggaagaaaggccgctttcagggcaaggccaccaaactgagccgacgggcagaggtgcacgcccttctccaggactatctgaacagtcagagtgctgaggaatagcagctcttgcccttgtatccttcctaggtgtttccttagaccagacagcatgtggccatggaaaaattttgctttctctagacataaatgatttattggcaaaaaagaaccactcaaaacaagagaatagataaaacataaaaata

SEQ ID NO: 2 - DNA, 1255 bp, Macropus eugenii
cttctctgcagaacaaatgaagaattctattggtggccagttctctgtagacactggggcacttgcagtcctggctatgacttgtgtgaacaggcagaacccagatgaaatgattagaagtaatatattatggcttgtggagaagatcctgaaggagaaaaaacaagatggcaccattggaaatatctatagcacgggagaagccatgcaggccctttttgtgactccggagtattataaggaaacagactgggactgtagtaaaactctgaaaactgtgctaagtcagattcctcagggaacattcagattgcccatcgctgcagcccaggtgttacctgccctggagggcaagacttacttggacataacacgtgtttcttgtcagtccacaccaggtcacctcagcatcaccccaaagcctacagaacccactcaccaattctacgagagtatctcagtcacttacagagtagtggactccatcaataacaacattaatgaatccaccattgtctcagtcccaaagggctctgtcttcctgaaggtgatggaggcagctcaaacaaaaagcaaaaccaagtttgggttcacaacaaagtcagaacaatggggagaatatatcacctctgttcatggtattaaggccagcagtgaaaacagaacctactggcagctcctgagtaatggtcaaccactgagccaaggagctgagagctttgtagtgtctgacggtgatcatctggaggtcaggctgagtagatattgaaggaagagacattggaagagtcatccaaccatagtatctagcctttccacctatggagtccactctaggattcctttgggaaacaaaagaccccttacctttctccttgcccatcttagcccccatgggaagtcaagcagctttgttctctctccttctcccccttgcctctctctctctctctctctctctctctctctctctctgtctgtatctctctctctcccacccgtctcccccatcatatctccccccttcacttgtctatcctctactagagcgtttgtcccatcaatcatacatactctccctcctctcgttccccctagatactatagcttctgtaccaatctcccacgctcgctttcccaccatttaaccgagatctacatctttatatccacataccactatacatacaccaatctcactccatacctcctcccgcccccttccctcatccaacccccctctcaccccacaccg

SEQ ID NO: 3 - DNA, 717 bp, Macropus eugenii
ccgccccaggccggccggaagtgacgctaggctggggttgccggaagaagtggcgaagttacttttcgcggggcgaacccgggggagcggcttggaggcggccggcgcgggcagtgggcacggggcagagggggtgaaggagacgggagacgcctttagggcagacgctgagaggagcctcccgggccggagcccaaactgtggcgtgcgcagccatgagctgggctctcctgcggtgacccaccatggcttcccctggcaccctcggcccccctgcagccccggcccccgccccgggaggtgatgataatggggcttccactacaacgtgctcagctgtgagggctgcaaaggctttttccggcgcagcgtggtgcggggcggtgcccagcgctacacttgtcgtgggggtggggcttgccacatgaatgccatcatgcgtcgcaagtgccaggagtgccgcctgcgcaagtgccgtgaggcgggcatgcgggagcagtgtgtcttgtctgaggagcagattcgcaagaaaaagattcagaaacaccagcccgtggggccagcgcagggctcaggggccccggctgctcctagggctcccatccctgggcctgcgggctccccaaccacatcctcagaagtgggagggccaggccctgccgatggcactggtgtccagctcacacctgaccagaagatcatgatccagcagcttgt

SEQ ID NO: 4 - DNA, 732 bp, Macropus eugenii
aagaacgtgcgaagagagagatgatcatgtattcgattaccgtagcgttctggctgctcaatagctggctctggtttcgccgctagaggtaacctcagctctctaacgtacttgtcataacagctggaaatataaaacatttgcaaacttctttgtttctgtctctgcattgtatgccgtttgataatccatgcactgaaaatgtatttctctgggaaagaacaaaggacctagaacagaactgaaaggacatcctgttgcttggttgtgtttctttggcttatatttgcagtaacacctacttaaaataacctccctttgcatgttttgtaaataggcctaaatttgtttttgaaaggatttgcttctcttgcctacatgagaacagactgtgctaattctctgggtagtagagagggcttataaagtggatttgcaaaagtatctgtaaaacaagagtagtgtctgtttttgtcctgtgtttgaaacactgttggatgaaatcgtttgtgattagacacttttgcctcagcttccctttagctgacatgaacactttggacattgcagcctggtgccagattcaaaagaactgagtcacatatttcataagtatcttcattaagcacatgtacagaataaattaaagaaattcttgctaacatttcagcagattatccaggggtgtgtgtgacatgtattttcttattttagtcttaattttgcacatgt

SEQ ID NO: 5 - DNA, 929 bp, Macropus eugenii
acctgatcaatatccagaagacacatgagcgaatgcagacagaaaataaaatctctccctattaccggacaaagcttcgaggtctctataccacagccaaggcagatgcagaggcagagtgcaatatcctgcgcagaccttagataagattgctgagatcaagactctgttggaagaaaggcgaattgcggctaaaatcgcgggattgtacaatgattcagaacctccacggaaaaccatgcgccgaggggtgctgatgactttgctgcaacagtcagccatgaccttgcctctatggattgggaaacctggggacaagcctccacctctgtgtggggccattccagcttcgggggactacgtagccaagcctggtgataaggtggctgctcgagtgaaagctgtggatggggatgagcagtggattctggctgaagtggttagttacagtcatgccaccaacaaatatgaggtggatgacattgatgaggaaggcaaagagagacacaccttgagccgaagacgtatcatcccactaccacaatggaaggccaatcctgagacagaccctgaagctttattccagaaggatcagcttgttctggcattgtatccccaaaccacctgtttttaccgggctttgattcacactcccccacagcggccccaggaagactactctgtcctgtttgaagacacctcctatgcagatggctactctcctcccctcaacgtggcccagaggtatgtggtggcttgcaaggagcccaagaagaagtgagctttggttcagctgaatcacggacttctatttctttgcctttgtttacctgactcttctgccatgctcttggatgggggaattaatgtatcattctctgcagacaataaatataccgtctgtcccccacagaataagacacaaaaaa

SEQ ID NO: 6 - DNA, 1055 bp, Macropus eugenii
aaaaggacaaaatcacagcattcattgttgaaagagattttggtggcattacaaatggtaaacctgagaacaaattaggcatccggggctctgacacttgtgaagttcattttgaaaatacaaaagtgcctgttgaaaatgtcattggagaagttggaggtggatttaagctagcaatgaatatcctcaacagtgggcgatttaccatgggaagtttagttgctgggatgctcaagaagttgattggaatgacagcagaatatgcgtgtacaaggaagcagttcaacagacctctaagtgaatttggattaattgaggaaaagtttgctctgttggccaagaaagcatatgtgatggagagtatggcctacctcactgccgggatgatggacagcccaggtttccctgattgctctctggaggcagcgatggtgaaggtgttcagctccgaagcagcctggatttgtgtaagtgaagccctacagattgttggaggcctgggatacatgaaagattacccctatgagcgctatctccgggactgcaggattctgctgatttttgagggaacaaatgagatcctccgcatgtatattgccatgacaggactgcagcatgcaggccaagtcttaacaaagaaaatcagagaaatgcagaaggggaacgtgagcgttgccatggaatatgtatcaagcaaacttcgagaaagcttgatcagaaaggtcgatcttggtctgacgggagtaaatggatcggtgcatcccagtgtggcggagagtgcaaaccgactagaagaaaatgtgtatcattttgggaaaatggtagaggtggtgctgaagcgatttggcaagagtatcgtggaagagcagttggccctgaagagaatagcggacatcgtgattaatctgtatgccatgactgccgtcctctccagagctagtcgttccgtccgcattgggctgcgttaccctgaccttgatgttctgctgaccaatattttctgccccgacgcatattttgagaattttacaacatgtctctgttcaaacaccttt

SEQ ID NO: 7 - DNA, 744 bp, Macropus eugenii
cagtactgtatttcacatggctggtttttgactggaacaccccaaagaaaggtggcaggagatcaaagtgggtgagaaattgggctgtgtggcggtatttccgagattattttcccatccggctggtgaagactcacaacttgctgacaacccggaattacatctttgggtatcacccccatggtatcatgggcctaggggccttttgcaacttcagcactgaggccacggaggttggaaagaaatttcctgggatccggccttatctggcaactttagctgggaacttcaggatccctgttttaagagaatatttaatgtctggaggtatctgccctgtcaaccgtgacaccattgactatctgctttccaagaatgggagcggcaatgctatcatcattgtggtggggggcgctgctgagtccctgaattccacaccaggaaagaatgctgtcactctgaagaaccggaagggctttgtgaagctagccctgagacacggggctgacttagttcccatctattcctttggggagaatgaggtttacaagcaaataatctttgaagaaggttcatggggccgatgggtccagaagaagttccagaaatacattggctttgctccctgccttttccacggtcggggcttcttctcttccatacttggggcctagtgccgtactctaaacccatcaccacagttgtgggtgagcccatcaccatccccaaacttgagcacccagc

SEQ ID NO: 8 - DNA, 2136 bp, Macropus eugenii
ggatcgcagggccgcgggcggagccgccgctcgggctgacttgactgttgggtgcagcttggcttcgggagtcatcttctggtcctccggctctccagagtcagtgtttcagctcccgtctctcctcccccgcgcctcctccggccggctccgcacccggatggcgccggcctcgctctagccggcgcgcctgcgcagtctcgcggcctggggtgggggtgcggcagctgcggctgctgagccgccggtttcctgccccacttccttctcctcgccctcctggtgcgtcggctcgctgggccggcgggatggcggcgctggggggccgtcggaggtctcctcagccgctgctcctgccgccgccgccgccgccgccgccgctgctgctgcttttgctgctgctgaccccgctcggggtccgagccgtgcccgaggaagacgccggcggccggaacaaggaacctccggcgcctcctcagcagctccagccgcaggcggccgaggctccgaggcccgagttggcccgcgccgagaaaggacctctaccaacttccccaattcatattaataatgaggatcctgctggccagactaatttgggatttatccatgcatttgtggctgccatatcagttattattgtttctgaattaggggataagacgttctttatagctgccatcatggcaatgagatataaccgtttgactgtgctagcaggtgctatgctggccctgggcctaatgacatgtttatctgttttgtttggctacgccaccacggttattcccagagtgtacacatactatgtatcaactgcattgtttgcaatttttggtatcagaatgcttagggaaggcttgaagatgagtccagatgagggtcaggaggaattggaagaagttcaagcagaaataaagaaaaaagatgaagaacttcaaagaaccaaactcttaaatggcccaggagatattgagactggtacaagcacaaccatacctcagaaaaagtggctgcattttatttctcctatttttattcaagctcttactttaacgttcttagcagaatggggtgatcgctctcaactaactacaattgttttggcagcaagagaggacccttatggtgtggcagtaggtggaacagtgggacattgcttatgcactggattggcagtaattggaggaagaatgatagcacaaaaaatatctgttaggactgttacgatcataggaggaatcgttttcttggcatttgccttctctgcattatttataagtccagactatggtttttaataagtcttctccatttgtatttagagaataagacagggtgttgcctatctttttgtatatagtgtatattaatagtgactatgaaaaatgctatattttattttatagcattgatttgtgagtttagcccactatatgatggcagttttacatgaaaaaaggataaccagtgattctatctgcaatgtggatttttttttttttttagatgggtccgttgttaaatgaatggtggtattttttttccctcctcatcctaccttccactcctgtacctagaaccagtgttctggcatctggtactaactcacagtctctagcattttttcctttcttctggataccatctggtaaactgaatagcttcatttttgagtgtgttcataattaagattgttttaagattggccatgtattaatgataggatatacctgaatatatatgactcaggaaaatatttgaaaaaatatttcctaaatactggcatttctaccccaagccagtaatctttttagaaattaggctttttggatgaaacattagagaggctgatttcactgctttttaactaacagtaagcaattcataggacttttgttttcctggcccactttttaaaaaattgggttggaaactcacaatttacagtaaatggtcaatatttgacaatgctgaattaccaaattaaaaaaagtaaaagactgtatttatattttctctatagcaaatattattaaatgtaatggaaatgatgtaaataaaaatagttgtaagaaaaaaaaaaaaaaaaaaaaaaaaaaaaaaaaaaaaaaaaaaaaaacccaaaaaaaaaaa

SEQ ID NO: 9 - DNA, 771 bp, Macropus eugenii
gaacaagccgagctcggcaggtgagaggtcccccaccggaggagaagccatgaccgctgacctgtgaccctgacctctcccccttcacttcctcccatgactcggtgatctcacccctccctcctgtgtctctcggatctgttatcgtctcctgtgtatacgggatcgctgaccttcagggatgtggttgtgaccttcagccatgaggagtgggggcatctagacccttcccagaaggctctctactgcgaggtgatgctggagaattacaggaacatggtctgcttgggacttgctgtttcccagcctgatgtgatggaccagttggaacgaggggaggtcccttggtctcctgaaggagaaggcgcatacagcagctccgcagctttgtgcctttggtcagtccaggataccaaggtccaaggccatggggccaaccccaacgtccctggggcatttcattgtggacctccttgaagggtttcaggccaccttcatgctttagattgaatccctcaaatcatcccgcccttactgacattctgcagtcttgtccctgtcaatcggcttcttctcagagacaggttgggtggcttcagagcccagagcatggagcctggactccggagacttgaatttaaatctggccttagacatcctgggccacccacttaacctttgtctgccctaagttctttacttgtgagatggagttccaattacacttaccttcctgacccaaccacatatattcttaagtcgttacccatt

SEQ ID NO: 10 - DNA, 761 bp, Macropus eugenii
  variation 708: n = g, a, t or c
gttttttgaagagcctgaagatcccagcagtaggtccttcttttcagaaattatttcttccatatccgacgtcaagtttagtcacagtggccgatacatgatgacaagggactacctttccgttaaagtgtgggatctcaacatggaaactaggcctgtggaaacatatcaggttcacgaatatctccggagcaaactttgttctctctatgagaatgactgcatctttgacaagtttgagtgctgctggaacggttccgatggcgccctcatgactgggtcctacaacaacttcttcagaatgtttgacagaagcacacggagggacgttactttggaagcatcaagagaaagcagcaaaccacgagccattttaaagccccgcaaagtttgtactgggggcaagagaaagaaagatgagatcagcgtggacagtctggactttaacaaaaagatcctacacacagcgtggcatccccttgagaacatcattgccgtcgcagccaccaacaacttgtacatattccaagacaaagtcaactagagaggtcagctcctctggggacattgtcttactcctgcctccggaagcctggtcgcttttctgttcaaagaaaaacattcttgtcctctccatgaagaacactgatgcattcatgtcccttgacaggtaaaggaaagaagctggcctggtttggtcattcaggngatcactctgctttcacagaagtgggaagaacccatgagaaacaaggttgatg

SEQ ID NO: 11 - DNA, 972 bp, Macropus eugenii
cctcgctcccctccggggccgccgcccagccccagccgaagcgggtctcgggcgcggtctcccggtcggacggctcgcagccgcccggtctgccgtcgcggttcccgcgcacgtcatcgagcagctccgggtttggcgaggaggcggccccggcggagcgagggcggcagccgagctgggcgtgcaggcgaggccgcagcccggcccgcgggcccgggagcctgagctctgatccgagctccggccagggcccgggcacctcgcgctgggcccgctccggcatggcggcctcgacggtgctgggcgccggcctggcgcggatcctcttctacccgacgctgctgtacacgctgctgcgcgaaaaggtgtcggggccggcgcaccgcgactggtaccaccgcatcgactccacggtgctgctgggcgccctgccgctgcgcagcctcacgccgcggctgatagagaaggagaacgtgcgaggagtgatcaccatgaatgaagaatatgagacaaggttcctgtgtaacacctttgaggaatggaaagcgcttggagtagagcaattgcgtctcagcacagtggatatgactggagtcccaactttggaaaacctaaaggaaaggtgtccagttcacactgaagtacatggcactagagaaaagtgtctacgttcattgcaaggctgggccgctccagaagtgccaccatggtggcagcctatttgatacaggtatacaagtggagtccagaagaggccgtgaaagctattgcccagatccgttcccacatccacatccgacctcggcaggtagagatcctcaaggaattctacaagcaagtcatggcctgaagcagaaggaactaacgcttcccaagaagtaccagttcataaaggaagtaggggactagcaagaactcttaagactcatactgctcattacagaataaagaaaatgcatttagtgcaaaaaa

SEQ ID NO: 12 - DNA, 1176 bp, Macropus eugenii
aaatttgtgaaaaagcaggctggttcggtccaaaattcccgggaatcacagatgttctgcctttccccctgtgttgcatttgaggatgtctttctttctggtgatggtcagcctacttctgctcactcagcatgcagtggctatcaaggaattaagggtccacccttacactgatgttaccttgccatgtcatttctccttcgtgaaaggcacagatgaccttgagttttcctgggaaagagaagacatcaaggaggaatttgaggtggaagatgacagggattattttcagtttttcaggttctatgattttgaggtgttttcaaaagtggtttaccagtttagaaataataaggaacaaacagaggagcagaactccatgtatgagaaaagagtgtctgtggatcccgcagaaattcctgagggaagtctctccctcttactaaagaatgtgaatttccaggatcaagcaatctacaagtgcttggctgtctcgtcaattggaaggggtgaaagcaaaattaaactaatcgtagaagattctgaggagcccccggtgcaatttaagcaaatcgatgatgaaaccgtggccacctgcgtctccacaggctggtactacctgccaaatgtcacttggctggaccgaggagagagggatttgtccaattacagcaccgtggagatcttggaggagcagatgaatggtgcatatagagtgttttctgtcttgaaatatcctgtcaaactcaatgagaaatacgtttgtcgcataatagagacggatgtcaactttcaaccatttaggactatccgcaagttccccagaagaaagtttcatcgaatgtatgattactattagcaacctgagtaaagctaagcggggacgccaaccatattcttctgacagacgacctggtttccttttctcagaggcaacattctttttgctactgatgtggctctccagacttctactcctccttaccagctgcctccccatgctcaagaaatctctgctccagcatcctgatcagttactgtctattactaaagagggctctaatcccagcttcacattactgccagaatttctctttggcagccaccttgtgcccttctctcaagccaggcagcttccacattctatcttctttttatgtatggtcctctctcatt

SEQ ID NO: 13 - DNA, 798 bp, Macropus eugenii
  variation 2, 11, 34, 35, 46, 506, 516, 518, 526, 565, 570, 587,588, 592, 594, 599, 602, 614, 615, 622, 631, 638, 649, 651, 658,662, 666, 669, 673, 678, 681, 683, 684, 687, 692, 694, 695, 696,701, 704, 705, 709, 714, 717, 723, 724, 729, 731, 737, 740, 743,746, 749, 752, 753, 760, 762, 764, 770, 771, 775, 782, 788, 791,792, 793, 795: n = g, a, t or c
anttgtcaaanagcagggctggttccggctcctnnaattcccgggnatacttcatgtatgtaattttcttctccagaattactgacatttattttgcttttaatttaaagaattttactctgaatttcagaaacaccaaataaaatgggcaccttcatatacataatagaacagaaagggaggattgtataggaaactgcaggtttcaatgtgcagtttgctttcattttatttttttgtatttaattctggactaaaggcagtggggcctagggagtagtgtttatccttggagccagaaaaacctgacctagcctggacctcggcttaagtacttcccagaaggaccagtcaaggccagcagaaagagctggcagagacgagaacaaacactgctgccttatcaccgtgggacagctataagaaggcaggccccatggcagcctggtcttttacttttgttcaccttccctgagaacagaatgcatcttctcctccctggccgncttcttggcnantgggcacntacctgttgatggtgcccaatacccaagcaactaaggcnctttncaaaatatgaatcaagnngtgncnttccnganagatgaaccccnnaaaacanctaccagcnaattaanggttgccactngntggcccngggntggnaancggnacccntgngnnatnaaacntnnnacatngcnngccntgatnaangacccnnaggcnantcccanacncanaancanagnnaatctcnangnttccanngtgnaaaatcncccttnccnnngngca

SEQ ID NO: 14 - DNA, 1257 bp, Macropus eugenii
  variation 1181: n = g, a, t or c
cccgcttcgcctagcatttcttactctctggccacggtctcctgcctctgcagcctcgtccctctccttccttgccatggagccggccacagacagcgagcatggcagccccccggagcccagtgcactgcagccccccgggggcagctcgcccttagccaccggctcccagtctcacccaggccgagagccgcagcaggaggagcggcatctccgtatcagtgaaagcggccagtttagtgacgggctggaggatagaggtttattagaaagcagcacaagactaaaacctcatgaagctcagaactacagaaaaaaggcattatgggtatcctggttctccatcattgttaccttggcgctggcagtggctgcctttactgtctccgtaatgaggtacagcgcctctgcttttgggtttgcttttgatgccatcttggatgtcttgtcttcggcaattgttctgtggcgctacagcaatgcagcggctgtacactctgcacacagggaatacatagcctgtgtgatcttgggggtgatattccttctgtcatctgtatgtatagtggtcaaagctatccatgacctttcaacaaggctgctcccagaagtggatgatttcctgttcagtgtttccattttaagtgggatcctatgcagtgtcttggctgtgttaaaatttatgttgggaaaggttctgacaagcagggctctgataacggatggcttcaactcacttgtaggcggagtgatgggtttctccattcttctcagtgcagaagtgttcaagcacaacgcatctgtctggtacctggatgggagcattggagtgctgattggcctgaccatatttgcatatggagtcaaattactcatcgacatggtgcctcgggtgaggcagacccgccattatgaaatgttcgaatgaaggtacctggtgggggcagagccagctgttccctatcctgcatcagtagcatgaggactctcaaaagtacaggcagatggtgccaacattatttaattgaacatccaatttcttttatggggattttcttttgtaaagtttattctaatgggaaatagatccatcaaaacagagaagtctgtgcactgctcatccttcagcaccacaggctccctttccctattaaagtgcatcactgaaccatgtatagganggagaggtgaggtcttacttttccatccctaatagtggacactttttgtctctagagtaagaatcttctatgggtt

SEQ ID NO: 15 - DNA, 724 bp, Macropus eugenii
cagcacccgatgcccgcttagccttcgaccttccaggaaaagaccatgtaacaatcaaccctgtactcagcacccagatgaccattgccaggacaactccccacactgccccctcgtggtccaggctcgcctctgcgtctacccctactacacagccacctgctgccgttcctgtgcccatgtccaggacagggcacccccagaacctgcctgagatgggactgaagtgacatttacagttatcttccctgccccagcatcagctgccccatggactctatccccttcctgcccttgacctgcctcatgggacactcagagaaataccagcaccttctatatttatcctgggaggagtctggggaacgcagtgtgacaggttgtggggagatagctgtgacagtgtgtgaggttgtgttcatgtacctgtgtgattgtatgtatatatgtcattatacatgtctgtccctgggtgctattgctggtgggactgtgtgacttcctgctaactagggtccctggccaagacagactgggatatatgaaatgccctgatgcgcctccccttgccccacctcttctaactgaagtgaaaccaagacaaaagttcattctggtcctttgattttgccctgaccagccctgttgcctaaatcaaactaaccatggtatatttttgcatgtcctgaacacaccttcaggcttgcacctttgaccccagcct

SEQ ID NO: 16 - DNA, 853 bp, Macropus eugenii
  variation 835, 850: n = g, a, t or c
gcatctcattcgggatataattaaccagcgttgtgggagcaacccattgattggaaggccaaataaggtgacaacagtggacagattccaaggtcagcagaacgactatattcttctttctctagtacgaactagagcagtgggccacctgagagatgttcgtcgtttagtggtggccatgtcaagagccagacttggactttatatctttgcaagagtgtccctcttccagaactgcttcgaactaactccagcttttaaccaactcacagcccgcccacttcatttacagataattcccacagagcatttccctactaccagaaagaatggagagaacccatcatcaccctgtgcagataataaaaaatatgcctcagatggcgaaactttgtgtacaacatgtacatgccttatgtatccagagcacgcatcattataatcaggctctgctacctccacctgccatggtagaaggaaagtgatacggttcagaatcaggaagcagaaatggaagttgaagtagaacctagccatccaccatcagagactaccccaaccacagctgaggctgttctcacaatggcagaggctgctcccaccacggtcacccctgcagcgtcagagggcacagagacagattccacacctgctccagcagagacagaagtcacacccatagcatcagagagtcatcagtaaagctggcatccccattggaagagaacactttattccttgctctctgaatgtcgggttacttttccgctttcctgttgcgtctcctactttgagaggactgacttaacactgtattcctattattgctacactntctggtgtatatagnttt

SEQ ID NO: 17 - DNA, 1094 bp, Macropus eugenii
gacagctcagtcccaggaactatggcaccagttactcaggtcagctttgtccttctcatcttcctactctctctgccgcccatcagcccgggaaccctgaagatgcgggtgccagtgaaatctgcagagctcaagagtcaaggaaaggattgctcccatatctggacagacaatccaaaatccccaagcggtgtgtacatcatcaagccagatggggcccccaatgccttccgggtttactgtgacatgggagaagatggtggctggacagtcttccagaggcggaccgggggcaatggcaagcctttggcgtttgaccgagtctggtgggagtacgttaatggctttggggatgtaaaaggtgaacactggctgggcctgtctaaagtttactccctgacgcaccagcctggcactcgcgcccaactgaaactggacttacacaactttgaaaacgagagccgccatgccctgtatgactctttccagatcagtgatgagtccagtttctataccctgagcctgggcaggtactctggaaatgcaggagacgccttcggtggtgaaaattggaaaggagcagcctcccaggtaggcagtgccttcagcactgtggacagggaccatgactcctgtgacccctgcatcagcggtgacatagcctttaatgagtgtagccaacagtatggaggggcaggctggtggttcagcgactgtggggtggccaatctccacggtgactggcaccctgaggggaaccacagaggttggagttctgacatccactggggaacctggagctccgtggattccctaaaagccaccaagatgaaggtgaaaactgtgaagacagcaaccaaggcttaggctccaaaggtccttccagctgccccaggaccagatagggagcacggtgtgcccagagagccaaacccggggtgcaagaggtggggggggcaaaggggcctgaggtgatccaatgggactaagaaattggggggcagggcctggagggcagagtgggcacacagtgcctgctccaaatgttggggaaacgatcaataaaaacagagattccaagccaaaaaaaaaaa

SEQ ID NO: 18 - DNA, 939 bp, Macropus eugenii
cacatcatggcggcggcggcggcggcagctagaggagctgctcccaccacccagccgccgccgccggggccccgctgaggagccgggaggacgggagcgggccccgccggccccggagccggcgtcggcgcgatgctgttctcgctgcgggagctggtgcagtggctgggcttcgccaccttcgagatcttcgtgcacgtgctggcgttgctcgtgttctccgtgctcctggcgctgcgcgtggatgagctggccccgggcctgtcgtggtggaacgtgttcgtgcccttcttcgccgcggacgggctcagcacctacttcaccaccatcgtgtcggtgcgcctcttccaggacggggagaagcggctggccgtgctgcgcctcttctggatcctcaccatcctcagcctcaagtttgtgtttgagatgctgctgtgccagaagctggtggagcagacgagggagctctggtttggccttatcatgtccccggtcttcatccttctgcagctgctcatgatccgggcttgtcgcgtcaattagcgctgctgggaggggagggagaaaggacgcgctgcgagccccagccctcccgagccggcggccggatcgcgggagccccggcgaggtgcaaggagtctcacttgcttgtgaagttctcagctttttttcgtgcttttcaccctaattactggagggtgggcagatgtgtggaaaaaacacttatcactaaatacaggtattcttttttcctttttttttctcctattttcattctccctgttttgagatgacttccggattggacagtgtttaattgcctggattgtaaagggagcttgctgttagaagttgctttttcaataaatgtactgttgggaaaaaaaacaacaacacccacaacaccccccccccccccttccaccaccctccatttcc

SEQ ID NO: 19 - DNA, 1034 bp, Macropus eugenii
ggcggatggggagggggcggggtgggactaaatgggcacttggcggctacgaactcggtagtgagaacttccccagccagatcccgccacgtcgggtcgcagcgggagcctcggagaagtcttgatcctggccaaagagctttccttacccacccctcgccaagaaaaatcattcacgtggtacacccttactggccccgaaacctccaactcagtgattatgtccaccaacgatagaccagtgtaggcagatcctctgcagtcttcttttctgtctcgggggcgctcctaggtgggcacctggctgttgtctggccaggggaggagagggagtggaagtcgaggtgccagctctgggcccctggcgtcgcctggcagtctgctggtttgccatgtgtgcctttgtgcacatggtgatcgaaggctggttcagcgtctacaaccaggacattgccagggatcaggccttcttgtcgcagctctggaaggagtactccaagggtgacagccgctacattctagcagacaacttcactgtgtgcatggagacagtgacagcctgggcgtggggcccactcagcttgtgggctgtgggtgcctttctgaaacagaggccagagcgattccttctccagcttgttatttccttaggccagctctatggtgatgtcctttatttcttcactgagtatcgacatggcttccagcatggggagctcggacatccactctacttttggttctactttgtcttcctgaattccttgtggattatcgtgccttctgtcctcctcctggatgcctggcgccaactggcctgtgcccagtcgctggcagatggtgctgctggtctccaaacccgaagaaggcagcatgagcatctccagtgaagatctccacttgtgggggagggggagctgggttgttctggaagcactttctcacattgagccatcgctccctcctgcccctgttgatttttgtagcattgcactgtgcaagtagcttcttcctcgcttgcccccacttcacgc

SEQ ID NO: 20 - DNA, 712 bp, Macropus eugenii
acaactcctgtggcttcggactggcagcagcctgcggtgaagctccctgcgctccgggctcgcagtgctacagtcttcctgcgccatgaactccaaccagacacatctgccccaagccctggggcaaatcaaccgcgccttttctccccccaactacgagctcctctcggaggagcgagagctgcggacgccgggacgggaatccgcctccaccatcatcaacgtccactcctacgagcctcctgccaatgatttcttggtctggtccgtgttcaacacgttgtacatgaacttttgctgcctggggtttatggcgctggtcttttcagtaaaggcccgggaccggaaggtggtgggtgacctaaacggggcccggagctatgggtccaccgccaagtgtctcaacatcttcgcgctgatcttctcccttctgatcgtcattgtgctcaccgcgctgctggccaccggcatcatcgccctcaagagggttgtcacgaacccgagtgagatccctcactactcagatttatttccaggcaattgacttaccgcacttccgccccatgtttaaacccagtctctgggtttaaaccccacccccatcttgtggcttttcttcattctgggtgtggcctctcttcacttgcctccgctcccccacctgggcccctttgtatccaataaaccactttgtttgtacaaaaaaaaaaaa

SEQ ID NO: 21 - DNA, 733 bp, Macropus eugenii
cccaaccagacatgcaacctgcaagacccctgtaaggatcacaatgatcagccagaggtgccaccccaccctgcttgctgggtcaagcactgctgtttccagaatcagcttgagttgatactctgattcaagtctgctctggtgccactagccaagtccggagtgtgagtcctgctctttggcacatgcaccatgagtcacagtaccagctccaggctggctgactctccacagctctccaagagcagcctcttgaacatcctgggcagcccttcacctgagcggatgggcctagctgactctctgccccccaccccacagagcagtaccccttctccaggacccccaccactgcctctgttgcctggtgggtccccagggttggatggtgactgggagagccgagaagagctgcgtcttcgagaactggaggaggcccgggcccgggcagcccagatggagaagaccatgcgctggtggtcagattgcacagccaattggagggagaagtggagtaaggtgagagctgaacgcaacaaggcacgggaagaggtgcgccaacttcggcagaagctggaggcgttgactaaagagctgacgggtgcccggcgggatcggcaagaggccctgaaccagagtgaacagctgagcaaggaggtggctcgacttaagggggcccggctgcaggaggaggaagagggggagaaccgtcaggagggagaggaggctcatg

SEQ ID NO: 22 - DNA, 835 bp, Macropus eugenii
cctttctgcatcctctcttctcccccacccgcctgcggtttagcccagcccagcccagcccctctctgctcggtgcagctccgcgcggcgcagcctagcggatccggacccagtgcctcgcagcgcatccccagcccagcctcgcctcgcctcctctagccaccatggcagatgctttcgtcggcacctggaagctgattgacagcaagaacttcgatgactacatgaaatccatcggggttggctttgccacaaggcaggtgggcaacatgaccaagcccaccaccatcatccagatcagtggggacaccatcactctcaagactcagagcactttcaagaacactgagatctccttcaagctaggagtagaatttgatgagacaacagctgatgacaggaaggtcaagtctctcgtcacactggatggaggtaaacttgtccacgtccagaagtgggacggacaggagaccacactcgttcgggagctccaggatggcaagctcattctgacactaacccatggcagtgcagtctgcgttaggacctacgagaaggaggcatcttgaggctggcccacatagtcacctgttctgccaacggctaccactgaactcaccaccagattgcctcatttcccacccgtccccctccccaccccagtgttttgtaccaattgactctggtcaaggaatcctccagaaatggaaaccatggtgctgtcctggtcctagttgaagcgcattctgtattttttttttttaaactgcctctgacacctagtcccaagtcaataaagcagatcccagtcaggcaaaaaaaaaaaaaaaaaaaa

SEQ ID NO: 23 - DNA, 881 bp, Macropus eugenii
gagaaacaagattggagatgcagggttggactggttgtgccaggggatcctgagccctgcctgcaacctgaagaccttgtggctttgggagtgtgacatcacagctgagggctgcagggccctcgcccaggtcctaaagaacaagccttgcttgacaaagctgagcctgatctgtaaccaactgggtgatgaaggggctgagctgctgtgcgaggcactgctggaccctggctgccagctggaggagctgtggctgaggacatgtggcttcacagtggctagctgtgccagcttctgcactgtcctggagaagaatcggaccctgaaggagctgcagctgagcaccaacatgctggaggacgtgggcatcgagcagatgagcaagggactcatgcatcctgactgcccagtccagagtctctggttgggtgactgtgagctctcagacgcctgctgtgagaccctggcttctgtcctcctcaccaaccacactctgaaggagttggacctcagcaacaacagaatgggtgacgtgggcatcaggcagctggtggccagcctccgacagcctgactgcaccctggagcagctggtcctgtttgacatttactggacagaggaggtggacaatgagctgaaggccctgcaggaggccaaacccagtctgcgcatcacatcctgagggcctggcggccctacccacactaagccttggctgtttcttgggttaggtgccctactctcccttggtgcactgagggcagcggcggaggggggggggggcagggagggcccacttgtttattttaagcccttcactggccttggtttaatgggtgcccaggagcggcccatgccctttaaattccttttttggccaa

SEQ ID NO: 24 - DNA, 956 bp, Macropus eugenii
  variation 786, 792, 831, 835, 836, 854, 872, 875, 884, 901, 920: n = g, a, t or c
ggcatctgtacttccacgggcatcattcgggattttgcaggcccctactatgatctcggaagacaatatggcctttgggaagcctgtgaagtactggaagttggacccttccaaagtgcatgccagccgaccaaatgcctgggacactgcagtccatgaggcatcagaggaatataaacatcgcatgcacaatctgtgctgtgataattgccactcccatgtggccctggccctgaatctgatgcgctacaacaacagctctcactggaacatggtgaaggtcggccttttctccctgatctatgggaagtatgtcagcttcggggcatttgtgaagacctggcttccctttgttcttcttttgggcattatcgtcactgctgcctttgtcttcaatctgcggtgatggccgccccctcagcctggcaggcctcggctggcaggagcctgggcctgacatacacacacacacactccctgccccgccctcccagagctggctgctaggcctagactcctacctcctgtggttgcacataccactccctccctgaagtactggagccaggctgtaagaatagcagccgcaactcaagagactgagatgatgaccctgcaaggggccagttacccctaattctgttttagccaagctggggcccttcctcatctgcccacttcccaggatggagcagctgacctcttgtcacctggtgggagctctcaggatgctctcagggaagggggctgggctctcctccaattcttcccttcctgatctttccnactggngcctgtagcctggcaaggctgcctctgccaccaccacangctnntccagacttttcttctcnatcctgtctccccacctnctncacatagcntcccactcctgtctcangagagggggcagagagcangcatacattcagtcactagcctaaactaaagccagg

SEQ ID NO: 25 - DNA, 862 bp, Macropus eugenii
  variation 8, 9, 10, 21, 39, 751, 759, 782, 807, 815, 840, 856: n = g, a, t or c
ccggtccnnnaattcccgggnatgccttttcctgatagntgtctctttctggaaaactccagtggagtgcgcgattggcttcgtcatcatcctcacgggaattcctatttatttcatcggtgtctggtggcagaataagcccaagtggctccttcagggcatcttctacacgacagtcctgtgccagaagctcatggaggtggttccacaggagtcctgagcacagtgacccttcgagagggaactcacggatgtcgcttggaaagaacacccccaccccccttcagaaaaacaaacaagtgaggccagcaatgtccattccctcatcccatcctcccgatggcctggctgaagccacctggccagccttccctcttcctctgctcaagcctggcagctgagggtgctttgaaagataaacccgcggtacaaacgaatctctcctggatacctcctcaggacccccagaggggacagggaacaagttgctgaattaggcattaagactttggatttgtgactgaaagtagatgttattttgggggagttttttgctttccttcattttattttatttttaacttaaggttcaggctagtagaacccggcgatacgatttctttttacaagtgagaatattattttttcaatgctgccaactgtacccacatacctctggatagcaccgacacactgactccaggtcaagcagcactcctttgttgaccttctcagtggtcttgtactttgngaaggggnctccaagacccacctccaggcanagtggaagactactgaactgatcanggtgattnatggggctgcctgatcccctgaggncgctccgtcccattgngtacgc

SEQ ID NO: 26 - DNA, 685 bp, Macropus eugenii
ctcttcctcctggcttccagcttctacaaaggtaccgtgccccgcctgggccgggtctgcttggacgtggccatcgtcttcgtcatctatgatgaagtggtcaaacttctcaacaaagtgtggaagacagactgaagcggggctactgctgctcagccctgaggcccaggagccggcggaggagagatcagcaggcggaggagaggccagcgggtggaggagagaccagcgctgtgttggggtgggccctcaggcccccccgcccaacctgtccccaagagccccatccctgccagggcccatggcagatgggggactgtgagcctaggcttcccagggaagggcctcccctggccctccctcccacccccactgtgagtagagcagaggtttggctgggccggcctttatgcaccaaccctttggcctcctcccctcaggccccactatcttctgagatcccagggcagagtcaagctcccccctctgcctctaggctcttatgattccagtgtgatttgtgccaaacagatcttgtcctgtgttttctatgttgtatctgtattctgtgctacatgttttgtgtgtatccttgtgtctgtctagtgtgtgtgtgtgtgtgtgtgtgcgagtccactgccggctctaccctatctaccaaaccaccaataaccccgtgcctttt

SEQ ID NO: 27 - DNA, 705 bp, Macropus eugenii
  variation 645: n = g, a, t or c
ggcagatatctctggggacacccagaaagccaagcagtttctgcctttcctccagcgagctggtcgttctgaagctgtggtggaatatgttttcagtggttctcgcctcaaactctacttgccaaaggaaacctgcctcattaccttcctacttgcaggcattgaatgtccccgtggagcccggaacctccctggcctggtacaggagagagaacccttcagtgaggaagcaacactcttcaccaaggagttggtactacagcgagaggtggaggtggaggtagagagcatggataaggctgggaacttcatcggctggctgcacatcgatggtgccaacctgtcagtctcactggtggagcacgcactatccaaggttcatttcactgctgagcgtagctcctactacaagtctttgctgtctgcagaacaagctgccaaacaaaagaaagagaaggtgtgggctcattatgaggagcagcccatggaagaggtatccccagtgctagaagaaaaagagcgttcagccagctacaagcctgtgtttgtgacagagatcactgatgacctgcacttctatgtccaggacgtggagacgggtacacagctagaaaagttgatggaaggcatgagaaatgacatcgncagccacccacccgtagagggatcctatgcccctcggcgaggagaattctgcattgccaa

SEQ ID NO: 28 - DNA, 715 bp, Macropus eugenii
  variation 681: n = g, a, t or c
ttgattcccagagtagtcagtatttttcccatttgttaggaaccaggcccacagcagtaaagtaatttaatatattaggattttagagttggaaaggaccttatagagcagctagttcaataatctttgtttcacagatgaagaaaattagaccttagaaaggttaaatgatttacccaaggtcacacaaaaaaatagtgacatcttgactaggatattgtttcttaattccttgttaaaggctctttccatcctacgccatggcctgcctggagtggttcctgaagatgactgaaaacattggaggctggccacagtaatactagggagccaaaagaactctgattccctctgataattcaaagtctagaatactgggccttgaagaagttgccatactgctggggaggaatcctaggcttagagtagcccagaagaaggaagtcaattgactatcagattctagttcttggaaccctcaggcatttcctattcatacctgcctgtatttccagctggctgtcttttccttttccaaccttgccagggcatctccaagcccttagagccttcagtgggaggaaatgtcagagaatgaaaaccacccagtcaagaattaggaaggcaactcagctggacgtttgaaactgggggggaggggagaggtgggtaggttgcttncccttcgttccctctccctacccttgctttaaat

SEQ ID NO: 29 - DNA, 728 bp, Macropus eugenii
aaataaatccctccttgggagaaacttgcccacaagccatagatatcttgttatacacaccaggacatcttgatccagcagaaaagattgaagatgctcatcccaagctgtggtgcgctttaagtgaaggcaaagtgattgtgtttgatgcgtcctcctggacaatccatcagcagtcctttaaagtaggcactgccaagttgaattgcatgctgatggcagagcagaatcaagtctggatcggctctcaggactcgattatttacattattaacatccatagtatgtcctgtaacaagcagctcactgatcatcgctcgagcatcatggatttaattgtagaagatggaaacaagggatctagtagtgaagtctattcatgcagtttggatggagtaatcattgcttggaatgtcagtaccctgaaagtgatcaggagatttcacctgccgtgccaaactctaacttccattaagcttcatattggccgcctctggtgctgtataggctataccatcatatctgtgacatcaaatggatctccttcttaagaactgaaagttgatgagcttttaaggaaattttcctccttttcttcttgttccacgcttttccggagcaatgacctgtcttgacagtttgttctggacacatagacctatatgtcttgatcctaattgacttctccaggtctctctataaatccatcttaattctgctctagattt

SEQ ID NO: 30 - skipped

SEQ ID NO: 31 - protein, 240 aa, Macropus eugenii
  SITE 227, 240: Xaa = any amino acid
QIVAIEQSVNEKIRNRLPVTVMELSLDDPEVEKVKGLGLPDDHAGPIRVITIEGVDSNMCCGTHVRNLSDLQVIKILGTEKGKKNKTNLVFLAGNRVLKWMEKSHGVEKALTALLKCGVEDHAEAVQKLQNSAKLLQKNNVNLLRDLAILTAHSLRDSPDRGGVIVLHRKDGDSEFMNIVANEFGIEDTLLFLTVGDEKGAGLFLLAGPFEAVDMLGPRVAELLQGXELGRKPLSGQATX

SEQ ID NO: 32 - protein, 127 aa, Macropus eugenii
KPTEPTHQFYESISVTYRVVDSINNNINESTIVSVPKGSVFLKVMEAAQTKSKTKFGFTTKSEQWGEYITSVHGIKASSENRTYWQLLSNGQPLSQGAESFVVSDGDHLEVRLSDIEGRDIGRVIQP

SEQ ID NO: 33 - protein, 157 aa, Macropus eugenii
MASPGTLAPLQPRPPPREVMKWGFHYNVLSCEGCKGFFRRSVVRGGAQRYTCRGGGACHMNAIMRRKCQECRLRKCREAGMREQCVLSEEQIRKKKIQKHQPVGPAQGSGAPAAPRAPIPGPAGSPTTSSEVGGPGPADGTGVQLTPDQKIMIQQLV

SEQ ID NO: 34 - protein, 57 aa, Macropus eugenii
  SITE 57: Xaa = any amino acid
MQRQKQRSLQMFYISSCYDKYVRELRLPLAAKPEPAIEQPERYGNRIHDHLSLRTFX

SEQ ID NO: 35 - protein, 157 aa, Macropus eugenii
  SITE 145: Xaa = any amino acid
MTLLQQSAMTLPLWIGKPGDKPPPLCGAIPASGDYVAKPGDKVAARVKAVDGDEQWILAEVVSYSHATNKYEVDDIDEEGKERHTLSRRRIIPLPQWKANPETDPEALFQKDQLVLALYPQTTCFYRALIHTPPQRPQEDYSVLXEDTFYADGYSPP

SEQ ID NO: 36 - protein, 238 aa, Macropus eugenii
KAYVMESMAYLTAGMMDSPGFPDCSLEAAMVKVFSSEAAWICVSEALQIVGGLGYMKDYPYERYLRDCRILLIFEGTNEILRMYIAMTGLQHAGQVLTKKIREMQKGNMSVAMEYVSSKLRESLIRKVDLGLTGVNGSVHPSVAESANRLEENVYHFGKMVEVVLKRFGKSIVEEQLALKRIADIVINLYAMTAVLSRASRSVRIGLRYPDLDVLLTNIFCPDAYFENFTTCLCSNTF

SEQ ID NO: 37 - protein, 248 aa, Macropus eugenii
  SITE 220: Xaa = any amino acid
VLYFTWLVFDWNTPKKGGRRSKWVRNWAVWRYFRDYFPIRLVKTHNLLTTRNYIFGYHPHGIMGLGAFCNFSTEATEVGKKFPGIRPYLATLAGNFRIPVLREYLMSGGICPVNRDTIDYLLSKNGSGNAIIIVVGGAAESLNSTPGKNAVTLKNRKGFVKLALRHGADLVPIYSFGENEVYKQIIFEEGSWGRWVQKKFQKYIGFAPCLFHGRGFFSSXTWGLVPYSKPITTVVGEPITIPKLEHPA

SEQ ID NO: 38 - protein, 229 aa, Macropus eugenii
AFVAAISVIIVSELGDKTFFIAAIMAMRYNRLTVLAGAMLALGLMTCLSVLFGYATTVIPRVYTYYVSTALFAIFGIRMLREGLKMSPDEGQEELEEVQAEIKKKDEELQRTKLLNGPGDIETGTSTTIPQKKWLHFISPIFIQALTLTFLAEWGDRSQLTTIVLAAREDPYGVAVGGTVGHCLCTGLAVIGGRMIAHKISVRTVTIIGGIVFLAFAFSALFISPDYGS

SEQ ID NO: 39 - protein, 166 aa, Macropus eugenii
NKPSSAGERSPTGGEAMTLTCDPDLSPFTSSHDSVISPLPPVSLDLLSSPVYTGSLTFRDVVVTFSHEEWGHLDPSQKALYCEVMLENYRNMVCLGLAVSQPDVMDQLERGEVPWSPEGEGAYSSSAALCLWSVQDTKVQGHGANPNVLGHFIVDLLEGFQATFML

SEQ ID NO: 40 - protein, 254 aa, Macropus eugenii
  SITE 222, 236, 254: Xaa = any amino acid
FFEEPEDPSSRSFFSEIISSISDVKFSHSGRYMMTRDYLSVKVWDLNMETRPVETYQVHEYLRSKLCSLYENDCIFDKFECCWNGSDGALMTGSYNNFFRMFDRSTRRDVTLEASRESSKPRAILKPRKVCTGGKRKKDEISVDSLDFNKKILHTAWHPLENIIAVAATNNLYIFQDKVNREVSSSGDIVLLLPPEAWSLFCSKKNILVLSMKNTDAFMSLXQVKERSWPGLVIQXITLLSQKWEEPMRNKVDX

SEQ ID NO: 41 - protein, 248 aa, Macropus eugenii
  SITE 248: Xaa = any amino acid
GAVSRSDGSQPPGLPSRFPRTSSSSSGFGEEAAPAERGRQPSWACRRGRSPARGPGSLSSDPSSGQGPGTSRWARSGMAASTVLGAGLARILFYPTLLYTLLREKVSGPAHRDWYHRIDSTVLLGALPLRSLTPRLIEKENVRGVITMNEEYETRFLCNTFEEWKALGVEQLRLSTVDMTGVPTLENLKKGVQFTLKYMALEKSVYVHCKAGALRSATMVAAYLIQVYKWSPEEAREAIAKIRFHIHX

SEQ ID NO: 42 - protein, 257 aa, Macropus eugenii
MSFFLVMVSLLLLTQHAVAIKELRVHPYTDVTLPCHFSFVKGTDDLEFSWEREDIKEEFEVEDDRDYFQFFRFYDFEVFSKVVYQFRNNKEQTEEQNSMYEKRVSVDPAEIPEGSLSLLLKNVNFQDQAIYKCLAVSSIGRGESKIKLIVEDSEEPPVQFKQIDDETVATCVSTGWYYLPNVTWLDRGERDLSNYSTVEILEEQMNGAYRVFSVLKYPVKLNEKYVCRIIETDVNFQPFRTIRKFPRRKFHRMYDYY

SEQ ID NO: 43 - skipped

SEQ ID NO: 44 - protein, 298 aa, Macropus eugenii
PLRLAFLTLWPRSPASAASSLSFLAMEPATDSEHGSPPEPSALQPPGGSSPLATGSQSHPGREPQQEERHLRISESGQFSDGLEDRGLLESSTRLKPHEAQNYRKKALWVSWFSIIVTLALAVAAFTVSVMRYSASAFGFAFDAILDVLSSAIVLWCTQAIHSLCDLGGDIPSVICMYVVKAIHDLSTRLLPEVDDFLFSVSILSGILCSVLAVLKFMLGKVLTSRALITDGFNSLVGGVMGFSILLSAEVFKHNASVWYLDGSIGVLIGLTIFAYGVKLLIDMVPRVRQTRHYEMFE

SEQ ID NO: 45 - protein, 70 aa, Macropus eugenii
  SITE 30: Xaa = any amino acid
APDARLAFDLPGKDHVTINPVLSTQMTIAXDNSPHCPLVVQARLCVYPYYTATCCRSCAHVQDRAPPEPA

SEQ ID NO: 46 - protein, 241 aa, Macropus eugenii
HLIRDIINQRCGSNPLIGRPNKVTTVDRFQGQQNDYILLSLVRTRAVGHLRDVRRLVVAMSRARLGLYIFARVSLFQNCFTPAFNQLTARPLHLQIIPTEHFPTTRKNGENPSHPVQIIKNMPQMANFVYNMYMLMIQSTHHYNQALLPPPAMVEESDTVQNQEAEMEVEVEPSHPPSETTPTTAEAVLTMAEAAPTTVTPAASEGTETDSTPAPAETEVTPKHQRVISKAGIPIGREHFI

SEQ ID NO: 47 - protein, 246 aa, Macropus eugenii
  SITE 241, 244: Xaa = any amino acid
EKQARQEAERREKAERAARLARETPQEASGPQIKELTDEEAERLQLEIDRRKGEESNQKGDGPAQPLNNSNSAVKQEADDEEEEEDEKDKGKLRPNAGNGADLPNYRWTQTLSELDLAVPFLVNFRLKGKDVVVDIQRRHLQVGLKGQPPLIAGELYNEVKVEESSWLIEDGKVVTVHLEKINKMEWWSRLVSTDPEIKHQKINPENSKLSDLDSETRSMVEKMMYDQRQKSMGFATSEEXKSXRF

SEQ ID NO: 48 - protein, 136 aa, Macropus eugenii
MLFSLRELVQWLGFATFEIFVHVLALLVFSVLLALRVDELAPGLSWWNVFVPFFAADGLSTYFTTIVSVRLFQDGEKRLAVLRLFWILTILSLKFVFEMLLCQKLVEQTRELCFGLIMSPVFILLQLLMIRACRAN

SEQ ID NO: 49 - protein, 134 aa, Macropus eugenii
  SITE 132: Xaa = any amino acid
MSSLHVVHPYWPRNLQLSDYVPNDRPVWQILAVFFSVSGALLVGTWLLSGQGRRGSGSRVPALGPWRRLAVCWFAMCAFVHMVIEGWFSVYNQDIARDQAFLSQLWKEYSKGDSRYILADNFTVCMETVTAXAW

SEQ ID NO: 50 - protein, 180 aa, Macropus eugenii
TTPVASDWQQPAVKLPALRARSATVFLAMNSNQTHLPQALGQINRAFSPPNYELLSEERELRTPGRESASTIINVHSYEPPANDFLVWSVFNTLYMNFCCLGFMALVFSVKARDRKVVGDLNGARSYGSTAKCLNIFALIFSLLIVIVLTALLATGIIALKRVVTNPSEIPHYSDLFPGN

SEQ ID NO: 51 - protein, 181 aa, Macropus eugenii
  SITE 181: Xaa = any amino acid
MSHSTSSRLADSPQLSKSSLLNILGSPSPERMGLADSLPPTPQSSTPSPGPPPLPLLPGGSPGLDGDWESREELRLRELEEARARAAQMEKTMRWWSDCTANWREKWSKVRAERNKAREEVRQLRQKLEALTKELTGARRDRQEALNQSEQLSKEVARLKGARLQEEEEGENRQEGEEAHX

SEQ ID NO: 52 - protein, 149 aa, Macropus eugenii
  SITE 133: Xaa = any amino acid
MADAFVGTWKLIDSKNFDDYMKSIGVGFATRQVGNMTKPTTIIQISGDTITLKTQSTFKNTEISFKLGVEFDETTADDRKVKSLVTLDGGKLVHVQKWDGQETTLVRELQDGKLILTLTHGSAVCVRTYEKEXILRLAHIVTCSANGYH

SEQ ID NO: 53 - protein, 235 aa, Macropus eugenii
  SITE 225, 231: Xaa = any amino acid
RNKIGDAGLDWLCQGILSPACNLKTLWLWECDITAEGCRALAQVLKNKPCLTKLSLICNQLGDEGAELLCEALLDPGCQLEELWLRTCGFTVASCASFCTVLEKNRTLKELQLSTNMLEDVGIEQMSKGLMHPDCPVQSLWLGDCELSDACCETLASVLLTNHTLKELDLSNNRMGDVGIRQLVASLRQPDCTLEQLVLFDIYWTEEVDNELKALQEAKPSLRIXILRAWXPYPH

SEQ ID NO: 54 - protein, 134 aa, Macropus eugenii
GICTSTGIIRDFAGPYYVSEDNMAFGKPVKYWKLDPSKVHASRPNAWDTAVHEASEEYKHRMHNLCCDNCHSHVALALNLMRYNNSSHWNMVKVGLFSLIYGKYVSFGAFVKTWLPFVLLLGIIVTAAFVFNLR

SEQ ID NO: 55 - skipped

SEQ ID NO: 56 - protein, 44 aa, Macropus eugenii
LFLLASSFYKGTVPRLGRVCLDVAIVFVIYDEVVKLLNKVWKTD

SEQ ID NO: 57 - protein, 235 aa, Macropus eugenii
  SITE 215, 235: Xaa = any amino acid
ADISGDTQKAKQFLPFLQRAGRSEAVVEYVFSGSRLKLYLPKETCLITFLLAGIECPRGARNLPGLVQEREPFSEEATLFTKELVLQREVEVEVESMDKAGNFIGWLHIDGANLSVSLVEHALSKVHFTAERSSYYKSLLSAEQAAKQKKEKVWAHYEEQPMEEVSPVLEEKERSASYKPVFVTEITDDLHFYVQDVETGTQLEKLMEGMRNDIXSHPPVEGSYAPRRGEFCIAX

SEQ ID NO: 58 - protein, 16 aa, Macropus eugenii
MACLEWFLKMTENIGG

SEQ ID NO: 59 - protein, 182 aa, Macropus eugenii
INPSLGETCPQAIDILLYTPGHLDPAEKIEDAHPKLWCALSEGKVIVFDASSWTIHQQSFKVGTAKLNCMLMAEQNQVWIGSQDSIIYIINIHSMSCNKQLTDHRSSIMDLIVEDGNKGSSSEVYSCSLDGVIIAWNVSTLKVIRRFHLPCQTLTSIKLHIGRLWCCIGYTIISVTSNGSPS

SEQ ID NO: 60 - skipped

SEQ ID NO: 61 - DNA, 1552 bp, Bos taurus
cgcagagacgctgccggagtgaagattgtcccgctttgctctccgtccgccgccgggtgtttcagtgagcgcgcatccccgcggcctgggagacagcagcgatggcgtttcggtgtcagcgggacagctacgccagagagttcaccaccaccgtggtctcctgccgtcccgcggagctgcacaccgaagaaagcaacggcaagaaggaagtgctgagcggtttccaagtggtactggaagacacgctgcttttccccgagggcgggggacagcctgatgaccgtggtacaatcaatgacatctctgtgctgagagtgactcgccggggcacccaggctgatcatttcacccagacgcccctgacccctgggactgaggttcaagtccgggtggactgggaacggaggtttgaccacatgcagcagcattcagggcagcatctcatcaccgcagttgctgatgatctttttgggctgaagacaacatcatgggagttggggcgactccggagtgtcattgagctggacagccccactgtgactgcagagcaagtagctgccatcgagcggagtgtcaatgaaaaaatccgagaccggctgccagtgaatgtgcgagaactgagcctggatgatccagaggtggagcaggtgaggggccggggtttgccagatgatcacgctgggcctattcgagttgttaccatccagagcgttgattccaacatgtgttgcgggacccatgtgagcaatctcagtgaccttcaggtcattaaaattctgggcactgagaaggggaaaaagaacaaaaccaacctgatcttcctggctgggaaccgggtgctgaagtggatggagaggagtcatggcattgagaaggcgctcacagctctgctgaagtgtggagcagaggaccacgtggaagcagtgaagaagctgcagaactccagcaagctcctgcagaagaacaacctgaatctgctcagagacctggctgtgcacattgcccacagccttaggaacagcccggactggggaggtgtgatcacattacacaggaaggacggcgattctgagttcatgaatatcatcgccaatgagattgggtcagaggagaccctcctcttcttaactgtgggcgatgagaaaggtgcgggactcttcttactggcaggaccagctgaggctgtggagaccctggggcccagagtgtctgaggtgctggaaggcaagggagctgggaagaaaggccgctttcagggcaaggccaccaagatgagccggcgggcagaggtgcaggcgcttctccaggactacatcagcacgcagagcgcagaggagtgaggactcgggtgcctcgctggtcctccgttgacaacgggcctcctgagaccacggaaagagtctcttggacaataaaatagtttgacttgaaaaaaaggtggtaccatgtatatttatatgacaagtgccgagtttatgcagtgtgttccagtgtgcttatgcagtcagtatctttgtgaataaacagctttcttcctttttttaaaaaattt

SEQ ID NO: 62 - DNA, 1266 bp, Bos taurus
  variation (98)...(254): n = g, a, t or c
  variation (401)...(426): n = g, a, t or c
  variation (557)...(747): n = g, a, t or c
atgagaccatcaggccagctccccctaacggggctgctattcttttctcttattccaagtcaactatgccagatctgtgtggtaaatgaaacgaactnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncggagagatggagttaacctcgggagagcttgccttgggtattctgggtttgggagcatgtgaaaatcaggatgaagagtttatacgtggtgctcgtctggttagcaaacttgaagccaaattccaagcagaaattcagaacatggnnnnnnnnnnnnnnnnnnnnnnnnnnaactactaccagctcagcctggccctgctgcctttgagtctgttcaacggcagctattcagtcaccagtgttacctgttacttcactcctgaaaataagaactattattttggggaccagttctcagtagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctctctttgtctcatcggactactacaaaaatgaagcaaattgcagagaaactctacgtgcagtatttgacaacatttctcggggagcattttacttgccgattactgcagcccagatcttacctgccttgatgggaaagacctatctggatgtcaccaacccttcttgtggcctcaatccagtcaaattcaacgcctccactgaaaagcctggtactgtgacacctacaaccgcacccttgaatatcttagtcaaatactctgtgcgaatcaacaagacaaatcgaacccaggtcgcattgcaggcaggttctaccaccgctatgagggaagcccacttcacagtggaggagacctcgtggggcccctacatcacctctgttcagggcataaaggccagcaataatgacagaacctactggaagcttctgagcaacggccagccactgagccaaggagctgggagtcatgttgtccaaaatggagacaatttggaggttcgttggagcaaatactaa

SEQ ID NO: 63 - DNA, 1806 bp, Bos taurus
aggctgctccgtgaccccaccatgtccacccccaccacgaattccgtggacacccccttgcctggaaatggccccagcactccctcttcttcacctgggggaaaggaggatggtcctgaaccatgccctggaggggcggatcctgatgtcccaagcactgatggggccgactcagcctccgttgtggtcatcctagacacagcagaggagccggagcgcaagcgaaagaagggcccagctccaaagatgctgggcgatgagctgtgccaagtgtgcggggacacagcctctggcttccactacaacgtgctcagctgtgaaggctgcaagggcttcttccgccgaagtgtgatccgaggaggggccgggcgctacgcctgccggggtggtggcacctgccagatggacgcgttcatgcggcgcaagtgccagcaatgccggctgcggaaatgcaaggaggcagggatgcgggagcagtgcgtcctctccaaagaacagatccggaagaagaagattcggaagcagcagcagcagcagcagcagcagtcgtcacccacggggccgggagtcagcagcagcagcccagcctctgggcctggggcctcccctggagggtccgacgggggtggccagggctctggagaaggtgaaggtgtccagttaacagccgctcaggaactaatgatccagcagttggtggcggcccagctgcagtgcaataaacgctccttctccgaccagcccaaagtcacgccctggcccttgggtgcagacccgcagtcccgtgatgctcgccagcagcgtttcgcccacttcacggagctggccatcatctccgtgcaggagatcgtggacttcgccaagcaggtgcctggcttcttgcagctcggtcgtgaagaccagatcgccctcctgaaggcctctaccatcgagatcatgctgctggagacggccagacgctacaaccacgagactgagtgcatcaccttcctaaaggacttcacctatagcaaggatgacttccaccgcgcgggcctgcaggtggaattcatcaaccccatctttgagttctcgcgggccatgaggcgactgggcctggacgacgccgagtacgccctgctcatcgccatcaacatcttctcagccgaccggcccaatgtgcaggagcccagccgggtcgaggccctgcagcagccctacgtggacgcactgttgtcttacacccgcatcaagaggccacaggaccagctgcgcttcccccgcatgctgatgaagcttgtgagcctgcgcacgctcagctccgtgcactcggagcaggtcttcgccctgcggctccaggacaagaagctgccacctttgctgtctgagatctgggacgtccacgagtgaggggcctgccccaccgccctgcacacccccaatgactggacactggggtaggaagggactgggccgggtctgggctcctgccctgcggcttggagctaaagccttggccccccgccagtcctcaggatgagccccagtcgggatccaggagcctccttgcctgccccactgagtcttcctgggagggctggagggggtcatgggtcctgacctctgacctctcccagcactcccagctgccctccccgcccagcttacacctcaagcctaccatgcagtgcaccttgaacagaggcaggagggcctgtggctctcccacagcccgagagaccacaggctccccactctggtccttttatttaataaaaactaaaaaaaaaaaacaaaaacaggaaaataaagtatgactagaaactt

SEQ ID NO: 64 - DNA, 1339 bp, Bos taurus
  variation (1319)...(1331): n = g, a, t or c
cacgcgtccggaagggtgggggaaatgggcaagaaaactaagctaatttttaaaatatgggtgttttgggaaaatgttgagctggggttttacaaaaatctgacttgagtgtttttctcttcgtttgctgtgcttgtgtgaacagcgtgacaccgtcgccaccgcaggttcgggtttgccctccccgtatgttacctgaagacggagctaatctttcctctgctcgtggcattttgtcgcttatccagtcttccactcgtagggcttaccagcagatcttggatgtgctggatgaaaatcgcaaacctgtgttgcgtggtgggtctgctgccgccacttctaatcctcatcatgacaacgtcaggtatggcatttcaaatatagatgcaatgattgaaggaacttcagaagacatgacagttgtagatgcagcttcattaagacgacagataatcaaactaaatagacgtctacaacttctggaagaggagaacaaagaacgggctaaaagagaaatggtcatgtattcaattactgtagcattctggctgcttaatagctggctctggtttcgccgctagaggtaacctcagctctcagaaatattgtctcaacagctggaaatataaagaatttgcaaacttctttgttttctgtctttgcattgtatgccgtttcacagtccacgccctgaaaatgtatttctcccagaaaggagggggaaggacttacatttgcagaagtaaaggtatattctgtcacttgaccatatttacttttaaccagttccgccataacacctacttaaaattcacactttgcatgttttgtaaataggatctagttggttttttttttaaggaatttttttttttttttgcctcttcagtctgcaccactaattctgagtgtgagaaagtgtatagaaaatgggtttagcaaaatgtctgtaaaggaaaaatagtacttcattttgtcctatttgtagtgatttctcaaaccctattagattaaacaatttagttaatagacatttttgcatccacattccaacattaacactactgaagtcctggtctggtgtcagatttaataaaatcaagctaccccatgtttcatgatcacctccaatataataattcctaacattttcagcggtttatctaaaatgtgggcaaaaatgtgcatttttatttttattttgcagatggttttctataaagtacatttttactaagtctgtgtgtgaatgatttaaaaaactacagaataaggtacaaatgtggtgtatttaataaactgtcaaccaaagctgtatnnnnnnnnnnnnngggggggc

SEQ ID NO: 65 - DNA, 1131 bp, Bos taurus
agactgtgtggggctcatcctgccgcgaatctcgaacgtaaagggcgttgtcatctgcagactctgaaaagcattcgaggggtggcgcgcctgctcccgccgtcgcagtttccagcgcgacgtgcttgttccacccgaggctctgtcggcgcccctgtataccatggcccttgtgtctgctgattcccgaattgcagaactgctcacagagctccatcagctgatcaagcaaacccaggaagagcgttcgaggagtgaacacaacttagtgaacatccagaagacccacgagcggatgcagactgagaacaagatctctccctattaccggacaaagctgcgtggcctctatacaactgccaaagccgatgcggaggctgagtgcaacatccttcggaaagccctggataagattgcagaaatcaagtctctgttggaagagaggcggattgcggccaagatcgcaggcctgtacaatgactcggagcctcctcggaagaccatgcgcaggggggtgctcatgaccctgctgcagcagtcagccatgaccctgcccctgtggatcgggaagcctggtgacaagcccccgcccctctgcggggccattccagcttctggggactacgtggccaaacctggagacaaggtggctgcccgggtgaaggccgtggatggggatgagcagtggatcctggccgaggtggtcagttacagccacgccaccaacaagtatgaggtagacgacattgacgaagaaggcaaagagagacacaccctgagccgccgccggatcatcccactaccccagtggaaggccaacccggagacagaccccgaagctttattccagaaggagcagcttgtgctggccctgtatccccagaccacctgcttctaccgcgccctgatccacacgcccccacaacggccccaggatgactattccgtcctgtttgaagacacctcctatgcagacggttactcccctcccctcaatgtagcccagcggtacgtggtggcttgtaaggagcccaagaaaaagtgaagtggcctagtagacttgcaatcagctgacaccctcaaagggggacgcacaagattttgtgatcaaataaatatttttcctccttg

SEQ ID NO: 66 - DNA, 2207 bp, Bos taurus
tcggcccggccgcgcgggcggggaggcggcagcagcagcagcagcaagagcgacttcgcgggcagcatgagcggctgtgggctcttcttgtgctcggtggcggcgcgcttctgccgcgccccagcggccttcaccgtgatccggcgcccgctgctcaccagtccgccgagccgagctttcgccaaggagctcttcctgggcaagatccggaagaaagaagctttcccgtttccagaggttagccgagatgaacttaatgagattaatcagtttctgggacctgtagaaaaattcttcaccgaagaagtggactctcgaaaaattgaccaggaggggaaaatcccagatgaaactttagagaaactgaagtccctggggctttttggaatgcaagtcccagaagaatatggtggcctgggcctctccaacaccatgtacgcgcggctggcagaggtcatcggcctggacgcctccattgctgtgactctggcagctcaccagtccatcggcctcaagggcatcatcttggccggcagcaaggagcagaaggccaggtacctgcccaggctggcgtccggggagcacgtggccgccttctgtctgacggagccagccagtgggagcgatgccgcctccatccggaccagggccacgctgagtgcagataagagtcactacgtcctcaacggctccaaggtctgggtcaccaatggaggactggccaacgtctttactgtgtttgcaaaaacggaggtcgttgattctgacggctcagtgaaggacaagatcacagcattcatagtagagagagactttggtggcgtcaccaacgggaaagccgaggataagatgggcatccgaggctccaacacctgcgaggtccactttgagaacaccagggtgcccgtggagaacgtcctaggagaagtcggaggtggctttaaggtggccgtgaacatcctcaacagtgggcgcttcagcatgggcagcatggtggccgggatgctcaagaagctgatcgaaatgaccgctgagtacgcctgcacgaggaaacagttcaacaggaacctcagtgagttcggcctgattcaggagaagttttccctgatggcgcagaaggcctacgtgatggaaagcatggcctacctcacggcggcgatgctggacgcacctggcttccccgactgctccatcgaggccgccatggtgaaggtgttcagctccgagggcgcctggacatgcgtgagcgaggcactgcagatccttggcggcttgggctacatgagggactacccgtacgagcgcttgctgcgcgacagccgcatcctgctcatcttcgagggaaccaacgagatcctccgcatgtacatcgccctgacgggcctgcagcatgctggccgcattctaactgcaagggtcaaggagctcaaacggggcaacgtgaccaccatcatggagaccgttggccagaggcttcgggactccctgggccgcacggtggacctgggactgactgggaagcttggagttgtgcaccccagccttgcggatggcgcccacaagctcgaggaaaacgtgtactactttggccgctctgtggagaccctgctgcttcgcttcggcaagaccatcgtggaggagcagctggtcttgaagcgagtggccaacgtgctcatcaacctgtacgggatgacggccgtgctgtctcgggccagccgctccatccgcctggggctgcgggcccacgaccatgaggttctgttggccaacatcttctgctcagaggcttatcaccagaatctcttcatcctgtctcagctggacaagcattctccagaaaacctggatgacatggtcaagaaggtgtcccagcagatcctggagaagcgggcctacatctgcacccaccccctagaccggacgtcctgaagagggcccgcgtccccacggccgccgccctggatacgccacagcagcccgaaggcggagaacgtggtttattacaaatcgactgtttctcctcttcacccgacctgtgggcactgctgctggatgtgttcttggcctgtgagtggacgcggggtaagcacggagctgcagagactcgccggctcggggggcacccagaggaggctggcgggcacaggtcctggcctgggggctacgtctgtgaggccgcacgttctccaagaaacagctcaaaactg

SEQ ID NO: 67 - DNA, 2460 bp, Bos taurus
  variation (2438)...(2460): n = g, a, t or c
ggccgtggccgtggccaaggctccgctttccgccggtgctccggcccgtctggcggcccgtccctccgcaccctcggccgtagcggtccccgcgctcccctgctcggcgcgaagccctggccccggcggccggggcatgggccaggggcgcggggtctggcggcttcccgcggggccccgacctgtactggcttcgtcatgaagaccctcatagccgcctactccggggtcctgcgaggcactggctccagcatcctctctgccctccaggacctgttttctgtcacttggctcaataggtccaaggtagagaagcagctccaagtcatctcggtgctacaatgggtcctgtctttcctcgtgctgggagtggcctgcagcgtcatcctcatgtacacattctgcaccgattgctggctcattgccgtgctctacttcacctggctggtgtttgactggaacacacccaagaaaggtggcaggaggtcacagtgggtccgaaactgggctgtgtggcgctactttcgagactactttcccattcagctggtgaagacacacaacttactgaccagcaggaactacatctttgggtaccatccccatggcatcatgggcctgggtgccttctgcaacttcagcacagaggccacagaagtaagcaagaagttccctggcataaggccctacctggccacgctggccggcaacttccggatgccagtgctgcgggagtacctgatgtctggaggcatctgcccagtgaaccgggacaccatagactacttgctttcaaagaatgggagtggcaatgccatcatcatcgtggtggggggcgcggctgaatccctgagctccatgcccggcaagaatgcagtcaccctgcgcaatcgcaagggctttgtgaaactggccctgcgccatggagccgacctggttcccacctactcctttggggagaatgaggtgtacaagcaggtgatctttgaggagggctcctggggccggtgggtgcagaagaagttccagaagtacattggctttgccccatgcatcttccatggtcgaggcctcttctcctctgacacctgggggctggtgccctactccaagcccatcaccactgtcgtgggcgagcccattaccatccccaggctggagcgcccgacgcagcaggacatcgacctgtaccacgccatgtacgtgcaagccctggtgaagctcttcgaccagcataagaccaagttcggcctcccggagaccgaggtcctggaggtgaactgagcctgtctgcaggggccagcttctgggaggaaccatctgcaaatcgttttctaccaagttctcaagtgctttttgttctgtaaatttggaagcatcatgggtgtctgtgggttatttaaaagaaattataataattttgttaaaccgttacaatgttaggtcttttttaagaaggagaaagggaatatttcaagctctctcagttccagtttgtcctgttccaggtgggcgctgccacatctgggcctttatggtttcacaacccctcttctctcctcccccaaattacagagaaaattcagtcctggttaactggggaagaaggacagtcattagtgactcgggccagttagattatttgccctttggccctgagggatgaggggcagaagcctcctctagtacaaacatctctatgcccagctacccaaggcttgactgcaggactaaaccctcttgccaaggaaagacacagaggaaacaggtgttcccatgctgaagagtgcagggataagcacctggaatattcaagcttgattctcctttctgccacccccatggccccagtctctgaaatctgagcctggactggcctccaaagagaggatcagggtggcagtgttcctgtgctggagaatggagctgtccagatgaaccatctcttgggttatcaagccccatcatgttctggttggagtgactggttttcctcagggggctgatgacacagacccagacccagcacagaccctgccttggcctgccagtggcctcgctttgcttaacccacatcctcagcagtggatgcgaggaaggggtcccctttcctgtttacagaggggcctgagcagtagactggtaccagaacagaagttccccgaacccaaacctcatgtatttgtgccttttctgagaggggggccaggggggacagctagtactctgtgtactctgctttctcttgatgagatcattataccatgtcagactttcgtatattcctaaatggataaatgaaaacaagtgtcctctatgagttattgttagggtcacattagcatcttgctgctgatgattggggagaccactgccccagctgggctgcctgctaagccctctcccactttctccttcagcccaaatggccctttgtnnnnnnnnnnnnnnnnnnnnnnn

SEQ ID NO: 68 - DNA, 1440 bp, Bos taurus
ccgggaacggctcggggtcgaagcttccagtcacccgcacttcctcctgagacgccggtgcgcggccgtctcggcgggcgggatggcggcggcggcccggggtagcggccgcgcatctgcgcccgggctgtttctggtcttgctggttcctttgctgtgggccccggccggggtccgggccgtacccgacgaagccctcagccaccggaacaaggaaccgccagcgcctgcccagcagctgcagccgcagcccgcggccgtgcagggccccgagccggcccgggtcgagaaaggatttacaccagctgctccagttcataccaataaagacgatccagctacccaaactaatttgggatttatccatgcatttgtggctgccatatcagtcatcattgtatctgaactgggtgataagacattttttatagcagccatcatggcaatgcgctataaccgtttgacagtattggctggtgctatgcttgccttgggcctaatgacgtgtttatcagttttgtttggttatgccaccactgtcatccccagggtgtatacatactatgtttcaactgcattatttgccatttttggcattagaatgcttcgggaaggcttaaagatgagtccagatgaaggtcaagaggaactagaagaagttcaagcagaattaaagaaaaaagatgaagaatttcaacgaaccaaactcttaaatggaccaggagatgttgaaacgggtacaagcacaacaatacctcagaaaaagtggctgcattttatttcacccatctttgttcaggctcttacattaacattcttagcagaatggggtgatcgctctcaactgactacaattgttttggcagctagagaggatccctatggtgtagcagtggggggaacagtgggacactgcctctgcactggcctggcagtcatcggaggaagaatgatagcacagaaaatctccgtcagaactgtgacaatcataggaggcatcgtatttttggcgtttgcattttctgcactatttataagtcccgattctggtttttaatgtgctattgttcatttgtatttagtttaagataggtaactttatgtacatagtgtatattacaactaaaagtgatggaaaatactgtattttgtagtactgatttgtgagtttgacccacttaatggaagtattatgtccaaaagaaataatcactgattgtatttgcaaaatggagttttaaaagaaaccggatatttgtgtgaatttttccttttctccagcataattatgttaacataaggtctccgtttttattttattggctttttctttttggctgggggtgaggtgggggtagtattatgtagagaactgttgttcaggggggtctgccacttgattttttttcagcactgactgacccctttgtaaagaatata

SEQ ID NO: 69 - DNA, 387 bp, Bos taurus
atggcagcttctcagggacggctgatgttccaggatgtgaccatagatttcactctagaggagtgggaatgcctggacctcgatcagcaggaattgtacaggaacgtgatgttagagaactatgggaacctggcctccttgggtcttgtggtctctaagccggacctggtcacctttttggagcaaatgaagaatccctgggatataaggagaacggacacaacagccacatacccatgtaggtgtgaatggctggagctgatgactcaggtgagaggtccaaggagcaaagcaattgaaatatatagaactttgaagactgatgatggtgataatgtaatggagcctgtaggtagttcagggctccctggaggaaaggagctcaag

SEQ ID NO: 70 - DNA, 1927 bp, Bos taurus
  variation (1911)...(1924): n = g, a, t or c
ggtgtgagggctcggaccatggtgcagtcccactggctcccctgcccccctctcctgtgagactggctgcggggagggaacatggatacttgtctgccggcttctggttcccacgcaagtaagcctgctgtcaatggaggaggacattgatacccgcaaaatcaacaacagtttcctgcgcgaccacagctatgcgaccgaagctgacattatctctacggtagaattcaaccacacgggagaattactagcgacaggggacaaggggggtcgggttgtaatatttcaacgagagcaggagagtaaaaatcaggttcatcgtaggggtgaatacaatgtttacagcacattccagagccatgaacccgagttcgattacctgaaaagtttagaaatagaagaaaaaatcaataaaataagatggctccctcagcagaatgcagcctacttccttctgtctactaatgataaaactgtgaagctgtggaaagtcagcgagcgtgataagaggccagaaggctacaatctgaaagatgaggagggccggctgcgagatcctgccaccatcaccaccttgcgggtacctgtcctgagacccatggacctgatggtggaggccaccccgcgaagagtatttgccaacgcgcacacatatcatatcaactctatatcagtcaacagcgactatgaaacctacatgtcagctgatgacctgagaattaacctatggaactttgaaataaccaatcagagttttaatatcgtggacatcaagccagccaacatggaggagctcacggaggtgatcacggcggctgagttccacccccaccactgcaacaccttcgtgtacagcagcagcaaagggacaatccggctgtgtgacatgcgggcgtcggcgctgtgtgaccggcacaccaagttttttgaagagccagaagatccaagcaacaggtcatttttctctgaaattatctcttcaatttcggatgtgaagttcagccacagtgggaggtatatcatgaccagagactatctgaccgtcaaagtctgggatctcaacatggaaaaccgccccatcgagacttaccaggttcacgactacctccgcagcaagctgtgctccctctacgaaaatgactgcatttttgataaatttgagtgtgtgtggaatgggtcagacagtgtcatcatgacaggctcctacaacaacttcttcaggatgtttgaccgaaacaccaaacgcgatgttacccttgaggcctcgagggaaaacagcaaaccccgagctatcctaaaaccacgcaaagtgtgcgtggggggcaagcggagaaaagatgagatcagtgtcgacagtctggactttagcaaaaagatcttgcatacagcttggcatccttcagaaaatattatagcagtggcggctacaaataacctatatatattccaggacaaggttaactaggaggacaagttattacctaataatctcacatactgaatactagtcaaacacgttcttaaatgtttctttgggtgttcatttgatgcctcgactttattttccctctgcaggagacaattggaatagaagtccatggagtccaaccttcccacatccccacttctaagaaacttttgtcaaacccagtaggttctgggacacttctgtttagaatcgagagctgccagctaacagtaattcttccatagttgacttgaatttctgatgcttttattgccccgttttctctggtgggtccagtgttttgtttctaggtgtctgctgagataaaatgaggttgtctgtagtatttaaagagaaaagagataagttttttttaattaagcaattccatttgattgaaaaaaaaaatcaacgaaaaataaacactgtttactcttaaaaannnnnnnnnnnnnncgg

SEQ ID NO: 71 - DNA, 831 bp, Bos taurus
  variation (1)...(19): n = g, a, t or c
  variation (810)...(831): n = g, a, t or c
nnnnnnnnnnnnnnnnnnntgggcagggatggcggccggcacgttgctggaggccggtctggcccgggtgctctactacccgacgctgctctacactgtgttccgcgggaagatgcccggccgggcgcaccgcgactggtaccaccgcatcgattccaccgtgctgctgggcgcgctgccgctgcggagcatgacgcgccggctggtccaggacgagaacgtgcgcggggtgatcaccatgaacgaggagtatgagacgaggttcctgtgcaactcctccaaggagtgggagaaagcaggagtggagcagttgcggctcagcacggtagacatgactggggtcccaaccctggctaacctccagaagggagtccagtttaccatcaggcaccagtcgctaggccattctgtctatgtgcactgtaaggccgggcgctccaggagcgccactatggtggccgcgtatctcattcaggtgtaccactggactccagaggaggccataagagccatcaccaagatccggtcccacatctacatcagacctggccagctagaagttctcaaagagttccacaaggtgaccactgcaggggcggccaagacggagattcatcacacacccctgacatgacacaggtggatcaggaagaatgcaacacagcactgcttcctacagaatgaaagtgcttaacaggaccaagggggcttaagcccagacttgacataaggaaatgtgctaagtgggggttaattttgctccctttgtcttgtttcattttcctgaaataacgctgttgtgtggctagnnnnnnnnnnnnnnnnnnnnnn

SEQ ID NO: 72 - DNA, 243 bp, Bos taurus
atgcgatacaataaatagtggtcgcccaggacctctttgggagacgtgtacggaaggtgggggagttcttattgttggttctgtctaaaagccggcatttggagtggcggatggactcttaaggtagggatattgaacagagcaaaacaggagcaacaagggcggcacgttagttgcatcgatagatgtccaaaggaacctaaacatcacgtattctagctagttaaagcaccttggccttct

SEQ ID NO: 73 - DNA, 600 bp, Bos taurus
  variation (570)...(600): n = g, a, t or c
cattgtgttgggcaacctgacaacaggatgagactcctggtgttggctgctctgctcacagtgggcgctggccaggccggcctcaactcgagggcattatggcagttcaacggcatgattaagtgcaagatccccagcagtgaaccattgctggatttcaacaactatggctgctattgtggcctgggtggatcagggacccctgtggatgatctggacaggtgctgccagacgcacgacaactgctacaaacaagccaagaaactggacagctgtaaagtcctcgtggacaatccctacaccaacaactactcctactcatgttctaacaatgagatcacctgcagcagcgaaaacaatgcctgtgaggccttcatctgcaactgtgaccgcaatgccgccatctgcttctcaaaggtgccatacaacaaggagcacaagaacctggacaagaagaactgctagatctgagtatcttcctgggaaattaggattccctttgggccccatggctttgaccttataccatgtcctccaataaagcacccattgaaaggccaaaaaaaaaannnnnnnnnnnnnnnnnnnnnnnnnnnnnnn

SEQ ID NO: 74 - DNA, 1582 bp, Bos taurus
  variation (869)...(892): n = g, a, t or c
atggagacggccgcgggcagcgagcgccgcagcaccccgggtcccgcagtcccgccgccgcccaggggccacgcgcccttggccaccgcctccggcccgctgagctccccggcgcgcgagccgccgcagcccgaggaggagcggcagctgcgtatcagcgagagcggccagttcagcgacgggctggaggaccgaggcttactagaaagcagcactcggctaaaacctcatgaagcccagaactacagaaagaaggcattgtgggtctcctggttctccattatcgtcacactggccctggcggtggctgcctttactgtctccgttatgaggtacagcgcctctgcttttgggtttgcatttgatgccatcctcgatgtcctgtcatcggcgattgtcctatggcgttatagcaatgcggctgctgtacactctgcccatagggagtacatagcctgtgtcatcttgggggtgatattccttctgtcatctgtgtgtatagtggtcaaagctatccatgacctctcgacgaagctgctcccggaagttgatgatttcctgttcagcgtctccattttaagtgggatcctgtgcagcatcctggctgtgctgaagttcatgttggggaaggttctgaccagcagagcactcataacagatgggtttaactcccttgtggggggcgtgatgggcttctccatccttctgagcgccgaggtgttcaagcacaactcggctgtctggtacctggacggcagcatcggcgtgctgatcggcctcaccatattcgcctacggggtcaaactcctcatcgacatggtgcccagggtgaggcagacgcgccactacgagatgtttgagtgaaggtnnnnnnnnnnnnnnnnnnnnnnnntgcatgaagactgtcaggaccaggagacgtttccacgcaggcagacggtgcccatgtttaattgaacatcagcttctctctcggaaagcttgtgctcaaaggaacgaggtctaccccgcaggtcatactgccctgctctcctcccaccaccacccctgcctgacaccgtaggacaacactcaccgagcggggaccctctgcgggaactgacctcgtccaccttctgctataaaacaggttcgtgtggggagggtacacgcccttctgtggtgctcccagcctgacatggatagtgactcaccaggcccacctcctcggggctccacgtgggccgagtctctggtctcctggcagattttaagagaggaccgtaccctttggtcctgcacccctttccagaaccatcgtgtagatcacctcctaatttctatccgtgtatttcattcgtttcatactgttttactaatcctcaatctaaacactggatttgttctaaaggagaccattctatttttaaagtacttagtgatagatgtatgagctttgcatggatcaaactcagccccaatcctttcttgtacgttcagagcccaaacatatatgtgtaaacacaggatcccttttggagagatgtgaaactacagtttatttgtaataaattatataatctatccaaaaatgcatagatct

SEQ ID NO: 75 - DNA, 3618 bp, Bos taurus
atggatccgccggcgggagccgccggccgcctgctctgccccgcgctgttgctgctcctgctgctgccgctgcccgcggacgcccggctcgccgccgctgccgccgaccccccaggcgggccccaggggcacggagccgagcgcatcctggcggtgccggtgcgcactgacgcccagggccgcttggtgtcccacgtggtgtcggcggcgacggccccagctggggtgcggacccgcagggccgcccctgcccagatcccggggctctctggaggcagcgaggaggaccccggtggccgcctcttctacaatgttacggtgtttggccgagacctgcacctgcggctgcggcccaacgcccgcctcgtggcgcctggggccacggtggagtggcagggagaatcgggtgccacccgcgtggagcccctgcttgggacctgcctctacgtcggagacgtcgcgggcctggctgaatcctcttccgtggcgctcagcaactgcgatgggctggctggcctgatccgtatggaagaggaggaattctttattgagcccctggagaaaggtctggcggcgaaggaggccgaacagggccgtgtacacgtggtgtatcatcgaccgaccacctccagaccccctcctctgggggggccacaggccctggacacagggatctccgcagacagcctggacagcctcagccgtgctctgggtgttctggaggagcgagtcaacagctccaggcggaggatgcgcaggcatgctgccgacgacgactacaacatcgaggtcctgcttggggtggacgactctgtggtccagttccacgggacggagcacgtgcagaagtacctgctcaccctcatgaacattgtcaacgaaatctatcacgatgagtccttgggggcccacatcaatgtcgtcctggtgcggataatcctgctgagctacgggaagtccatgagtctcattgagattgggaacccctctcaaagtctggagaatgtttgccgctgggcctacctccagcagaagccagacactgatcacgacgagtaccacgatcacgccatattcctcacacggcaggacttcgggccctcgggcatgcaaggctatgctcctgtcactgggatgtgccaccccgtccgcagctgcacgctgaaccacgaggacggcttctcctctgcgttcgtggtggcccacgagactggccatgtgctgggcatggagcatgatgggcagggcaaccgctgcggtgacgaggtgcggctgggcagcatcatggcgcccctggtgcaggcagccttccatcgcttccactggtcccgctgcagccagcaggagctgagccgctacctgcactcctatgactgcctgcgggatgaccccttcacccacgactggccggcgctgccccagctccccgggctgcactactccatgaacgagcagtgccgtttcgactttggccttggttacatgatgtgtaccgcgttccggaccttcgacccgtgcaaacagctgtggtgcagccaccctgacaacccctacttttgcaagacaaagaaggggccacccctggatgggaccatgtgtgcgcctggcaagcactgctttaaaggacactgcatctggctgacacctgacattctcaaacgagatggcaactggggtgcctggagtcccttcggctcctgctcgcgtacctgcggcacaggtgtgaagttcaggacccgtcagtgcgacaacccacacccagccaatgggggccgcacatgctcgggcctcgcctacgatttccagctctgcaactcgcaggactgccctgacgcgctggccgacttccgcgaggagcagtgccggcagtgggacctgtacttcgagcatggtgacgctcaacaccactggctgccccacgagcaccgggacgccaaggagcggtgtcatctctactgtgagtccaaggagaccggggaggtggtgtccatgaagcgtatggtgcatgacgggacacgctgttcctacaaggacgccttcagcctctgcgtgcgtggggactgcaggaaggtgggctgtgacggggtgatcggctccagcaagcaggaggacaagtgtggtgtgtgcggaggggacaactcccactgcaaggtggtcaagggcacgttctcgcgctcgcccaagaagcttggttacatcaagatgtttgagatcccggcaggagccagacacctgctaatccaggaagcagacaccaccagccatcacctggccgtcaaaaacctggagacaggcaagttcattttaaatgaggagaatgacgtggatcccaactccaagaccttcatcgccatgggcgtggagtgggagtaccgggatgaggacggccgggagacgctgcagaccatgggccccctccacggcaccatcactgtgctggtcatcccagagggggacgcccgcatctcactgacctacaagtacatgatccatgaggactcgctcaatgtggatgacaacaacgtcctggaagacgactctgtgggctatgagtgggccctgaagaagtggtcgccctgctccaagccctgcggtggagggtcccaattcaccaagtatggctgccgccggaggctggaccacaagatggtgcaccgaggcttctgcgactccgtctcaaagcccaaagccatccgccggacctgcaacccacaggagtgctcccagcccgtgtgggtcacgggtgagtgggagccgtgcagccggagctgtgggcggacaggcatgcaggttcgctctgtgcgctgtgttcagcctctgcacaacaacaccacccgctccgtgcacaccaagcactgcaatgacgctcgacccgagggccgccgggcctgcaaccgcgagctgtgccctggccggtggcgggctggatcctggtcccagtgctcagtaacctgtggaaacggcacccaggaacggccagtgctctgccgaactgcggacgacagtttcggggtgtgccgggaggagcggcctgagacggcaaggatctgcaggcttggcccctgtccccgaaacacctctgacccctccaagaagagctacgtggtccagtggctatcccgaccggaccccaactcgccagtccaggagacctcgtcaaagggccgctgccaaggtgacaagtcagtgttctgtaggatggaagtcttgtctcgttattgctccatcccaggctacaataagctgtgctgcaagtcctgtaacccgcacgacaacctcactgatgtggacgacagggcagagccaccctctgggaagcacaatgacattgaagagctcatgcccaccctttcagtgcccactctagtcatggaggtgcagcctccgccaggcatacccctggaggtgcctctcaatacttccagcaccaatgccaccgaggaccatccagaaaccaatgctgtggatgtgccctacaaaatccctggcctggaagatgaagtccagccacccaacctgatccctcgacgaccgagcccatatgaaaagaccagaaaccaaagaatccaagagctcattgatgagatgaggaagaaagagatgctcggaaagttctaa

SEQ ID NO: 76 - DNA, 3042 bp, Bos taurus
atgaatggggcagaagcgggagagggcgacgctcttgcttctctagctcagagccgacaccttgcatgcacttccggccttgttgtgttccgcttcccgaagaatgtacaagctgcggtacgccttcctaaatcaagcagccgcaatcgcctcttaggggaagttcatcagattcaccgctgctcagagcggtcctttcttttccattggagcggagagagtgctgccatgacggcccctgctcagcccaagaaaatcgtggcccctacggtgtcccagatcaacgcagagttcgtgacccagttagcatgtaaatattgggcaccccatatcaagaaaaagtcaccctttgatataaaggtgattgaagatatatatgagaaagaaattgtcaaatcaagccagtatcttgaaaattacctgtggatgaattattctccagaagtgtccagcaaagcctatttaatgtcaatctgctgtatggtgaatgaaaaattcagagaaaatgtccctgcatgggaggccctactccccacaagacgctggtttaataccatcctggatgactcccacgttttggttcactgttacctttccaatcttgttcgtagagaagaggatggccatcttttttcccagctcttggacatgcttaagttctatactggttttgagattaacgaccagactggaaacgctctgacagagaatgagatgacaactattcactatgatagaatcacttctctccaggtgtctcgtcatgagcgtcgaatttctcagattcagcagttgaaccagatgcctttgtatccaactgagaagatcatatgggatgaaaatattgtcccaactgaatactattctggggaaggccttcggaagcatgatgtatgctttttaattaccgtgcgtcccacaaaaccttatggtaccaagtttgaccgaagaagaccttttattgagcaggttggcctagtttatgtcagaggctgtgaaatccagggcatgctagatgataaagggcgtgtcattgaagacggacctgaacccagacccagtcttagaggagaatcaaggacttttagagtgtttttggatccaaatcagtatcaacaagatatgaccaatactatacaaaatggagcagaagatgtgtatgaaactttcaatgtaataatgagaagaaaaccaaaggaaaataactttaaggctgtgctggagactatccggaacctgatgaatactgattgtgtggtacctgactggctgcatgacatcattttaggttatggggatccaagtagtgcacattattccaaaatgcccaatcagattgccacccttgatttcaatgatacatttctctccattgaacacttaaaagccagctttcctggtcataatgttaaagtaactgtggatgacccagctctacagataccccctttcaggataacttttccagtaagaagtggaaaggggaagaagcggaaagatgtagacggggaagatgaagacactgaggaggcaaaaactttaattgtcgaacctcatgttattcctaatagaggtccttatccttacaaccaacccaaacggttctcacttcacgaccatgtcgtgcatttagcagctaccctggtgcatgttgtgggcccaccaggtacaggaaaaacagatgtggcagttcaaattatatccaacatctaccacaatttcccagagcagaggactctgattgttactcattccaatcaggtaatgtctcgttgggaagagtatatcagcaaagtgaaaaacaaaagtaacacgatgccagatattactgaagtctccgctttcttcccttttcatgagtactttgcaaatgcccctcaacccatttttaaaggccgatcttatgaagaagacatggaaattgctgaaggctgtttcaggcatattaagaaaatctttactcagcttgaggaattcagagcctctgaactgcttcgaagtggactagacagatctaaataccttttggtgaaagaagccaaaatcattgctatgacctgtactcatgctgccttaaaacgacatgatttagtcaagttaggtttcaagaatcctcaggatggttttagccgactaaaacgctggattatgattggtgatcatcaccagttacctccagtcattaagaacatggcctttcagaagtattcaaacatggaacagtccctcttcactcgctttgtcagggttggagttcctactgtggaccttgatgcccaggggagggccagagcaagcttgtgcaacctctacaactggcggtataagaatctaggaaacttaccccatgtgcagctcttgctggagttcagtacagccaatgctggcttgctctatgacttccagctcattaatgttgaagattttcagggagttggagaatctgagcctaatccttacttttatcagaatcttggagaggcagaatacgtagtggcactttttatgtacatgtgcttacttggctaccctgctgacaagatcagcattctgacaacgtataatgggcaaaagcatcttattcgtgacatcatcaacagacgatgcgggagcaatccattgattggaagaccaaataaggtgacaactgttgacagatttcaaggtcaacagaacgactacattcttctgtctctagtacgaaccagggcagtgggccatctgaggtatactttattacagctaccacctgctatggtagaagaaagtgaggaagttcaaagtcaggaaacagaattggaaacagaagaagaggccatgtctgctcaagctgacattgtacctgatgaagcaacagatgccagctccagtcaggaaacctcagcctctgagactgaaaccacccccaaccagacaggagccagctccagtccagaggctatccctgctgagtctgagatcactgggacaggaccggtaactgtaccatcggagaataacacccctcatgatgtcacatccactcccggagaaaccgagtag

SEQ ID NO: 77 - DNA, 1618 bp, Bos taurus
gtcgacccacgcgtccgggaaatggttggagacctactttggcttatggaaaacgagaggaggcgcgatctcgttttccttggggcggggggtaggggcgggatgggtgccagcccctgaaagtgccggaagaagagagaccagaaactggggtctctctccacccatatgtgggtcgggaaaggatggaaccaaaaggacctttacaatatgggcctaattttatacgcctgaggagataaacccacgcaagtttaatagtcgaaatcgtccagttggtcagtgggagcatcatcagtcagtaattaataccagcttgaattcatgccctgctggtcttagaagagccggatgattttcatcaaaattgaatctcaagaaatgttatctgaaaccatagtctctgaattccctgtgtatgtactctcttctctcatcagcgacactgttaagttcttccaatgggaaaaatggcgaagatgtttagtttcatccttgtcaccactgctctggtaatgggcaggggaagctcggctctggagaactgcctccaggagcaggcgcggctcagagcccaagtgtacctgctggagacccgggtcaagcagcaacaagtcaagatctcgcagcttttgcatgagaaacaagtccagctccttgataaaggagaggagaatagtgtcattgaccttggaggaaagaggcaatacgcagattgttcagagattttcaatgacgggtacaagcagagtggattttataaaatcaaacctctccagagcccagcagaattctctgtttattgtgacatgtctgatggaggaggatggactgtaattcagagacgatctgatggcagtgaaaactttaacagagactggagtgactatgaaaatggctttggaaattttgtccaaaaaaatggtgaatattggttgggtaatagaaatcttcacttattgaccacacaaggagattacacattaaaaatcgaccttgcagattttgagaaaaatagccgttatgctcaatataaaaatttcaaagttggagatgaaaagaattcctatgatttacatattggggaatattctggaaccgctggagactcccttactggaaattttcatcctgaagtgcaatggtgggccagtcaccaaagaatgaaattcagcacgtgggacagagacaacgacaactacgaagggaactgtgcgaaagaggatcagtctggctggtggtttaacaggtgtcactctgcaaacctgaatggcttctaccacaagggtccctacaccgctaaaacggacaatgggattgtctggcacacctggcatggatggtggtattccttgaaatctgtggttatgaaaattaggccaaatgatttcattccaaatattgtttaattgtccctgctggagtttcagttctgcagtttatcttggtttaagtgatttgaaaaatgtagcaaatctgaatattcacatgtatgatgatggcaattgttatgtgcactacttttctttctcacttttcttacttcaggtactttccatacagcaaatatgtgataattaccaaataaatgtagattatattaataaaaatcgaaaaa

SEQ ID NO: 78 - DNA, 1369 bp, Bos taurus
  variation (1356)...(1358): n = g, a, t or c
  variation (1361)...(1363): n = g, a, t or c
tagctcggcagctgggacgctcggccgggccctggcccgcgcccaaccggcgcgatgctcttctcgctccgggagctggtgcagtggctgggcttcgccaccttcgagatcttcgtgcacctgctggccctactggtgttctccgtgctgctggccctgcgtgtggacggcctggctcctggcctctcctggtggaacgtcttcgtgcccttcttcgctgctgacgggctcagcacctacttcaccaccatcgtctctgtgcgactcttccaggatggagaaaagcggctggccgtgctccgccttttctgggtcctcacagttctcagtctcaagttcgtcttcgagatgttgttgtgccagaagctggtggagcaaacgcgagagctctggttcggcctgatcacgtctccggtcttcattctcctgcagctactcatgatccgtgcctgccgggtcaactagcctcgcagaagataggctggagtgccgagacccacgccgagtccccatgtgtaccgcaccagaggagaaatctgagtctgagagctagcttgtgccctgatgcgtgcctggttttcgatcagtcatgtctgaaattgttccctcagctgggctaaaaagagaagccttgattcttaaagtgtattctctcttatttcatgctttgaatagctaattttgaactgagatcctgagaaggatcctgagaaagccagacaatcctgaactcctgacagaactgcatataagtaaaatatcctcatgggctcttttgagtattttcatggatcctggcaaagtacgccgtgtgcaaaggctacaaagctggactcgggtttgccctggccagcagtgctgacctgagtgcttgctgtccccatctccccggacaagactttcttagatgcttgagctctaaaaagtgtaaaccagcttgtgtcttcttcccagtggcactgcggaggatggctttttcttcattccagcccaggcagacaggaggatgttagatgagcgtgacccggactctatctggagaactgtcgcctgagaaaagaacttgctttcaaggcactgcttggcttcctatcccagcttttccaccaccttgtggccggcagtgttagcgcacttgagacatttaggccactctaagggaccagagagaggaacaggtgttgtgatgtgaataggcattgagacctacacatgggaagtttgcagttgtctcagcccacgcttgtcactctgagggctttggtttgatgccggttaggtgccacatgagcacatttgctgagatgaaaataaaataagggaatgccttgctgagacaaaaaacggacgcgtggnnncannngggcgg

SEQ ID NO: 79 - DNA, 1080 bp, Bos taurus
ggcccggcctgtgagaggttggattttgacctcgcgtccgcccgcgcgccccgagcgatcttagtgagacccgggagcctgtgggagaggccgctgcaaacgcgctgttgacctacaaagacatgaccaccaacaccagccccatgcacccctactggcctcggcatctgagactagacaactttgtgcctaatgactaccccacctggcatatcctggctggcctattctccgtctctggagtcttagttgtggccacatggctgttgtcagggcgtgctgcggtcgtcccactggggacttggcggagactgtccctgtgctggtttgcagtatgtgggttcattcacttggtgattgagggctggttcagcctctaccacgcggaccttctcggagaccaagccatcttatctcaactctggaaagagtatgccaagggagacagccgatacatcctgaatgataacttcatgatatgcatggagaccgtcacggcttacctgtggggaccactcagcctgtgggtggtgattgcttttctccgccaccaacccctccgctttgtcctacagcttgtggtctctatgggtcaggtatatggagatgtgctctatttcctgacagagtaccgtgatggattccagcacggggagctgggccacccactctacttctggttttactttgttttcttgaactccctgtggctggtggtgcccggactcctcatactggattctataaagcaacttgctcatgcccagagcatactggatgccaaagcacccaaagccaagagcaagcagaactaaagagtgatgaactaggcttaaacaccggctattgaggaactctccacctaccagaagatcccaatccttgctcccacaggtcggaaggacaaattgaattgatcctgtcaagctcaggttgatgggtgggcagtgggaagaaaggagccatgcggagccactgtcaggagccaataggacaaaggtacaagagaacctaggaagtctgtgatggggacaattttttaaatcaggaaataaagatcttgacccctaaaaaaaaaaa

SEQ ID NO: 80 - DNA, 670 bp, Bos taurus
ccggcccaggaaaaggaaacttaggagaaatcgaaactcctgggtaaaggagggcccccccaagtgtgtggtggcatctggaccgcagttgctcatctggactgcagttgctccgtccccaccatgaaccgcacatcccagctcttactcactggggcccacggggcggtgcccccagcctatgaggtgctcaaggaggagcacgaggtggccgtgctgggggcgccccagagccaggcgcccctgacgaccacggtgatcaacatccgcagcgacaccgccgtgcccgaccacatcgtgtggtccctgttcaacaccatcttcatgaactggtgctgcctgggcttcgtggcattcgcctactctgtgaagtctagggaccggaagatggtcggcgacatcactggggcccagagctacgcctccaccgccaaatgcctgaacatctgctccctggtcctgggcatccttctgactgtcgtcctcatcgtcctcgtgtccaccggctccctgatgatcgttcaggcagtctcggagctcatgcagaactacggaggccactaggcctgcccacagcccgaggcagtcgcccctttccccgcagcctatccaggcacctgcccccgtgaaataaaaggagggtttgtgtgtgaaaaaaaaaaaaaaaaaa

SEQ ID NO: 81 - DNA, 2026 bp, Bos taurus
  variation (1875)...(1929): n = g, a, t or c
atgagccacgggccgagcccacggctggccgagtccccgcagctgtccaagggcagcctcctgaccatcctgggcagcccgtcgcccgagcgcatggggccggccgactcgcttccgcccacgcctcccagcggcacgccctcgccagggccgccacccgcgctgccgctgccgcccacgcccgcactgctggccgacggggactgggagagccgcgaggagctgcggctgcgggagctggaggaggcgcgcgcgcgggcggcgcagatggagaagaccatgcgctggtggtcggactgcaccgccaattggcgcgagaagtggagcaaggtgcgcgccgagcgcaaccgcgcacgcgaggaggtgcgtcagctgcgccagcgcctcgacgcgctcaccaaggagctggcgggggctcggcgcgagcgccaagaagcgcagggcgagagcgaggcgcggggccgcgagctggcgcggctgcggggcgcccggggaggcgtggacaggacgcccgatgggcccgagacggagccggaaagggaacaggagccggtgccggtgcgcgacgtgggatccggatgcgagagaccgcagggcagccaggagctcgagctgatggagagcctgctgaagaacagaccagaggagccagagggctgctgggaggtgcgcagcgcgggggccggggccccacggggcagctcgggccgccaggagcgcggccgcctgccctgggaggacaccaccgtcgaggaggatgcctccaagctgaccgccctgcggctacggctggatgagtcccagaaggtgctgctcaaggaacgagaggataaaatggcactgagcaggaacatcgagaagctggagggagagctcagccagtggaagatcaagtacgaggaactgagcaagaccaagcaggagatgctcaagcaactcagcatactgaaggaggcccaccaggacgagctgggccgcatgtccgaagacctggaggatgagctgggtgcgcgttccagcatggacaggaagatggcggagctgaggggcgagatggagcggctgcaggcggagaacgctgccgagtggggtcgccgggagcggctggagacggagaaactgggccttgagcgggagaacaagaagctgcgggctcaagtcggggacctggaggaggcgctggcccggcgtcggcggcagacggccagtgctctggactgcgacctgcgggcaagccaggctgcgctctttgagaagaacaaggagctggccgacctgaagcacgtgcacggcaagctgaagaagcagttccaggagaaggtggcggagctggcccatgccaaccggcgggtggagcagcacgaggctgaggtgaagaagctgcggctgcgagtggaagagctcaagaaggagctggcccaggctgaggacgagttggacgaggcccataaccaggcgcggaagctgcagcggtcgctggacgagcagacggagcagagcgagaacctgcaagtgcagctggaacacgtgcagtccaggctccgccgacagcagcagaatgcccccctctttgggaagatccgcagtgctcgctttggtgccgaggaggcaggggacggagccagcgacctggacgaggacgaggacctgcagatccaggtggcttagacctgccagggctaggcaggactgggcgccaccccccaaccctgggtgcccaggctgccccgccactcgaactgatgcagctgtccgtgctgactctgggagcagaccccagtccctgccacagacaacttccttctctcccacggtgggcattgatggtcctgccttactgacactccatggaagggaagccgtggcctacgtttnnnnnnnnnnnnnnnnnnnnnnnnnnnnnnnnnnnnnnnnnnnnnnnnnnnnnnngcctggggtctttggacttatttggttttatagacacaggaccactccaaggcccagtcaggtgcccccacccacccaggactttcttgctggggga

SEQ ID NO: 82 - DNA, 969 bp, Bos taurus
ccacgcgtccggcagaacagagctgtccttgagtcttgaagagccggctgaaaggacggaatttcggaaggagagtgaggaggcaacggccgacaggagcaaaggctcggagctgtgcgcagagcccagcttcaccatggtggacgccttcgtgggtacctggaagttagtggacagcaagaatttcgatgactacatgaagtcactcggtgtcggttttgctaccaggcaggtgggcaatatgaccaagcctaccacaatcatcgaagtgaatggggacacagtcatcataaaaacacaaagcaccttcaagaacacagagatcagcttcaagctgggagtcgagttcgatgagaccacagcagatgacaggaaagtcaagtccatcgtgacgctggatggcggcaaacttgtccacgtgcagaagtggaatggacaagagacatcacttgtgcgggaaatggttgacgggaaactcattctgacactcacccatggcactgcagtttgcactcgtacttacgagaaacaggcatgacctgccctctccttccactgactgctcttctgccagtgggctactcctggactcggcaccagattgcctcatttttctcctctggcattttgtataaatccaccttgactggggaaattctcctggggtcaggtggcaccagcctggatccagttccgttctcattgtgtctgtttgtttttttaactgcatccaaagggtgctctgaggtcaataaagcagagccaaggccccccagttgcctttatgcctttggtgacatgattctgggattctcagtttctgtgtgtgtcagagagtaggcagaaaagactgctactcaggaagcagcactggatgagactgaaatggacagtctcagggacagtgacagctgatcaccttacatgtacaaaaaataaaacagctgtacctacaccctgccttcacag

SEQ ID NO: 83 - DNA, 1647 bp, Bos taurus
  variation 10, 13, 17, 19, 20, 22, 23, 26: n = g, a, t or c
  variation (1635)...(1647): n = g, a, t or c
tatgaatggntgnatgngnngnncgncctcgcgtccgcctgggcctcgccctggccgtgaccctaactcccgcctggcggaagacgcggctcggccctcaggtctgcggcggctggaggaaacccttcacttcacaccatgaagctggacatccagtgtgagcagctcagcgacgcccgctggacggagctgctgcctctgatccagcagtacgaggtggtcaggctggacgactgcggcctcacggaggtgcgctgcaaggacatcgggtccgcgctgcaggccaacgcgtccctgacggagctcagcctgcgcaccaacgagctcggcgacggcggcgtgctcctggtgctccagggcctgcagagccccacctgcaagatccagaagctcagcctccaaaactgctgcctgacggaggctggctgcggagtcctgccgggcgtgctgcgctccctgcccaccctgcgggagctgcacctcagcgacaacccgctgggggatgccggcctgcggctgctctgtgaggggctcctggacccccggtgccgcctggagaagctgcagctggagtactgcagcctgacagccgccagctgcgagcccctgggcctgcgtgtgctcagggccacgcgggacctgaaggagctcgtggtgagcaacaatgacatcggcgaggccggggtccaggcgctgtgccggggcctggcggagtctgcttgccagctggagactctcaaattggagaactgcggcctcacggcggccaactgcaaagacctgtgtgggatcgtggcctcccaggcctcgctgaaggaccttggacctgggcagcaaccggctgggcgacgcgggccttgcggagctgtgccccgggctgctgagccccagctcccagctcaaggaccctgtggctctgggagtgtgacctcaccgtcagcggctgcagagagctctgccgcgtcctccaggccaaggaggccctgaaggagctgagtctggcgggcaacagcctgggggacgagggcgcccagctgctgtgcgagagcctgctgcagcccggctgccagctggagtccctgtgggtgaagtcctgcgggtttacggccgcctgctgccagcacttcagctctatgctgacccagaacaagcatctcttggagctgcagctgagcagcaacccgctgggcgacgcgggcgtccacgtgctgtgccaggccctgggccagccgggcactgtgctgcgggtgctctgggtgggcgactgtgagctgacgaacagcagctgtggcggcctggcctcactcctgctggccagccccagcctgcgggagctggacctgagcaataacggcctgggcgaccccggcgtcctgcagctgctgggcagcctggagcagcccgcctgcagcctggagcagctggtcctgtacgacatctactggaccgaggcagtggacgagcgcctgcgggctgtggaggagagcaagcctggcctgcggatcatctcctgagccccgtcccccacggggcgttaactgagaaaaggtcaagtcagctcatttctggcggagaatcttaggcactttattaaaactcctttttggcagcaggaaaaatttttcctgttttccnnnnnnnnnnnnn

SEQ ID NO: 84 - DNA, 1666 bp, Bos taurus
gcgcagcatggcggaagcggaagggagttcgccgctcctgctgccgccgccgctggctgccccgctcgggatggcggaagtggaggcgccgacggcggccgagacggacaagaagcaacttagcggtgctggcagcggcgccatggacgtggagcggagccgcttcccctactgcgtggtgtggacgcccatcccggtgctcacgtggtttttccccatcatcggccacatgggcatctgcacatccacaggagtcattcgggactttgctggcccctactttgtgtcggaagacaacatggcctttgggaagcctgccaagtactggaagctggaccccgctcaggtctacgccagcgggcccaacgcgtgggacaccgctgtgcacgacgcctctgaggagtacaagcaccgcatgcacaatctctgctgtgacaactgccactcgcacgtggccttggccctgaacctgatgcgctacaacaacagcaccaactggaacatggtgacgctctgcttcttctgcctgctctatgggaagtacgtcagcgtcggcgccttcgtgaagacctggctgccttttgtccttctcctgggcatcatcctgaccatcagcttggtcttcaacctgcggtgatggccgctccgtggccctgggcccacccagctcttgaggaggcagccgccaccccccttttcaccccagattgtttgtcctcgccccgaaaggcagggatgggcctggtgggtttgacccggggtctgggctaggtgggatggagtaaaaggctgagggtgcgcttggggcagaggcatttgcattttgtgcctaaccggtcgctccgaagctcccggacccctccatcccaggctcgtgacccccatcctcgaggcaccctttgttcatctgtcggaaaagccttagtttccctctggagctgggctccctctcagcccagtgcctggctggggggagaagggcatttggactcaccagccagggcagccctggggccagagctgcgcttgaagcccattctcctgccagggctctggtctcgggacgagggcaccctgagaagtctccgtgagctcggcgccccagggcctgccttctccactggccactggaatggcccagcaggactggcgggcccccccccagatgtttgtcagaagctgcgctcggggtgctttgggccccactgcggacagcctccagcccatgcagcactctcttcccactctcccagggcctgcaagcccttgggcctatcttcccctgcagtttggggcctgggctcccccaaccaggctggagaaaggagtgatatacccagggtgacccctgcagatcagagcagtgtcacaaaacttggcctcagccccaggtgtggacttgacactggtgactggacgacctccaagacattgcgggggtggctggtgcagccagcgccatcagagggctgaaggggagctgcttctgcccgcctcccacggctcccccttctctccaagatgtggacgggcccgtgccgccccccctcacctctgctccacagattcctttgtgcgagcctccctggacaggacaataaagagttttaactccaaaaaaaaaaaaaaaaacctggggggggggccggaccccatttcgctttaa

SEQ ID NO: 85 - DNA, 4039 bp, Bos taurus
cccacgcgtccggagccggttctctccggtgggcggcgcgcgccgggtgtccggactttcccggccctgcccggcccgaccctgcgcggcagcatggcgggctcaggccccaagcggcgcgcggcggcggccaccctcccggcgaacgagaaggaggaacggcaggcgcgggagcggatgctggcggcgcggggcgcggacggcgcggagggcgaggtgaccctgcagcgcaacatcaccctgctgaacggcgtggccatcatcgtgggcaccatcatcggctcgggcatcttcgtgacgcccaccggagtgctcaaagaggcgggctcgcccgggctggcgctggtggtttgggccgtgtgtggcgtgttctccatcgtgggcgcgctctgctacgccgagctgggaaccaccatcaccaagtcgggcggcgactacgcctacatgctggaggtgtacggctcgctgcccgccttcctcaagctgtggatcgagctgctcatcatccggccctcctcgcagtacatcgtggcgctcgtcttcgccacctacctgctcaagccgatcttccccacctgccctgtccccgaggaggctgccaagctcgtggcctgcctctgcgtgctgctactcacggccgtgaactgttacagcgtgaaggccgccacccgcgtccaggacgccttcgccgctgccaagctgctggcgctggccttgatcatcctgctcggcttcatccagatcgggaagggtgacgtagccaatctggatcccaagtcctcctttgaaggcaccaaactggacgtgggaaacatcgtgctggcgctgtacagcggcctctttgcctatgggggatggaattacttgaattttgtcacagaggagatgattaatccctacagaaacctgcccctggccattatcatctcgctcccgatcgtcaccctggtgtacgtgctgacgaacctggcctacttcaccacactgacccccgaacagatgctcacgtcggaggccgtggccgtggacttcgggaactaccacttgggcgtcatgtcctggatcatccccgtgttcgtgggcctgtcctgcttcggctcggtcaatgggtccctcttcacgtcctccaggctgttcttcgtgggggcccgggagggccatctgccctccatcctctccatgatccacccacggctgctgacgcccgtaccctcactggtgttcacgtgcatcatgaccctgctctatgccttctccaaagatatcttctccgtcatcaacttcttcagcttcttcaactggctctgcgtggccctggccattgctggcatgctctggcttcgctaccagaaaccggagctggagcggcccatcaaggttcatctggccctcccagtgttcttcatcctggcctgcctcttcctgatcgccgtctccttctggaagacccccgtggagtgtggcatcggcttcaccatcatcctcagcggcctgcccgtctacttccttggggtctggtggagagacaagcccaagtggctgctccagagcatcttttctacgacggtcctgtgccagaagctcatgcacgtggttccccaggagacgtaagcgaccagaaccaggaggccaccctgagagagcaacttggtcctaccatccaggcagctctgccagggcccccgctgtccctgccccagggggcggccgggccacggtgaaccctggtacgaatttagtccccgaaggtggatgtccgcagatacctctcgagcgtggaggcaggaacatcggaccaaggaggctgcaggcttcctcccgaactgggcctcctcccttctcacctttgtttatttgtattatttttttttaacttaagtcttgcgtcaggttgacattaccaagacaattatttttttaagaaatggggtggaagtttctccccaaacgctgtctgcagcacagcgttcttccgaggggccaggtcctgtcaccagattctggacggtgacctggacacatgtgcttggcttccctgtttctccgttgttgtagatgcagtgactttcgtgaaactaacctccgggcttacgtctcgttgccttaaagccctgcgctagctccctcccccggagaagagagagaaagcctagaggcttgtagaagcagggccttcgggctggtggacagcggtgccttggcacctgctgtgggcagggggctccaagcagaggtcaggggcagtgggccaccccgggcctcccgtcagtacgtgcagttgtgggggcggactacaccctgtcactcagaacatctccaaggtcctgtctgtacaccccaccctgaacgccagcctgagcaaaggcccgaggagtccggtggctcctgtggcagatggtgggcagtgcagcctcaggcctgttctgcatcagttttggaatcaagctctagctctttcaaaccctcacacttgaaatctgtaagcagacccctcgacccctcaggggcttcttatctgtgtcccatgaggctgcacccccccaccccaggcctcaagggctgggtcacagccctggcagctgtctggggtgctcccagctcctggccgaggccccctggccccgcccccgtcacctgtccctggcgcccagcctccgctccccagctgcacctgctccggaggccgcctgagtcctgctggggccagcacgcacacccggcctccccccactcccaacaagaggctgtgcttttgttgttgtagttcccatagcgttctccgcggaaggcagccaaggcccagaagctcgtctggcgtttctgggcgggctctctgtcttccacggagcccgcttggggagcagtgtcatccacggtcagtctcccttaggccctgcctcagcctatgacccctgatcttctcaccagggtggggctgagcagagcccttgggcctcgttgctcagctgcagccaaaaaagctgctgctggttcccaggggccacgtgacctccgcgtggccagtccaccgtcacccgcctgggctctggggccccatgtggtccgataggcatagagagagcagcttaggggggcccaggatggacattgaggctgaagtggaggcagccagatgtccagcccttctccccatgctggtcaggagaccctgccagcctgcagctcccaaaacagcatccaccgttgtctcctcaccaagcgaatccttctaggcgaatccttctaggccacggtccacacgtcaggggcccggctcagcgtctggaggcagaacgtcaccgaggaagccagaggcctgatttgagactgttggggcctcagttttcttgggtgtacatggggcactgccctctgcctctccacggggctccagcagcctcatggcagatttcggcctcctcccgcgggactctgaaggcggcacttgggttacctcgtggtccaccaccggctctcaatgtgacctggcggagcacaggggccaggtgactgtccagagcttggtggcctccctgggatgtgcacctaggggtggggtactgtcacccctgagctccctttatacggaaagaccaggggtgtggctccccagcatctgtgaaggcaggctgggggcccaggcaccctggttctgctgcgttggccttctgggccgactccactcctgaccacactactgacaggtgtcctgagcgttcctgcttgtgggttactttttttttaagccaggttttggcataacgcgcatgtttgtccagccgtcgtgcttcacttcaccctcacgtctccctgaggggagggcgccgtgtttatttattgtggggcgataagcagccgtgccgccccgcgggttccggccgcgtcagccgccagccacgatgtcgtgtgtgacttaacgcctttctctgctgttaacgctaacgtgttatgcttagctgctttgggatggggagttttgtgttttgttggggtaatttgttttttaagaattggaattataagcaaaaaaaaaaaaaaa

SEQ ID NO: 86 - DNA, 1465 bp, Bos taurus
cgccgccatggctgcggcgcccgcaccccgcgctctgacctctgccgcgcccgcgccggggaaggccgcactcactcacccggggaaggcgatcctggcaggcggcctggcgggcggtatcgaaatctgcatcactttccccaccgagtacgtgaagacgcagctgcagctggacgagcgctcgcacccgccgcgctaccggggcatcggggactgcgtacggcagacagtccgcagccatggcgtcctgggcctgtaccgcggcctcagctccctgctctacgggtccatccccaaggcggccgtcaggttcgggacgttcgaatttctcagcaaccacatgcgggacgcccagggacggctggacagcacgcgcgggctgctgtgcggcctgggcgccggagtggccgaggccgtggtggtcgtgtgccccatggagaccatcaaggtgaaatttatccatgaccagacctctgccagccccaaataccgaggattcttccacggggtcagggagatcgtgcgggaacaagggctgaaggggacgtatcaaggcctcacggccaccgtgctgaagcagggatccaatcaggccatccgcttcttcgtcatgacttccctgcgcaactggtaccgaggagacaaccccaacaaacccatgaacccactgatcactggtgtgttcggagccatcgcgggcgcagccagtgtcttcgggaacactccactggacgtgatcaagacccggatgcagggactggaagcacacaagtaccggaacacgttggactgcggcctgcagatcctgaggaacgagggactcaaggcgttctacaagggcactgtcccgcgcctcggccgggtctgcctggacgtggccatcgtgttcatcatctacgacgaggtggtgaagcttctcaacaaagtgtggaggacggactgagccctggggtctgcgcgcgggccgtccccagcgccccctcttacaattccagcgcagtcgtgccaaaaggcccttccctgtcccgcgtgctccgtggcctgggtgtgccgtgccgggtcctcacgtcctctgtggtctgtatccaccaccacagtgtctgtgtccagctgagactgtgtgtccctctggcctgtgaatctgcccgcttgtccacgtgcttgtgtgcctgtgttccgtgtttcgtgtcctgtgacccggggtgcccgtgtggccggggtcacgtcccagcccggtgccttccaccctgggcagcaggggcagtgccccggcccaccacagctgcctccgggcctcagcctggcctcactgcattccaggggctgcgtggcccccgcttttcccgccactggccttaatggccctcgggccctccctccgcccgggacagggtggcacctgccactctcaggaccacccttccaaggcaataaaccggatcctgttgcaaaaaaaaaaaaaaaa

SEQ ID NO: 87 - DNA, 3484 bp, Bos taurus
ctctttcgctcccagtgctgctgctgctccagtgagcgcccagcgcgtccgtcccgtccatcgcccacagctggtcgcccgcccgctcctctccgcggcggcctctcaccaacatcgacacccacattgacacgtcctagcgggccaccgccttaatcgctggttttccatcgctacacatggcctcctccgcgcagagcggcggctcctccgggggacccgcggtccccaccgtgcaacggggcatcgtcaagatggtgctctcagggtgtgccatcattgtccgcgggcagcctcgtgggggtcctcctcctgagaggcagatcaaccttagcaacattcgcgctggaaacctcgcccgcagggcagctgtcgcacaacccgatgcaaaagacaccccggacgagccctgggcatttcctgctcgagagttccttcggaagaagctgattgggaaagaagtatgcttcacgatagaaaacaagactccgcaggggcgagagtatggcatgatctaccttggaaaagataccaatggggaaaacattgcagaatcactggttgcagagggcttggccacccggagagaaggcatgagagctaacaatcctgagcagaaccggcttgcagaatgcgaggagcaagcgaaagcatctaagaaagggatgtggagtgaggggaacggttcacatactatccgggacctcaagtataccattgagaacccaaggcacttcgtggactctcaccaccagaagcctgtcaatgctatcatcgagcacgtgcgggacggcagtgtggtcagggccctgctcctgccagattactacctggtcacggtcatgctgtcagggatcaagtgcccaacttttcgacgggaagcggatggaagtgaaacaccagaaccttttgctgcagaagccaaattttttactgagtctcgactgcttcagagagatgtccagatcatcctggagagctgccacaaccagaacattctgggtacaattctgcacccgaacggcaacatcacagagctgctcctgaaggaaggttttgcccgctgtgtggattggtcgattgcagtctacacacggggcgcagaaaagctgagggcagctgagaggtttgccaaggagcgcaggctgagaatatggagagactacgtggctcccactgctaatttggaccaaaaggacaaacagtttgttgccaaggtgatgcaggtgctcaacgctgacgccatcgtcgtgaagctgaactctggggactacaagaccatccacctgtccagcatcagaccgccaagactggagggggagaatacacaggataagaacaagaaactccgtcccctctatgacattccctacatgtttgaagcccgggaatttctgcggaagaagctcattgggaagaaggtcaatgtgactgtggactacattagaccagccagcccagccacagatacggtgcccgccttttcagagcgtacctgtgccaccgtcaccattggaggaataaacattgccgaggcccttgtcagcaaaggtctagccacggtgatcagataccggcaggatgacgatcagcgatcctctcactatgatgaactgctcgctgccgaggccagagctattaagaatggcaaaggattgcatagcaagaaggaagtgcctatccaccgggtcgcagacatatctggggacacccaaaaagcaaagcagtttctgcccttccttcagcgggcaggtcgttctgaagctgtggtggagtatgtgttcagtggttctcgtctcaagctgtatttgccaaaggaaacttgccttatcaccttcttgcttgcgggtatcgaatgccccagaggagcccggaacctcccaggcttggtgcaggaaggggagccattcagtgaggaagcaacgcttttcaccaaggagctggtgctgcagcgagaggtagaggtggaggtggagagcatggacaaggccggcaacttcatcggctggctgcacatcgacggcgccaacctgtccgtcttgctggtggagcacgcgctctccaaggtccacttcaccgccgagcgcagcgcctactacaaatccctgctgtctgccgaggaggccgccaagcagaagaaggagaaggtctgggcccactacgaggagcagccggtggaggagttgatgcccgtgctggaggagaaggagcgctcggccagctacaagccggtgttcgtgacggagatcaccgacgacctgcacttctacgtgcaggacgtggagacaggcacccagctggagaagctgatggagaacatgcgcaatgacattgctagccaccctcccgtcgagggctcctacgccccccgccggggcgaattctgcatcgccaaattcgtagacggagaatggtaccgtgcccgagtggagaaagtcgagtctcctgccaaagtacacgtcttctacatcgactacggcaacagggagatcctgccgtccacccgcctgggcactttgccacctgccttcagcacccgcgtactaccagctcaagccacggagtatgcctttgccttcatccaggtgccccaagatgaggatgctcggactgacgcggtggacagcgtggtgcgggacatccagaacacgcagtgcctgctcaacgtggagcacctgagcgccggctgcccgcacgtcaccctgcagttcgctgactccaagggggacgtggggctgggcctggtgaaggaggggctggtcatggtggaggtgcgcaaggaaaagcagttccagaaagtgatcacagaatacctgaatgcccaggagtcagccaagagcgccaggctgaacctgtggcgctatggagacttccgagccgacgatgcagatgagtttggctacagccgctaaggagggccccgggtctgacccccagcacagctcacgccagcaccccttcctctgccggcagggtgttttcagctccagacctcaaatcaggggcatagattgggtccagctttgcttcagtgtctgggaatgtctcgtaggacggcatctgggcagggagtgggaagagcccccccccaaccacagctttcagggggcagacattgtcttccaaaaggacaggcactgccctctactgtctctccccgctgattttgtttctatttggaggtttgtgggggttttgttctttttttttttaattgtcctcaaatcaggaagaaacatgaaagactttgtcctaatggggggcttaatcctggccccaaggccagctgccgactggcactccacgtccgtcccagaccgcccttctccgcggctctctgtccagctgttgattatgtgatttttctgatacgtccattctcaaatgccagcgtgtccacatctgcccctttgcccagccctccctatttctgtatttaaagcttttgaggcccaataaaatagtacatgctgtctgcaaaaaa

SEQ ID NO: 88 - DNA, 2162 bp, Bos taurus
atggcccccatgggcatccgcctgtccccgctgggggtggccgtgttctgcctgctggggctcggcgtgctctaccacctctattcaggcttcctggccggccgcttcagcctctttggcctgggcggcgagccgggcggcggggcggcggggcccgcggggcccgcggcctcggccgacgggggcacggtggacctgcgcgagatgctggccgtatcggtgctggcggccgtgcgcggcggcgaggaggtgcggcgcgtacgcgagagcaacgtcctccacgagaagtccaaggggaagacgcgcgagggagcggacgacaagatgaccagtggagacgtgttgtccaaccgcaagatgttctacctactcaagaccgccttccccagcgtgcagatcaatacggaggaacatgtggatgcatctgatcaagaggtgatcttgtgggatcggaagattcctgaggacatcctaaaggaaatagccactccccaggaagtgccggcagagagcgtcaccgtctggattgacccactggatgccacacaggagtacacggtagaggatcttcggaagtatgttactactatggtgtgtgtggctgtaaatggtaaacctgtgctaggagtaatacataaaccattttctgaatatacagcctgggcgatggtagacggtggatcaaatgtgaaagcccgcacttcttacaatgagaagaccccaaggattgtcgtgtctcgctcgcattcaggaatggtcaaacaggttgctcttcagactttcggaaaccagactacgatcatcccagcgggcggtgctggttataaagttttagcactcttggatgtgcctgataagagtcaagaaaaagctgatctatatatccatgtgacatacatcaaaaagtgggatatctgtgctggcaacgccatcttgaaagccctcggggggcacatgaccaccctgagtggtgaagaaattagctacactggttccgacggcattgaaggggggctcctggccagcatcaggatgaaccatcaggctctggtcagaaagctcccggatctggagaagacgggacataagtaagccaagcttgatgacaggtcacagccccgccagagctgaacggtcggcctgacgtgctgaggcttcaaagcggcggtggagactatgcatggtttaggccatcccgaactttttaaagtatttatgaagcatctgagacttcttttccctgtaataggatgcagggtcagggagaatgggttgcttcatgtctcaagtattgtctttatttttgagactattttcgtacggttgtcatacacaaggcgcatatatatatatttgtgaattaaaatctatagctgagtctacattgttatgagtcaccattttcacacaatatcatgaatcttcagtttgttaatactttcatatagaattgggctgaagaaaggattgttttgtgtaggtgtttaatactactatacagttatatctcattgaaaataaaataattggggatttttacccctaattcagatagaaagcacaagaagctgcagattcattaatatgcaacagattctctgttaatgaatatttcttatggattaaaatagaaaaagctcaaattgtttttttttctttgaaattttaataacagtttcaccagctagtagagaatatggatatatgacggttgcattgtaatttctgtgtgtgtgtgtattgtttcgttttggtttgtttttataaagagacgtgtgtttgtacctatgggttcaacatttctgccatcttgctaatcagtttcacttgccctgtggatggtgcatggttctgtcctttccctcctgacccaaatttaatctttaacttctaaggtttgtattaagatttcagggcttaacgttagaattttggtactttttctcattaggggtaaaatatgctataaggcattataaaaatataatgttattctgttattaactgaaagttatttgaagttacatgaattgaactagccttcattatttttaaaaaccttgttttagtacttttaaaaaaatttgccatgtctgtagggattccctctttatcaagagtgagttaaggtggcttttgccattaataaatggatctgatct

SEQ ID NO: 89 - DNA, 3006 bp, Bos taurus
aactctgcttcccagggggcttgtgtgtggcctctgagcccagagaggactgtgtccactttctggtgctgaccgacatctgcggcaacagaacctacggcgtggtggcgcagtactaccggcccctgcacgatgagtactgcttctacaacggcaggacgcactgggaggcgctggggccgggcgggagcgcggccggctgcttcgtgcccttcgcggtgtgcgtggtctccaggttcccctattacaacgccctcaaggactgtctttcctgtttattgactcatctgaagttctgtaaagatttcgaagttgacaatcacataaaagattttgctgcaaaactatctttaatacctagcccaccacctggaccactccatttgatatttaacatgaaaccactacagatcgtgtttccctcgagagcggaccccgaaagccccctcatcgacctggaccttcacctgcccttgctgtgcttcagacctgagaaggtgctacagatcctcagctgcctcctgacggagcaacgcgtcgtcttcttctcgtccagctgggctctgctgacgcttgtggccgagtgcttcatggcctacctgcacccgctgcagtggcagcacaccttcgtgcccatcctgtctggacagatgctggacttcctcatggcgccgacctccttcctcatgggctgccacctcgaccactttgaggaggtcagcaaggaagctgatggtttaattctgataaacatcgacagtgggaacatcacctactctaacgatgaagaggttgatgttcccgacatccctctcctggccgcacagacgttcattcagagggtccagagccttcagctgcaccacgagctggacctggctcacctctgcgccagcacagacgtgaacgagggccgggcctccaggcgggcctggcagcagcagctcaactgccagatccagcagatgaccctgcagctgctcgtcagcatcttcagggaagtgaagaaccacttgaattatgaacacagagtgttcaatagtgaagagtttctcaaaacaagagctctgggggaccagcagttttataaacaggtcctggatacatacatgtttcacgccttcctcaaggcccggctcagcagaaggatggacgcctttgcccagatggacctccacacacagtccgaggaggacaggataaacgggatgctcctgagtccgaggagacccacgatcgagaagatggccagccggaagtcctcagtctcgcacacggcccaccggcgcatggtggtcagcatgcccaacctgcaggacatcacggtgcctgagctgccgccccggaactcgtcgcttcggaagatacagaccacggagtatgggagcagccacacagttctggacgtgccccccaagtcgacgcacaccttcaagatcccagaaatccacttcccgctggtggcccagtgcgtgcaggcattctacgcggactgcgtggcccagctgagtaaagccatgggctcactggcccccgagaactcagtgctgctggccaggtacttctacctgagggggctggtgcagctgatgcagggccagctgctggatgctctgctggacttccagagcctgtacaagaccgatgtgcgtgtcttccccaccgacctcgtccagacgaccgtggagtccctgtcggcacccgagcgcacgcaggctgagcgcatgcctgagctgcgccggctcatcagcgaggtcatggacaggcccgcggacgtccccaaggccgacgaccgcgtcaagaacttcgagctgcccaagaagcacatgcagctggacgatttcgtgaagagggtgcaggagtcgggcatcgtgaaggacaccgtcatcatacaccggctgttcgaggcgctgactgtaggacacgagaaacagattgacccagaaacattcaaagatttctacaactgctggaaggaggcagaagcagaggcgcaggaggtcagcctgcctttgtcggtgatggagcagctggacaaaagtgagtgtgtgtacaagctgtcgcgctccgtcaagacaaatcgcggggtgggcaagatcgccatgacccagaagcgcctgttccttctcacggaggggcggccaggctacgtggagatctccaccttcaggaacatagaggatgtcagaagcaccatggccacttttctgctcctgagaattcccactttaaaaatcaaaacgacgtccaagaaagaagtctttgaagctaacctaaagacggagtgtgacctctggcacctgatggtgaaggagatgtgggccgggaggaagctggccgacgaccacaaggacccccagtacgtacagcaggcgctgacccacattctcttgatggacgcggtcgtcggcacgctgcagtcaccgggagccatctatgcggcctccaagttgtcctactttgataggatgaagaatgaaatgcccatggccattccgaagacgacctcggaaaccctgaaacacaagatcaacccctcggagggggagaccaccccacgagccgtcgacgtgctgctctacaccccagggcatcttgacccagcagaaaaagtggaggatgctcaccccaaattatggtgcgcactgaatgagggcaaggtgatcgtatttgacgcttcctcctggacgattcaccagcactgctttagagtgggcacttctaagctgaactgcatggtgatggcggagcagagccaggtgtgggtcggctcggaggactcggtcatctacatcatcaatgtccacagcatgtcctgcaacaagcagctcaccgaccaccgctccagcgtcacgggcctggctgtgcaggacggggagcaggcgcccagcaccgtgtactcgtgcagcgccgacgggactgtcctggcgtggaacgcaagctccctgcgggtgaccggcaggttccaggtgcccggtggagggctctcggccatcagactgcacg

SEQ ID NO: 90 - DNA, 1 bp, Bos taurus
h

SEQ ID NO: 91 - protein, 422 aa, Bos taurus
MAFRCQRDSYAREFTTTVVSCRPAELHTEESNGKKEVLSGFQVVLEDTLLFPEGGGQPDDRGTINDISVLRVTRRGTQADHFTQTPLTPGTEVQVRVDWERRFDHMQQHSGQHLITAVADDLFGLKTTSWELGRLRSVIELDSPTVTAEQVAAIERSVNEKIRDRLPVNVRELSLDDPEVEQVRGRGLPDDHAGPIRVVTIQSVDSNMCCGTHVSNLSDLQVIKILGTEKGKKNKTNLIFLAGNRVLKWMERSHGIEKALTALLKCGAEDHVEAVKKLQNSSKLLQKNNLNLLRDLAVHIAHSLRNSPDWGGVITLHRKDGDSEFMNIIANEIGSEETLLFLTVGDEKGAGLFLLAGPAEAVETLGPRVSEVLEGKGAGKKGRFQGKATKMSRRAEVQALLQDYISTQSAEEEDSGASLVLR

SEQ ID NO: 92 - protein, 421 aa, Bos taurus
  SITE (33)...(85): Xaa = any amino acid
  SITE (134)...(142): Xaa = any amino acid
  SITE (186)...(249): Xaa = any amino acid
MRPSGQLPLTGLLFFSLIPSQLCQICVVNETNXXXXXXXXXXXXXXXXXXXXXXXXXXXXXXXXXXXXXXXXXXXXXXXXXXXXXGEMELTSGELALGILGLGACENQDEEFIRGARLVSKLEAKFQAEIQNMXXXXXXXXXNYYQLSLALLPLSLFNGSYSVTSVTCYFTPENKNYYFGDQFSVXXXXXXXXXXXXXXXXXXXXXXXXXXXXXXXXXXXXXXXXXXXXXXXXXXXXXXXXXXXXXXXXALFVSSDYYKNEANCRETLRAVFDNISRGAFYLPITAAQILPALMGKTYLDVTNPSCGLNPVKFNASTEKPGTVTPTTAPLNILVKYSVRINKTNRTQVALQAGSTTAMREAHFTVEETSWGPYITSVQGIKASNNDRTYWKLLSNGQPLSQGAGSHVVQNGDNLEVRWSKY

SEQ ID NO: 93 - protein, 455 aa, Bos taurus
MSTPTTNSVDTPLPGNGPSTPSSSPGGKEDGPEPCPGGADPDVPSTDGADSASVVVILDTAEEPERKRKKGPAPKMLGDELCQVCGDTASGFHYNVLSCEGCKGFFRRSVIRGGAGRYACRGGGTCQMDAFMRRKCQQCRLRKCKEAGMREQCVLSKEQIRKKKIRKQQQQQQQQSSPTGPGVSSSSPASGPGASPGGSDGGGQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPSRVEALQQPYVDALLSYTRIKRPQDQLRFPRMLMKLVSLRTLSSVHSEQVFALRLQDKKLPPLLSEIWDVHE

SEQ ID NO: 94 - protein, 201 aa, Bos taurus
  SITE 74: Xaa = any amino acid
MAELVKSKYGQVTEYTFTSANVSPSPSFLGEIHFQGVDCETAYNAKTENKEVCKFFIFPAVETIFLRAEVTSSXRNQSQLLSSQNATVIEYMTISLLARSLFSSSRSCRRLFSLIICRLNEAASTTVMSSEVPSIIASIFEMPYLTVVMMRIRSGGSRPTTQHRFAIFIQHIQDLLVSPTSGRLDKRQNATSRGKISSVFR

SEQ ID NO: 95 - protein, 293 aa, Bos taurus
MALVSADSRIAELLTELHQLIKQTQEERSRSEHNLVNIQKTHERMQTENKISPYYRTKLRGLYTTAKADAEAECNILRKALDKIAEIKSLLEERRIAAKIAGLYNDSEPPRKTMRRGVLMTLLQQSAMTLPLWIGKPGDKPPPLCGAIPASGDYVAKPGDKVAARVKAVDGDEQWILAEVVSYSHATNKYEVDDIDEEGKERHTLSRRRIIPLPQWKANPETDPEALFQKEQLVLALYPQTTCFYRALIHTPPQRPQDDYSVLFEDTSYADGYSPPLNVAQRYVVACKEPKKK

SEQ ID NO: 96 - protein, 715 aa, Bos taurus
  SITE 618, 705, 715: Xaa = any amino acid
MSGCGLFLCSVAARFCRAPAAFTVIRRPLLTSPPSRAFAKELFLGKIRKKEAFPFPEVSRDELNEINQFLGPVEKFFTEEVDSRKIDQEGKIPDETLEKLKSLGLFGMQVPEEYGGLGLSNTMYARLAEVIGLDASIAVTLAAHQSIGLKGIILAGSKEQKARYLPRLASGEHVAAFCLTEPASGSDAASIRTRATLSADKSHYVLNGSKVWVTNGGLANVFTVFAKTEVVDSDGSVKDKITAFIVERDFGGVTNGKAEDKMGIRGSNTCEVHFENTRVPVENVLGEVGGGFKVAVNILNSGRFSMGSMVAGMLKKLIEMTAEYACTRKQFNRNLSEFGLIQEKFSLMAQKAYVMESMAYLTAAMLDAPGFPDCSIEAAMVKVFSSEGAWTCVSEALQILGGLGYMRDYPYERLLRDSRILLIFEGTNEILRMYIALTGLQHAGRILTARVKELKRGNVTTIMETVGQRLRDSLGRTVDLGLTGKLGVVHPSLADGAHKLEENVYYFGRSVETLLLRFGKTIVEEQLVLKRVANVLINLYGMTAVLSRASRSIRLGLRAHDHEVLLANIFCSEAYHQNLFILSQLDKHSPENLDDMVKKVSQQILEKRAYICTHPLDXDVLKRARVPTAAALDTPQQPEGGERGLLQIDCFSSSPDLWALLLDVFLACEWTRGKHGAAETRRLGGHPEEAGGHRSWPGGYVCEAXTFSKKQLKTX

SEQ ID NO: 97 - protein, 361 aa, Bos taurus
MKTLIAAYSGVLRGTGSSILSALQDLFSVTWLNRSKVEKQLQVISVLQWVLSFLVLGVACSVILMYTFCTDCWLIAVLYFTWLVFDWNTPKKGGRRSQWVRNWAVWRYFRDYFPIQLVKTHNLLTSRNYIFGYHPHGIMGLGAFCNFSTEATEVSKKFPGIRPYLATLAGNFRMPVLREYLMSGGICPVNRDTIDYLLSKNGSGNAIIIVVGGAAESLSSMPGKNAVTLRNRKGFVKLALRHGADLVPTYSFGENEVYKQVIFEEGSWGRWVQKKFQKYIGFAPCIFHGRGLFSSDTWGLVPYSKPITTVVGEPITIPRLERPTQQDIDLYHAMYVQALVKLFDQHKTKFGLPETEVLEVN

SEQ ID NO: 98 - protein, 317 aa, Bos taurus
MAAAARGSGRASAPGLFLVLLVPLLWAPAGVRAVPDEALSHRNKEPPAPAQQLQPQPAAVQGPEPARVEKGFTPAAPVHTNKDDPATQTNLGFIHAFVAAISVIIVSELGDKTFFIAAIMAMRYNRLTVLAGAMLALGLMTCLSVLFGYATTVIPRVYTYYVSTALFAIFGIRMLREGLKMSPDEGQEELEEVQAELKKKDEEFQRTKLLNGPGDVETGTSTTIPQKKWLHFISPIFVQALTLTFLAEWGDRSQLTTIVLAAREDPYGVAVGGTVGHCLCTGLAVIGGRMIAQKISVRTVTIIGGIVFLALHFLHYL

SEQ ID NO: 99 - protein, 129 aa, Bos taurus
MAASQGRLMFQDVTIDFTLEEWECLDLDQQELYRNVMLENYGNLASLGLVVSKPDLVTFLEQMKNPWDIRRTDTTATYPCRCEWLELMTQVRGPRSKAIEIYRTLKTDDGDNVMEPVGSSGLPGGKELK

SEQ ID NO: 100 - protein, 460 aa, Bos taurus
MDTCLPASGSQQVSLLSMEEDIDTRKINNSFLRDHSYATEADIISTVEFNHTGELLATGDKGGRVVIFQREQESKNQVHRRGEYNVYSTFQSHEPEFDYLKSLEIEEKINKIRWLPQQNAAYFLLSTNDKTVKLWKVSERDKRPEGYNLKDEEGRLRDPATITTLRVPVLRPMDLMVEATPRRVFANAHTYHINSISVNSDYETYMSADDLRINLWNFEITNQSFNIVDIKPANMEELTEVITAAEFHPHHCNTFVYSSSKGTIRLCDMRASALCDRHTKFFEEPEDPSNRSFFSEIISSISDVKFSHSGRYIMTRDYLTVKVWDLNMENRPIETYQVHDYLRSKLCSLYENDCIFDKFECVWNGSDSVIMTGSYNNFFRMFDRNTKRDVTLEASRENSKPRAILKPRKVCVGGKRRKDEISVDSLDFSKKILHTAWHPSENIIAVAATNNLYIFQDKVN

SEQ ID NO: 101 - protein, 201 aa, Bos taurus
MAAGTLLEAGLARVLYYPTLLYTVFRGKMPGRAHRDWYHRIDSTVLLGALPLRSMTRRLVQDENVRGVITMNEEYETRFLCNSSKEWEKAGVEQLRLSTVDMTGVPTLANLQKGVQFTIRHQSLGHSVYVHCKAGRSRSATMVAAYLIQVYHWTPEEAIRAITKIRSHIYIRPGQLEVLKEFHKVTTAGAAKTEIHHTPLT

SEQ ID NO: 102 - protein, 237 aa, Bos taurus
MLPDILVRVLQRNRELRAHPPTDVTLPCRFFFVEGTENLEFAWKREDIKEEYEVEDDKESYQFFRYYDFFEVFSQLVYQFSNNTEQLEEQSSLYEGRVSVDLEEISEGTLSLLLRNVDFMDEAIYKCSAITPNGRGQSTNKQIVERDLSNRSTVEVLEEQMHGFYRVFSVLKYPVKLNEKYVCHPTKTDVNNQPFRIIRKYPRPGWISLQSKGLSRVFSNTTVQKHQFFGAQPSSQS

SEQ ID NO: 103 - protein, 145 aa, Bos taurus
MRLLVLAALLTVGAGQAGLNSRALWQFNGMIKCKIPSSEPLLDFNNYGCYCGLGGSGTPVDDLDRCCQTHDNCYKQAKKLDSCKVLVDNPYTNNYSYSCSNNEITCSSENNACEAFICNCDRNAAICFSKVPYNKEHKNLDKKNC

SEQ ID NO: 104 - protein, 287 aa, Bos taurus
  SITE 148: Xaa = any amino acid
METAAGSERRSTPGPAVPPPPRGHAPLATASGPLSSPAREPPQPEEERQLRISESGQFSDGLEDRGLLESSTRLKPHEAQNYRKKALWVSWFSIIVTLALAVAAFTVSVMRYSASAFGFAFDAILDVLSRRLSYGVIAMRLLYTLPIXEYIACVILGVIFLLSSVCIVVKAIHDLSTKLLPEVDDFLFSVSILSGILCSILAVLKFMLGKVLTSRALITDGFNSLVGGVMGFSILLSAEVFKHNSAVWYLDGSIGVLIGLTIFAYGVKLLIDMVPRVRQTRHYEMFE

SEQ ID NO: 105 - protein, 1205 aa, Bos taurus
MDPPAGAAGRLLCPALLLLLLLPLPADARLAAAAADPPGGPQGHGAERILAVPVRTDAQGRLVSHVVSAATAPAGVRTRRAAPAQIPGLSGGSEEDPGGRLFYNVTVFGRDLHLRLRPNARLVAPGATVEWQGESGATRVEPLLGTCLYVGDVAGLAESSSVALSNCDGLAGLIRMEEEEFFIEPLEKGLAAKEAEQGRVHVVYHRPTTSRPPPLGGPQALDTGISADSLDSLSRALGVLEERVNSSRRRMRRHAADDDYNIEVLLGVDDSVVQFHGTEHVQKYLLTLMNIVNEIYHDESLGAHINVVLVRIILLSYGKSMSLIEIGNPSQSLENVCRWAYLQQKPDTDHDEYHDHAIFLTRQDFGPSGMQGYAPVTGMCHPVRSCTLNHEDGFSSAFVVAHETGHVLGMEHDGQGNRCGDEVRLGSIMAPLVQAAFHRFHWSRCSQQELSRYLHSYDCLRDDPFTHDWPALPQLPGLHYSMNEQCRFDFGLGYMMCTAFRTFDPCKQLWCSHPDNPYFCKTKKGPPLDGTMCAPGKHCFKGHCIWLTPDILKRDGNWGAWSPFGSCSRTCGTGVKFRTRQCDNPHPANGGRTCSGLAYDFQLCNSQDCPDALADFREEQCRQWDLYFEHGDAQHHWLPHEHRDAKERCHLYCESKETGEVVSMKRMVHDGTRCSYKDAFSLCVRGDCRKVGCDGVIGSSKQEDKCGVCGGDNSHCKVVKGTFSRSPKKLGYIKMFEIPAGARHLLIQEADTTSHHLAVKNLETGKFILNEENDVDPNSKTFIAMGVEWEYRDEDGRETLQTMGPLHGTITVLVIPEGDARISLTYKYMIHEDSLNVDDNNVLEDDSVGYEWALKKWSPCSKPCGGGSQFTKYGCRRRLDHKMVHRGFCDSVSKPKAIRRTCNPQECSQPVWVTGEWEPCSRSCGRTGMQVRSVRCVQPLHNNTTRSVHTKHCNDARPEGRRACNRELCPGRWRAGSWSQCSVTCGNGTQERPVLCRTADDSFGVCREERPETARICRLGPCPRNTSDPSKKSYVVQWLSRPDPNSPVQETSSKGRCQGDKSVFCRMEVLSRYCSIPGYNKLCCKSCNPHDNLTDVDDRAEPPSGKHNDIEELMPTLSVPTLVMEVQPPPGIPLEVPLNTSSTNATEDHPETNAVDVPYKIPGLEDEVQPPNLIPRRPSPYEKTRNQRIQELIDEMRKKEMLGKF

SEQ ID NO: 106 - protein, 1013 aa, Bos taurus
MNGAEAGEGDALASLAQSRHLACTSGLVVFRFPKNVQAAVRLPKSSSRNRLLGEVHQIHRCSERSFLFHWSGESAAMTAPAQPKKIVAPTVSQINAEFVTQLACKYWAPHIKKKSPFDIKVIEDIYEKEIVKSSQYLENYLWMNYSPEVSSKAYLMSICCMVNEKFRENVPAWEALLPTRRWFNTILDDSHVLVHCYLSNLVRREEDGHLFSQLLDMLKFYTGFEINDQTGNALTENEMTTIHYDRITSLQVSRHERRISQIQQLNQMPLYPTEKIIWDENIVPTEYYSGEGLRKHDVCFLITVRPTKPYGTKFDRRRPFIEQVGLVYVRGCEIQGMLDDKGRVIEDGPEPRPSLRGESRTFRVFLDPNQYQQDMTNTIQNGAEDVYETFNVIMRRKPKENNFKAVLETIRNLMNTDCVVPDWLHDIILGYGDPSSAHYSKMPNQIATLDFNDTFLSIEHLKASFPGHNVKVTVDDPALQIPPFRITFPVRSGKGKKRKDVDGEDEDTEEAKTLIVEPHVIPNRGPYPYNQPKRFSLHDHVVHLAATLVHVVGPPGTGKTDVAVQIISNIYHNFPEQRTLIVTHSNQVMSRWEEYISKVKNKSNTMPDITEVSAFFPFHEYFANAPQPIFKGRSYEEDMEIAEGCFRHIKKIFTQLEEFRASELLRSGLDRSKYLLVKEAKIIAMTCTHAALKRHDLVKLGFKNPQDGFSRLKRWIMIGDHHQLPPVIKNMAFQKYSNMEQSLFTRFVRVGVPTVDLDAQGRARASLCNLYNWRYKNLGNLPHVQLLLEFSTANAGLLYDFQLINVEDFQGVGESEPNPYFYQNLGEAEYVVALFMYMCLLGYPADKISILTTYNGQKHLIRDIINRRCGSNPLIGRPNKVTTVDRFQGQQNDYILLSLVRTRAVGHLRYTLLQLPPAMVEESEEVQSQETELETEEEAMSAQADIVPDEATDASSSQETSASETETTPNQTGASSSPEAIPAESEITGTGPVTVPSENNTPHDVTSTPGETE

SEQ ID NO: 107 - protein, 324 aa, Bos taurus
  SITE 24, 253: Xaa = any amino acid
MLSETIVSEFPVYVLSSLISDTVXVLPMGKMAKMFSFILVTTALVMGRGSSALENCLQEQARLRAQVYLLETRVKQQQVKISQLLHEKQVQLLDKGEENSVIDLGGKRQYADCSEIFNDGYKQSGFYKIKPLQSPAEFSVYCDMSDGGGWTVIQRRSDGSENFNRDWSDYENGFGNFVQKNGEYWLGNRNLHLLTTQGDYTLKIDLADFEKNSRYAQYKNFKVGDEKNSYDLHIGEYSGTAGDSLTGNFHPEXAMVGQSPKNEIQQWDRDNDNYEGNCAKEDQSGWWFNRCHSANLNGFYHKGPYTLKRTMGLSGTPGMDGGIP

SEQ ID NO: 108 - protein, 136 aa, Bos taurus
MLFSLRELVQWLGFATFEIFVHLLALLVFSVLLALRVDGLAPGLSWWNVFVPFFAADGLSTYFTTIVSVRLFQDGEKRLAVLRLFWVLTVLSLKFVFEMLLCQKLVEQTRELWFGLITSPVFILLQLLMIRACRVN

SEQ ID NO: 109 - protein, 264 aa, Bos taurus
  SITE 230: Xaa = any amino acid
MTTNTSPMHPYWPRHLRLDNFVPNDYPTWHILAGLFSVSGVLVVATWLLSGRAAVVPLGTWRRLSLCWFAVCGFIHLVIEGWFSLYHADLLGDQAILSQLWKEYAKGDSRYILNDNFMICMETVTAYLWGPLSLWVVIAFLRHQPLRFVLQLVVSMGQVYGDVLYFLTEYRDGFQHGELGHPLYFWFYFVFLNSLWLVVPGLLILDSIKQLAHAQSILDAKAPKAKSKQXLKSDELGLNTGYEELSTYQKIPILAPTGRKDKLN

SEQ ID NO: 110 - protein, 146 aa, Bos taurus
MNRTSQLLLTGAHGAVPPAYEVLKEEHEVAVLGAPQSQAPLTTTVINIRSDTAVPDHIVWSLFNTIFMNWCCLGFVAFAYSVKSRDRKMVGDITGAQSYASTAKCLNICSLVLGILLTVVLIVLVSTGSLMIVQAVSELMQNYGGH

SEQ ID NO: 111 - protein, 555 aa, Bos taurus
  SITE (313)...(387): Xaa = any amino acid
MSHGPSPRLAESPQLSKGSLLTILGSPSPERMGPADSLPPTPPSGTPSPGPPPALPLPPTPALLADGDWESREELRLRELEEARARAAQMEKTMRWWSDCTANWREKWSKVRAERNRAREEVRQLRQRLDALTKELAGARRERQEAQGESEARGRELARLRGARGGVDRTPDGPETEPEREQEPVPVRDVGSGCERPQGSQELELMESLLKNRPEEPEGCWEVRSAGAGAPRGSSGRQERGRLPWEDTTVEEDASKLTALRLRLDESQKVLLKEREDKMALSRNIEKLEGELSQWKIKYEELSKTKQEMLKQXXXXXXXXXXXXXXXXXXXXXXXXXXXXXXXXXXXXXXXXXXXXXXXXXXXXXXXXXXXXXXXXXXXXXXXXXXXGDLEEALARRRRQTASALDCDLRASQAALFEKNKELADLKHVHGKLKKQFQEKVAELAHANRRVEQLFAEVKKLRLRVEELKKELAQAEDELDEAHNQARKLQRSLDEQTEQSENLQVQLEHVQSRLRRQQQNAPLFGKIRSARFGAEEAGDGASDLDEDEDLQIQVA

SEQ ID NO: 112 - protein, 133 aa, Bos taurus
MVDAFVGTWKLVDSKNFDDYMKSLGVGFATRQVGNMTKPTTIIEVNGDTVIIKTQSTFKNTEISFKLGVEFDETTADDRKVKSIVTLDGGKLVHVQKWNGQETSLVREMVDGKLILTLTHGTAVCTRTYEKQA

SEQ ID NO: 113 - protein, 456 aa, Bos taurus
  SITE (143)...(314): Xaa = any amino acid
MKLDIQCEQLSDARWTELLPLIQQYEVVRLDDCGLTEVRCKDIGSALQANASLTELSLRTNELGDGGVLLVLQGLQSPTCKIQKLSLQNCCLTEAGCGVLPGVLRSLPTLRELHLSDNPLGDAGLRLLCEGLLDPRCRLEKLXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXVKSCGFTAACCQHFSSMLTQNKHLLELLLSSNPLGDAGVHVLCQALGQPGTVLRVLWVGDCELTNSSCGGLASLLLASPSLRELDLSNNGLGDPGVLQLLGSLEQPACSLEQLVLYDIYWTEAVDERLRAVEESKPGLRIIS

SEQ ID NO: 114 - protein, 209 aa, Bos taurus
MAEAEGSSPLLLPPPLAAPLGMAEVEAPTAAETDKKQLSGAGSGAMDVERSRFPYCVVWTPIPVLTWFFPIIGHMGICTSTGVIRDFAGPYFVSEDNMAFGKPAKYWKLDPAQVYASGPNAWDTAVHDASEEYKHRMHNLCCDNCHSHVALALNLMRYNNSTNWNMVTLCFFCLLYGKYVSVGAFVKTWLPFVLLLGIILTISLVFNLR

SEQ ID NO: 115 - protein, 505 aa, Bos taurus
MAGSGPKRRAAAATLPANEKEERQARERMLAARGADGAEGEVTLQRNITLLNGVAIIVGTIIGSGIFVTPTGVLKEAGSPGLALVVWAVCGVFSIVGALCYAELGTTITKSGGDYAYMLEVYGSLPAFLKLWIELLIIRPSSQYIVALVFATYLLKPIFPTCPVPEEAAKLVACLCVLLLTAVNCYSVKAATRVQDAFAAAKLLALALIILLGFIQIGKGDVANLDPKSSFEGTKLDVGNIVLALYSGLFAYGGWNYLNFVTEEMINPYRNLPLAIIISLPIVTLVYVLTNLAYFTTLTPEQMLTSEAVAVDFGNYHLGVMSWIIPVFVGLSCFGSVNGSLFTSSRLFFVGAREGHLPSILSMIHPRLLTPVPSLVFTCIMTLLYAFSKDIFSVINFFSFFNWLCVALAIAGMLWLRYQKPELERPIKVHLALPVFFILACLFLIAVSFWKTPVECGIGFTIILSGLPVYFLGVWWRDKPKWLLQSIFSTTVLCQKLMHVVPQET

SEQ ID NO: 116 - protein, 311 aa, Bos taurus
MAAAPAPRALTSAAPAPGKAALTHPGKAILAGGLAGGIEICITFPTEYVKTQLQLDERSHPPRYRGIGDCVRQTVRSHGVLGLYRGLSSLLYGSIPKAAVRFGTFEFLSNHMRDAQGRLDSTRGLLCGLGAGVAEAVVVVCPMETIKVKFIHDQTSASPKYRGFFHGVREIVREQGLKGTYQGLTATVLKQGSNQAIRFFVMTSLRNWYRGDNPNKPMNPLITGVFGAIAGAASVFGNTPLDVIKTRMQGLEAHKYRNTLDCGLQILRNEGLKAFYKGTVPRLGRVCLDVAIVFIIYDEVVKLLNKVWRTD

SEQ ID NO: 117 - protein, 910 aa, Bos taurus
  SITE 792: Xaa = any amino acid
MASSAQSGGSSGGPAVPTVQRGIVKMVLSGCAIIVRGQPRGGPPPERQINLSNIRAGNLARRAAVAQPDAKDTPDEPWAFPAREFLRKKLIGKEVCFTIENKTPQGREYGMIYLGKDTNGENIAESLVAEGLATRREGMRANNPEQNRLAECEEQAKASKKGMWSEGNGSHTIRDLKYTIENPRHFVDSHHQKPVNAIIEHVRDGSVVRALLLPDYYLVTVMLSGIKCPTFDGKRMEVKHQNLFAAEAKFFTESRLLQRDVQIILESCHNQNILGTILHPNGNITELLLKEGFARCVDWSIAVYTRGAEKLRAAERFAKERRLRIWRDYVAPTANLDQKDKQFVAKVMQVLNADAIVVKLNSGDYKTIHLSSIRPPRLEGENTQDKNKKLRPLYDIPYMFEAREFLRKKLIGKKVNVTVDYIRPASPATDTVPAFSERTCATVTIGGINIAEALVSKGLATVIRYRQDDDQRSSHYDELLAAEARAIKNGKGLHSKKEVPIHRVADISGDTQKAKQFLPFLQRAGRSEAVVEYVFSGSRLKLYLPKETCLITFLLAGIECPRGARNLPGLVQEGEPFSEEATLFTKELVLQREVEVEVESMDKAGNFIGWLHIDGANLSVLLVEHALSKVHFTAERSAYYKSLLSAEEAAKQKKEKVWAHYEEQPVEELMPVLEEKERSASYKPVFVTEITDDLHFYVQDVETGTQLEKLMENMRNDIASHPPVEGSYAPRRGEFCIAKFVDGEWYRARVEKVESPAKVHVFYIDYGNREILPSTRLGTLPPAFSTRYYQLXATEYAFAFIQVPQDEDARTDAVDSVVRDIQNTQCLLNVEHLSAGCPHVTLQFADSKGDVGLGLVKEGLVMVEVRKEKQFQKVITEYLNAQESAKSARLNLWRYGDFRADDADEFGYSR

SEQ ID NO: 118 - protein, 363 aa, Bos taurus
MAPMGIRLSPLGVAVFCLLGLGVLYHLYSGFLAGRFSLFGLGGEPGGGAAGPAGPAASADGGTVDLREMLAVSVLAAVRGGEEVRRVRESNVLHEKSKGKTREGADDKMTSGDVLSNRKMFYLLKTAFPSVQINTEEHVDASDQEVILWDRKIPEDILKEIATPQEVPAESVTVWIDPLDATQEYTVEDLRKYVTTMVCVAVNGKPVLGVIHKPFSEYTAWAMVDGGSNVKARTSYNEKTPRIVVSRSHSGMVKQVALQTFGNQTTIIPAGGAGYKVLALLDVPDKSQEKADLYIHVTYIKKWDICAGNAILKALGGHMTTLSGEEISYTGSDGIEGGLLASIRMNHQALVRKLPDLEKTGHK

SEQ ID NO: 119 - protein, 1125 aa, Bos taurus
  SITE 521, 1125: Xaa = any amino acid
MAEAAPHHPALPSGLLELCALLGAPRDSLRCSEQVAQRKGVKGPSFLDPEVLSVFVPPFVMKEDSQPAASNCTTPGKNRKRSFRKKRERPRVELGKGVPGGPRGPDPEDVNIPNGVDLLALPQLCFPGGLCVASEPREDCVHFLVLTDICGNRTYGVVAQYYRPLHDEYCFYNGRTHWEALGPGGSAAGCFVPFAVCVVSRFPYYNALKDCLSCLLTHLKFCKDFEVDNHIKDFAAKLSLIPSPPPGPLHLIFNMKPLQIVFPSRADPESPLIDLDLHLPLLCFRPEKVLQILSCLLTEQRVVFFSSSWALLTLVAECFMAYLHPLQWQHTFVPILSGQMLDFLMAPTSFLMGCHLDHFEEVSKEADGLILINIDSGNITYSNDEEVDVPDIPLLAAQTFIQRVQSLQLHHELDLAHLCASTDVNEGRASRRAWQQQLNCQIQQMTLQLLVSIFREVKNHLNYEHRVFNSEEFLKTRALGDQQFYKQVLDTYMFHAFLKARLSRRMDAFAQMDLHTQSEEXQDKRDAPESEETQIEKMASRKSSVSHTAHRRMVVSMPNLQDITVPELPPRNSSLRKIQTTEYGSSHTVLDVPPKSTHTFKIPEIHFPLVAQCVQAFYADCVAQLSKAMGSLAPENSVLLARYFYLRGLVQLMQGQLLDALLDFQSLYKTDVRVFPTDLVQTTVESLSAPERTQAERMPELRRLISEVMDRPADVPKADDRVKNFELPKKHMQLDDFVKRVQESGIVKDTVIIHRLFEALTVGHEKQIDPETFKDFYNCWKEAEAEAQEVSLPLSVMEQLDKSECVYKLSRSVKTNRGVGKIAMTQKRLFLLTEGRPGYVEISTFRNIEDVRSTMATFLLLRIPTLKIKTTSKKEVFEANLKTECDLWHLMVKEMWAGRKLADDHKDPQYVQQALTHILLMDAVVGTLQSPGAIYAASKLSYFDRMKNEMPMAIPKTTSETLKHKINPSEGETTPRAVDVLLYTPGHLDPAEKVEDAHPKLWCALNEGKVIVFDASSWTIHQHCFRVGTSKLNCMVMAEQSQVWVGSEDSVIYIINVHSMSCNKQLTDHRSSVTGLAVQDGEQAPSTVYSCSADGTVLAWNASSLRVTGRFQVPGGGLSAIRLHX

SEQ ID NO: 120-150 - skipped (31 entries)

SEQ ID NO: 151 - DNA, 1035 bp, Bos taurus
atgtctttcctcagtgtgctggccgtcatcgtcttggtaggggtcacaaccaagagcctggctggattgctgggctgtggtgcgatggggctgggggctcaggtgctgtgcctgcaggtactgcggggctacgctgccacgggggacctgggctgcctctgtggcagacctctcccagggtatgtccgccccgatctagcaccaagctgcagtgtaggcattattagcatcatcattattctggctggagcaattgtatttatcatcggttttggtatttcaggaagacgctccatcacagtcaccactctcacctcagctgggaacatcggggaagatggcatcctgagttgcacttttgaacctgacatcaaacttgctgatatcgtgatacagtggctgaaggaaggtgtcatgggcttggtccatgagttcaaagaaggcaaagatgtcctgtcagaccaggatgaaatgttcagaggccggacagcagtgttttctgatcaagtaatagttggtaatgcctctctgaggctgaaaaatgtacaacttacggatgctggcacctataaatgttacatcatcacttctaagggcaaggggaatgctaacctcgaatataaaactggagccttcagcatcccagaggtgaatgtggactataacgccagctcggagagcttacgatgtgaggctccccgatggtttccccagcccacagtcgtctgggcatcccaagttgaccagggagccaacttctcagaagtctccaacactagctttgagctgaactctgagaatgtgaccatgaaggttgtgtccatactctacaacgtcacaatcaacaacacttactcctgtatgattgaaaacaacattgccaaagccacaggggatatcaaagtgacagactcggagattaaaaggcgaagtcacctacagctgctgaactcaaaggcttccctgcgtgtctctgcttcttctgccatcgactgggtactcttgccgctctgcttttatctgacgctaaaataa

SEQ ID NO: 152 - protein, 344 aa, Bos taurus
MSFLSVLAVIVLVGVTTKSLAGLLGCGAMGLGAQVLCLQVLRGYAATGDLGCLCGRPLPGYVRPDLAPSCSVGIISIIIILAGAIVFIIGFGISGRRSITVTTLTSAGNIGEDGILSCTFEPDIKLADIVIQWLKEGVMGLVHEFKEGKDVLSDQDEMFRGRTAVFSDQVIVGNASLRLKNVQLTDAGTYKCYIITSKGKGNANLEYKTGAFSIPEVNVDYNASSESLRCEAPRWFPQPTVVWASQVDQGANFSEVSNTSFELNSENVTMKVVSILYNVTINNTYSCMIENNIAKATGDIKVTDSEIKRRSHLQLLNSKASLRVSASSAIDWVLLPLCFYLTLK

SEQ ID NO: 153 - DNA, 4065 bp, Bos taurus
atgggaataagcagagaaggcattccatggatgccagaagcaaagtatcacacatttgtaagggtcaccgtcactgtgatccatgcaggaggcagccaccgcttccctccactgggagagcgtgaccctgctacatcaaaacaagagcaaggaagaggaactggcctttgctcctggagatttgtcagcttcacctccgtgaattttgtccgtcatcagcacaatcttgctaggaagcacgtgactgcccaaagccggcaggttggaggactcaagagagtacctacttcacaggcaaacttcccaaagagtggtttagaccggctggcctcgcctgcgggtgttcataaaacccctgtggagcttaggaacgagatcacccagactggggaccagacatctgcctcaaccctcaccgtgcctcccccaagcactgcccttctctctgtgatactgggacgatgtcttcccagtgcatttttgccttcagcagctgctggatctgtgccagcgcacctcaccttctggtccaggaaccgcaaggcacaaggagcctgccaagctgggaagacatcacagaactcagtcctggtcgcccgtttgctgccagatttcttccccagcaacccccgatgtggggcgcggggctcgcccacgccgctcgggtaccggcgccacccaaccccttggcgcagctcgcggccaccccgcctgggctggcgcagcgctcgcaccgcccactcggagccggtcgggatttttgggtcccggagaccgaatcccagccgggtcgagggacagccctggcgcgatggctccctcggaacccggctgggaccccctcggcttcgcccccctggagcggtccgcaggccccagttgtgtctgatgctgcttctgtccctccctcagctctgcctggatcaggaggtgttgtctggacactctcttcagacaccccccgaagagagccagggccctgaaggggtctggggtccttgggaccagtgggcctcttgctcccagccctgcggggttggggtacagcgccgaagccggacatgtcagctccctacaactcaactccaccagggcctgccccttccaccccggcccccaagacatccagaagccctgcttccccggggtcaaggcaccagaccgcagacttcccgagaaacccttcccctatacaggccaccacctcgaggaagaggtggcccccttcgaggtcctgcttctcaattagggagagaggaggctccggagactcaaggagctaggaggtcccgggttcgagaccccatcaagccaggaatgtttggctatgggagggtgccctttgctttgccgctccatcggaaccgcaggcatccccgaagaccacccagatctgaactctctcaaacctcagatcttccatccctgactccgagaacagagccatcttcctcaaaccacacccagaaaacagaactgtctcccacagaaccttctgcccacaccctcccaccccaagcagaacccccaagccctgaagctgctcagacagccgtgccctctagagccaggcctgcccccacagggccacaccccagagcccaggcctctggcacagagtcctcctttcattccccatccccgggagaaggtagctcctttcacctgtcccttcagccaagaaggccaagttcccagggttgggccagccccaggctggcagacagacaccctaatcctttcctttctgtgccttggggccgaggccagcagagccaggagcagtggagacctggggggaatctccacgggtccctcacggagtctgcccctccccagccagatggctggctgcctctgctgagctctggcccccactccagcccactctggagcctctttgctcccagtagccctgtcccaagatgttctggggagagtgaacagctgagagcctgcagccaagcgccctgccccgctgagcagccagacccccgggccctgcagtgtgcagcctttgactcccaggagttcatgggccaactgtaccagtgggagcccttcacggaagttcagggttctcagcgctgtgagctgaactgccgtccccgtggcttccgcttctatgtccgtcacacccaaaaggtccaggacgggaccctgtgtcagcctggatccctggacatctgtgtggctggacgctgtctgagccctggctgtgatggcatccttggctctggccggcatccagatggctgtggcgtctgtgggggtgatgattctacctgtcgcctcgtcttggggaacctcactgagcggggtggccctctgggttatcagaagatcttgtccattcctgccggggcctcccgactccagattgcccagctccggcccagctccaactaccttgcacttcgaggtcctgggggccggtccatcatcaatggaaactgggctgtggatccccctgggtcctataccgccggcgggactgtcttccggtacaaccgtcctccccgggaggagggcgctggggagagcctgtccgcagaaggccccaccacccagcccgtggatgtctacatgatcttccaggaggaaaacccaggtgttttctatcagtatgtcatctcttcacctcccccaaaccttgagaaccccactccagagccccgtgttcctcaactccagcctgagattttgagggtagagccccctcctgtttcagcgcctcgccctgcccgcaccccaggcaccctccagcggcaggtgaggatcccccagatgcccgccccaccccctcccagaacacccctggggtctccagccggatactggaaacgagttggacactcggagtgctcggcatcctgtgggaaaggtgtttggcgccccatcttcctctgcatttctcgagagtcaggagaggagctggatgaacgcagctgtgccatgggtgccaggcccccagcctcccaggagccctgccacggccccccgtgcccaccatactgggaggccggcgagtggacgtcctgcagccgttcgtgtggacccggcacccagcaccgtcagctacgctgccggcaggagtttgggggtggcggctcctcagtgcccctagagcgctgcgggcacctgccccgacccaacatcacccagccctgccagctgcgcctctgtggccattgggaggttcgctcaccctggagtcagtgctctgtgcgatgcgggcgcggtcagaggagccggcaggtccgctgtgtcggcaacaacggggatgaagtgagtgagcaggagtgcgcctcagggcccccgcggccccccagcagagaggcctgcgacatggggccctgcaccacggcctggttccacagcgactggagctccaagtgctcagcggagtgtgggacgggaatccagagacgttctgtggtctgccttgggagtggggaggcccacgggataagccaggaggaagcaggggcaggagccggtgagcagacctgtgcacccggaagccggccccctgacatgcgtgcctgcagtctggggccctgtgaagtgtcgtggtgctggtacaccgggccctgggcagagtgctcctcagagtgcggctctggcacacagcgtagagacgtcatctgtgtgtccaaactggggactgagttcaacgtgacttctcctagcaactgttcccaccttcccaggccccctgccctgcagccctgtcaagggcaggactgccaggaccggtggttttctacaccctggagtccgtgttctcgctcctgccagggtggcgtgcagacaagggaggtccagtgcctgactgccaaccagaccctcagcatccgatgccctcttcacctgcggcccgccaggaaacgatcctgtaacagccagccctgcagccagcgccctgatgatcaatgcaaggacagctctccacactgccccctggtggtgcaggcccggctctgcgtctatccctactacacagccacctgttgccgctcttgtgcccatgtcctggagcggtcttccccagagcccgcctga

SEQ ID NO: 154 - protein, 1354 aa, Bos taurus
MGISREGIPWMPEAKYHTFVRVTVTVIHAGGSHRFPPLGERDPATSKQEQGRGTGLCSWRFVSFTSVNFVRHQHNLARKHVTAQSRQVGGLKRVPTSQANFPKSGLDRLASPAGVHKTPVELRNEITQTGDQTSASTLTVPPPSTALLSVILGRCLPSAFLPSAAAGSVPAHLTFWSRNRKAQGACQAGKTSQNSVLVARLLPDFFPSNPRCGARGSPTPLGYRRHPTPWRSSRPPRLGWRSARTAHSEPVGIFGSRRPNPSRVEGQPWRDGSLGTRLGPPRLRPPGAVRRPQLCLMLLLSLPQLCLDQEVLSGHSLQTPPEESQGPEGVWGPWDQWASCSQPCGVGVQRRSRTCQLPTTQLHQGLPLPPRPPRHPEALLPRGQGTRPQTSRETLPLYRPPPRGRGGPLRGPASQLGREEAPETQGARRSRVRDPIKPGMFGYGRVPFALPLHRNRRHPRRPPRSELSQTSDLPSLTPRTEPSSSNHTQKTELSPTEPSAHTLPPQAEPPSPEAAQTAVPSRARPAPTGPHPRAQASGTESSFHSPSPGEGSSFHLSLQPRRPSSQGWASPRLADRHPNPFLSVPWGRGQQSQEQWRPGGNLHGSLTESAPPQPDGWLPLLSSGPHSSPLWSLFAPSSPVPRCSGESEQLRACSQAPCPAEQPDPRALQCAAFDSQEFMGQLYQWEPFTEVQGSQRCELNCRPRGFRFYVRHTQKVQDGTLCQPGSLDICVAGRCLSPGCDGILGSGRHPDGCGVCGGDDSTCRLVLGNLTERGGPLGYQKILSIPAGASRLQIAQLRPSSNYLALRGPGGRSIINGNWAVDPPGSYTAGGTVFRYNRPPREEGAGESLSAEGPTTQPVDVYMIFQEENPGVFYQYVISSPPPNLENPTPEPRVPQLQPEILRVEPPPVSAPRPARTPGTLQRQVRIPQMPAPPPPRTPLGSPAGYWKRVGHSECSASCGKGVWRPIFLCISRESGEELDERSCAMGARPPASQEPCHGPPCPPYWEAGEWTSCSRSCGPGTQHRQLRCRQEFGGGGSSVPLERCGHLPRPNITQPCQLRLCGHWEVRSPWSQCSVRCGRGQRSRQVRCVGNNGDEVSEQECASGPPRPPSREACDMGPCTTAWFHSDWSSKCSAECGTGIQRRSVVCLGSGEAHGISQEEAGAGAGEQTCAPGSRPPDMRACSLGPCEVSWCWYTGPWAECSSECGSGTQRRDVICVSKLGTEFNVTSPSNCSHLPRPPALQPCQGQDCQDRWFSTPWSPCSRSCQGGVQTREVQCLTANQTLSIRCPLHLRPARKRSCNSQPCSQRPDDQCKDSSPHCPLVVQARLCVYPYYTATCCRSCAHVLERSSPEPA

SEQ ID NO: 155 - DNA, 3897 bp, Bos taurus
atgacggcccctgctcagcccaagaaaatcgtggcccctacggtgtcccagatcaacgcagagttcgtgacccagttagcatgtaaatattgggcaccccatatcaagaaaaagtcaccctttgatataaaggtgattgaagatatatatgagaaagaaattgtcaaatcaaggtttgctatcagaaagataatgctcttggaatttagccagtatcttgaaaattacctgtggatgaattattctccagaagtgtccagcaaagcctatttaatgtcaatctgctgtatggtgaatgaaaaattcagagaaaatgtccctgcatgggagacttttaagaagaagccagatcacttcccattctttttcaagtgcatcttgaaagcagcattagctgaaactgatggtgaattttcactccatgaacagacagtcttactgctttttcttgaccactgcttcaatagtttggaagtagacttgatacggagtcaagtacagcagcttatctctctcccgatgtggatgggcttacagcctgcacgattggaattagaattaaaaaagacacctaagctaagaaaattctggaacttgattaaaaagaatgatgaaaagatggatccagaagcaagagaacaagcctatcaagaaagaagatttctttcacaactcatccagaagtttatctctgtgctgaaatcagtcccactttctgatcctgtcactatggacaaagttcattactgtgaaagatttattgaacttatgattgatctagagctcttggacatgcttaagttctatactggttttgagattaacgaccagactggaaacgctctgacagagaatgagatgacaactattcactatgatagaatcacttctctccagagagctgcttttgcacatttccctgaactctatgattttgctctctcaaatgtggcagaagtagatactcgggaatccttggtcaagttttttggacctcttagttccaataccctccaccaggtggcatcatacctctgcttgttaccaacccttcctaaagctgaagacacgacttttgataaagggtttcttctagaattgttggtgtctcgtcatgagcgtcgaatttctcagattcagcagttgaaccagatgcctttgtatccaactgagaagatcatatgggatgaaaatattgtcccaactgaatactattctggggaaggccttcggaagcatgatgtatgctttttaattaccgtgcgtcccacaaaaccttatggtaccaagtttgaccgaagaagaccttttattgagcaggttggcctagtttatgtcagaggctgtgaaatccagggcatgctagatgataaagggcgtgtcattgaagacggacctgaacccagacccagtcttagaggagaatcaaggacttttagagtgtttttggatccaaatcagtatcaacaagatatgaccaatactatacaaaatggagcagaagatgtgtatgaaactttcaatgtaataatgagaagaaaaccaaaggaaaataactttaaggctgtgctggagactatccggaacctgatgaatactgattgtgtggtacctgactggctgcatgacatcattttaggttatggggatccaagtagtgcacattattccaaaatgcccaatcagattgccacccttgatttcaatgatacatttctctccattgaacacttaaaagccagctttcctggtcataatgttaaagtaactgtggatgacccagctctacagataccccctttcaggataacttttccagtaagaagtggaaaggggaagaagcggaaagatgtagacggggaagatgaagacactgaggaggcaaaaactttaattgtcgaacctcatgttattcctaatagaggtccttatccttacaaccaacccaaacgtaatacaattcagttcactcatacacagatagaggccatccgtgctggaatgcagcctgggctaactatggttgtgggcccaccaggtacaggaaaaacagatgtggcagttcaaattatatccaacatctaccacaatttcccagagcagaggactctgattgttactcattccaatcaggccctgaaccagttgtttgagaaaatcatggctttagacattgacgagcgccacctcctgcgtcttggtcatggagaagaagagctggagacagagaaagatttcagcagtattatttcaggaatttattgccagtttagctggactttattctggttggtgaaaaacaaaagtaacacgatgccagatattactgaagtctccgctttcttcccttttcatgagtactttgcaaatgcccctcaacccatttttaaaggccgatcttatgaagaagacatggaaattgctgaaggctgtttcaggcatattaagaaaatctttactcagcttgaggaattcagagcctctgaactgcttcgaagtggactagacagatctaaataccttttggtgaaagaagccaaaatcattgctatgacctgtactcatgctgccttaaaacgacatgatttagtcaagttaggtttcaagtatgacaacattttaatggaggaagctgctcagattctggagatagaaacttttatacctcttcttctacagaatcctcaggatggttttagccgactaaaacgctggattatgattggtgatcatcaccagttacctccagtcattaagaacatggcctttcagaagtattcaaacatggaacagtccctcttcactcgctttgtcagggttggagttcctactgtggaccttgatgcccaggggagggccagagcaagcttgtgcaacctctacaactggcggtataagaatctaggaaacttaccccatgtgcagctcttgctggagttcagtacagccaatgctggcttgctctatgacttccagctcattaatgttgaagattttcagggagttggagaatctgagcctaatccttacttttatcagaatcttggagaggcagaatacgtagtggcactttttatgtacatgtgcttacttggctaccctgctgacaagatcagcattctgacaacgtataatgggcaaaagcatcttattcgtgacatcatcaacagacgatgcgggagcaatccattgattggaagaccaaataaggtgacaactgttgacagatttcaaggtcaacagaacgactacattcttctgtctctagtacgaaccagggcagtgggccatctgagggatgtccgtcgcttagtggttgccatgtctagagccagacttggactctatatcttcgccagagtatccctcttccagaactgttttgaactaactccagctttcagccagctcacagcccgcccacttcatttgcatataattccaacagaacctttcccaaccagtagaaagaatggagagagaccatcccatgaagtacagataataaagaatatgccccagatggcaaactttgtatataatatgtatatgcatttgatacagaccacacatcattatcatcagactttattacagctaccacctgctatggtagaagaaagtgaggaagttcaaagtcaggaaacagaattggaaacagaagaagaggccatgtctgctcaagctgacattgtacctgatgaagcaacagatgccagctccagtcaggaaacctcagcctctgagactgaaaccacccccaaccagacaggagccagctccagtccagaggctatccctgctgagtctgagatcactgggacaggaccggtaactgtaccatcggagaataacacccctcatgatgtcacatccactcccggagaaaccgagtag

SEQ ID NO: 156 - protein, 1298 aa, Bos taurus
MTAPAQPKKIVAPTVSQINAEFVTQLACKYWAPHIKKKSPFDIKVIEDIYEKEIVKSRFAIRKIMLLEFSQYLENYLWMNYSPEVSSKAYLMSICCMVNEKFRENVPAWETFKKKPDHFPFFFKCILKAALAETDGEFSLHEQTVLLLFLDHCFNSLEVDLIRSQVQQLISLPMWMGLQPARLELELKKTPKLRKFWNLIKKNDEKMDPEAREQAYQERRFLSQLIQKFISVLKSVPLSDPVTMDKVHYCERFIELMIDLELLDMLKFYTGFEINDQTGNALTENEMTTIHYDRITSLQRAAFAHFPELYDFALSNVAEVDTRESLVKFFGPLSSNTLHQVASYLCLLPTLPKAEDTTFDKGFLLELLVSRHERRISQIQQLNQMPLYPTEKIIWDENIVPTEYYSGEGLRKHDVCFLITVRPTKPYGTKFDRRRPFIEQVGLVYVRGCEIQGMLDDKGRVIEDGPEPRPSLRGESRTFRVFLDPNQYQQDMTNTIQNGAEDVYETFNVIMRRKPKENNFKAVLETIRNLMNTDCVVPDWLHDIILGYGDPSSAHYSKMPNQIATLDFNDTFLSIEHLKASFPGHNVKVTVDDPALQIPPFRITFPVRSGKGKKRKDVDGEDEDTEEAKTLIVEPHVIPNRGPYPYNQPKRNTIQFTHTQIEAIRAGMQPGLTMVVGPPGTGKTDVAVQIISNIYHNFPEQRTLIVTHSNQALNQLFEKIMALDIDERHLLRLGHGEEELETEKDFSSIISGIYCQFSWTLFWLVKNKSNTMPDITEVSAFFPFHEYFANAPQPIFKGRSYEEDMEIAEGCFRHIKKIFTQLEEFRASELLRSGLDRSKYLLVKEAKIIAMTCTHAALKRHDLVKLGFKYDNILMEEAAQILEIETFIPLLLQNPQDGFSRLKRWIMIGDHHQLPPVIKNMAFQKYSNMEQSLFTRFVRVGVPTVDLDAQGRARASLCNLYNWRYKNLGNLPHVQLLLEFSTANAGLLYDFQLINVEDFQGVGESEPNPYFYQNLGEAEYVVALFMYMCLLGYPADKISILTTYNGQKHLIRDIINRRCGSNPLIGRPNKVTTVDRFQGQQNDYILLSLVRTRAVGHLRDVRRLVVAMSRARLGLYIFARVSLFQNCFELTPAFSQLTARPLHLHIIPTEPFPTSRKNGERPSHEVQIIKNMPQMANFVYNMYMHLIQTTHHYHQTLLQLPPAMVEESEEVQSQETELETEEEAMSAQADIVPDEATDASSSQETSASETETTPNQTGASSSPEAIPAESEITGTGPVTVPSENNTPHDVTSTPGETE

SEQ ID NO: 157 - DNA, 339 bp, Bos taurus
atggcctttgggaagcctgccaagtactggaagctggaccccgctcaggtctacgccagcgggcccaacgcgtgggacaccgctgtgcacgacgcctctgaggagtacaagcaccgcatgcacaatctctgctgtgacaactgccactcgcacgtggccttggccctgaacctgatgcgctacaacaacagcaccaactggaacatggtgacgctctgcttcttctgcctgctctatgggaagtacgtcagcgtcggcgccttcgtgaagacctggctgccttttgtccttctcctgggcatcatcctgaccatcagcttggtcttcaacctgcggtga

SEQ ID NO: 158 - protein, 112 aa, Bos taurus
MAFGKPAKYWKLDPAQVYASGPNAWDTAVHDASEEYKHRMHNLCCDNCHSHVALALNLMRYNNSTNWNMVTLCFFCLLYGKYVSVGAFVKTWLPFVLLLGIILTISLVFNLR

SEQ ID NO: 159 - DNA, 937 bp, Bos taurus
atggctgcggctcgcgcaccccgcgctctgacctctgcctcgcctgggtctgggaaagccaaactgacacacccagggaaggcgatcctagcaggcggcctggctggcggcatcgaaatctgcatcactttccccaccgagtacgtgaagacgcagctgcagctggacgagcgctcgcacccgccgcgctaccggggcatcggggactgcgtgcggcagacggttcgcagccatgggctcctgggcctgtaccgcggccttagctccctgctctacggctccatccccaaggcggccgtcaggttcgggacgttcgaatttctcagcaaccacatgcgggacgcccagggacggctggacagcacgcgcgggctgctgtgcggcctgggcgccggagtgccagaggccgtggtggtcgtgtgccccatggagaccatcaaggtgaaatttatccatgaccagacctctgccagccccaaataccgaggattcttccacggggtcagggagatcgtgcgggaacaggggctgaaggggacgtatcaaggcctcacagccaccgtgctgaagcagggatccaatcagggcatccgcttcttcgtcatgacttccctgcgcaactggtaccgaggagacaaccccaacaaacccatgaacccactgatcactggtgtgttcggagccatcgcaggcgcagccagtgtcttcgggaacactccactggacgtgatcaagacccggatgcagggactggaagcacacaagtaccggaacacgttggactgcggcctgcagatcctgaggaacgagggactcaaggcgttctacaagggcactgtcccgcgcctcggccgagtctgcctggacgtggccatcgtgttcatcatctacgacgaggtggtgaagcttctcaacaaagtgtggaaggcggactgag

SEQ ID NO: 160 - protein, 311 aa, Bos taurus
MAAARAPRALTSASPGSGKAKLTHPGKAILAGGLAGGIEICITFPTEYVKTQLQLDERSHPPRYRGIGDCVRQTVRSHGLLGLYRGLSSLLYGSIPKAAVRFGTFEFLSNHMRDAQGRLDSTRGLLCGLGAGVPEAVVVVCPMETIKVKFIHDQTSASPKYRGFFHGVREIVREQGLKGTYQGLTATVLKQGSNQGIRFFVMTSLRNWYRGDNPNKPMNPLITGVFGAIAGAASVFGNTPLDVIKTRMQGLEAHKYRNTLDCGLQILRNEGLKAFYKGTVPRLGRVCLDVAIVFIIYDEVVKLLNKVWKAD

SEQ ID NO: 161 - DNA, 1071 bp, Bos taurus
atgggggccagctcaggagccagcgcaggggcccgggaaaggcagggaacaggagcggagggaggccctgacttggtctgcagtggtgggggcgcagtctggagagtggtgctggcggggctgccacagagcacagactgctgggccaggaatgccgctgacaacgtctccgccactcccgggcagcctccccaccaggctcagaaagtccctgttgctcattccagttcccggggcccaggcctctctttccagtggaaggacactcactcgtaccacgttagaggcaggagcatggttttacgggtctgggcccactacgaggagcagccggtggaggagttgatgcccgtgctggaggagaaggagcgctcggccagctacaagccggtgttcgtgacggagatcaccgacgacctgcacttctacgtgcaggacgtggagacaggcacccagctggagaagctgatggagaacatgcgcaatgacattgctagccaccctcccgtcgagggctcctacgccccccgccggggcgaattctgcatcgccaaattcgtagacggagaatggtaccgtgcccgagtggagaaagtcgagtctcctgccaaagtacacgtcttctacatcgactacggcaacagggagatcctgccgtccacccgcctgggcactttgccacctgccttcagcacccgcgtgctaccagctcaagccacggagtatgcctttgccttcatccaggtgccccaagatgaggatgctcggactgacgcggtggacagcgtggtgcgggacatccagaacacgcagtgcctgctcaacgtggagcacctgagcgccggctgcccgcacgtcaccctgcagttcgctgactccaagggggacgtggggctgggcctggtgaaggaggggctggtcatggtggaggtgcgcaaggaaaagcagttccagaaagtgatcacagaatacctgaatgcccaggagtcagccaagagcgccaggctgaacctgtggcgctatggagacttccgagccgacgatgcagatgagtttggctacagccgctaa

SEQ ID NO: 162 - protein, 356 aa, Bos taurus
MGASSGASAGARERQGTGAEGGPDLVCSGGGAVWRVVLAGLPQSTDCWARNAADNVSATPGQPPHQAQKVPVAHSSSRGPGLSFQWKDTHSYHVRGRSMVLRVWAHYEEQPVEELMPVLEEKERSASYKPVFVTEITDDLHFYVQDVETGTQLEKLMENMRNDIASHPPVEGSYAPRRGEFCIAKFVDGEWYRARVEKVESPAKVHVFYIDYGNREILPSTRLGTLPPAFSTRVLPAQATEYAFAFIQVPQDEDARTDAVDSVVRDIQNTQCLLNVEHLSAGCPHVTLQFADSKGDVGLGLVKEGLVMVEVRKEKQFQKVITEYLNAQESAKSARLNLWRYGDFRADDADEFGYSR

SEQ ID NO: 163 - DNA, 1002 bp, Bos taurus
atgaacttccaggtgacaggcctgggcctggacaaaattaagctggacagtccccagtccttcctggaccaggaggaggtggaggaggccgaagatgggcagctgctggagcccgaggcttggcgaacctatgtggagcgccgcaacgctctgcaagagttcctgacctcagacctgagcccccacctgctcaagcgccaccacgcccgcatggagctcctcaagaagtgctcctactacatcgagatcctccccaagcacctggctctgggcgaccagaacccactggtgctgcccaccaccatgttccagctcatcgacccctggaagttccagcgcatgaagaaagtgggcgccgcccagaccaagatccagctcctgctgcttggggacctgctggagcagctggaccatggccgctccgagctggatgccctgctcgagtcgccagaccctcggcccttcctggcaggctgggggctcgtggagcagcgcctggcggacctgtcggctgtcatggacagcttcctggccatgatggtgccggggcgcctacacatcaagcaccgcctagtgtctgacattggcgccaccaagattccccacatccggctcatgctgagcaccaagatgcccgttatgtttgaccgcaaggagtcggtggcccaccaggactgggtcagcctgcgctggtttgtcaccatccagcaggctgtgccggagcagtttgagctgcgctacaagctgctggacccacggacacagcaggagtgcatgcagtgtggcatcatccccgtggccgcctgcgcctttgacatccgaaacctgttgcccaaccgtgcctacaagttcactgtcaagagggcggagagctacactctggtgtatgagccctggagggacagcctcaccctgcacacccggccagggccccctgaagggctcgcccccagtaggctgggcaagctaggcctgtccctgaccacaccttccgagagatga

SEQ ID NO: 164 - protein, 333 aa, Bos taurus
MNFQVTGLGLDKIKLDSPQSFLDQEEVEEAEDGQLLEPEAWRTYVERRNALQEFLTSDLSPHLLKRHHARMELLKKCSYYIEILPKHLALGDQNPLVLPTTMFQLIDPWKFQRMKKVGAAQTKIQLLLLGDLLEQLDHGRSELDALLESPDPRPFLAGWGLVEQRLADLSAVMDSFLAMMVPGRLHIKHRLVSDIGATKIPHIRLMLSTKMPVMFDRKESVAHQDWVSLRWFVTIQQAVPEQFELRYKLLDPRTQQECMQCGIIPVAACAFDIRNLLPNRAYKFTVKRAESYTLVYEPWRDSLTLHTRPGPPEGLAPSRLGKLGLSLTTPSER

SEQ ID NO: 165 - DNA, 2331 bp, Bos taurus
atggacctccacacacagtccgaggaggacaggataaacgggatgctcctgagtccgaggagacccacgatcgagaagatggccagccggaagtcctcagtctcgcacacggcccaccggcgcatggtggtcagcatgcccaacctgcaggacatcacggtgcctgagctgccgccccggaactcgtcgcttcggaagatacagaccacggagtatgggagcagccacacagttctggacgtgccccccaagtcgacgcacaccttcaagatcccagaaatccacttcccgctggtggcccagtgcgtgcaggcattctacgcagactgcgtggcccagctgagtaaagccatgggctcactggcccccgagaactcagtgctgctggccaggtacttctacctgagggggctggtgcagctgatgcagggccagctgctggatgctctgctggacttccagagcctgtacaagaccgatgtgcgtgtcttccccaccgacctcgtccagacgaccgtggagtccctgtcggcacccgagcgcacgcaggctgagcgcatgcctgagctgcgccggctcatcagcgaggtcatggacaggcccgcggacgtccccaaggccgacgaccgcgtcaagaacttcgagctgcccaagaagcacatgcagctggacgatttcgtgaagagggtgcaggagtcgggcatcgtgaaggacaccgtcatcatacaccggctgttcgaggcgctgactgtaggacacgagaaacagattgacccagaaacattcaaagatttctacaactgctggaaggaggcagaagcagaggcgcaggaggtcagcctgcctttgtcggtgatggagcagctggacaaaagtgagtgtgtgtacaagctgtcgcgctccgtcaagacaaatcgcggggtgggcaagatcgccatgacccagaagcgcctgttccttctcacggaggggcggccaggctacgtggagatctccaccttcaggaacatagaggatgtcagaagcaccatggccacttttctgctcctgagaattcccactttaaaaatcaaaacgacgtccaagaaagaagtctttgaagctaacctaaagacggagtgtgacctctggcacctgatggtgaaggagatgtgggccgggaggaagctggccgacgaccacaaggacccccagtacgtacagcaggcgctgacccacattctcttgatggacgcggtcgtcggcacgctgcagtcaccgggagccatctatgcggcctccaagttgtcctactttgataggatgaagaatgaaatgcccatggccattccgaagacgacctcggaaaccctgaaacacaagatcaacccctcggagggggagaccaccccacgagccgtcgacgtgctgctctacaccccagggcatcttgacccagcagaaaaagtggaggatgctcaccccaaattatggtgcgcactgaatgagggcaaggtgatcgtatttgacgcttcctcctggacgattcaccagcactgctttagagtgggcacttctaagctgaactgcatggtgatggcggagcagagccaggtgtgggtcggctcggaggactcggtcatctacatcatcaatgtccacagcatgtcctgcaacaagcagctcaccgaccaccgctccagcgtcacgggcctggctgtgcaggacggggagcaggcgcccaggatttcaatatgcaaccttaacttctcactacataccttcaataactattacaccacttcatgtttaatggacacagtgcacagcagtagaattaattccatttacctttctttccccttcctgttgttgttaaatgccatattttgttgctatgattacagcatcctgatggtgaacatgaatggatcttcttgtcaaaagctgaagatcactgggagctttaaagagacaggcacctccttcctgggattccagctcatgcctgagcaggagcagctctgggcggcctgtgctgggtaccccgatgtctatgtctggagcctgcaggacccagcccagcccccgcagaggatccacctccaggactgctctgagatcaactgcatgatcagggtgaagaagcagatctgggtaggcaccacggggctgtcgcaggggcagctgaaggggaagatctatgtgatggatgcggagaggaggacggtggagaaggagctggtggcccacgcggacacggtgaagacactgtgctccgcggaggacagatacgtgctgagcggcgccggccctgaggaggggaagatcgccatctggaaggtcgagtga

SEQ ID NO: 166 - protein, 776 aa, Bos taurus
MDLHTQSEEDRINGMLLSPRRPTIEKMASRKSSVSHTAHRRMVVSMPNLQDITVPELPPRNSSLRKIQTTEYGSSHTVLDVPPKSTHTFKIPEIHFPLVAQCVQAFYADCVAQLSKAMGSLAPENSVLLARYFYLRGLVQLMQGQLLDALLDFQSLYKTDVRVFPTDLVQTTVESLSAPERTQAERMPELRRLISEVMDRPADVPKADDRVKNFELPKKHMQLDDFVKRVQESGIVKDTVIIHRLFEALTVGHEKQIDPETFKDFYNCWKEAEAEAQEVSLPLSVMEQLDKSECVYKLSRSVKTNRGVGKIAMTQKRLFLLTEGRPGYVEISTFRNIEDVRSTMATFLLLRIPTLKIKTTSKKEVFEANLKTECDLWHLMVKEMWAGRKLADDHKDPQYVQQALTHILLMDAVVGTLQSPGAIYAASKLSYFDRMKNEMPMAIPKTTSETLKHKINPSEGETTPRAVDVLLYTPGHLDPAEKVEDAHPKLWCALNEGKVIVFDASSWTIHQHCFRVGTSKLNCMVMAEQSQVWVGSEDSVIYIINVHSMSCNKQLTDHRSSVTGLAVQDGEQAPRISICNLNFSLHTFNNYYTTSCLMDTVHSSRINSIYLSFPFLLLLNAIFCCYDYSILMVNMNGSSCQKLKITGSFKETGTSFLGFQLMPEQEQLWAACAGYPDVYVWSLQDPAQPPQRIHLQDCSEINCMIRVKKQIWVGTTGLSQGQLKGKIYVMDAERRTVEKELVAHADTVKTLCSAEDRYVLSGAGPEEGKIAIWKVE

SEQ ID NO: 167 - DNA, 517 bp, Macropus eugenii
ggacaccaagaggatgaggggcttaaccatgcaggtactcctattgctggggctgctcagcctgatgactccacttggctatgcccaggaccagccataccaggatgtgctgaatagattcattcaggaatacaacacaaagtcagaatcagaaagcctctttcgtctctcagtcctgaatctgccatcacaggagagcaacgatcctactgctccacaacttctgaaattcaccatcagagagactgtgtgctctaaaagtgaacatcgcaatccagaagaatgtgatttcaagaaaaatgggctggtggaagagtgcattggaacagttgacctggattcctccagtccctctgttgatatttcctgtgatgggcctgaaaaggtcaagagaggatttgggaagaagttgaggaagagactgaagaaatttcggaacagcattaagaaaagattaaagaactttaacgttgtaattcctatcccactgccagggtagcctcctgtatgcgactga

SEQ ID NO: 168 - DNA, 626 bp, Macropus eugenii
  variation (596)...(597): n = g, a, t or c
  variation (606)...(608): n = g, a, t or c
ggggaccaagaggatgagggactcaaccatgcaggtactcctattggtgctggggctactcagcctgatgacttcacttgcctgtgcccaggaccagccataccagatgtgctgaatagattcattcaggaatacaacacaaagtcagaatcagaaagcctctttcgtctctcagtcctgaatttgccaccggaggagagcaacgatcctgctgttccacttctgaaattcaccatcagagagactgtgtgccccaaaactgaacatcgcaatgcagatgaatgtgacttcaagaaaaatgggctggtgaaacaatgcatttgaaccattgacctggattctcccaatccttctgttgatatttcctgtgataggcccgcgaaggtcaagagaggactttgggagagtttgaagaggaaagcgacgaaactcggggatgatattaggaatacattaaggaactttaaaattaaatttcctgtcccccgccaggggtagacacgtgtatgagactgagcctcagtatccatcttaaggccttgtactatgccttggtctgttcagattaataaacgccaattttactctctctccanngaaaaaaannnaaaaaaaaaaaaaaaaaa

SEQ ID NO: 169 - DNA, 872 bp, Macropus eugenii
ggacaccaagaggatgaggggcttaaccatgcaggtactcctattgcctttggggctgctcagcctgatgactccacttggctatgcccaggaccagccataccaggatgtgctgaatagattcattcaggaatacaacacaaagtcagaatcagaaagcctctttcgtctctcagtcctgaatctgccatcacaggagagcaacgatcctactgctccacaacttctgaaattcaccatcagagagactgtgtgctctaaaagtgaacatcgcaatccagaagaatgtgatttcaagaaaaatggggtgagagtgtcctgggctgggaagggattctgactgaaggaggctctttctggggatatgcaccccttggggcaggaggggttctgagctctgcttttctgctgccctgaggaaagtgatttgtcccacatgagaagcagaaaaactctcccaggggactggctggggacaaagcttgagcttgttgtcagtcctggcctgtgagggacagacattacagactggcagtcctgaactctctctgccacccttcacagctggtggaagagtgcattggaacagttgacctggattcctccagtccctctgttgatatttcctgtgatgggcctgaaaaggtcaagagaggatttgggaagaagttgaggaagagactgaagaaatttcggaacagcattaagaaaagattaaagaactttaacgttgtaattcctatctcactgccagggtagcctcctgtatgcgactgagcctcagttctctatcttaaggccttgcactataccttggtctgctcagattaataaacctcaattttactctcagaaaaaaaaaaaaaaaaaaa

SEQ ID NO: 170 - DNA, 474 bp, Bos taurus
atggagaccccgagggccagcctctccctgggacggtggtcactgtggctactgctgctggggctagcgctgccctcggcgcagcgcccaggccctcagctacagggaggccgtgcttcgtgtctgatggagtcagcatcaatgaagaagtcctcagaagctaatctctaccgcctcctggagctagacccacctcccaaggaggacgatgagaacccaaacatcccgaagcctgtgagcttcagggtgaaggagaccgtgtgccccaggacgagccagcagtccccggagcagtgtgacttcaaggagaatgggctgctgaaatattgcgtggggacagtgagccggtgggaccaggtcgggagtaacttcgacatcacctgtgctgcgccccagagtgtcgggggacttcaagcctgggtaggaagatattacgtacttggaaaaagtatggcccaattattgttccaataa

SEQ ID NO: 171 - protein, 157 aa, Bos taurus
METPRASLSLGRWSLWLLLLGLALPSAQRPGPQLQGGRASCLMESASMKKSSEANLYRLLELDPPPKEDDENPNIPKPVSFRVKETVCPRTSQQSPEQCDFKENGLLKYCVGTVSRWDQVGSNFDITCAAPQSVGGLQAWVGRYYVLGKSMAQLLFQ

SEQ ID NO: 172 - DNA, 609 bp, Bos taurus
atggagaccccgagggccagcctctccctgggacggtgttcactgtggctactgctgctgggactagtgctgccctcggccagcgcccaggccctcagctacagggaggccgtgcttcgtgccgtggatcagttcaatgagcggtcctcagaagctaatctctaccgcctcctggagctagaccctacacccaatgatgtgagttatgaagggatctgggcagggcggccagctagttggggggcagggagacagatcagaggaagaagaatgagcccaaatccagtttcccctactttgacccatgaccaggacttggacccaggcaccagaaagcctgtgagcttcagggtgaaggagaccgattgccccaggacaagccagcagcccctggagcagtgtgacttcaaggagaatggggtgagcctgggagcgggggagaagaatggcagatgcttcccagggattggacagcgggcttggggacgattaccaattcctgggaggaggccgggaggttatggccacaggggtttcagtttgaccttgaacctccccttccagctggtgaaaacagtgtgtgggacagcacgacatcagcataactga

SEQ ID NO: 173 - protein, 202 aa, Bos taurus
METPRASLSLGRCSLWLLLLGLVLPSASAQALSYREAVLRAVDQFNERSSEANLYRLLELDPTPNDVSYEGIWAGRPASWGAGRQIRGRRMSPNPVSPTLTHDQDLDPGTRKPVSFRVKETDCPRTSQQPLEQCDFKENGVSLGAGEKNGRCFPGIGQRAWGRLPIPGRRPGGYGHRGFSLTLNLPFQLVKTVCGTARHQHN

SEQ ID NO: 174 - DNA, 464 bp, Bos taurus
atggagacccagagggccagcctctccctggggcgctggtcgctgtggctgctgctgctgggactagtgctgccctcggccagcgcccaggccctcagctacagggaggccatgcttcgtgctgtggatcagctcaatgagcggtcctcagaagctcatctctaccgcctcctggagctagacccgcctccctaggacatgagctggggaggggaccggggacgagcctttctcctgcctgctttggccacactgttgccccttcactctggctgtacctcctgtcaggaaggcacttttccctctaggtgggttcccacctcttccaggaaagcttcccagacctgggtcatctcccaacaccaggcttcctgtcttagcatctctgctgtgggaataggcaccctgcacacctggctcaagttccctggacttctgggagctccagggatgggggggtcaca

SEQ ID NO: 175 - protein, 153 aa, Bos taurus
METQRASLSLGRWSLWLLLLGLVLPSASAQALSYREAMLRAVDQLNERSSEAHLYRLLELDPPPDMSWGGDRGRAFLLPALATLLPLHSGCTSCQEGTFPSRWVPTSSRKASQTWVISQHQASCLSISAVGIGTLHTWLKFPGLLGAPGMGGS

SEQ ID NO: 176 - DNA, 543 bp, Bos taurus
atggagacccagagggccagcctctccctggggcgctggtcgctgtggctgctgctgctgggactagtgctgccctcggccagcgcccaggccctcagctacagggaggccatgcttcgtgctgtggatcagctcaatgagcggtcctcagaagctcatctctaccgcctcctaggagctagcacccgtcctccctcaggacatgagctggggaggggtaccggggacgagcctttctcctgccctgctttggccacactgttgccccttcactctggctgtacctcctgtcaggaaggcacttttccctctaggtgggttcccacctcttccaggaaagccgtgtcccgagacctgggtcatctcccagcaccaggctatccctgtcttagcatctctgctgtgggaataggcaccctgcacacctggctcaggttccctggacttctgggcagctccagggatgggggggtcacaggctctgtgaggtgacttccctgccaatgtcctctccgcaacctcggtgtctctctgccaggagga

SEQ ID NO: 177 - protein, 163 aa, Bos taurus
METQRASLSLGRWSLWLLLLGLVLPSASAQALSYREAMLRAVDQLNERSSEAHLYRLLGASTRPPSGHELGRGTGDEPFSCPALATLLPLHSGCTSCQEGTFPSRWVPTSSRKAVSRDLGHLPAPGYPCLSISAVGIGTLHTWLRFPGLLGSSRDGGVTGSVR

SEQ ID NO: 178 - DNA, 718 bp, Macropus eugenii
ccagttctcagtgccttgggtattactgaagccaggtttccaagatttgcagccagattggtctttgaactgtggcaagatggggaaagacctgctgatcattttatccgtattctttataatggagttgatgttacattccacacctctttttgccaggaacaccacaagcattccagcaagcctatgtgtcctcttgaagattttgttcactttgtcaaaagggacatgttttcagtcttaaatagtagtagttattatgatgcatgtcatagaaaaacattttaaaagaaaatcattctgtatttgtgaaatttgtgctggtacagaagatgaccacttgagattctttctgattcaaaagggtacgacagtattgctttgaaaatacttagaaatccttgcttgggaccatataggttgagtgacttgaatagcaaataatttactatgatttggtacatgatttagttcattaatttggtgcagttctacttcattcagaaacagaagtactctgctgtaaattgatattgcttggaataaatgattttcctccttatgccagaatagataagtttaggttgccaattgagaatttcattctcttgactgccatggtactataatatgtaaccaacaaacctcatccacaatgtttcttaatcttgtgcagttatagttactccttaaatgaaattattcctaaattggatgt

SEQ ID NO: 179 - protein, 77 aa, Macropus eugenii
  SITE 4: Xaa = any amino acid
MSLXDKVNKIFKRTHRLAGMLVVFLAKRGVECNINSIIKNTDKMISRSFPILPQFKDQSGCKSWKPGFSNTQGTENW

SEQ ID NO: 180 - DNA, 1272 bp, Bos taurus
atgcttttccgcaatcgcttcgtgctgctgctggccctggctgcactgctggccttcgtgagcctcagcctgcagttcttccacctgatcccagtgttggcaaccaagaatggagtgaccagcaagagtcgaaagagaatcatgcctgacccggcaacggagccccccgtgatggacccggtatatgaagcccttttgtactgcaacattccaggtgtggctgagcgcagcatggaaggccacgccccacatcattttaagctggtctctgcgcacgtgttcattcgacacggggacaggtacccactgtacgccattcccaaaacgaagcgcccggaaattgactgcactctggtggctaacaggaagccgtatcactctaaactggaggcttttgtgaatcacatgtcaaaaggacctggagccgctttcgaaagccccttgcactcgctgcctctttactccagtcaccagctgtgtgagatgggagagctcacgcagacaggggtcgtgcagcacctgcagaatggccagctgctgagggacatctacttgaagaaacacaagctcctgccgagtgactgggctgcagagcagcttcacttagagaccactggcaagagccggacgctgcagagcgggctggctctgctctatggctttctgccagattttgactggaagaaggtttatttcaggcaccagcccagtgctctgttctgctctgggaactgctattgcccactgaggaaccagtacctggagaaggagcagcgtcggcagtacctattacgtttgaataacaggcagctggaaaggacctacgaggagatggcccggatcgtggacatccccaccaagcagctccgggctgccaaccccatagactccctgctctgcctcttctgccacaacgtcagcttcccctgcaccaagaatggctgtatcaccatggaccacttcaaggtgatcaagacgcatcagatagaggatgagagagccttacgggagccagattcccacggatttgcagccaggttggtctttgagctctggcaggacgagaaaccccgggagcactccatccggatcctctatgacggtgttgacatcacgttctacacttcattctgccaggaccaccacaggcggtctcccaagcccatgtgtcccctcgaaaatttagtccgcttcatcaaaaaggacatgtttgtggccctgggtggtggtagtaccaattattatgaagcatgtcacagggaagggttctaa

SEQ ID NO: 181 - protein, 423 aa, Bos taurus
MLFRNRFVLLLALAALLAFVSLSLQFFHLIPVLATKNGVTSKSRKRIMPDPATEPPVMDPVYEALLYCNIPGVAERSMEGHAPHHFKLVSAHVFIRHGDRYPLYAIPKTKRPEIDCTLVANRKPYHSKLEAFVNHMSKGPGAAFESPLHSLPLYSSHQLCEMGELTQTGVVQHLQNGQLLRDIYLKKHKLLPSDWAAEQLHLETTGKSRTLQSGLALLYGFLPDFDWKKVYFRHQPSALFCSGNCYCPLRNQYLEKEQRRQYLLRLNNRQLERTYEEMARIVDIPTKQLRAANPIDSLLCLFCHNVSFPCTKNGCITMDHFKVIKTHQIEDERALREPDSHGFAARLVFELWQDEKPREHSIRILYDGVDITFYTSFCQDHHRRSPKPMCPLENLVRFIKKDMFVALGGGSTNYYEACHREGF

SEQ ID NO: 182 - skipped

SEQ ID NO: 183 - DNA, 990 bp, Macropus eugenii
  variation 962: n = g, a, t or c
cggactcgttttctttgtatcctcagttcatgttccatcttcgaagatccccatttcttcaagtgtttaataacagtccagatgaatcatcttattaccgacatcattttgccagacaagacctgacccagtctctcatcatgatccagcctattctttattcttattctttccatggtccacctgagccagtacttttagatagcagcagcattttggctgacagaattctactgatggatacctttttccaaattgtcatctatcttggtgagaccatagcccagtggcgtaaagctggctatcaagacatgcctgaatatgaaaatttcaagcatcttctgcaagctccattagatgatgcccaggatatcctgcaaactcgattcccaatgccaagatacatcaacactgaacacggaggtagtcaggctcggttcctcttgtccaaagtgaatccatctcagacacacaataatttgtatgcttggggacaggaatctggagctccaatattaacagatgatgttagtctgcaggtgttcatggatcatttgaaaaagttggcagtatcaagtgcctcctaaattgagaaagtagcagtgatgtttagaaagaagacaataactagactatgttcatagctactcaaaatatttactacattccttttactttattgcaagattttaaaatgtcatcatgtactccatacattcaaaagcagtttttttacatagttcttttgatttttaatttatttgtattctttttattttgttctatctttttcatgaaccgtaatgtagttcattgatgttttttggaacttatagatctttatcagctttctttaccctaacattataattgtatgaactatgggaaactttttgtagctccctcagatgaaggaataatggtcatgaagaaaataaactggaaggggagagtactagctnaaaaaaaaaaaaaaacaaaaaaaaaaaa

SEQ ID NO: 184 - protein, 194 aa, Macropus eugenii
DSFSLYPQFMFHLRRSPFLQVFNNSPDESSYYRHHFARQDLTQSLIMIQPILYSYSFHGPPEPVLLDSSSILADRILLMDTFFQIVIYLGETIAQWRKAGYQDMPEYENFKHLLQAPLDDAQDILQTRFPMPRYINTEHGGSQARFLLSKVNPSQTHNNLYAWGQESGAPILTDDVSLQVFMDHLKKLAVSSAS

SEQ ID NO: 185 - DNA, 456 bp, Bos taurus
atgtgtgtcagaaaacgagcttggtgtcggtggcacaagtcagtggaaaatctgtggcctgaaccccacaacgacgctcgcatctactttgaagtagtcaaccagcacaacgcctggatcccgcaaggaggccgaggctccatccagtttatcatgcactaccagcactccagcacccagcagcgcatcggtgtgaccaccgttgcccgcaatcgggcagatgtgcagagccagctgaagcacatcgaagtggatctcacccagtccctcatcatgattcagcccatcctgtattcctactccttccacgggcccccagagcctgtgctcctggacagcagcagcattcttggtgacagaattccgctcatgaacactttcttccaaattgtcatttatctttgtgagaccatagcccagtggcggaaagaaagctaccaggacgtgcccgagtag

SEQ ID NO: 186 - protein, 151 aa, Bos taurus
MCVRKRAWCRWHKSVENLWPEPHNDARIYFEVVNQHNAWIPQGGRGSIQFIMHYQHSSTQQRIGVTTVARNRADVQSQLKHIEVDLTQSLIMIQPILYSYSFHGPPEPVLLDSSSILGDRIPLMNTFFQIVIYLCETIAQWRKESYQDVPE

SEQ ID NO: 187 - skipped

SEQ ID NO: 188 - protein, 162 aa, Macropus eugenii
MRGLTMQVLLLVLGLLSLMTPLGYAQDQPYQDVLNRFIQEYNTKSESESLFRLSVLNLPSQESNDPTAPQLLKFTIRETVCSKSEHRNPEECDFKKNGLVEECIGTVDLDSSSPSVDISCDGPEKVKRGFGKKLRKRLKKFRNSIKKRLKNFNVVIPIPLPG

SEQ ID NO: 189 - skipped

SEQ ID NO: 190 - DNA, 960 bp, Bos taurus
  variation (954)...(960): n = g, a, t or c
ccaagaatggctgtatcaccatggaccacttcaaggtgatcaagacgcatcagatagaggatgagagagagaggcaggagaagaagctgtacctggggtacgcgctcctgggtgcccaccccatcctgaaccaaactgtcagccgcatgcagcacacagcagagggcaagaaggaggcgctcttcaccctctactctgctcatgacgtgactctggccccgattctcagtgccttgggccttacgggagccagattcccacggtttgcggccaggttggtctttgagctctggcaggacagggagaaaccccgggagcactccatccggatcctctatgacggtgttgacatcacgttctacacttcattctgccaggaccaccacaggcggtctcccaggcccatgtgtcccctcgaaaatttagtccgcttcatcaaaaaggacatgtttgtggccctgggtggtggtagtaccaattattatgaagcatgtcacagggaagggttctaaaaggtacacgatgcagcgctgagaatctatgccaattcaaaaggatgggaaggtccacttctagttctgttggctgcttaagttacaaggtggtttttaaaggccagaaatcatgcttggggcccatgtggatttggggttgaacagggagtatgttactgtaattgggtacgtgagtaacttgagacacagtggtcagttcacagcagaaaggaaggtactttattccagcgagactttacttgatgccagaataatatttcagtgcccaaagctgccatttcaaatgtgtattctttagatctgccttggtactgtgtgatggaatcagcaaacttcagccaaagctcctcttcatctgggacaacctcaccttgtccttgttcagtgaatggtttcctgaagtgattcatttaaaacgggtgggcaaactttttgcatnnnnnnn

SEQ ID NO: 191 - DNA, 600 bp, Bos taurus
  variation (592)...(600): n = g, a, t or c
agactgggcaccatggagaccccgagggccagcctctccctgggacggtggtcactgtggctgctgctgctgggactagcgctgccctcggccagcgcccaggccctcagctacagggaggccgtgcttcgtgctgtggatcagctcaatgagcaatcctcagaacctaacatttaccgtcttctcgagctggaccagcctcctcaggatgatgaagacccagacagcccgaagcgggtgagcttcagggtgaaggagaccgtgtgctccaggaccacccagcagccccccgagcagtgtgacttcaaggagaatgggctgctgaaacgctgtgaggggacagtcaccctggaccaggtcaggggtaacttcgacatcacctgtaataatcaccagagcatcaggattacaaagcagccatgggcaccaccacaggcagcccgattatgtcgcattgtagtgataagggtttgcagataatcagctcttgtagatttctgtcataagccagcaaagagtcctaagggttttcttactctgcctcaagctactggatctgaaaaataaattcttgtgacatgaaaaaaaaaannnnnnnnn

SEQ ID NO: 192 - DNA, 2580 bp, Bos taurus
  variation 2046: n = g, a, t or c
  variation (2299)...(2496): n = g, a, t or c
  variation (2571)...(2580): n = g, a, t or c
cacgcgtccgttcagaattaagtatatttaaagatacacaagacagcatgtttgctgattgacagttgtgactcattacagcaggctcgtggcattggggaggaatgtggtatcattggtggaattgctgtcctctgtgtgcacaatcatttcaacttggagaaagcaggacatgtggcatcatctttggaacaagacatgctgcttctcaaggagggttttattaaagttctggttggttcacagaagccatggagaaccgtgtaccagcttttgtgtactggaatccctgtagggttttctctgggtgaatgataaccctgccgtttcttcctctaagttttagttgtgtcctttgtaagagggtggttcttagtggttgttaactgtgtgctcttgtgtaattctgtctcagacctcaagggagctgaaggttgcgggagccatcggaccctgtgtgtctctaaatgtgaaaggaccgtgtgtgtcagaaaacgagcttggtgtcggtggcacgagccagtggaaaatctgtggcctggatcccacgacaacgctcggcatctactttgaagtagtcaaccagcacaatgccccgatcccgcaaggaggccgaggcgccatccagttcgtcacgcactaccagcactccagcacccagcggcgcatccgtgtgaccaccgttgcccgcaattgggcagacgtgcagagccagctgaagcacatcgaagcggcgtttgaccaggaggcggcggcggtgctgatggcacggctgggggtgttccgagccgagacggaggaggggccggatgtgctccggtggctggaccgacagctcatccggctgtgtcagaagtttggacagtataacaaagaagaccccatgtcatttaggttatcagattccttctctctgtatcctcagttcatgttccatcttagaaggtctccattccttcaagtcttcaacaatagccctgacgagtcttcgtattatcggcaccactttgcccggcaggacctcacccagtccctcatcatgatccagcccatcctgtattcctactctttccacggccccccagagcccgtgctcctggacagcagcagcattcttgctgacagaattctgctcatggacaccttcttccaaattgtcatttatcttggtgagaccatagcccagtggcggaaggcaggctaccaggacatgcccgagtatgaaaacttcaagcatctcctgcaggcgcccctggacgatgcacaggagatcctgcaggcgcggttcccgatgccacggtacatccacacagagcacggtggcagccaggctcgatttcttttgtccaaagtgaatccgtctcagacacacaataacctgtatgcttggggacaggaaacgggagcgcccatcctcactgatgacgttagcctgcaggtgttcatggaccatctgaagaagctggctgtctccagtgcttgttgagttaaggatataggcaggaaatggaagaaaacactgtcattttgtgttcacaaatgtttataaaggcttaacattccttttactttgccaatggattttaataaataatcaaaggaagtgttagtaactatttagattttaatttatttatattccttatctgtgcccttttcttactctctgaaattttaaggtcataaacattttggtattggtagatgtttatatgctttttgtatcctaacttttagagtctgtataaaatcagagctaatgtattttggcaacttgcttacatgaaaatcaatgatcagaaaggaaataaatctggatggagaaagcattggttaaaatagtgctaatacatatttgatttctgaagtctctgtaagtcttgagtatgttttatgtaaattctccaacaagtaattcctgaacttaatcttcatttactgtccaaatattaacagataaggaaacgtaagaaataatgtgcagctgttgaggctgagtggtctggagtggcactgtccctccctcacctgagatacagagaacancattggtggttaccagtggaggagggatggggaagggtgaaatggattaaggggggagggggtctgttgtactgtgatggagggaaactagggattgtggtagtgaagtgactgcagtgcatacagaacttgaattataatgtacatgtgaaacatgttataaaccaacgttacctcaattataaaaattctagctattctacccagtggagttcattcttgttttgaggttatgtcccctttcacataggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnattgctacgacagctgtcatccaggaatttcttttgttggactcctctgttagtttccaggagtaataaggaccnnnnnnnnnn

SEQ ID NO: 193-206 - skipped (14 entries)

SEQ ID NO: 207 - protein, 108 aa, Macropus eugenii
MRGLTMQVLLLVLGLLSLMTPLGYAQDQPYQDVLNRFIQEYNTKSESESLFRLSVLNLPSQESNDPTAPQLLKFTIRETVCSKSEHRNPEECDFKKNGVRVSWAGKGF

SEQ ID NO: 208 - DNA, 591 bp, Bos taurus
agactgggcaccatggagaccccgagggccagcctctccctgggacggtggtcactgtggctgctgctgctgggactagcgctgccctcggccagcgcccaggccctcagctacagggaggccgtgcttcgtgctgtggatcagctcaatgagcaatcctcagaacctaacatttaccgtcttctcgagctggaccagcctcctcaggatgatgaagacccagacagcccgaagcgggtgagcttcagggtgaaggagaccgtgtgctccaggaccacccagcagccccccgagcagtgtgacttcaaggagaatgggctgctgaaacgctgtgaggggacagtcaccctggaccaggtcaggggtaacttcgacatcacctgtaataatcaccagagcatcaggattacaaagcagccatgggcaccaccacaggcagcccgattatgtcgcattgtagtgataagggtttgcagataatcagctcttgtagatttctgtcataagccagcaaagagtcctaagggttttcttactctgcctcaagctactggatctgaaaaataaattcttgtgacatgaaaaaaaaaa

SEQ ID NO: 209-214 - skipped (6 entries)

SEQ ID NO: 215 - protein, 161 aa, Bos taurus
MRDSTMQVLLLVLGLLSLMTSLACAQDQPYQDVLNRFIQEYNTKSESESLFRLSVLNLPPEESNDPAVPLLKFTIRETVCPKTEHRNADECDFKKNGLVKQCIGTIDLDSPNPSVDISCDRPAKVKRGLWESLKRKATKLGDDIRNTLRNFKIKFPVPRQG

SEQ ID NO: 216 - DNA, 1238 bp, Macropus eugenii
  variation 1152: n = g, a, t or c
acagtgctggggcaagagcgtccggacacgctgagcagctggagggcggccagtggccccaggtggggaacctgtccgtgaggtcctggggcctggcttccctgccatcccccctcctgcatcccctcctaagtccttggtcttgtcatagttcaggaccttccactgttggttcactgcctgccagcgtttgaagatgcggtagacagaagagccctcatggacaatgagaccaatgcagacaacgtccataaagccgtacatgccatagcaaatctggaagagcaggtcctgccgctgccacttggctgatgggctgagtgaagaccagatgagccaagagatgctggcgacaaggaaaagggagcccaggacgatggctgcaatctggaccttctcaatgactgtcagggagatggcctgccactgcagtggattcttggtgcttattgccaggacctggtacttgaaatagcagagttcacagctccaggatccccgctcactgatccatcggatgaggcaaggctggtgggtgcaccgaacagaaccatcacaccgacatgggctcaagagctcccccctgcatggctccgccggggccggctccgacgcccagataacggtccgaccccgccccacgcggcgcggcccccagcgccggaggaggcccaggatggacatccctcggtatgaaccagtggccgagatcggtgttggtgcctatgggacggtgtacaaggcccgtgacccacacagcggccacttcgtggcactcaagagcgtgaaagtcccgacgggaggaggaagtggagggggcctgcctgtcagcactgtccgggaggtggccctccttaggaggctggaggcctttgagcatcccaacgtggtccggctgatggatgtctgtgccactgcccgaactgaccgagagaccaaggtgaccctagtgtttgaacacgtggaccaagacctgaggacgtatctggacaaggcccctccaccagggctgcccttggagaccattaaggacctaatgagacagttcttaagaggcctagactttcttcatgccaactgtattgtccaccgtgacttgaagccagaaaacatacttgtgactagtagtggaacagtcaagctggctgactntggcttagccaggatctacagctgccagatggcactcacacctgtggtagtcaccctctggtacagggctcctgaggttctcctgc

SEQ ID NO: 217 - protein, 190 aa, Macropus eugenii
  SITE 26, 190: Xaa = any amino acid
MVLFGAPTSLASSDGSVSGDPGAVNXCYFKYQVLAISTKNPLQWQAISLTVIEKVQIAAIVLGSLFLVASISWLIWSSLSPSAKWQRQDLLFQICYGMYGFMDVVCIGLIVHEGSSVYRIFKRWQAVNQQWKVLNYDKTKDLGGDAGGGMAGKPGPRTSRTGSPPGATGRPPAAQRVRTLFPSTVAYTFX

SEQ ID NO: 218 - DNA, 1041 bp, Bos taurus
atgctcaagtctcggctccgcatgttcctgaacgagctgaagctgctggtgctgacgggcggggggcggccccgggccgagccgcagccccgggggggcgggggaggcggctgcggctgggcgcccttcgccggctgctcggcccgggacggcgacggagacgaagaggagtactacgggtcagagccgagagcccggggcctggccggcgacaaggagccgcgggccggacccccgccgccgcacgcgccgccgccgccgcccccgggcgctctggacgccctgtcgctcagcagcagcctggacagcgggctccgaacccctcagtgccgaatctgcttccaaggccctgagcagggggagctcctgagcccctgccgctgcgacggctcagtgcgctgcactcaccagccctgcctcatccgctggatcagcgagaggggctcctggagctgtgagctctgctacttcaagtaccaggtcctggcaatcagcaccaagaaccccttgcagtggcaggccatctccctgacggtcatcgagaaggtccagattgcagccatagttctgggctcactcttcctcgtcgccagcatctcctggctcatttggtcctcacttagcccttcagccaagtggcaacggcaagatctgctctttcagatctgctatggcatgtacggcttcatggatgtcgtctgcataggcctcatcgtccatgaaggctcctccgtctaccgtatcttcaagcgctggcaggcagtaaaccagcagtggaaggtcctgaattatgacaagaccaaggacataggaggagatgcagggggagggacggcagggaagccgggccccaggacctcacggacgggcccccctactggggccaccagccgccccccggctgcccagcgcatgcggacgctcttgcctcagcgctgtggctacacaatcctgcatctccttggacagctgcggccaccagatgcccgttccagttccaattctggccgtgaggttgtcatgagggtcaccacggtctga

SEQ ID NO: 219 - protein, 346 aa, Bos taurus
MLKSRLRMFLNELKLLVLTGGGRPRAEPQPRGGGGGGCGWAPFAGCSARDGDGDEEEYYGSEPRARGLAGDKEPRAGPPPPHAPPPPPPGALDALSLSSSLDSGLRTPQCRICFQGPEQGELLSPCRCDGSVRCTHQPCLIRWISERGSWSCELCYFKYQVLAISTKNPLQWQAISLTVIEKVQIAAIVLGSLFLVASISWLIWSSLSPSAKWQRQDLLFQICYGMYGFMDVVCIGLIVHEGSSVYRIFKRWQAVNQQWKVLNYDKTKDIGGDAGGGTAGKPGPRTSRTGPPTGATSRPPAAQRMRTLLPQRCGYTILHLLGQLRPPDARSSSNSGREVVMRVTTV

SEQ ID NO: 220 - protein, 155 aa, Bos taurus
METPRASLSLGRWSLWLLLLGLALPSASAQALSYREAVLRAVDQLNEQSSEPNIYRLLELDQPPQDDEDPDSPKRVSFRVKETVCSRTTQQPPEQCDFKENGLLKRCEGTVTLDQVRGNFDITCNNHQSIRITKQPWAPPQAARLCRIVVIRVCR